(19) United States
(12) Patent Application Publication
(10) Pub. No.: US 2026/0265841 A1
(43) Pub. Date: Sep. 10, 2026

(54) METHODS AND KITS USEFUL FOR DETERMINING DISEASE LOCATION IN AN INDIVIDUAL SUFFERING FROM INFLAMMATORY BOWEL DISEASE

(71) Applicant: The Chinese University of Hong Kong, Hong Kong (CN)

(72) Inventors: Siew Chien NG, Hong Kong (CN); Ka Leung Francis CHAN, Hong Kong (CN); Jingwan ZHANG, Hong Kong (CN); Jiaying ZHENG, Hong Kong (CN); Wing Yan TANG, Hong Kong (CN)

(21) Appl. No.: 19/554,687

(22) Filed: Mar. 3, 2026

Related U.S. Application Data
(60) Provisional application No. 63/766,981, filed on Mar. 5, 2025.

(51) Int. Cl. C12Q 1/689; A61K 31/4402; A61K 31/4985; A61K 31/519; A61K 31/58; A61K 35/00; A61K 35/741; A61K 35/742; A61K 35/744; A61K 45/06; G16H 50/20

(52) U.S. Cl. CPC C12Q 1/689; A61K 31/4402; A61K 31/4985; A61K 31/519; A61K 31/58; A61K 35/741; A61K 35/742; A61K 35/744; A61K 45/06; G16H 50/20; A61K 2035/115; C12Q 2600/118; C12Q 2600/156

(57) ABSTRACT

In some embodiments, provided is a method for identifying a stool sample with an altered microbiome associated with IBD with disease above TI comprising the steps of: a) determining the relative abundance of a set of bacterial species in the stool sample; b) comparing the relative abundance of each bacterial species in the set of bacterial species from the individual with the relative abundance of each bacterial species in the set of bacterial species in a reference data set to generate a risk score; and c) determining the stool sample as having an altered microbiome associated with IBD with disease above the TI if the risk score is higher than a cutoff value. Other embodiments are described herein. In some embodiments, provided methods, kits, products and uses of compositions provide a cost-effective, non-invasive method to support clinical decision making, and hence to help improve disease management regarding disease above TI.

### CROSS-REFERENCE TO RELATED APPLICATION

This application claims benefit under 35 U.S.C. § 119 (e) of U.S. Provisional Application having Ser. No. 63/766,981 filed Mar. 5, 2025, the entire contents of which is/are hereby incorporated by reference herein.

### FIELD OF INVENTION

This application relates to methods and kits useful for determining disease location in an individual suffering from Inflammatory Bowel Disease (IBD).

### BACKGROUND OF INVENTION

Ulcerative colitis (UC) and Crohn's disease (CD) are subtypes of inflammatory bowel disease (IBD). UC primarily affects the colon, while CD can present in isolated areas, such as the ileum or colon, or in combined sites, known as ileocolonic disease. Due to the possible involvement of ileum, CD patients often require additional procedures, including computed tomography, ultrasonography, magnetic resonance imaging, and upper gastrointestinal endoscopy to locate and assess the affected areas.

Although pANCA (perinuclear anti-neutrophil cytoplasmic antibody) is more commonly observed in UC patients and ASCA (anti-Saccharomyces cerevisiae antibody) is more commonly observed in CD patients, their sensitivity and specificity to differentiate IBD subtypes are limited. These markers also cannot identify the location of disease involvement. Some studies have reported that host gene variants (e.g., NOD2 and HLA) are associated with IBD subphenotypes, including disease location. However, these genes often exhibit heterogeneity among different ethnic populations.

There is a need for effective and convenient methods to determine disease location in IBD patients.

### SUMMARY OF INVENTION

Disclosed herein are novel compositions, kits, methods and uses that are useful for determining the risk of, diagnosing, preventing, or treating disease above the terminal ileum (TI) in an individual suffering from Inflammatory Bowel Disease (IBD).

In some embodiments, provided is a method for determining the risk of, diagnosing, preventing, or treating disease above the terminal ileum (TI) in an individual suffering from Inflammatory Bowel Disease (IBD), including the steps of: (a) determining the relative abundance of a set of bacterial species in a stool sample from the individual, wherein the set of bacterial species includes three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis; (b) comparing the relative abundance of each bacterial species in the set of bacterial species from the individual with the relative abundance of each bacterial species in the set of bacterial species in a reference data set to generate a risk score; (c) determining the individual as having increased risk for or is suffering from disease above the TI if the risk score is higher than a cutoff value, or determining the individual as not having increased risk for or is not suffering from disease above the TI if the risk score is equal to or lower than the cutoff value; and (d) if the individual is determined to have an increased risk for or is suffering from disease above the TI, optionally treating the individual with an appropriate systemic therapy for disease above the TI, wherein the appropriate systemic therapy is selected from the group consisting of systemic corticosteroids, thiopurines, parenteral methotrexate, non-JAK inhibitor biologic agents, oral small molecule therapeutic agents for treating disease above the TI, and any combination thereof; or if the individual is determined to not have an increased risk for or is not suffering from disease above the TI, optionally treating the individual with an appropriate localized therapy, wherein the appropriate localized therapy is selected from the group consisting of sulfasalazine, enteric release budesonide, controlled ileal release (CIR) budesonide, oral methotrexate, an oral Janus kinase (JAK) inhibitor, or any combination thereof.

In some embodiments, provided is a kit for determining the risk of or diagnosing disease above terminal ileum (TI) in an individual suffering from Inflammatory Bowel Disease (IBD), including a reagent for detecting a set of bacterial species, wherein the set of bacterial species includes three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

In some embodiments, provided is a composition including Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

In some embodiments, provided is a method of preventing or treating disease above terminal ileum (TI) in an individual suffering from Inflammatory Bowel Disease (IBD), comprising the step of: administering the composition according to any one of the embodiments as described herein to the individual.

In some embodiments, provided is a method for identifying a stool sample with an altered microbiome associated with IBD with disease above terminal ileum (TI) including the steps of: (a) determining the relative abundance of a set of bacterial species in the stool sample, wherein the set of bacterial species includes three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis; (b) comparing the relative abundance of each bacterial species in the set of bacterial species from the individual with the relative abundance of each bacterial species in the set of bacterial species in a reference data set to generate a risk score; and (c) determining the stool sample as having an altered microbiome associated with IBD with disease above the TI if the risk score is higher than a cutoff value.

In some embodiments, provided is a computer program product for determining the risk, diagnosing, or suggesting treatment for disease above terminal ileum (TI) in an individual suffering from Inflammatory Bowel Disease (IBD), wherein the computer program product includes a computer readable medium encoded with computer executable code, wherein the computer executable code is configured to enable the execution of the steps of: (a) determining the relative abundance of a set of bacterial species in a stool sample from the individual, wherein the set of bacterial species includes three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis; (b) comparing the relative abundance of each bacterial species in the set of bacterial species from the individual with the relative abundance of each bacterial species in the set of bacterial species in a reference data set to generate a risk score; (c) determining the individual as having increased risk for or is suffering from disease above the TI if the risk score is higher than a cutoff value, or determining the individual as not having increased risk for or is not suffering from disease above the TI if the risk score is equal to or lower than the cutoff value; and (d) if the individual is determined to have an increased risk for or is suffering from disease above the TI, suggesting an appropriate systemic therapy for disease above the TI, wherein the appropriate systemic therapy is selected from the group consisting of systemic corticosteroids, thiopurines, parenteral methotrexate, non-JAK inhibitor biologic agents, oral small molecule therapeutic agents for treating disease above the TI, and any combination thereof, or if the individual is determined to not have an increased risk for or is not suffering from disease above the TI, suggesting an appropriate localized therapy, wherein the appropriate localized therapy is selected from the group consisting of sulfasalazine, enteric release budesonide, controlled ileal release (CIR) budesonide, oral methotrexate, an oral Janus kinase (JAK) inhibitor, or any combination thereof.

Other example embodiments will be described below.

### Advantages

There are many advantages of the invention. In certain embodiments, the present disclosure provides novel methods and kits for assessing risk and diagnosing whether there is disease involvement above terminal ileum in an individual suffering from Inflammatory Bowel Disease (IBD) by using the bacterial markers as described herein. By determining the relative abundance of these bacterial markers in fecal samples and generating a risk or diagnostics score, the disclosed techniques can provide a cost-effective, non-invasive method to support clinical decision making, and hence to help improve disease management.

For example, the choice of treatment differs according to disease location or disease phenotype. In some embodiments, when the subject is determined to have an increased risk of, or is suffering from, disease above the terminal ileum (TI), the method further comprises administering an appropriate systemic therapy selected to provide therapeutic bioavailability in disease above the TI. In such embodiments, localized therapies are avoided due to reduced bioavailability or limited efficacy in disease above the TI. In some embodiments, the appropriate systemic therapy is selected from the group consisting of systemic corticosteroids (e.g. prednisolone), thiopurines (e.g. azathioprine, 6-mercaptopurine), parenteral methotrexate, biologic agents other than JAK inhibitors (also referred to as “non-JAK inhibitor biologic agents”, (e.g. infliximab, adalimumab, vedolizumab, ustekinumab, risankizumab, mirikizumab, or guselkumab)), oral small molecule therapeutic agents capable of achieving systemic exposure sufficient to treat disease involvement above the TI (also referred to as “oral small molecule therapeutic agents for treating disease above the TI”), and any combination thereof.

In some embodiments, when the subject is determined to not have an increased risk of, and is not suffering from, disease above the TI, the method further comprises administering a localized therapy appropriate for disease confined to the colon, terminal ileum, or right colon. Such localized therapies are selected based on their known effectiveness in colonic or ileocolonic Crohn's disease and their limited systemic exposure.

In some embodiments, the localized therapy is selected from the group consisting of sulfasalazine, enteric release budesonide, and controlled ileal release (CIR) budesonide. In some embodiments, the localized therapy further comprises oral methotrexate or an oral Janus kinase (JAK) inhibitor, including upadacitinib, where oral administration provides adequate therapeutic exposure for disease not extending into the disease above the TI.

In some embodiments, the methods as described herein can be applied to determine whether there is disease above the terminal ileum in individuals with unknown IBD subtypes, diagnosed CD patients, and diagnosed ulcerative colitis (UC) patients who are suspected of actually having CD or those whose disease may have evolved from CD to UC.

In some embodiments, the present disclosure provides compositions comprising a set of underrepresented bacteria in disease above the TI that can be used to treat CD patients with disease above the TI. After an individual suffering from IBD is being determined to have increased risk for or is suffering from disease above the TI, the individual can undergo treatment through IMT or supplementation of the disclosed compositions in the form of probiotics/microbial consortium pills.

### BRIEF DESCRIPTION OF FIGURES

FIG. 1 shows principal coordinates analysis (PCoA) with Bray-Curtis dissimilarity comparison showing the diverse microbial composition in colonic IBD, ileocolonic CD, and ileal CD according to an example embodiment.

FIG. 2 is a stacked bar chart and boxplot showing the relative abundance of the six most abundant phyla in ileal CD, ileocolonic CD and colonic IBD according to an example embodiment.

FIG. 3 shows the importance generated by Boruta, association coefficients of bacterial abundance and prevalence generated by MaAsLin3 in the ileum and ileocolonic groups (relative to the colon group), prevalence and mean relative abundance in colonic, ileocolonic and ileal group of 32 selected bacterial markers according to an example embodiment.

FIG. 4A is a graph showing the overall performance of a random forest model with 15 bacterial markers for differentiating disease locations according to an example embodiment.

FIG. 4B is a graph showing the subgroup analysis of performance of the random forest model by centers according to an example embodiment.

FIG. 5 is a diagram showing the workflow of the test algorithm for diagnosing ileum involvement in an individual suffering from IBD according to an example embodiment.

FIG. 6A is a diagram showing microbial diversity (both shannon index and number of observed species) in CD patients with disease above terminal ileum (DA_TI) and CD patients with no disease above TI (NDA_TI).

FIG. 6B shows Principal coordinates analysis (PCoA) with Bray-Curtis dissimilarity comparison showing the diverse microbial composition in DA_TI and NDA_TI group according to an example embodiment.

FIG. 7 is a boxplot showing the relative abundance of the two phylum and seven genus that enriched in DA_TI group, and nine genus that depleted in DA_TI group according to an example embodiment.

FIG. 8 is a chart showing the importance generated by Boruta, association coefficients of bacterial abundance generated by MaAsLin2 in the DA_TI group (relative to NDA_TI group), prevalence in NDA_TI group and DA_TI group of 18 selected bacterial markers according to an example embodiment.

FIG. 9A is a diagram showing the overall performance of model with 18 bacterial markers for differentiating disease locations in the discovery cohort according to an example embodiment.

FIG. 9B is a diagram showing the overall performance of model with 18 bacterial markers for differentiating disease locations in the discovery cohort together with UC samples according to an example embodiment.

FIG. 9C is a diagram showing the overall performance of model with 18 bacterial markers for differentiating disease locations in the independent cohort according to an example embodiment.

FIG. 10 is a diagram showing the workflow of the test algorithm for diagnosing whether there is disease above TI or not in an individual suffering from IBD according to an example embodiment.

### DETAILED DESCRIPTION

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing,” is replaced with “consisting essentially of” or “consisting of”. These alternate embodiments that use “consisting of” or “consisting essentially of” are understood to be narrower embodiments of the “comprising”, “including,” or “containing,” embodiments.

For example, alternate embodiments of “a composition comprising A, B, and C” would be “a composition consisting of A, B, and C” and “a composition consisting essentially of A, B, and C.” Even if the latter two embodiments are not explicitly written out, this disclosure/application includes those embodiments. Furthermore, it shall be understood that the scopes of the three embodiments listed above are different.

For the sake of clarity, “comprising”, including, and “containing”, and any related forms are open-ended terms which allows for additional elements or features beyond the named essential elements, whereas “consisting of” is a closed end term that is limited to the elements recited in the claim and excludes any element, step, or ingredient not specified in the claim.

For the sake of clarity, “characterized by” or “characterized in” (together with their related forms as described above), does not limit or change the nature of whether the list of terms following it are open or closed. For example, in a claim directed towards “a composition comprising A, B, C, and characterized in D, E, and F”, the elements D, E, and F are still open-ended terms and the claim is meant to include other elements due to the use of the word “comprising” earlier in the claim.

“Consisting essentially of” limits the scope of a claim to the specified materials, components, or steps (“essential elements”) that do not materially affect the essential characteristic(s) of the claimed invention. In some embodiments, the essential characteristics are the basic and novel characteristic(s) of the claimed invention. For example, in some embodiments, the essential elements of a composition of the disclosure can be “Xmg to Ymg” of compound A. Even if the composition includes additional excipients, as long as the additional excipients do not materially affect the essential characteristics of the compound, e.g., in compound A's ability to bind to XX target or to treat YY disease, then such embodiment that “consists essentially of compound A” still includes compositions with the aforementioned additional excipients.

As used herein, the singular forms “a”, “an” and “the” are intended to include the plural forms as well, unless the context clearly indicates otherwise. Where a range is referred in the specification, the range is understood to include each discrete point within the range. For example, 1-7 means 1, 2, 3, 4, 5, 6, and 7.

As used herein and in the claims, an “effective amount”, is an amount that is effective to achieve at least a measurable amount of a desired effect. In some embodiments, the amount may be effective to increasing amino acid biosynthesis, carbohydrate degradation, cofactor, carrier, and/or vitamin biosynthesis in a cell.

As used herein, the term “composition” refers to a formulation containing one or more active ingredient(s). In some examples, a composition includes one or more bacterial species and optionally an acceptable carrier. In some examples, the composition is used as a medicament capable of treating a disease or condition in a subject in need. In some embodiments, the acceptable carrier is a pharmaceutical acceptable carrier. In some embodiments, the carrier is a dietary formulation, food, or beverage. In some examples, the composition is used as a nutritional supplement, dietary formulation, functional food, medical food, food, or beverage for use in maintaining general health in a subject. In some examples, the composition is in the form of probiotics/microbial consortium pills.

As used herein and in the claims, the term “prevent”, “preventing”, “preventive”, “preventative” or “prevention” refers the methods of reducing the risk of the onset, relapse or spread of a disease or disorder or one or more of their symptoms.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “terminal ileum” (“TI”) refers to the distal-most 10 cm (or around 10 cm) segment of ileum immediately proximal to the ileocecal valve. As used herein, the term “disease above the terminal ileum (TI)” or “DA_TI” refers to disease involvement in any gastrointestinal tract segment proximal (oral) to the TI, excluding the TI. In contrast, when it is described that there is “no disease above the TI” or “NDA_TI” in the individual, it means that there is no disease involvement in any gastrointestinal tract segment proximal (oral) to the TI. A segment is defined as being involved with disease if there are any erosions/ulcers noted on endoscopy or capsule endoscopy or any increase in bowel wall thickness, presence of stricture, fistula or abscess based on clinical assessment such as esophagogastroduodenoscopy (EGD), capsule endoscopy, computed tomography enterography (CTE), magnetic resonance enterography (MRE), and/or intestinal ultrasound.

As used herein, the term “ileum involvement” refers to a condition where ileum is affected by a disease, inflammation, or a pathological process. This involvement could manifest as inflammation, ulcers, strictures, or other abnormalities specifically present in the ileum. For example, when referring to ileum involvement in an individual suffering from Inflammatory Bowel Disease (IBD), it means that the disease process of IBD affects the ileum of the individual. In some embodiments, ileum involvement refers to ileal disease (e.g. ileal CD), ileocolonic disease (e.g. ileocolonic CD), or both. In some embodiments, ileum involvement includes jejunum, and proximal ileal involvement.

As used herein, the term “sequencing” refers to a technology that includes obtaining sequence information from one or more nucleic acid molecules by determining the identity and/or order of at least some nucleotides within each nucleic acid molecule.

As used herein, the term “bacterial markers”, “bacterial species markers”, “bacterial biomarkers” or “bacterial species biomarkers” refers to pre-selected bacterial species and/or genetic sequences. For example, the relative abundance of deoxyribonucleic acid (DNA) fragments can be measured for each bacterial biomarkers in a fecal sample of each of the subjects.

As used herein, the term “relative abundance” when used in the context of describing the presence of a particular bacterial species in relation to all bacterial species present in the same sample (e.g. in a stool sample of an individual), refers to the relative amount of the bacterial species out of the amount of all bacterial species in that particular sample. For instance, the relative abundance of one particular bacterial species can be determined by comparing the quantity of DNA specific for this species (e.g., determined by quantitative polymerase chain reaction) in one given sample with the quantity of all bacterial DNA (e.g., determined by quantitative polymerase chain reaction (PCR) and/or sequencing based on the 16s rRNA sequence) in the same sample.

As used herein, the terms “amplification” or “amplify” as used herein includes methods for copying a target nucleic acid sequence, thereby increasing the number of copies of the target nucleic acid sequence. Amplification may be exponential or linear, for example, by polymerase chain reaction (PCR), real-time PCR, digital droplet PCR, or other nucleic-acid amplification methods known in the art. The sequences amplified in this manner form an “amplification product.”

As used herein, the term “set of primers” refers to a pair of primers that are both directed to a target nucleic acid sequence. In some embodiments, a set of primers that is directed to a particular target nucleic acid sequence contains a forward primer and a reverse primer, each of which hybridizes under a suitable condition to a different strand (sense or antisense) of the target nucleic acid sequence.

As used herein, the term “probe” refers to a nucleic acid molecule attached to at least one detectable label, wherein the nucleic acid molecule comprises a probe sequence capable of hybridizing to at least a portion of a target nucleic acid sequence. Example of detectable labels include, but are not limited to, radioactive isotopes, enzyme substrates, co-factors, ligands, chemiluminescent or fluorescent agents, haptens, and enzymes.

As used herein, the term “fluorophore-quencher probe” refers to a nucleic acid molecule labelled with a fluorophore and a quencher, wherein the nucleic acid molecule comprises a probe sequence capable of hybridizing to at least a portion of a target nucleic acid sequence. In some embodiments, the fluorophore is attached to the 5′-end of the nucleic acid molecule and the quencher is attached to the 3′-end of the nucleic acid molecule. During the PCR amplification process, if the fluorophore-quencher probe binds to the target nucleic acid sequence, the fluorophore is separated from the quencher, leading to the release of fluorescence that can be detected and quantified.

As used herein, the term “markers”, “bacterial markers”, “bacterial species markers”, “bacterial biomarkers” or “bacterial species biomarkers” refers to pre-selected bacterial species and/or genetic sequences to be used in the methods/kits as described herein. For example, the relative abundance of deoxyribonucleic acid (DNA) fragments can be measured for each bacterial biomarkers in a fecal sample of each of the subjects.

As used herein, the term “reference data set” refers to dataset generated from a cohort of subjects having a clear diagnosis of CD, UC, DA_TI, NDA_TI and/or IBD. It can be generated by measuring the relative abundance for each of the “bacterial markers” for each subject in the cohort. The diagnosis of subjects in the cohort can be determined by a standard method. For example, a diagnosis can be made by combination of clinical, biochemical, stool, endoscopic, cross-sectional imaging, and histological investigations according to international guidelines.

As used herein, the term “training data set” refers to a subset of the “reference data set”. It includes the known health classifications for the subjects and the feature vectors for the subjects, which can be used to train a machine learning model.

As used herein, the term “tuning” refers to the process of adjusting the parameters or hyperparameters of a machine learning algorithm or model to optimize its performance on a given task or dataset. In some examples, during the tuning process, different values or combinations of values for the parameters are explored and evaluated to find the settings that yield the best results. The goal is to fine-tune the model's performance by finding the optimal configuration that minimizes errors, maximizes accuracy, or achieves the desired outcome.

Although the description referred to particular embodiments, the disclosure should not be construed as limited to the embodiments set forth herein.

In some embodiments, provided is a method for determining the risk of, diagnosing, preventing, or treating disease above the terminal ileum (TI) in an individual suffering from Inflammatory Bowel Disease (IBD), including the steps of: (a) determining the relative abundance of a set of bacterial species in a stool sample from the individual, wherein the set of bacterial species includes three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis; (b) comparing the relative abundance of each bacterial species in the set of bacterial species from the individual with the relative abundance of each bacterial species in the set of bacterial species in a reference data set to generate a risk score; (c) determining the individual as having increased risk for or is suffering from disease above the TI if the risk score is higher than a cutoff value, or determining the individual as not having increased risk for or is not suffering from disease above the TI if the risk score is equal to or lower than the cutoff value; and (d) if the individual is determined to have an increased risk for or is suffering from disease above the TI, optionally treating the individual with an appropriate systemic therapy for disease above the TI, wherein the appropriate systemic therapy is selected from the group consisting of systemic corticosteroids, thiopurines, parenteral methotrexate, non-JAK inhibitor biologic agents, oral small molecule therapeutic agents for treating disease above the TI, and any combination thereof, or if the individual is determined to not have an increased risk for or is not suffering from disease above the TI, optionally treating the individual with an appropriate localized therapy, wherein the appropriate localized therapy is selected from the group consisting of sulfasalazine, enteric release budesonide, controlled ileal release (CIR) budesonide, oral methotrexate, an oral Janus kinase (JAK) inhibitor, or any combination thereof.

In some embodiments, the set of bacterial species includes a group of three bacterial species selected from Table 3, a group of four bacterial species selected from Table 5, or a group of five bacterial species selected from Table 7.

In some embodiments, the reference data set is obtained by determining relative abundances of each bacterial species in the set of bacterial species in stool samples from a group of subjects including patients with Crohn's Disease (CD) in a reference cohort, wherein the patients with CD include patients with disease above the TI and patients without the disease above TI.

In some embodiments, the comparing step in step (b) is performed using a machine learning model to generate the risk score.

In some embodiments, the machine learning model is constructed by (i) obtaining the reference data set by determining relative abundances of each bacterial species in the set of bacterial species in stool samples from a group of subjects including patients with CD in a reference cohort, wherein the patients with CD include patients with disease above the TI and patients without disease above the TI; (ii) obtaining a validation data set by determining relative abundances of each bacterial species in the set of bacterial species in stool samples from a second group of subjects including patients with CD and patients with Ulcerative Colitis (UC) in a validation cohort, wherein the patients with CD include patients with disease above the TI and patients without disease above the TI; (iii) dividing the reference data set into a training data set and a test data set; (iv) training a candidate model with the training data set and evaluating the performance of the candidate model using the validation data set; (v) optionally tuning the candidate model by adjusting hyperparameters in the candidate model; (vi) repeating step (iv) and optionally step (v) to produce a set of candidate models, and selecting an optimal candidate model among the set of candidate models; (vii) evaluating the performance of the optimal candidate model using the test data set; (viii) optionally training a final model with the reference data set using a set of hyperparameters corresponding to the optimal candidate model to produce the final model; and (ix) using the optimal candidate model of step (vii) or if step (viii) is used to train the final model, using the final model of step (viii) as the machine learning model for the comparing step in step (b) to generate the risk score.

In some embodiments, the machine learning model is a random forest model, and wherein the risk score in step (b) is generated by the following steps: (b1) generating an ensemble of decision trees by the random forest model using the relative abundance of the set of bacterial species in the reference data set; and (b2) running the relative abundance of the set of bacterial species from the individual along the ensemble of decision trees to generate the risk score.

In some embodiments, the optimal candidate model is the candidate model with the highest performance among the set of candidate models.

In some embodiments, the performance of the candidate model is evaluated by Area Under the Curve (AUC) in Receiving Operating Characteristic (ROC) curve of the candidate model.

In some embodiments, the cutoff value is in the range of 0.3 to 0.6.

In some embodiments, the cutoff value is determined by the following steps: (c1) calculating a Youden's index using the risk score data in the reference data set; and (c2) determining the cutoff value based on the Youden's index.

In some embodiments, the set of bacterial species consists essentially of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

In some embodiments, the set of bacterial species includes Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

In some embodiments, the oral JAK inhibitor is upadacitinib.

In some embodiments, provided is a kit for determining the risk of or diagnosing disease above terminal ileum (TI) in an individual suffering from Inflammatory Bowel Disease (IBD), including a reagent for detecting a set of bacterial species, wherein the set of bacterial species includes three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

In some embodiments, the set of bacterial species includes a group of three bacterial species selected from Table 3, a group of four bacterial species selected from Table 5, or a group of five bacterial species selected from Table 7.

In some embodiments, the reagent includes one or more sets of primers and optionally one or more fluorophore-quencher probes, wherein each set of primers is used for amplifying a target nucleic acid sequence belonging to a bacterial species in the set of bacterial species via PCR to produce an amplification product, and each fluorophore-quencher probe is used for detecting the amplification product of each set of primers.

In some embodiments, the set of bacterial species consisting essentially of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

In some embodiments, the set of bacterial species includes Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

In some embodiments, provided is a composition including Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

In some embodiments, provided is a method of preventing or treating disease above terminal ileum (TI) in an individual suffering from Inflammatory Bowel Disease (IBD), including the step of: administering the composition of any one of the embodiments as described herein to the individual.

In some embodiments, provided is a method for identifying a stool sample with an altered microbiome associated with IBD with disease above terminal ileum (TI) including the steps of: (a) determining the relative abundance of a set of bacterial species in the stool sample, wherein the set of bacterial species includes three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis; (b) comparing the relative abundance of each bacterial species in the set of bacterial species from the individual with the relative abundance of each bacterial species in the set of bacterial species in a reference data set to generate a risk score; and (c) determining the stool sample as having an altered microbiome associated with IBD with disease above the TI if the risk score is higher than a cutoff value.

In some embodiments, the set of bacterial species includes a group of three bacterial species selected from Table 3, a group of four bacterial species selected from Table 5, or a group of five bacterial species selected from Table 7.

In some embodiments, the reference data set is obtained by determining relative abundances of each bacterial species in the set of bacterial species in stool samples from a group of subjects including patients with Crohn's Disease (CD) in a reference cohort, wherein the patients with CD include patients with disease above the TI and patients without disease above the TI.

In some embodiments, the comparing step in step (b) is performed using a machine learning model to generate the risk score.

In some embodiments, the machine learning model is constructed by (i) obtaining the reference data set by determining relative abundances of each bacterial species in the set of bacterial species in stool samples from a group of subjects including patients with CD in a reference cohort, wherein the patients with CD include patients with disease above the TI and patients without disease above the TI; (ii) obtaining a validation data set by determining relative abundances of each bacterial species in the set of bacterial species in stool samples from a second group of subjects including patients with CD and patients with Ulcerative Colitis (UC) in a validation cohort, wherein the patients with CD include patients with disease above the TI and patients without disease above the TI; (iii) dividing the reference data set into a training data set and a test data set; (iv) training a candidate model with the training data set and evaluating the performance of the candidate model using the validation data set; (v) optionally tuning the candidate model by adjusting hyperparameters in the candidate model; (vi) repeating step (iv) and optionally step (v) to produce a set of candidate models, and selecting an optimal candidate model among the set of candidate models; (vii) evaluating the performance of the optimal candidate model using the test data set; (viii) optionally training a final model with the reference data set using a set of hyperparameters corresponding to the optimal candidate model to produce the final model; and (ix) using the optimal candidate model in step (vii) or if step (viii) is used to train the final model, using the final model in step (viii) as the machine learning model for the comparing step in step (b) to generate the risk score.

In some embodiments, the machine learning model is a random forest model, and wherein the risk score in step (b) is generated by the following steps: (b1) generating an ensemble of decision trees by the random forest model using the relative abundance of the set of bacterial species in the reference data set; and (b2) running the relative abundance of the set of bacterial species from the individual along the ensemble of decision trees to generate the risk score.

In some embodiments, the optimal candidate model is the candidate model with the highest performance among the set of candidate models.

In some embodiments, the performance of the candidate model is evaluated by Area Under the Curve (AUC) in Receiving Operating Characteristic (ROC) curve of the candidate model.

In some embodiments, the cutoff value is in the range of 0.3 to 0.6.

In some embodiments, the cutoff value is determined by the following steps: (c1) calculating a Youden's index using the risk score data in the reference data set; and (c2) determining the cutoff value based on the Youden's index.

In some embodiments, the set of bacterial species consists essentially of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

In some embodiments, the set of bacterial species includes Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

In some embodiments, the relative abundance of the set of bacterial species is determined by metagenomic sequencing, quantitative PCR (qPCR) or droplet digital PCR (ddPCR).

In some embodiments, provided is a composition including Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

In some embodiments, provided is the use of the composition according to any one of the embodiments as described herein for the manufacture of a medicament for the treatment of disease above terminal ileum (TI) in an individual suffering from Inflammatory Bowel Disease (IBD).

In some embodiments, provided is a computer program product for determining the risk, diagnosing, or suggesting treatment for disease above terminal ileum (TI) in an individual suffering from Inflammatory Bowel Disease (IBD), wherein the computer program product includes a computer readable medium encoded with computer executable code, wherein the computer executable code is configured to enable the execution of the steps of: (a) determining the relative abundance of a set of bacterial species in a stool sample from the individual, wherein the set of bacterial species includes three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis; (b) comparing the relative abundance of each bacterial species in the set of bacterial species from the individual with the relative abundance of each bacterial species in the set of bacterial species in a reference data set to generate a risk score; (c) determining the individual as having increased risk for or is suffering from disease above the TI if the risk score is higher than a cutoff value, or determining the individual as not having increased risk for or is not suffering from disease above the TI if the risk score is equal to or lower than the cutoff value; and (d) if the individual is determined to have an increased risk for or is suffering from disease above the TI, suggesting an appropriate systemic therapy for disease above the TI, wherein the appropriate systemic therapy is selected from the group consisting of systemic corticosteroids, thiopurines, parenteral methotrexate, non-JAK inhibitor biologic agents, oral small molecule therapeutic agents for treating disease above the TI, and any combination thereof, or if the individual is determined to not have an increased risk for or is not suffering from disease above the TI, suggesting an appropriate localized therapy, wherein the appropriate localized therapy is selected from the group consisting of sulfasalazine, enteric release budesonide, controlled ileal release (CIR) budesonide, oral methotrexate, an oral Janus kinase (JAK) inhibitor, or any combination thereof.

In some embodiments, the set of bacterial species includes a group of three bacterial species selected from Table 3, a group of four bacterial species selected from Table 5, or a group of five bacterial species selected from Table 7.

In some embodiments, the reference data set is obtained by determining relative abundances of each bacterial species in the set of bacterial species in stool samples from a group of subjects including patients with Crohn's Disease (CD) in a reference cohort, wherein the patients with CD include patients with disease above the TI and patients without disease above the TI.

In some embodiments, the comparing step in step (b) is performed using a machine learning model to generate the risk score.

In some embodiments, the machine learning model is constructed by (i) obtaining the reference data set by determining relative abundances of each bacterial species in the set of bacterial species in stool samples from a group of subjects including patients with CD in a reference cohort, wherein the patients with CD include patients with disease above the TI and patients without disease above the TI; (ii) obtaining a validation data set by determining relative abundances of each bacterial species in the set of bacterial species in stool samples from a second group of subjects including patients with CD and patients with Ulcerative Colitis (UC) in a validation cohort, wherein the patients with CD include patients with disease above the TI and patients without disease above the TI; (iii) dividing the reference data set into a training data set and a test data set; (iv) training a candidate model with the training data set and evaluating the performance of the candidate model using the validation data set; (v) optionally tuning the candidate model by adjusting hyperparameters in the candidate model; (vi) repeating step (iv) and optionally step (v) to produce a set of candidate models, and selecting an optimal candidate model among the set of candidate models; (vii) evaluating the performance of the optimal candidate model using the test data set; (viii) optionally training a final model with the reference data set using a set of hyperparameters corresponding to the optimal candidate model to produce the final model; and (ix) using the optimal candidate model of step (vii) or if step (viii) is used to train the final model, using the final model of step (viii) as the machine learning model for the comparing step in step (b) to generate the risk score.

In some embodiments, the machine learning model is a random forest model, and wherein the risk score in step (b) is generated by the following steps: (b1) generating an ensemble of decision trees by the random forest model using the relative abundance of the set of bacterial species in the reference data set; and (b2) running the relative abundance of the set of bacterial species from the individual along the ensemble of decision trees to generate the risk score.

In some embodiments, the optimal candidate model is the candidate model with the highest performance among the set of candidate models.

In some embodiments, the performance of the candidate model is evaluated by Area Under the Curve (AUC) in Receiving Operating Characteristic (ROC) curve of the candidate model.

In some embodiments, the cutoff value is in the range of 0.3-0.6.

In some embodiments, the cutoff value is determined by the following steps: (c1) calculating a Youden's index using the risk score data in the reference data set; and (c2) determining the cutoff value based on the Youden's index.

In some embodiments, the set of bacterial species consisting essentially of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

In some embodiments, the set of bacterial species includes Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

Examples 1~4 describe a study related to determining ileum involvement in subjects with IBD. Examples 5-8 describe a study related to determining whether there is disease involvement above terminal ileum (TI) or not in subjects with IBD.

### Example 1: Study Cohort for Ileum Involvement

In this embodiment, a transethnic, multi-center study was conducted to identify gut bacteria signatures and evaluate their potential as biomarkers for stratifying IBD subphenotypes.

A multi-ethnicity cohort was established by recruiting subjects from China (Kunming, Beijing, Guangzhou, Shanghai), Malaysia, Thailand, Taiwan, China, Hong Kong, China, Canada, Australia, Germany and United States. Patients with CD and UC were diagnosed according to standard criteria of endoscopy, radiology, and histology. Crohn's Disease Activity Index (CDAI≤150, inactive; >150, active) scores or Harvey-Bradshaw Index (HBI≤4, inactive; >4, active) for patients with CD and Mayo scores (≤2, inactive; >2, active) or Simple Clinical Colitis Activity Index (SCCAI≤2, inactive; >2, active) for patients with UC were collected. Information on the disease location of CD patients was collected according to Montreal classification, which categorizes locations as L1 (ileal), L2 (colonic), L3 (ileocolonic), and L4 (upper gastrointestinal). L4 is used as a modifier when upper gastrointestinal involvement occurs alongside L1, L2, or L3. Disease behavior was also assessed, including B1 (non-stricturing, non-penetrating), B2 (stricturing), B3 (penetrating), p (perianal). Patients were excluded if they had used antibiotics within the past month, had known current sepsis (excluding uncomplicated infections such as influenza), had known history of severe organ failure (including decompensated cirrhosis, malignant disease, kidney failure, epilepsy, active serious infection, acquired immunodeficiency syndrome), had major bowel surgery in the last 6 months (excluding colonoscopy/procedure related to perianal disease), presence of an ileostomy or stoma, or current pregnancy.

### Example 2: Methods

### Fecal DNA Extraction and Metagenomic Sequencing

Fecal microbial DNA was extracted from 200 mg stool sample using the DNeasy PowerSoil Pro kit (Qiagen) according to manufacturer instruction. After the quality control procedures by Qubit 2.0, agarose gel electrophoresis, and Agilent 2100, metagenomic libraries were prepared using Nextera DNA Flex Library Preparation kit (Illumina, San Diego, CA), pooled, and sequenced at Novogene (Beijing, China) using the Illumina NovaSeq6000 targeting ~6 Gb of sequence per sample with 150 base pair of paired-end reads.

### Sequencing Data Processing and Microbiome Analysis

Adapters were removed using trimmomatic-0.39 in paired-end mode. Sequencing reads were trimmed when the average quality per base drops below 20 with a 4-base wide sliding window. Sequencing reads shorter than 50 bases were discarded. Host contaminating reads were filtered using Kneaddata (version 0.10.0) with the human reference genome (hg37decv0.1). Samples of low read count after quality filtration (<1 million reads) were excluded from our analysis. Furthermore, microbiome profiling was performed using MetaPhlAn4 (version 4.0.6). Mapping reads to clade-specific markers gene and annotation of species pangenomes was done through Bowtie2 (version 2.4.2). Relative abundance table of taxa were acquired for further analysis.

### Biomarker Selection and Model Construction

Microbiome Multivariable Associations with Linear Models (MaAsLin3, version 0.99.3) were performed to determine the multivariable association between bacterial species and metadata. Boruta was used to select biomarkers for classification. Random Forest algorithm was used to build diagnostic models. Subjects with colonic IBD (UC and colonic CD) and ileal CD from the multi-ethnic cohort were used in model construction and validation. These subjects were then randomly divided into discovery and validation cohorts in a 7:3 ratio.

### Example 3: Results

### Microbiome Profiling

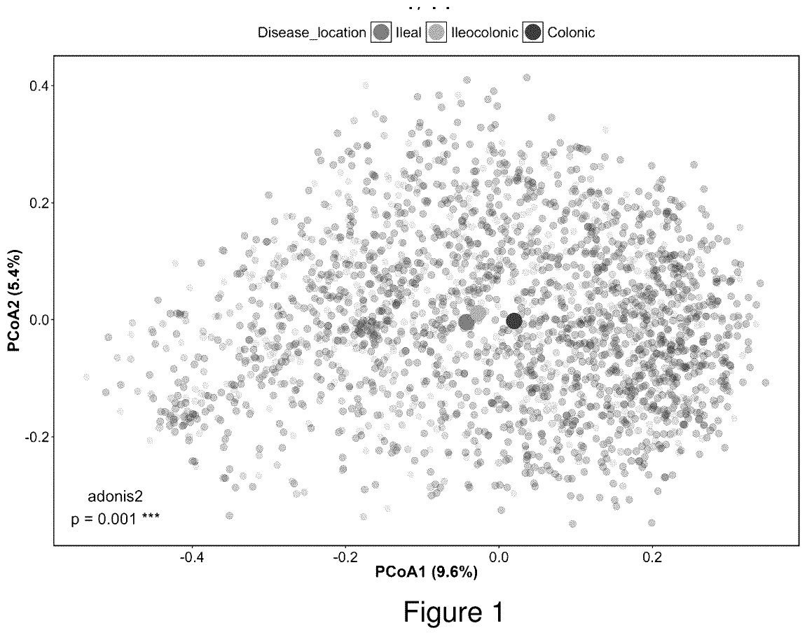

In the analysis, colonic CD and UC were combined as colonic IBD. Referring now to FIG. 1, which shows the principal coordinates analysis (PCoA) with Bray-Curtis dissimilarity comparison showing the diverse microbial composition in colonic IBD, ileocolonic CD, and ileal CD. As shown in FIG. 1, Gut microbiome composition of ileal and ileocolonic CD clustered away from that of colonic IBD (P<0.001).

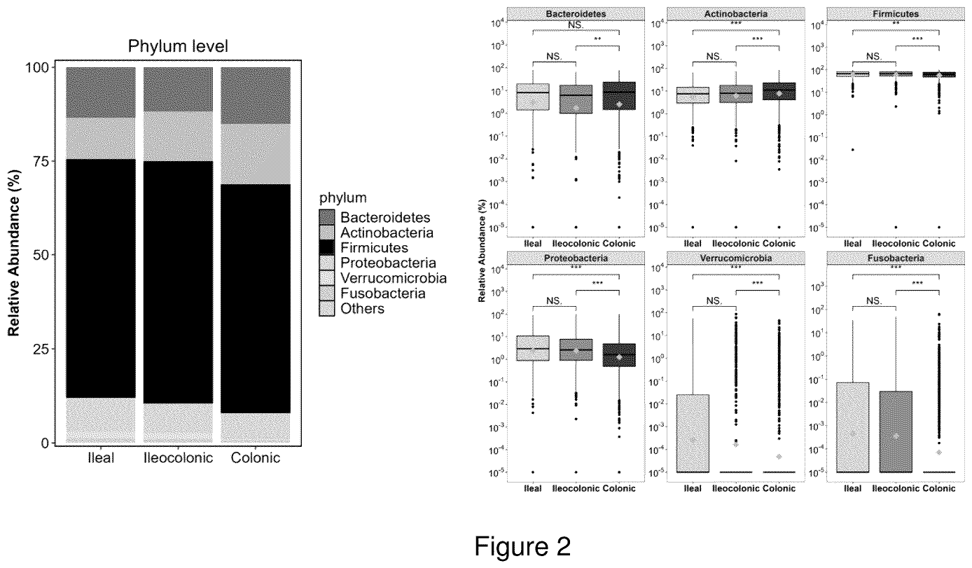

FIG. 2 is a stacked bar chart and boxplot showing the relative abundance of the six most abundant phyla in ileal CD, ileocolonic CD and colonic IBD. ‘Others’ includes phyla not shown in the figure. P values were calculated using two-sided Wilcoxon rank-sum test. *P<0.05, **P<0.01, ***P<0.001. “NS” means not significant.

As shown in FIG. 2, compared to colonic IBD, ileocolonic CD was characterised by decreased abundance of Actinobacteria and increased abundance of Firmicutes, Verrucomicrobia, Proteobacteria, and Fusobacteria, while ileocolonic CD showed no significant difference from ileal CD for all phyla.

Identification of Bacterial Species Associated with Ileum Involvement Using MaAsLin3

After adjusting for cohort region and read number, 162 bacterial species were found to be significantly associated with ileum involvement in ileal CD compared to colonic IBD, (adjusted P<0.2, Table A).

TABLE A
List of bacterial species that are associated with ileum involvement

| | | Coeffi- | Coeffi- |
|---|---|---|---|
| | | cients | cients |
| | | using | using |
| | | abun- | preva- |
| No | Bacterial species | dance | lence |
| 1 | Peptostreptococcus_anaerobius | −3.60 | −1.57 |
| 2 | Peptoniphilus_harei | −2.89 | −0.51 |
| 3 | Bacteroides_SGB14754 | −2.54 | −0.45 |
| 4 | Phocaeicola_coprocola | −1.97 | −0.56 |
| 5 | GGB47687_SGB2286 | −1.93 | −0.03 |
| 6 | Bifidobacterium_bifidum | −1.83 | −0.38 |
| 7 | Drancourtella_sp_An57 | −1.59 | −1.39 |
| 8 | Christensenellaceae_bacterium_NSJ_53 | −1.56 | −0.22 |
| 9 | GGB79973_SGB14341 | −1.50 | 0.07 |
| 10 | Lachnoclostridium_phocaeense | −1.27 | −0.92 |
| 11 | GGB12785_SGB19823 | −1.26 | 0.11 |
| 12 | Bifidobacterium_adolescentis | −1.26 | −0.38 |
| 13 | Parvimonas_micra | −1.24 | −0.04 |
| 14 | GGB9186_SGB14125 | −1.22 | 0.25 |
| 15 | Faecalibacterium_SGB15346 | −1.16 | −0.60 |
| 16 | Lachnoclostridium_sp_An138 | −1.15 | −0.71 |
| 17 | Faecalicoccus_pleomorphus | −1.10 | −0.78 |
| 18 | Anaerococcus_obesiensis | −1.10 | −1.03 |
| 19 | GGB9619_SGB15067 | −1.09 | −0.82 |
| 20 | GGB3817_SGB5182 | −1.08 | −0.97 |
| 21 | Clostridiales_bacterium_Choco116 | −1.06 | −1.05 |
| 22 | Actinomyces_johnsonii | −1.03 | 0.00 |
| 23 | Enorma_massiliensis | −1.00 | −0.83 |
| 24 | Eubacterium_sp_ER2 | −0.95 | −1.47 |
| 25 | Acidaminococcus_provencensis | −0.93 | 0.67 |
| 26 | Bacteroides_cellulosilyticus | −0.92 | 0.00 |
| 27 | GGB4456_SGB6141 | −0.85 | −1.10 |
| 28 | Candidatus_Schneewindia_gallinarum | −0.83 | 0.71 |
| 29 | Bifidobacterium_pullorum | −0.83 | −0.93 |
| 30 | Massilioclostridium_coli | −0.83 | −0.43 |
| 31 | Blautia_obeum | −0.80 | −0.54 |
| 32 | Bifidobacterium_longum | −0.78 | −0.52 |
| 33 | GGB3538_SGB4728 | −0.76 | −1.20 |
| 34 | Candidatus_Cibionibacter_quicibialis | −0.73 | −0.44 |
| 35 | Clostridium_SGB6179 | −0.73 | −0.48 |
| 36 | GGB3033_SGB4029 | −0.72 | −1.07 |
| 37 | Streptococcus_gordonii | −0.70 | −0.37 |
| 38 | Roseburia_faecis | −0.69 | −0.65 |
| 39 | Bacteroides_ovatus | −0.68 | 0.07 |
| 40 | GGB14025_SGB14906 | −0.67 | −0.66 |
| 41 | GGB3510_SGB4687 | −0.64 | −0.59 |
| 42 | Faecalibacterium_prausnitzii | −0.62 | −1.05 |
| 43 | Streptococcus_sanguinis | −0.61 | −0.27 |
| 44 | Blautia_hydrogenotrophica | −0.60 | −0.63 |
| 45 | Phocaeicola_vulgatus | −0.58 | 0.06 |
| 46 | Eubacterium_ventriosum | −0.56 | −0.34 |
| 47 | Desulfovibrio_SGB5077 | −0.54 | −0.50 |
| 48 | Lacrimispora_saccharolytica | −0.53 | −0.50 |
| 49 | GGB9614_SGB15049 | −0.52 | −0.45 |
| 50 | Turicibacter_sanguinis | −0.51 | −0.46 |
| 51 | Catenibacillus_scindens | −0.51 | −0.46 |
| 52 | Coprococcus_SGB4669 | −0.50 | −0.55 |
| 53 | GGB6020_SGB14985 | −0.49 | −0.98 |
| 54 | Romboutsia_timonensis | −0.48 | −1.40 |
| 55 | Lachnospira_eligens | −0.47 | −0.54 |
| 56 | GGB3032_SGB4027 | −0.46 | −0.82 |
| 57 | Candidatus_Pseudoruminococcus_{—} | −0.45 | −1.09 |
| | merdavium | | |
| 58 | Clostridiales_bacterium_KLE1615 | −0.42 | −0.37 |
| 59 | Blautia_glucerasea | −0.42 | −0.43 |
| 60 | GGB9453_SGB14844 | −0.40 | −0.77 |
| 61 | GGB3463_SGB4621 | −0.39 | −0.49 |
| 62 | Haemophilus_parainfluenzae | −0.36 | −0.51 |
| 63 | Oscillibacter_sp_ER4 | −0.34 | −0.38 |
| 64 | Alistipes_finegoldii | −0.33 | 0.40 |
| 65 | Anaeromassilibacillus_sp_An250 | −0.27 | −0.47 |
| 66 | GGB9621_SGB15073 | −0.25 | −0.99 |
| 67 | GGB34900_SGB14891 | −0.25 | −0.77 |
| 68 | GGB3746_SGB5089 | −0.24 | −0.42 |
| 69 | Anaerobutyricum_hallii | −0.23 | −0.31 |
| 70 | Streptococcus_australis | −0.22 | −0.41 |
| 71 | Dorea_formicigenerans | −0.21 | −0.35 |
| 72 | GGB3256_SGB4303 | −0.20 | −0.49 |
| 73 | Mesosutterella_multiformis | −0.18 | −0.51 |
| 74 | Blautia_sp_MSK_20_85 | −0.17 | −0.58 |
| 75 | Streptococcus_parasanguinis | −0.15 | −0.34 |
| 76 | Clostridium_sp_AF27_2AA | −0.15 | −0.67 |
| 77 | Megasphaera_micronuciformis | −0.11 | 0.61 |
| 78 | Flavonifractor_plautii | −0.10 | −0.44 |
| 79 | Lachnospiraceae_bacterium_Marseille_{—} | −0.10 | −0.34 |
| | Q4251 | | |
| 80 | Anaerofustis_stercorihominis | −0.10 | −0.34 |
| 81 | Blautia_luti | −0.09 | −0.53 |
| 82 | Streptococcus_infantis | −0.09 | −0.42 |
| 83 | Pseudoflavonifractor_gallinarum | −0.08 | −0.50 |
| 84 | Lachnoclostridium_sp_An131 | −0.08 | −0.63 |
| 85 | GGB9453_SGB14845 | −0.07 | −0.40 |
| 86 | GGB3534_SGB4722 | −0.07 | −1.25 |
| 87 | Anaerostipes_butyraticus | −0.02 | −0.90 |
| 88 | Dielma_fastidiosa | −0.02 | −0.66 |
| 89 | Harryflintia_acetispora | 0.00 | 0.42 |
| 90 | Mogibacterium_diversum | 0.00 | −0.30 |
| 91 | Blautia_faecis | 0.02 | −0.36 |
| 92 | Clostridium_saudiense | 0.04 | −0.36 |
| 93 | Eubacterium_rectale | 0.04 | −0.36 |
| 94 | Anaerostipes_hominis | 0.04 | 0.54 |
| 95 | Ruminococcus_bicirculans | 0.05 | −0.43 |
| 96 | Roseburia_inulinivorans | 0.13 | −0.49 |
| 97 | Dorea_sp_AF36_15AT | 0.15 | −0.39 |
| 98 | Merdimonas_faecis | 0.15 | −0.84 |
| 99 | Clostridium_spiroforme | 0.16 | −0.80 |
| 100 | Clostridia_bacterium_UC5_1_1D1 | 0.16 | −0.32 |
| 101 | Anaerostipes_hadrus | 0.17 | −0.46 |
| 102 | Veillonella_rogosae | 0.18 | −0.42 |
| 103 | Anaerostipes_caccae | 0.19 | 0.37 |
| 104 | Negativibacillus_massiliensis | 0.19 | −0.56 |
| 105 | Anaerobutyricum_soehngenii | 0.19 | 0.34 |
| 106 | Coprococcus_catus | 0.21 | −0.36 |
| 107 | Intestinibacillus_sp_Marseille_P6563 | 0.24 | −0.61 |
| 108 | Amedibacterium_intestinale | 0.27 | −0.58 |
| 109 | Dysosmobacter_welbionis | 0.29 | −0.61 |
| 110 | Holdemanella_porci | 0.31 | −0.88 |
| 111 | GGB38744_SGB14842 | 0.35 | −0.50 |
| 112 | Bilophila_wadsworthia | 0.35 | 0.32 |
| 113 | Faecalibacillus_intestinalis | 0.35 | −0.48 |
| 114 | Enterococcus_SGB6173 | 0.35 | −0.78 |
| 115 | Lacrimispora_celerecrescens | 0.38 | −0.60 |
| 116 | Escherichia_marmotae | 0.39 | 0.82 |
| 117 | Fusobacterium_varium | 0.39 | 0.82 |
| 118 | Phascolarctobacterium_faecium | 0.40 | 0.66 |
| 119 | GGB3537_SGB4727 | 0.41 | 0.52 |
| 120 | Desulfovibrio_SGB5076 | 0.41 | −0.98 |
| 121 | Klebsiella_pneumoniae | 0.41 | 0.60 |
| 122 | Ruminococcus_lactaris | 0.41 | −0.37 |
| 123 | Pseudoflavonifractor_capillosus | 0.41 | −0.79 |
| 124 | GGB3444_SGB4594 | 0.42 | −0.45 |
| 125 | Sanguibacter_SGB15121 | 0.43 | −0.51 |
| 126 | Anaerotruncus_colihominis | 0.46 | 0.38 |
| 127 | Evtepia_gabavorous | 0.48 | 0.53 |
| 128 | Ruthenibacterium_lactatiformans | 0.51 | −0.07 |
| 129 | Roseburia_intestinalis | 0.54 | −0.40 |
| 130 | Enterocloster_citroniae | 0.55 | 0.44 |
| 131 | Turicibacter_bilis | 0.56 | −0.89 |
| 132 | Massilimicrobiota_sp_An134 | 0.58 | −0.68 |
| 133 | Lachnospiraceae_bacterium_NSJ_29 | 0.59 | 0.02 |
| 134 | Coprobacillus_cateniformis | 0.61 | 0.48 |
| 135 | Clostridium_sp_1001270J_160509_D11 | 0.63 | −0.45 |
| 136 | Pseudoflavonifractor_sp_An184 | 0.64 | −0.61 |
| 137 | Clostridium_fessum | 0.69 | 0.11 |
| 138 | Erysipelatoclostridium_ramosum | 0.72 | 0.59 |
| 139 | Faecalibacterium_SGB15315 | 0.73 | −0.63 |
| 140 | Enterocloster_aldenensis | 0.74 | 0.58 |
| 141 | Clostridium_SGB4750 | 0.78 | 0.31 |
| 142 | Fusobacterium_mortiferum | 0.79 | 0.86 |
| 143 | Mediterraneibacter_glycyrrhizinilyticus | 0.79 | −0.01 |
| 144 | Faecalibacillus_faecis | 0.82 | −0.62 |
| 145 | Veillonella_parvula | 0.93 | 0.13 |
| 146 | Dialister_hominis | 0.93 | −0.81 |
| 147 | Ruminococcus_torques | 0.98 | 0.04 |
| 148 | Enterocloster_bolteae | 1.02 | 0.50 |
| 149 | Clostridium_scindens | 1.04 | 0.06 |
| 150 | Blautia_caecimuris | 1.07 | −0.23 |
| 151 | Candidatus_Pseudobutyricicoccus_{—} | 1.24 | −0.44 |
| | lothianensis | | |
| 152 | Fusobacterium_nucleatum | 1.25 | 0.78 |
| 153 | Ruminococcus_gnavus | 1.30 | 0.15 |
| 154 | Akkermansia_muciniphila | 1.31 | 0.60 |
| 155 | Faecalimonas_umbilicata | 1.38 | 0.79 |
| 156 | Escherichia_coli | 1.42 | 1.31 |
| 157 | Enterobacter_hormaechei | 1.53 | 0.08 |
| 158 | Allisonella_histaminiformans | 1.55 | 0.03 |
| 159 | Enterocloster_clostridioformis | 1.60 | 0.80 |
| 160 | Phascolarctobacterium_SGB4573 | 1.66 | 0.13 |
| 161 | Fusobacterium_ulcerans | 1.83 | 0.77 |
| 162 | GGB9480_SGB14874 | 1.97 | 0.05 |

### Select Bacterial Biomarkers for Location Classification Using Boruta

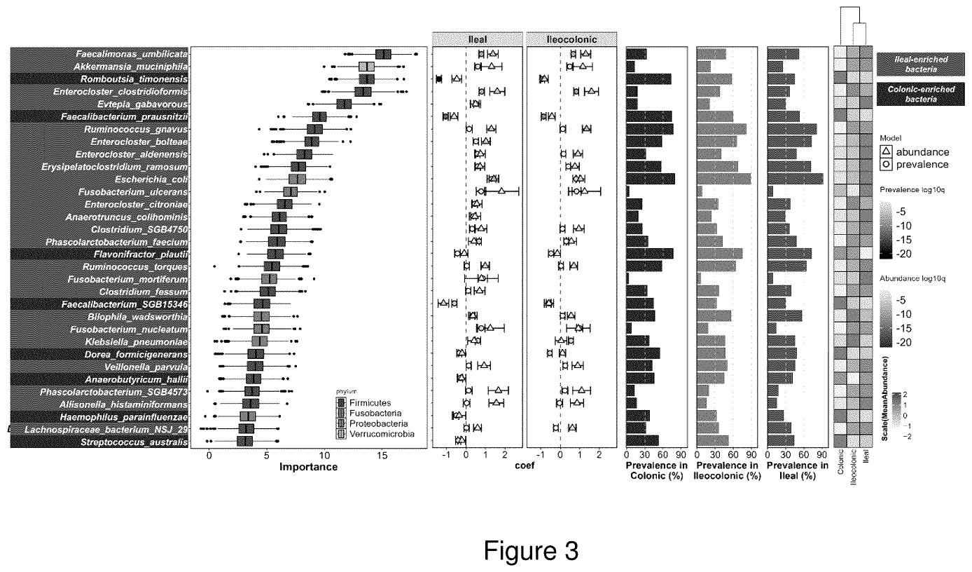

FIG. 3 shows the importance generated by Boruta, association coefficients of bacterial abundance and prevalence generated by MaAsLin3 in the ileum and ileocolonic groups (relative to the colon group), prevalence and mean relative abundance in colonic, ileocolonic and ileal group of 32 selected bacterial markers. As shown in FIG. 3, feature selection analysis (Boruta) identified 32 bacterial markers from the phyla Firmicutes, Fusobacteria, Proteobacteria, and Verrucomicrobia among which 24 were enriched and 8 were reduced in ileal CD compared to colonic IBD.

The 24 species enriched in ileal CD are Faecalimonas umbilicate, Akkermansia muciniphila, Enterocloster clostridioformis, Evtepia gabavorous, Ruminococcus gnavus, Enterocloster bolteae, Enterocloster aldenensis, Erysipelatoclostridium ramosum, Escherichia coli, Fusobacterium ulcerans, Enterocloster citroniae, Anaerotruncus colihominis, Clostridium SGB4750, Phascolarctobacterium faecium, Ruminococcus torques, Fusobacterium mortiferum, Clostridium fessum, Bilophila wadsworthia, Fusobacterium nucleatum, Klebsiella pneumoniae, Veillonella parvula, Phascolarctobacterium SGB4573, Allisonella histaminiformans, Lachnospiraceae bacterium NSJ-29.

The 8 species reduced in ileal CD are Romboutsia timonensis, Faecalibacterium prausnitzii, Flavonifractor plautii, Faecalibacterium SGB15346, Dorea formicigenerans, Anaerobutyricum hallii, Haemophilus parainfluenzae, Streptococcus australis.

### Random Forest Model for Location Classification

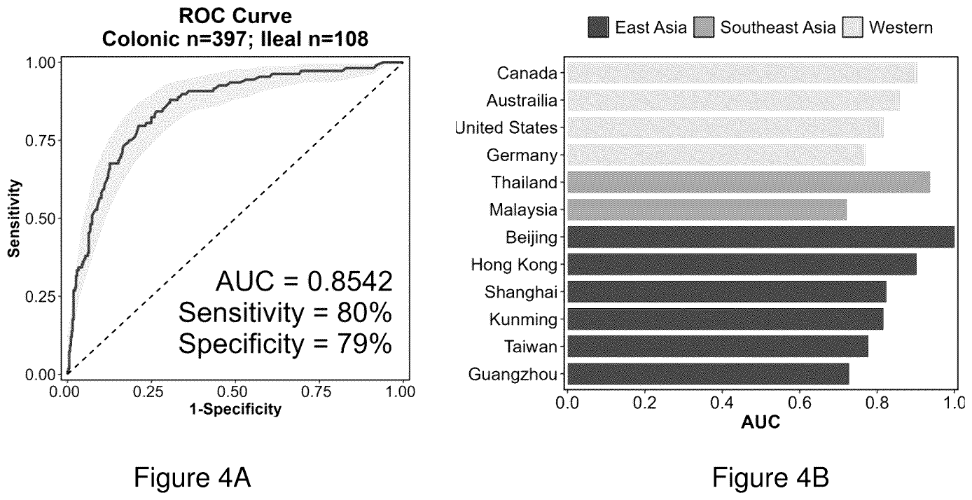

Referring now to FIG. 4A, which shows the overall performance of a random forest model based on 15 bacterial markers for differentiating disease locations. The random forest model based on 15 bacteria markers achieved an area under the curve (AUC) of 0.85 (Sensitivity: 80%, Specificity: 79%) in differentiating ileal CD from colonic IBD. Referring to FIG. 4B, subgroup analysis showed robustness of the model with AUCs ranging from 0.72-1.00 across 12 international cohorts, suggesting reproducible and universal role of these bacteria markers.

The 15 bacterial markers are:

12 species enriched in ileal CD: Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, Allisonella histaminiformans.

3 species reduced in ileal CD: Romboutsia timonensis, Faecalibacterium prausnitzii, Haemophilus parainfluenzae.

### Summary of Results

In this study, metagenomic sequencing was performed on 2,208 stool samples from patients with IBD (1,060 UC; 1,148 CD) from 12 international cohorts across North America, Europe, Asia and Australia. Amongst CD patients, 344, 464 and 340 had ileal (L1), colonic (L2) and ileocolonic (L3) disease, respectively. Colonic CD and UC were combined as colonic IBD. MaAsLin3 was used to assess bacterial associations with disease location, Boruta for biomarker selection, and Random Forest for model construction.

The results show that gut microbiome composition of ileal and ileocolonic CD clustered away from that of colonic IBD (P<0.001). Compared to colonic IBD, ileocolonic CD was characterised by decreased abundance of Actinobacteria and increased abundance of Firmicutes, Verrucomicrobia, Proteobacteria, and Fusobacteria, while ileocolonic CD showed no significant difference from ileal CD for all phyla. After adjusting for confounders, 162 bacterial species were significantly associated with disease location (adjusted P<0.2). Feature selection analysis identified 32 bacterial markers from the phyla Firmicutes, Fusobacteria, Proteobacteria, and Verrucomicrobia among which 24 were enriched in ileal disease and 8 were enriched in colonic disease (FIG. 3). A classification model based on 15 bacteria markers achieved an area under the curve (AUC) of 0.85 in differentiating ileal from colonic disease (FIG. 4A). Subgroup analysis showed robustness of the model with AUCs ranging from 0.72-1.00 across 12 international cohorts, suggesting reproducible and universal role of these bacteria markers (FIG. 4B).

This large-scale transethnic metagenomic analysis identified the universal location-specific bacteria signatures in IBD, supporting the role of gut microbiota in disease subphenotyping. This study provides novel approaches for disease stratification, diagnosis and future personalized intervention.

### Example 4: Workflow

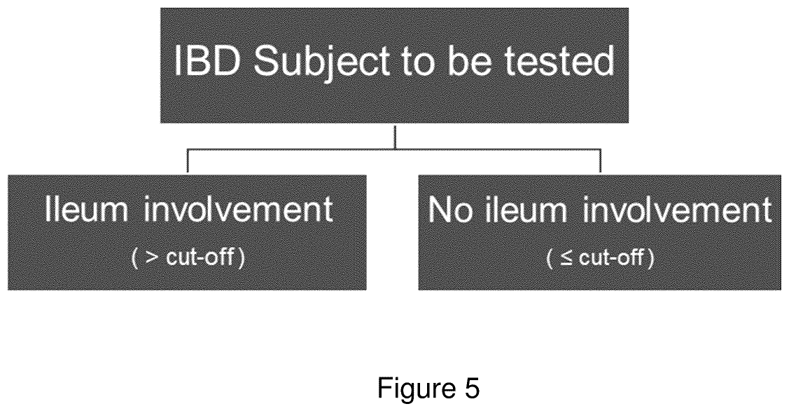

The workflow of the test algorithm for diagnosing ileum involvement in an individual suffering from IBD according to an example embodiment are described herein. Referring now to FIG. 5, using the relative abundance of 15 bacterial biomarkers (Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, Allisonella histaminiformans, Romboutsia timonensis, Faecalibacterium prausnitzii, Haemophilus parainfluenzae) determined by metagenomic sequencing as the inputs for the location classification model, the probability of ileum involvement will be generated for each IBD subject to be tested. Predetermined cut-off value is then applied to determine the IBD patients have ileum involvement or not. If the probability of ileum involvement is greater than the cut-off value (“cut-off”) (range: 0.1-0.3), the subject will be considered to have ileal involvement, otherwise, it will be considered to have no ileal involvement. In this embodiment, the cut-off value is 0.213.

In some embodiments, the test can be applied to individuals with unknown IBD subtypes, diagnosed CD patients, and diagnosed ulcerative colitis UC patients who are suspected of actually having CD or those whose disease may have evolved from CD to UC.

### Example 5: Study Cohort for Disease Above Terminal Ileum

In this study, patients with Crohn's Disease (CD) were recruited from Hong Kong, China (n=260), Taiwan, China (n=70), Thailand (n=79), the United States (n=54), and Canada (n=78). Patients with CD were diagnosed according to standard criteria of endoscopy, radiology, and histology. Patients with CD were systematically evaluated for involvement above terminal ileum (TI) or not using esophagogastroduodenoscopy (EGD), capsule endoscopy, computed tomography enterography (CTE), magnetic resonance enterography (MRE), and/or intestinal ultrasound.

Based on these definitions, all 541 patients with CD were classified into two groups: DA_TI (n=210) and NDA_TI (n=331). In addition, 641 patients with Ulcerative colitis (UC) were recruited from Hong Kong, China (n=340), Taiwan, China (n=74), Thailand (n=95), the United States (n=35), and Canada (n=97). An independent validation cohort of CD patients from Hong Kong, China was evaluated using the same criteria, in which 70 patients were classified as DA_TI and 79 patients as NDA_TI.

### Example 6: Methods

### Fecal DNA Extraction and Metagenomic Sequencing

Fecal microbial DNA was extracted from 200 mg stool sample using the DNeasy PowerSoil Pro kit (Qiagen) according to manufacturer instruction. After the quality control procedures by Qubit 2.0, agarose gel electrophoresis, and Agilent 2100, metagenomic libraries were prepared using Nextera DNA Flex Library Preparation kit (Illumina, San Diego, CA), pooled, and sequenced at Novogene (Beijing, China) using the Illumina NovaSeq6000 targeting ~6 Gb of sequence per sample with 150 base pair of paired-end reads.

### Sequencing Data Processing and Microbiome Analysis

Adapters were removed using trimmomatic-0.39 in paired-end mode. Sequencing reads were trimmed when the average quality per base drops below 20 with a 4-base wide sliding window. Sequencing reads shorter than 50 bases were discarded. Host contaminating reads were filtered using Kneaddata (version 0.10.0) with the human reference genome (hg37decv0.1). Samples of low read count after quality filtration (<1 million reads) were excluded from our analysis. Furthermore, microbiome profiling was performed using MetaPhlAn4 (version 4.0.6). Mapping reads to clade-specific markers gene and annotation of species pangenomes was done through Bowtie2 (version 2.4.2). Relative abundance table of taxa were acquired for further analysis.

### Biomarker Selection and Model Construction

Microbiome Multivariable Associations with Linear Models (MaAsLin2) were performed to determine the multivariable association between bacterial species and metadata. Boruta was used to select biomarkers for classification. Random Forest algorithm was used to build diagnostic models. The data of 541 patients with CD were then randomly divided into training set and test set in a 7:3 ratio. The data of UC patients and the independent cohort were used for evaluating the model performance.

### Example 7: Results

### Microbiome Profiling

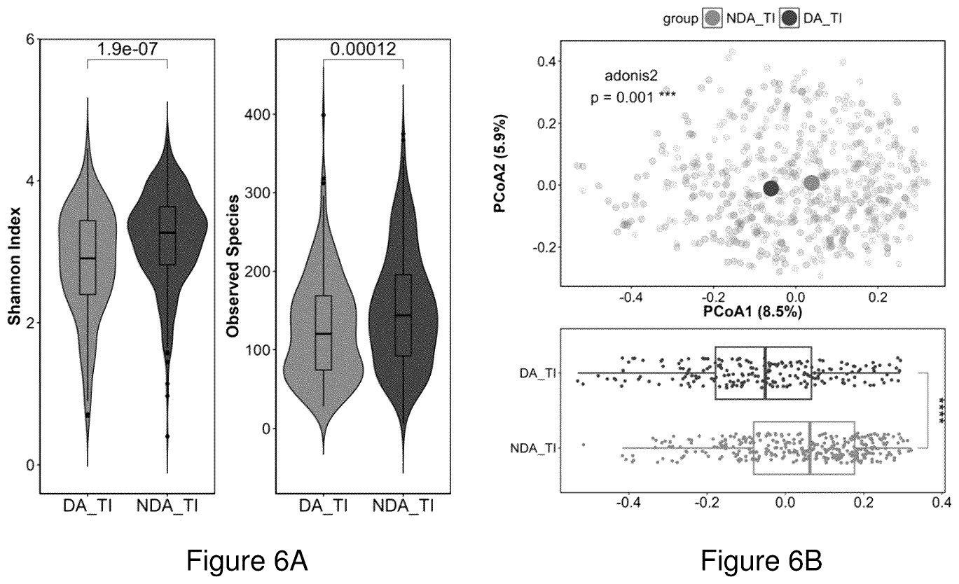

Referring now to FIG. 6A, the microbial diversity (both shannon index and number of observed species) of CD patients with disease above terminal ileum (DA_TI) are significantly lower than CD patients with no disease above TI (NDA_TI) (P<0.001). FIG. 6B shows the principal coordinates analysis (PCoA) with Bray-Curtis dissimilarity comparison showing the diverse microbial composition in DA_TI and NDA_TI group. As shown in FIG. 6B, gut microbiome composition of DA_TI clustered away from that of NDA_TI (P<0.001).

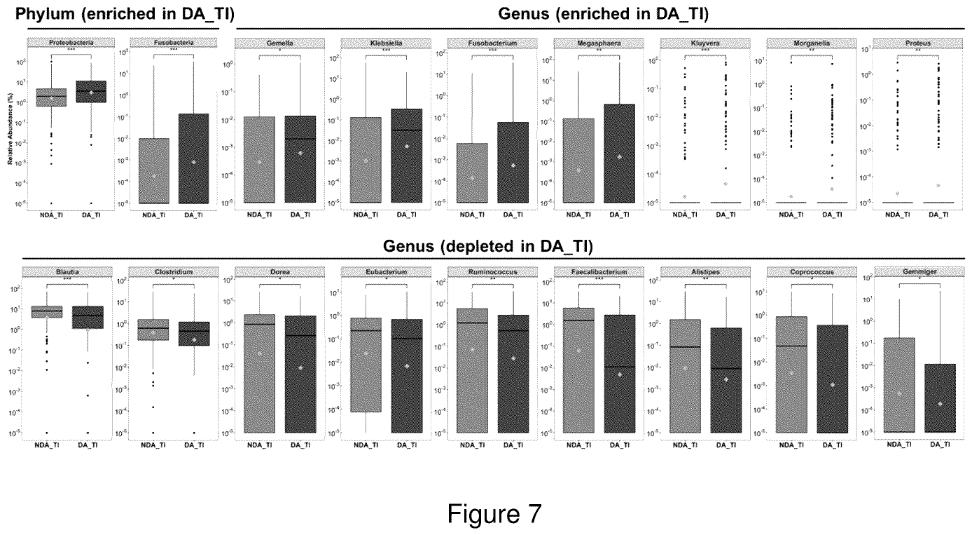

FIG. 7 is a boxplot showing the relative abundance of the two phylum and seven genus that enriched in DA_TI group, and nine genus that depleted in DA_TI group. P values were calculated using two-sided Wilcoxon rank-sum test. *P<0.05, **P<0.01, ***P<0.001.

As shown in FIG. 7, compared to NDA_TI group, DA_TI was characterised by increased abundance of Proteobacteria and Fusobacteria. At genus level, DA_TI was characterised by increased abundance of Gemella, Klebsiella, Fusobacterium, Megasphaera, Kluyvera, Morganella, and Proteus, while NDA_TI was characterised by increased abundance of Blautia, Clostridium, Dorea, Eubacterium, Ruminococcus, Faecalibacterium, Alistipes, Coprococcus, and Gemmiger.

Identification of Bacterial Species Associated with DA_TI Group Using MaAsLin2

Using MaAsLin2, 207 bacterial species were found to be significantly associated with the DA_TI group (adjusted P<0.25, Table 1). Among these, 175 were negatively correlated with the DA_TI group, while 32 were positively correlated with the DA_TI group.

TABLE 1
List of bacterial species that are associated with DA_TI group.

| Feature | Metadata | Coef |
|---|---|---|
| Romboutsia_timonensis | DA_TI_depleted | −0.870541382 |
| Eubacterium_rectale | DA_TI_depleted | −0.768285005 |
| Faecalibacterium_prausnitzii.t_{——}SGB15342 | DA_TI_depleted | −0.679293117 |
| Anaerostipes_hadrus | DA_TI_depleted | −0.667141969 |
| Faecalibacterium_prausnitzii.t_{——}SGB15316 | DA_TI_depleted | −0.655970659 |
| Anaerobutyricum_hallii | DA_TI_depleted | −0.646189934 |
| Clostridiaceae_bacterium.t_{——}SGB4269 | DA_TI_depleted | −0.597800871 |
| Lawsonibacter_asaccharolyticus | DA_TI_depleted | −0.561067701 |
| Dorea_sp_AF36_15AT | DA_TI_depleted | −0.539097151 |
| Dysosmobacter_welbionis | DA_TI_depleted | −0.532865082 |
| Roseburia_intestinalis | DA_TI_depleted | −0.500163809 |
| Blautia_obeum.t_{——}SGB4811 | DA_TI_depleted | −0.493251638 |
| Blautia_wexlerae | DA_TI_depleted | −0.491851825 |
| Eggerthella_lenta | DA_TI_depleted | −0.46118479 |
| Ruminococcus_bicirculans | DA_TI_depleted | −0.453186573 |
| Blautia_massiliensis | DA_TI_depleted | −0.446200504 |
| Clostridium_leptum | DA_TI_depleted | −0.443777011 |
| Dysosmobacter_sp_NSJ_60 | DA_TI_depleted | −0.442316896 |
| Candidatus_Cibionibacter_quicibialis | DA_TI_depleted | −0.441852033 |
| Roseburia_inulinivorans | DA_TI_depleted | −0.439504422 |
| Clostridia_bacterium_UC5_1_1D1 | DA_TI_depleted | −0.433281252 |
| Phocaeicola_dorei | DA_TI_depleted | −0.429502773 |
| Flavonifractor_plautii | DA_TI_depleted | −0.429048081 |
| Lachnospiraceae_bacterium_NSJ_29 | DA_TI_depleted | −0.417402729 |
| Ruminococcus_bromii | DA_TI_depleted | −0.413830796 |
| Alistipes_putredinis | DA_TI_depleted | −0.411637939 |
| Fusicatenibacter_saccharivorans | DA_TI_depleted | −0.402934282 |
| Lacrimispora_celerecrescens | DA_TI_depleted | −0.380125844 |
| Coprococcus_comes | DA_TI_depleted | −0.377773752 |
| Eubacterium_ventriosum | DA_TI_depleted | −0.371302979 |
| Ruminococcus_torques.t_{——}SGB4563 | DA_TI_depleted | −0.365931619 |
| Faecalibacterium prausnitzii.t_{——}SGB15318 | DA_TI_depleted | −0.361206068 |
| Lachnospira_eligens | DA_TI_depleted | −0.360839151 |
| Bacteroides_uniformis | DA_TI_depleted | −0.356741832 |
| Clostridium_sp_AF34_10BH | DA_TI_depleted | −0.353388907 |
| GGB45432_SGB63101 | DA_TI_depleted | −0.351470496 |
| Turicibacter_bilis | DA_TI_depleted | −0.350867154 |
| Clostridium_sp_AT4 | DA_TI_depleted | −0.344265325 |
| Streptococcus_gordonii | DA_TI_depleted | −0.341906005 |
| Clostridium_sp_AF36_4 | DA_TI_depleted | −0.339991441 |
| Streptococcus_thermophilus | DA_TI_depleted | −0.337005802 |
| Bifidobacterium_longum | DA_TI_depleted | −0.331245618 |
| Clostridium_butyricum | DA_TI_depleted | −0.325575109 |
| Hydrogenoanaerobacterium_saccharovorans | DA_TI_depleted | −0.324597242 |
| Blautia_caecimuris | DA_TI_depleted | −0.321625416 |
| Clostridiales_bacterium.t_{——}SGB15159 | DA_TI_depleted | −0.321456208 |
| GGB2653_SGB3574 | DA_TI_depleted | −0.319125839 |
| Mediterraneibacter_glycyrrhizinilyticus | DA_TI_depleted | −0.317698129 |
| Phocaeicola_vulgatus | DA_TI_depleted | −0.31415972 |
| GGB3653_SGB4964 | DA_TI_depleted | −0.312047962 |
| Bacteroides_caccae | DA_TI_depleted | −0.311402016 |
| Faecalibacterium_prausnitzii.t_{——}SGB15332 | DA_TI_depleted | −0.310507171 |
| Faecalibacterium_SGB15346 | DA_TI_depleted | −0.307895999 |
| Roseburia_hominis | DA_TI_depleted | −0.307010704 |
| Blautia_hydrogenotrophica | DA_TI_depleted | −0.305057019 |
| Bacteroides_ovatus | DA_TI_depleted | −0.303338851 |
| Alistipes_onderdonkii | DA_TI_depleted | −0.300881556 |
| Firmicutes_bacterium_AF16_15 | DA_TI_depleted | −0.300740308 |
| Parabacteroides_distasonis | DA_TI_depleted | −0.298487767 |
| Roseburia_faecis | DA_TI_depleted | −0.293944457 |
| Bacteroides_stercoris | DA_TI_depleted | −0.292855476 |
| Clostridium_spiroforme | DA_TI_depleted | −0.289776082 |
| Agathobaculum_butyriciproducens | DA_TI_depleted | −0.288377319 |
| Faecalibacillus_intestinalis | DA_TI_depleted | −0.287104279 |
| Clostridium_scindens.t_{——}SGB4630 | DA_TI_depleted | −0.287091228 |
| GGB52130_SGB14966 | DA_TI_depleted | −0.283788558 |
| Vescimonas_coprocola | DA_TI_depleted | −0.281775689 |
| Streptococcus_mutans | DA_TI_depleted | −0.280952017 |
| Sellimonas_intestinalis | DA_TI_depleted | −0.278933189 |
| Alistipes_finegoldii | DA_TI_depleted | −0.27881767 |
| Dorea_formicigenerans | DA_TI_depleted | −0.276337887 |
| Ruminococcus_torques.t_{——}SGB4608 | DA_TI_depleted | −0.276084657 |
| GGB9770_SGB15390 | DA_TI_depleted | −0.272132204 |
| Blautia_SGB4815 | DA_TI_depleted | −0.266984177 |
| Blautia_glucerasea.t_{——}SGB4816 | DA_TI_depleted | −0.266683422 |
| Mediterraneibacter_butyricigenes | DA_TI_depleted | −0.266311895 |
| Collinsella_SGB14861 | DA_TI_depleted | −0.265929268 |
| Alistipes_communis | DA_TI_depleted | −0.265533041 |
| Dorea_longicatena.t_{——}SGB4581 | DA_TI_depleted | −0.260755232 |
| GGB9453_SGB14844 | DA_TI_depleted | −0.258695361 |
| Clostridiales_bacterium_KLE1615 | DA_TI_depleted | −0.256670296 |
| GGB38744_SGB14842 | DA_TI_depleted | −0.253452096 |
| Blautia_faecis | DA_TI_depleted | −0.252512917 |
| GGB9719_SGB15272 | DA_TI_depleted | −0.24905567 |
| Monoglobus_pectinilyticus | DA_TI_depleted | −0.244709091 |
| Clostridiaceae_bacterium.t_{——}SGB4721 | DA_TI_depleted | −0.241256608 |
| Candidatus_Avimonas_narfia | DA_TI_depleted | −0.238340112 |
| Blautia_obeum.t_{——}SGB4810 | DA_TI_depleted | −0.237749283 |
| GGB2980_SGB3962 | DA_TI_depleted | −0.236050664 |
| Clostridiales_bacterium.t_{——}SGB15143 | DA_TI_depleted | −0.233952637 |
| Lacrimispora_amygdalina | DA_TI_depleted | −0.231707424 |
| GGB9501_SGB14898 | DA_TI_depleted | −0.231315964 |
| Eubacteriaceae_bacterium | DA_TI_depleted | −0.230370111 |
| Lactococcus_lactis | DA_TI_depleted | −0.230197653 |
| GGB9699_SGB15216 | DA_TI_depleted | −0.230186323 |
| GGB3444_SGB4594 | DA_TI_depleted | −0.229191902 |
| Enterococcus_SGB6173 | DA_TI_depleted | −0.228778273 |
| Coprococcus_catus | DA_TI_depleted | −0.228220064 |
| Phocaeicola_massiliensis | DA_TI_depleted | −0.226070176 |
| Anaerotruncus_colihominis | DA_TI_depleted | −0.221673844 |
| Christensenellaceae_bacterium_NSJ_63 | DA_TI_depleted | −0.221164484 |
| Eubacterium_ramulus | DA_TI_depleted | −0.220678484 |
| Clostridium_saudiense | DA_TI_depleted | −0.220548184 |
| Massiliimalia_timonensis | DA_TI_depleted | −0.219060579 |
| Lachnotalea_sp_AF33_28 | DA_TI_depleted | −0.218992783 |
| Clostridium_sp_Marseille_P3244 | DA_TI_depleted | −0.21871857 |
| Candidatus_Pararuminococcus_gallinarum | DA_TI_depleted | −0.218651898 |
| Dorea_longicatena.t_{——}SGB4582 | DA_TI_depleted | −0.217770157 |
| Adlercreutzia_equolifaciens | DA_TI_depleted | −0.216744361 |
| Negativibacillus_massiliensis | DA_TI_depleted | −0.216672811 |
| Clostridium_sp_AM49_4BH | DA_TI_depleted | −0.21548419 |
| Dysosmobacter_sp_BX15 | DA_TI_depleted | −0.211027885 |
| Lachnospira_pectinoschiza | DA_TI_depleted | −0.209315098 |
| Anaeromassilibacillus_sp_An250 | DA_TI_depleted | −0.208103097 |
| Lachnospiraceae_bacterium.t_{——}SGB4782 | DA_TI_depleted | −0.207852312 |
| Massilioclostridium_coli | DA_TI_depleted | −0.207628617 |
| Clostridiaceae_bacterium_AF18_31LB | DA_TI_depleted | −0.203820079 |
| GGB51647_SGB4348 | DA_TI_depleted | −0.201730181 |
| Parabacteroides_merdae | DA_TI_depleted | −0.199403686 |
| GGB3256_SGB4303 | DA_TI_depleted | −0.198878977 |
| Gordonibacter_urolithinfaciens | DA_TI_depleted | −0.198463418 |
| Blautia_hansenii | DA_TI_depleted | −0.196983152 |
| Waltera_intestinalis | DA_TI_depleted | −0.195472906 |
| Lachnospiraceae_bacterium_Marseille_Q4251 | DA_TI_depleted | −0.195071923 |
| GGB4456_SGB6141 | DA_TI_depleted | −0.194124912 |
| GGB3005_SGB3996 | DA_TI_depleted | −0.193454151 |
| Lachnospiraceae_bacterium.t_{——}SGB4781 | DA_TI_depleted | −0.193097552 |
| Blautia_glucerasea.t_{——}SGB4804 | DA_TI_depleted | −0.191246369 |
| Pseudoflavonifractor_gallinarum | DA_TI_depleted | −0.191209909 |
| Catenibacillus_scindens | DA_TI_depleted | −0.184556713 |
| Alistipes_ihumii | DA_TI_depleted | −0.17832397 |
| Barnesiella_intestinihominis | DA_TI_depleted | −0.178073612 |
| Eubacterium_callanderi | DA_TI_depleted | −0.176778873 |
| Clostridiaceae_bacterium_Marseille_Q3526 | DA_TI_depleted | −0.173937402 |
| Blautia_producta | DA_TI_depleted | −0.173629762 |
| Amedibacillus_dolichus | DA_TI_depleted | −0.169832703 |
| Massilimicrobiota_sp_An134 | DA_TI_depleted | −0.16637799 |
| GGB42994_SGB4746 | DA_TI_depleted | −0.165661279 |
| Alistipes_shahii | DA_TI_depleted | −0.160867497 |
| Ruminococcus_sp_AF41_9 | DA_TI_depleted | −0.160847756 |
| Eisenbergiella_tayi | DA_TI_depleted | −0.159781999 |
| GGB2987_SGB3969 | DA_TI_depleted | −0.155846645 |
| Clostridium_sp_AF20_17LB | DA_TI_depleted | −0.154531752 |
| Isoptericola_variabilis | DA_TI_depleted | −0.151725255 |
| Bacteroides_intestinalis | DA_TI_depleted | −0.150371159 |
| GGB3819_SGB5184 | DA_TI_depleted | −0.148899867 |
| Merdimonas_faecis | DA_TI_depleted | −0.14449999 |
| GGB2982_SGB3964 | DA_TI_depleted | −0.143639881 |
| Hydrogeniiclostidium_mannosilyticum | DA_TI_depleted | −0.141894955 |
| GGB9760_SGB15373 | DA_TI_depleted | −0.140457842 |
| Blautia_luti | DA_TI_depleted | −0.139257313 |
| Turicibacter_sanguinis | DA_TI_depleted | −0.137140196 |
| GGB3463_SGB4621 | DA_TI_depleted | −0.136464641 |
| Blautia_stercoris | DA_TI_depleted | −0.135893636 |
| Dorea_sp_AF24_7LB | DA_TI_depleted | −0.135151097 |
| Butyricicoccus_sp_AM29_23AC | DA_TI_depleted | −0.135045609 |
| Granulicatella_adiacens | DA_TI_depleted | −0.134691451 |
| Rothia_mucilaginosa | DA_TI_depleted | −0.130484095 |
| Mogibacterium_sp_NSJ_24 | DA_TI_depleted | −0.125735412 |
| GGB10485_SGB49305 | DA_TI_depleted | −0.125400551 |
| Roseburia_sp_AF02_12 | DA_TI_depleted | −0.12537416 |
| Gemmiger_formicilis | DA_TI_depleted | −0.125087369 |
| GGB51959_SGB72479 | DA_TI_depleted | −0.121243895 |
| Dielma_fastidiosa | DA_TI_depleted | −0.12033248 |
| Enterocloster_lavalensis | DA_TI_depleted | −0.119738736 |
| Clostridium_sp_AM33_3 | DA_TI_depleted | −0.118185616 |
| GGB3510_SGB4687 | DA_TI_depleted | −0.116031673 |
| Actinomyces_SGB17154 | DA_TI_depleted | −0.113942004 |
| GGB3534_SGB4722 | DA_TI_depleted | −0.113409379 |
| Anaerofustis_stercorihominis | DA_TI_depleted | −0.10845207 |
| Candidatus_Allochristensenella_caecavium | DA_TI_depleted | −0.106960164 |
| GGB9534_SGB14937 | DA_TI_depleted | −0.104291097 |
| Intestinimonas_butyriciproducens | DA_TI_depleted | −0.099649585 |
| GGB3033_SGB4029 | DA_TI_depleted | −0.097003357 |
| Senegalimassilia_anaerobia | DA_TI_depleted | −0.079687052 |
| Streptococcus_mitis.t_{——}SGB8180_group | DA_TI_enriched | 0.082593865 |
| Streptococcus_australis | DA_TI_enriched | 0.090628246 |
| Streptococcus_constellatus | DA_TI_enriched | 0.091390362 |
| Actinomyces_dentalis | DA_TI_enriched | 0.094107283 |
| GGB12785_SGB19823 | DA_TI_enriched | 0.110636988 |
| Gemella_haemolysans | DA_TI_enriched | 0.122762176 |
| Alistipes_sp_AF17_16 | DA_TI_enriched | 0.125558905 |
| Acidaminococcus_provencensis | DA_TI_enriched | 0.137640965 |
| Allisonella_histaminiformans | DA_TI_enriched | 0.145029585 |
| Streptococcus_mitis.t_{——}SGB8168 | DA_TI_enriched | 0.158508421 |
| Clostridium_scindens.t_{——}SGB4631 | DA_TI_enriched | 0.16245169 |
| Fusobacterium_varium | DA_TI_enriched | 0.162587652 |
| TM7_phylum_sp_oral_taxon_352 | DA_TI_enriched | 0.169999791 |
| Gemella_sanguinis | DA_TI_enriched | 0.172416229 |
| Proteus_mirabilis | DA_TI_enriched | 0.1770225 |
| Actinomyces_sp_ICM47 | DA_TI_enriched | 0.205762244 |
| Klebsiella_quasipneumoniae | DA_TI_enriched | 0.227081951 |
| Fusobacterium_nucleatum | DA_TI_enriched | 0.236769709 |
| Enterococcus_faecalis | DA_TI_enriched | 0.238252456 |
| Bifidobacterium_breve | DA_TI_enriched | 0.249680492 |
| Morganella_morganii | DA_TI_enriched | 0.260103561 |
| Limosilactobacillus_mucosae | DA_TI_enriched | 0.276141502 |
| Megamonas_funiformis | DA_TI_enriched | 0.281328394 |
| Megasphaera_micronuciformis | DA_TI_enriched | 0.288149447 |
| Enterococcus_avium | DA_TI_enriched | 0.292733764 |
| Phascolarctobacterium_SGB4573 | DA_TI_enriched | 0.307136476 |
| Megasphaera_sp_NM10 | DA_TI_enriched | 0.321057918 |
| Olsenella_sp_AF16_14LB | DA_TI_enriched | 0.351360393 |
| Bacteroides_fragilis | DA_TI_enriched | 0.360321037 |
| Eggerthella_sinensis | DA_TI_enriched | 0.383575246 |
| Klebsiella_pneumoniae | DA_TI_enriched | 0.473019286 |
| Escherichia_coli | DA_TI_enriched | 0.754325436 |

### Select Bacterial Biomarkers for Location Classification Using Boruta

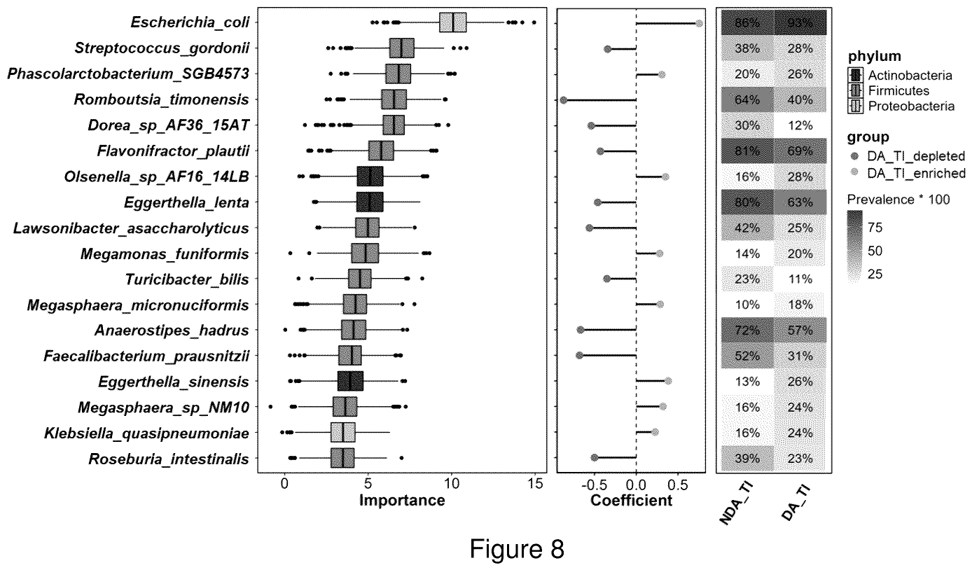

FIG. 8 shows the importance generated by Boruta, association coefficients of bacterial abundance generated by MaAsLin2 in the DA_TI group (relative to NDA_TI group), prevalence in NDA_TI group and DA_TI group of 18 selected bacterial markers. Feature selection analysis (Boruta) identified 18 bacterial markers from the phyla Actinobacteria, Firmicutes, and Proteobacteria among which 8 were enriched and 10 were reduced in DA_TI group compared to NDA_TI group.

The 8 species enriched in DA_TI group are Escherichia coli, Phascolarctobacterium SGB4573, Olsenella sp. AF16_14LB, Megamonas funiformis, Megasphaera micronuciformis, Eggerthella sinensis, Megasphaera sp. NM10, and Klebsiella quasipneumoniae.

The 10 species depleted in DA_TI group are Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

### Random Forest Model for Location Classification

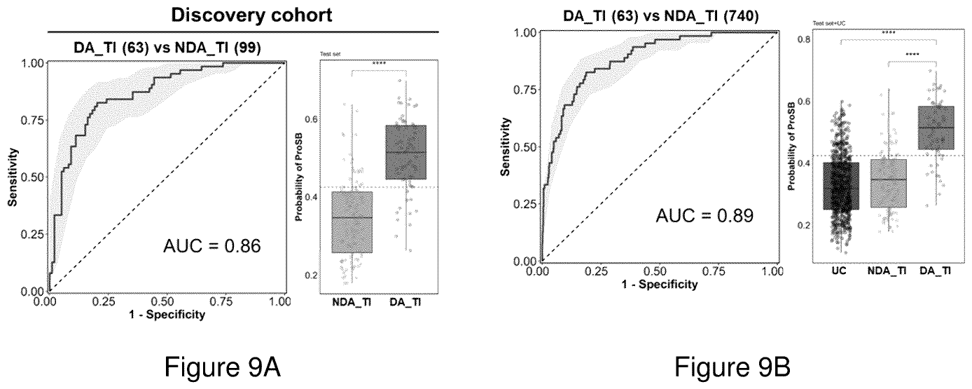

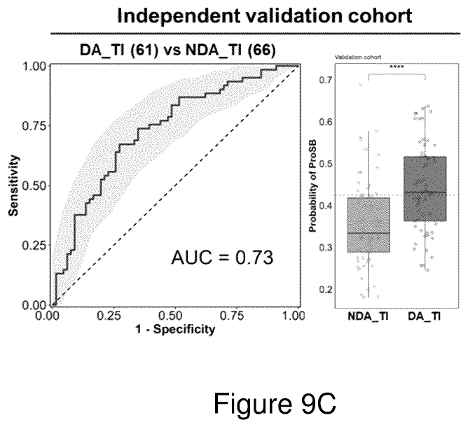

FIGS. 9A-C show the overall performance of the model with 18 bacterial markers for differentiating disease locations in the discovery cohort (FIG. 9A), in the discovery cohort together with UC samples (FIG. 9B), and in the independent cohort (FIG. 9C) respectively. Referring to FIG. 9A, a random forest model based on 18 bacteria markers achieved an area under the curve (AUC) of 0.87 (Sensitivity: 83%, Specificity: 80%) in differentiating DA_TI group from NDA_TI group. FIG. 9B shows that with 641 UC patients included in the NDA_TI group, the model could distinguish between the DA_TI group and the NDA_TI group (including UC patients) with an AUC of 0.89 (Sensitivity: 83%, Specificity: 81%). FIG. 9C shows that in the independent cohort, the model achieved an AUC of 0.73.

### Location Classification Using Single and Combined Bacterial Markers

In this embodiment, each bacterial marker was individually tested to evaluate its performance in location classification. Table 2 shows that among the single markers, the highest AUC was observed for Escherichia coli (AUC=0.65), followed by Romboutsia timonensis (AUC=0.63) and Faecalibacterium prausnitzii (AUC=0.61).

TABLE 2
Performance of single bacterial markers

| Bacterial marker | Abbreviations | AUC |
|---|---|---|
| Escherichia_{—}coli | EC | 0.65 |
| Romboutsia_{—}timonensis | RT | 0.63 |
| Faecalibacterium prausnitzii | FP | 0.61 |
| Lawsonibacter_{—}asaccharolyticus | LA | 0.60 |
| Anaerostipes_{—}hadrus | AH | 0.60 |
| Roseburia intestinalis | RI | 0.59 |
| Dorea_sp_AF36_15AT | DS | 0.59 |
| Eggerthella_{—}lenta | EL | 0.58 |
| Flavonifractor_{—}plautii | FPL | 0.58 |
| Eggerthella_{—}sinensis | ES | 0.56 |
| Streptococcus_{—}gordonii | SG | 0.56 |
| Olsenella_sp_AF16_14LB | OS | 0.56 |
| Turicibacter_{—}bilis | TB | 0.56 |
| Megasphaera_sp_NM10 | MS | 0.55 |
| Phascolarctobacterium_SGB4573 | PS | 0.54 |
| Megasphaera_{—}micronuciformis | MM | 0.54 |
| Klebsiella_{—}quasipneumoniae | KQ | 0.54 |
| Megamonas_{—}funiformis | MF | 0.54 |

The random combinations of 18 bacterial markers were used in the machine learning model to evaluate the performance for location classification. Referring to Table 3, when choosing 3 markers from the 18 markers, 28 out of 816 (3.43%) total possible combinations achieved AUCs>=0.75, and 372 out of 816 (45.59%) total possible combinations achieved AUCs>=0.7. For the sake of clarity, the bacteria species combinations shown in Table 3 are represented by abbreviations of the bacteria species as per Table 2. For example, “EC+MF+AH” refers to the combination of “Escherichia coli, Megamonas_funiformis, and Anaerostipes_hadrus”.

As shown in Table 4, among these 372 combinations, Escherichia coli appeared most frequently in 111 combinations, followed by Faecalibacterium prausnitzii which appeared 85 times, Dorea sp_AF36_15AT appeared 84 times, Turicibacter_bilis appeared 83 times, and Eggerthella sinensis appeared 81 times.

TABLE 3
List of combinations of 3 bacterial markers and the performance.

| Number | | | Best_{—} | | |
|---|---|---|---|---|---|
| of | | | Threshold_{—} | Sensitivity_{—} | Specificity_{—} |
| markers | Combinations | AUC | Range | Range | Range |
| 3 | EC + MF + AH; RT + DS + AH; EC + LA + KQ; | 0.7 | 0.296- | 31.75%- | 32.32%- |
| | DS + EL + TB; EC + FP + KQ; SG + AH + KQ; | | 0.5116 | 95.24% | 96.97% |
| | PS + AH + FP; RT + EL + TB; EC + LA + RI; | | | | |
| | SG + PS + EL; MM + AH + FP; PS + LA + AH; | | | | |
| | PS + DS + LA; EL + FP + ES; AH + ES + KQ; | | | | |
| | SG + RT + MS; SG + MM + ES; EC + MM + AH; | | | | |
| | PS + RT + RI; SG + FPL + FP; DS + TB + MM; | | | | |
| | DS + FP + KQ; FPL + MF + FP; LA + ES + MS; | | | | |
| | PS + LA + FP; EC + MS + KQ; RT + LA + MF; | | | | |
| | PS + TB + AH; DS + FPL + MS; FPL + LA + FP; | | | | |
| | FPL + TB + MS; EC + MF + RI; RT + EL + MS; | | | | |
| | SG + EL + RI; SG + FP + RI; DS + LA + KQ; | | | | |
| | DS + MM + ES; DS + MM + FP; FPL + LA + KQ; | | | | |
| | PS + TB + MM; LA + MM + AH; SG + PS + RT; | | | | |
| | SG + LA + MS; DS + FPL + AH; EC + FPL + AH; | | | | |
| | EC + FPL + MS; RT + ES + KQ; DS + MM + AH; | | | | |
| | EL + TB + KQ; PS + TB + RI; SG + MF + AH; | | | | |
| | PS + RT + EL; EL + FP + KQ; SG + FP + MS; | | | | |
| | RT + TB + RI; EC + RT + MF; LA + FP + RI; | | | | |
| | EL + TB + AH; PS + DS + TB; PS + FPL + FP; | | | | |
| | FPL + AH + ES; EC + EL + MM; EC + MM + RI; | | | | |
| | FPL + ES + RI; FP + MS + KQ; EC + LA + MF; | | | | |
| | EC + LA + MM; SG + FP + KQ; EL + MF + FP; | | | | |
| | EC + EL + LA; SG + AH + MS; RT + DS + TB; | | | | |
| | EC + OS + TB; TB + AH + KQ; MM + ES + RI; | | | | |
| | DS + MF + AH; EC + EL + AH; FPL + FP + RI; | | | | |
| | RT + TB + ES; DS + MF + MS; EC + DS + EL; | | | | |
| | EC + EL + KQ; EC + EL + MS; FPL + EL + MF | | | | |
| 3 | EC + FPL + KQ; EC + PS + FP; FPL + TB + RI; | 0.71 | 0.3064- | 31.75%- | 32.32%- |
| | EC + LA + FP; RT + FP + MS; EC + FPL + LA; | | 0.5059 | 95.24% | 96.97% |
| | FPL + LA + ES; PS + AH + ES; TB + MM + AH; | | | | |
| | MF + TB + ES; LA + MF + FP; LA + MM + MS; | | | | |
| | DS + FP + ES; EC + FP + RI; SG + DS + MS; | | | | |
| | SG + FPL + EL; RT + FP + KQ; RT + KQ + RI; | | | | |
| | PS + RT + ES; DS + MF + FP; MM + AH + ES; | | | | |
| | EC + PS + RT; LA + TB + KQ; RT + LA + AH; | | | | |
| | DS + AH + RI; SG + FPL + RI; LA + FP + KQ; | | | | |
| | SG + RT + ES; DS + LA + MS; FPL + EL + FP; | | | | |
| | EL + TB + RI; EC + RT + KQ; RT + MS + RI; | | | | |
| | EC + PS + KQ; DS + FPL + FP; DS + EL + ES; | | | | |
| | MF + TB + FP; SG + DS + ES; SG + RT + EL; | | | | |
| | PS + EL + RI; EC + PS + OS; SG + FPL + KQ; | | | | |
| | EC + SG + EL; SG + FPL + MF; SG + LA + MM; | | | | |
| | EC + AH + KQ; EC + RT + EL; DS + MS + RI; | | | | |
| | PS + FPL + TB; DS + FP + RI; EC + RT + MM; | | | | |
| | RT + DS + MS; SG + FP + ES; EC + FPL + RI; | | | | |
| | AH + FP + KQ; LA + MS + KQ; EC + RT + AH; | | | | |
| | EL + AH + FP; EC + LA + MS; TB + MM + RI; | | | | |
| | RT + AH + ES; TB + FP + KQ; DS + EL + MF; | | | | |
| | RT + MF + FP; MF + FP + ES; PS + TB + FP; | | | | |
| | LA + AH + KQ; EL + ES + RI; SG + RT + FPL; | | | | |
| | DS + LA + AH; FPL + MM + FP; LA + MM + ES; | | | | |
| | EL + FP + MS; FPL + EL + MM; EC + MS + RI; | | | | |
| | PS + DS + FPL; PS + DS + AH; RT + LA + MM; | | | | |
| | EL + ES + KQ; FPL + AH + KQ | | | | |
| 3 | EC + DS + FPL; EC + TB + RI; RT + DS + LA; | 0.72 | 0.2944- | 41.27%- | 36.36%- |
| | EC + EL + TB; RT + TB + FP; EC + RT + MS; | | 0.5166 | 93.65% | 92.93% |
| | EC + DS + KQ; EC + PS + LA; RT + FPL + LA; | | | | |
| | EC + RT + RI; SG + LA + TB; FPL + LA + TB; | | | | |
| | EC + SG + FPL; DS + FPL + LA; EC + TB + KQ; | | | | |
| | RT + EL + FP; DS + LA + RI; TB + FP + MS; | | | | |
| | PS + MM + ES; LA + TB + RI; PS + DS + ES; | | | | |
| | DS + EL + FP; RT + FPL + FP; DS + LA + MM; | | | | |
| | LA + AH + FP; EL + AH + ES; SG + FPL + ES; | | | | |
| | RT + DS + FP; TB + ES + MS; EC + PS + RI; | | | | |
| | EC + FPL + FP; DS + AH + MS; MF + TB + AH; | | | | |
| | EC + SG + KQ; SG + LA + RI; PS + RT + LA; | | | | |
| | LA + MM + FP; EC + LA + AH; RT + FP + RI; | | | | |
| | RT + DS + EL; DS + MF + RI; SG + TB + FP; | | | | |
| | EC + FPL + EL; SG + RT + RI; DS + AH + FP; | | | | |
| | EC + AH + FP; EC + SG + MF; LA + ES + KQ; | | | | |
| | FPL + FP + KQ; FPL + MF + ES; EC + RT + TB; | | | | |
| | EC + ES + MS; SG + AH + RI; RT + EL + RI; | | | | |
| | FPL + MM + ES; RT + EL + KQ; DS + FP + MS; | | | | |
| | SG + MM + AH; PS + DS + RI; SG + DS + AH; | | | | |
| | SG + DS + FP; SG + LA + KQ; EC + DS + MM; | | | | |
| | PS + EL + MM; RT + FPL + RI; EC + EL + FP; | | | | |
| | MF + AH + FP; DS + EL + AH; SG + RT + DS; | | | | |
| | PS + FPL + ES; TB + MM + FP; RT + FPL + EL; | | | | |
| | SG + PS + AH; EL + MM + ES; EC + PS + EL; | | | | |
| | PS + TB + ES; EC + FPL + MM; DS + FPL + MM | | | | |
| 3 | SG + DS + LA; EC + FPL + TB; EC + DS + AH; | 0.73 | 0.3108- | 47.62%- | 44.44%- |
| | DS + FPL + EL; EC + SG + RT; DS + LA + ES; | | 0.521 | 92.06% | 87.88% |
| | EC + MM + ES; PS + LA + ES; EC + DS + RI; | | | | |
| | DS + FPL + KQ; EC + SG + TB; EC + PS + AH; | | | | |
| | SG + RT + FP; EC + FPL + MF; DS + TB + RI; | | | | |
| | EC + RT + LA; AH + FP + ES; EC + FP + ES; | | | | |
| | RT + LA + MS; RT + LA + FP; FPL + TB + KQ; | | | | |
| | EC + DS + MS; EC + RT + FPL; EC + DS + LA; | | | | |
| | DS + AH + ES; LA + FP + ES; LA + AH + ES; | | | | |
| | EL + TB + FP; RT + DS + RI; EC + PS + MS; | | | | |
| | SG + LA + ES; FPL + TB + MM; DS + TB + MS; | | | | |
| | SG + EL + ES; EL + TB + MS; SG + FPL + TB; | | | | |
| | EC + SG + AH; EC + PS + MM; EC + DS + FP; | | | | |
| | EC + RT + DS; FPL + EL + ES; EC + FP + MS; | | | | |
| | EC + TB + MS; TB + MM + MS; EC + EL + ES; | | | | |
| | RT + EL + LA; TB + AH + ES; EC + SG + MS; | | | | |
| | EC + FPL + ES; LA + ES + RI; TB + FP + RI; | | | | |
| | LA + TB + AH; EC + PS + MF; EC + PS + FPL; | | | | |
| | EC + TB + AH; SG + TB + AH; EC + ES + RI; | | | | |
| | SG + DS + RI; SG + FPL + LA; FPL + ES + KQ; | | | | |
| | LA + FP + MS; PS + EL + TB; RT + DS + FPL; | | | | |
| | EL + ES + MS; EC + MF + TB; EC + MF + FP; | | | | |
| | FPL + TB + FP; EC + RT + FP; RT + LA + TB; | | | | |
| | TB + ES + RI; RT + LA + RI; EC + ES + KQ | | | | |
| 3 | RT + EL + ES; RT + FP + ES; EC + TB + MM; | 0.74 | 0.312- | 49.21%- | 51.52%- |
| | SG + EL + FP; EL + TB + MM; RT + LA + ES; | | 0.5152 | 90.48% | 88.89% |
| | EC + PS + DS; DS + LA + FP; DS + FPL + RI; | | | | |
| | SG + AH + ES; LA + TB + ES; EC + RT + ES; | | | | |
| | EC + LA + ES; RT + FPL + ES; EC + SG + DS; | | | | |
| | SG + AH + FP; FPL + EL + TB; EC + SG + MM; | | | | |
| | TB + FP + ES; RT + ES + RI; DS + FPL + MF; | | | | |
| | FPL + MF + TB; DS + TB + AH; EC + SG + RI; | | | | |
| | FPL + FP + ES; RT + LA + KQ; EC + AH + ES; | | | | |
| | EC + DS + MF; EC + MF + ES; DS + LA + TB | | | | |
| 3 | EC + DS + TB; DS + FPL + ES; TB + AH + FP; | 0.75 | 0.3399- | 61.90%- | 47.47%- |
| | SG + DS + FPL; EC + TB + ES; DS + TB + ES; | | 0.464 | 93.65% | 83.84% |
| | EC + SG + FP; LA + TB + MM; EC + LA + TB; | | | | |
| | EC + SG + PS; SG + LA + AH; SG + LA + FP; | | | | |
| | LA + TB + FP; EC + DS + ES; EC + TB + FP; | | | | |
| | DS + ES + RI; DS + FPL + TB; SG + RT + LA; | | | | |
| | DS + TB + FP; EC + PS + TB | | | | |
| 3 | EC + PS + ES; FPL + TB + ES; EC + SG + ES; | 0.76 | 0.3865- | 58.73%- | 51.52%- |
| | PS + EL + ES; LA + TB + MS | | 0.4922 | 92.06% | 86.87% |
| 3 | EL + TB + ES; EC + SG + LA | 0.77 | 0.4269- | 61.9% | 81.82%- |
| | | | 0.4387 | | 88.89% |
| 3 | TB + MM + ES | 0.78 | 0.3867 | 52.38% | 91.92% |

TABLE 4
The frequency of the 18 markers in the 372 combinations

| Bacterial marker | Freq |
|---|---|
| Escherichia_{—}coli | 111 |
| Faecalibacterium prausnitzii | 85 |
| Dorea_sp_AF36_15AT | 84 |
| Turicibacter_{—}bilis | 83 |
| Eggerthella_{—}sinensis | 81 |
| Lawsonibacter_{—}asaccharolyticus | 79 |
| Flavonifractor_{—}plautii | 73 |
| Romboutsia_{—}timonensis | 70 |
| Streptococcus_{—}gordonii | 66 |
| Anaerostipes_{—}hadrus | 63 |
| Eggerthella_{—}lenta | 61 |
| Roseburia intestinalis | 52 |
| Phascolarctobacterium_SGB4573 | 47 |
| Megasphaera_{—}micronuciformis | 43 |
| Klebsiella_{—}quasipneumoniae | 42 |
| Megasphaera_sp_NM10 | 42 |
| Megamonas_{—}funiformis | 32 |
| Olsenella_sp_AF16_14LB | 2 |

Referring to Table 5, when choosing 4 markers from the 18 markers, 647 out of 3060 (21.14%) total possible combinations achieved AUCs>=0.75, and 2207 out of 3060 (72.12%) total possible combinations achieved AUCs>=0.7. For the sake of clarity, the bacteria species combinations shown in Table 5 are represented by abbreviations of the bacteria species as per Table 2. For example, “MF+AH+ES+KQ” refers to the combination of “Megamonas funiformis, Anaerostipes hadrus, Eggerthella sinensis, and Klebsiella_quasipneumoniae”.

As shown in Table 6, among these 2207 combinations, Escherichia coli appeared most frequently in 627 combinations, followed by Dorea_sp_AF36_15AT which appeared 559 times, Faecalibacterium prausnitzii appeared 554 times, Turicibacter bilis appeared 548 times, and Lawsonibacter_asaccharolyticus appeared 542 times.

TABLE 5
List of combinations of 4 bacterial markers and the performance.

| Number | | | Best_{—} | | |
|---|---|---|---|---|---|
| of | | | Threshold_{—} | Sensitivity_{—} | Specificity_{—} |
| markers | Combinations | AUC | Range | Range | Range |
| 4 | MF + AH + ES + KQ; EC + OS + LA + KQ; | 0.7 | 0.2676- | 36.51%- | 35.35%- |
| | SG + DS + OS + AH; DS + ES + MS + KQ; | | 0.5785 | 95.24% | 95.96% |
| | EC + OS + TB + AH; EL + TB + AH + RI; | | | | |
| | RT + LA + MF + KQ; LA + FP + KQ + RI; | | | | |
| | EC + SG + OS + TB; PS + FPL + LA + MF; | | | | |
| | RT + EL + TB + MM; RT + TB + AH + KQ; | | | | |
| | EL + LA + MF + TB; EC + RT + OS + KQ; | | | | |
| | EC + OS + EL + KQ; SG + MM + FP + MS; | | | | |
| | FPL + EL + MM + AH; EL + AH + FP + MS; | | | | |
| | LA + MM + AH + RI; SG + PS + DS + EL; | | | | |
| | FPL + MF + MM + FP; PS + DS + LA + MS; | | | | |
| | EL + LA + MS + KQ; EL + MF + TB + AH; | | | | |
| | RT + DS + LA + MM; RT + EL + MM + MS; | | | | |
| | FPL + MM + AH + KQ; EC + EL + MF + AH; | | | | |
| | EC + EL + KQ + RI; EC + MF + KQ + RI; | | | | |
| | PS + DS + OS + AH; EC + PS + OS + FP; | | | | |
| | PS + RT + MF + FP; PS + FPL + MF + KQ; | | | | |
| | DS + OS + LA + RI; SG + OS + LA + AH; | | | | |
| | FPL + EL + LA + KQ; PS + RT + DS + MF; | | | | |
| | PS + DS + MS + KQ; DS + OS + TB + MM; | | | | |
| | DS + MF + AH + KQ; EC + MM + AH + MS; | | | | |
| | EC + OS + TB + MM; RT + TB + MM + RI; | | | | |
| | EC + MF + AH + RI; SG + RT + TB + MS; | | | | |
| | PS + RT + MM + FP; PS + MM + AH + RI; | | | | |
| | RT + FPL + MM + KQ; RT + TB + MS + KQ; | | | | |
| | EL + MF + MM + FP; SG + RT + AH + MS; | | | | |
| | SG + LA + MF + TB; PS + RT + FPL + MS; | | | | |
| | FP + ES + KQ + RI; PS + RT + EL + AH; | | | | |
| | EL + TB + MM + AH; EC + SG + OS + KQ; | | | | |
| | EL + LA + MM + AH; FPL + LA + MM + MS; | | | | |
| | EC + EL + MF + MM; EC + OS + ES + KQ; | | | | |
| | DS + EL + LA + KQ; AH + FP + MS + KQ; | | | | |
| | FPL + EL + MS + RI; OS + TB + MM + ES; | | | | |
| | EC + MM + MS + RI; SG + FPL + AH + MS; | | | | |
| | RT + EL + AH + RI; PS + EL + MM + AH; | | | | |
| | EL + LA + MF + FP; PS + RT + FPL + MM; | | | | |
| | FPL + LA + MS + KQ; EC + OS + TB + MS; | | | | |
| | PS + FPL + MF + AH; PS + FPL + TB + AH; | | | | |
| | RT + FPL + MF + KQ; MF + FP + MS + KQ; | | | | |
| | RT + TB + MM + KQ; LA + MF + AH + RI; | | | | |
| | RT + DS + MM + ES; RT + EL + TB + AH; | | | | |
| | FPL + EL + LA + AH; MF + TB + MS + RI; | | | | |
| | EC + RT + OS + MS; EL + MF + FP + KQ; | | | | |
| | SG + DS + MF + ES; FPL + MF + MS + KQ; | | | | |
| | FPL + LA + MF + RI; EC + MM + MS + KQ; | | | | |
| | DS + MM + KQ + RI; FPL + EL + KQ + RI; | | | | |
| | EL + TB + MS + RI; FPL + EL + LA + MF; | | | | |
| | EC + SG + FPL + OS; PS + RT + FPL + AH; | | | | |
| | EC + EL + LA + RI; PS + FPL + MF + MS; | | | | |
| | FP + ES + MS + RI; EC + OS + FP + KQ; | | | | |
| | RT + OS + FP + KQ; MF + AH + ES + RI; | | | | |
| | EC + PS + OS + EL; RT + MF + TB + RI; | | | | |
| | EC + OS + LA + AH; DS + FPL + OS + MM; | | | | |
| | SG + PS + DS + TB; SG + RT + EL + AH; | | | | |
| | RT + FPL + EL + AH; RT + OS + FP + RI; | | | | |
| | MF + TB + AH + RI; TB + AH + KQ + RI; | | | | |
| | PS + MM + MS + RI; PS + MF + AH + KQ; | | | | |
| | EC + FPL + OS + TB; SG + TB + MM + KQ; | | | | |
| | SG + TB + ES + KQ; PS + RT + MS + KQ; | | | | |
| | RT + FPL + MS + RI; SG + FPL + AH + RI; | | | | |
| | SG + EL + TB + MM; EL + MM + MS + KQ; | | | | |
| | SG + EL + MF + ES; SG + EL + TB + AH; | | | | |
| | PS + FP + MS + RI; EL + LA + MM + FP; | | | | |
| | LA + MF + MM + FP; EC + RT + OS + AH; | | | | |
| | LA + MF + AH + KQ; LA + AH + KQ + RI; | | | | |
| | MF + TB + AH + KQ; EC + RT + MF + MM; | | | | |
| | RT + DS + MF + TB; FPL + EL + MF + KQ; | | | | |
| | SG + PS + FP + RI; MM + AH + FP + RI; | | | | |
| | PS + MM + AH + MS; DS + LA + MF + KQ; | | | | |
| | AH + FP + KQ + RI; FPL + EL + FP + RI; | | | | |
| | EL + FP + KQ + RI; PS + MM + FP + MS; | | | | |
| | RT + OS + EL + FP; DS + EL + LA + MF; | | | | |
| | EC + EL + AH + RI; SG + MF + MS + RI; | | | | |
| | PS + FPL + LA + AH; PS + TB + MS + KQ; | | | | |
| | FPL + MM + FP + MS; RT + OS + LA + ES; | | | | |
| | EL + MF + ES + RI; SG + RT + TB + KQ; | | | | |
| | SG + MF + AH + KQ; PS + TB + FP + RI; | | | | |
| | PS + EL + LA + RI; PS + TB + MM + KQ; | | | | |
| | PS + AH + MS + KQ; EC + RT + OS + EL; | | | | |
| | PS + EL + FP + RI; PS + AH + MS + RI; | | | | |
| | EC + SG + OS + MF; DS + OS + EL + LA; | | | | |
| | EL + AH + MS + KQ; AH + ES + KQ + RI; | | | | |
| | SG + ES + KQ + RI; TB + AH + MS + RI; | | | | |
| | SG + PS + DS + KQ; DS + EL + MM + RI; | | | | |
| | FPL + OS + TB + FP; PS + FPL + AH + FP; | | | | |
| | SG + RT + OS + RI; SG + MM + MS + KQ; | | | | |
| | PS + TB + KQ + RI; LA + AH + MS + RI; | | | | |
| | EC + DS + EL + MM; DS + FPL + OS + MF; | | | | |
| | RT + FPL + OS + FP; EC + EL + MM + MS; | | | | |
| | PS + RT + OS + LA; PS + FPL + MS + KQ; | | | | |
| | RT + FPL + MF + AH; SG + MF + TB + MS; | | | | |
| | DS + FPL + OS + ES; EC + OS + FP + RI; | | | | |
| | SG + OS + TB + AH; FPL + MS + KQ + RI; | | | | |
| | EC + OS + FP + MS; PS + DS + MM + FP; | | | | |
| | RT + OS + AH + FP; PS + DS + TB + KQ; | | | | |
| | EL + MF + TB + RI; SG + TB + KQ + RI; | | | | |
| | PS + DS + LA + MM; EC + MM + AH + RI; | | | | |
| | EC + FPL + AH + MS; SG + FPL + OS + LA; | | | | |
| | PS + LA + MS + RI; FPL + EL + AH + FP; | | | | |
| | EL + LA + ES + RI; SG + RT + MF + KQ; | | | | |
| | TB + MM + KQ + RI; DS + FPL + OS + FP; | | | | |
| | FPL + AH + ES + MS; PS + OS + EL + ES; | | | | |
| | EC + OS + AH + KQ; DS + EL + KQ + RI; | | | | |
| | EC + PS + FPL + OS; PS + DS + MM + KQ; | | | | |
| | PS + EL + MF + AH; EC + OS + ES + RI; | | | | |
| | RT + MM + AH + FP; PS + EL + AH + MS; | | | | |
| | EL + MF + TB + KQ; PS + MF + AH + RI; | | | | |
| | RT + TB + MM + ES; EL + LA + MM + KQ; | | | | |
| | SG + EL + MF + TB; SG + MF + MM + FP; | | | | |
| | RT + DS + MF + MS; PS + RT + TB + AH; | | | | |
| | MM + FP + KQ + RI; FPL + LA + AH + RI; | | | | |
| | EC + DS + OS + MM; EC + OS + MF + ES; | | | | |
| | EC + OS + AH + ES; FPL + LA + MF + KQ; | | | | |
| | SG + RT + TB + MM; PS + MF + AH + MS; | | | | |
| | SG + OS + LA + TB; PS + FPL + MM + FP; | | | | |
| | FPL + AH + FP + MS; LA + MF + ES + KQ | | | | |
| 4 | EC + RT + MF + TB; SG + FPL + MS + RI; | 0.71 | 0.2718- | 38.10%- | 35.35%- |
| | MF + MM + AH + FP; LA + MF + TB + MS; | | 0.5503 | 98.41% | 93.94% |
| | FPL + MF + AH + ES; PS + RT + DS + AH; | | | | |
| | EC + LA + MF + KQ; PS + RT + TB + ES; | | | | |
| | RT + DS + MM + MS; DS + OS + TB + FP; | | | | |
| | EL + LA + TB + MM; MF + TB + AH + MS; | | | | |
| | PS + RT + TB + FP; PS + DS + AH + KQ; | | | | |
| | PS + FPL + LA + FP; TB + MM + ES + KQ; | | | | |
| | SG + PS + LA + MM; SG + DS + MM + FP; | | | | |
| | SG + EL + ES + MS; RT + AH + FP + KQ; | | | | |
| | DS + EL + MM + AH; DS + MM + AH + KQ; | | | | |
| | EL + LA + ES + MS; PS + FPL + LA + MM; | | | | |
| | PS + TB + ES + KQ; FPL + EL + MM + RI; | | | | |
| | MM + AH + FP + MS; EC + MM + FP + KQ; | | | | |
| | SG + FPL + MF + MS; PS + RT + FP + MS; | | | | |
| | EC + MM + KQ + RI; RT + DS + TB + KQ; | | | | |
| | RT + EL + AH + KQ; PS + FPL + EL + MS; | | | | |
| | PS + MM + KQ + RI; SG + FP + ES + MS; | | | | |
| | SG + PS + TB + MS; PS + LA + MF + FP; | | | | |
| | RT + DS + MM + RI; MM + AH + ES + RI; | | | | |
| | SG + PS + FPL + AH; RT + AH + ES + RI; | | | | |
| | SG + PS + AH + MS; RT + FPL + AH + KQ; | | | | |
| | EC + EL + MS + RI; SG + RT + DS + MF; | | | | |
| | SG + DS + MM + RI; PS + RT + EL + MM; | | | | |
| | RT + DS + MS + KQ; PS + ES + KQ + RI; | | | | |
| | RT + AH + KQ + RI; MF + AH + FP + MS; | | | | |
| | EC + PS + OS + RI; EC + DS + OS + MF; | | | | |
| | EC + OS + LA + ES; RT + FPL + TB + AH; | | | | |
| | SG + FPL + MF + KQ; EC + DS + OS + FP; | | | | |
| | EC + DS + OS + KQ; EC + MF + MM + AH; | | | | |
| | RT + FPL + TB + KQ; EC + EL + LA + MM; | | | | |
| | EC + AH + FP + RI; DS + AH + MS + KQ; | | | | |
| | SG + TB + MS + KQ; RT + DS + MM + FP; | | | | |
| | EC + OS + MF + TB; PS + DS + FPL + AH; | | | | |
| | DS + LA + MM + KQ; PS + DS + EL + KQ; | | | | |
| | FPL + LA + MM + AH; SG + MS + KQ + RI; | | | | |
| | RT + FPL + MS + KQ; FPL + MF + FP + MS; | | | | |
| | PS + DS + FPL + MS; RT + ES + MS + KQ; | | | | |
| | DS + FPL + AH + RI; FPL + TB + MS + RI; | | | | |
| | PS + ES + MS + RI; PS + MM + AH + FP; | | | | |
| | EC + PS + OS + AH; SG + RT + FPL + MM; | | | | |
| | PS + EL + MF + FP; FPL + MF + ES + RI; | | | | |
| | LA + MF + ES + MS; SG + FPL + EL + AH; | | | | |
| | EC + PS + OS + LA; SG + MF + MM + AH; | | | | |
| | DS + FPL + OS + EL; MF + AH + FP + KQ; | | | | |
| | EC + AH + KQ + RI; SG + FPL + MM + AH; | | | | |
| | EC + OS + TB + ES; SG + OS + LA + FP; | | | | |
| | PS + DS + LA + MF; PS + FPL + ES + MS; | | | | |
| | SG + RT + FPL + AH; PS + DS + FP + MS; | | | | |
| | FPL + EL + MS + KQ; MF + AH + ES + MS; | | | | |
| | SG + PS + ES + KQ; PS + EL + LA + MM; | | | | |
| | SG + RT + MF + MS; SG + EL + AH + KQ; | | | | |
| | PS + RT + AH + KQ; RT + DS + MF + AH; | | | | |
| | PS + FPL + MS + RI; PS + EL + FP + MS; | | | | |
| | PS + MF + TB + AH; RT + LA + AH + MS; | | | | |
| | FPL + FP + MS + RI; EC + SG + RT + OS; | | | | |
| | EC + RT + OS + FP; PS + RT + FP + KQ; | | | | |
| | PS + DS + FP + KQ; RT + DS + EL + TB; | | | | |
| | RT + OS + TB + FP; FPL + EL + LA + MM; | | | | |
| | FPL + EL + MF + AH; EL + ES + KQ + RI; | | | | |
| | EC + RT + OS + LA; SG + EL + MF + RI; | | | | |
| | EC + EL + FP + RI; SG + PS + KQ + RI; | | | | |
| | PS + RT + MF + ES; EC + FPL + AH + RI; | | | | |
| | RT + EL + MF + TB; SG + PS + EL + TB; | | | | |
| | SG + MM + KQ + RI; PS + FPL + AH + KQ; | | | | |
| | RT + EL + MM + KQ; EC + AH + MS + RI; | | | | |
| | SG + EL + LA + MS; PS + DS + FP + RI; | | | | |
| | EL + MM + FP + MS; TB + ES + MS + KQ; | | | | |
| | EC + EL + LA + KQ; EC + EL + MF + MS; | | | | |
| | PS + RT + MM + KQ; PS + DS + MF + TB; | | | | |
| | OS + TB + AH + FP; EL + LA + MM + ES; | | | | |
| | EC + RT + OS + RI; RT + MM + ES + KQ; | | | | |
| | PS + RT + DS + EL; PS + LA + MS + KQ; | | | | |
| | PS + FP + ES + KQ; SG + PS + TB + MM; | | | | |
| | PS + LA + FP + KQ; FPL + LA + FP + MS; | | | | |
| | FPL + MM + AH + ES; PS + AH + KQ + RI; | | | | |
| | DS + EL + TB + KQ; PS + FPL + EL + KQ; | | | | |
| | DS + ES + KQ + RI; EC + LA + MM + RI; | | | | |
| | PS + MF + AH + FP; DS + EL + MF + MS; | | | | |
| | EC + RT + MF + KQ; FPL + EL + MF + MS; | | | | |
| | EC + DS + OS + EL; PS + RT + DS + KQ; | | | | |
| | SG + PS + MF + RI; PS + FPL + KQ + RI; | | | | |
| | DS + EL + MS + RI; MF + TB + MM + AH; | | | | |
| | SG + EL + AH + RI; FPL + AH + ES + RI; | | | | |
| | PS + MF + ES + RI; FPL + LA + MF + AH; | | | | |
| | EC + MF + AH + MS; SG + RT + MM + AH; | | | | |
| | DS + EL + LA + MS; FPL + EL + FP + MS; | | | | |
| | LA + MF + FP + KQ; TB + AH + MS + KQ; | | | | |
| | SG + PS + LA + MS; FPL + MF + AH + FP; | | | | |
| | EL + LA + FP + KQ; RT + OS + LA + TB; | | | | |
| | EL + MF + FP + RI; EC + PS + OS + MS; | | | | |
| | SG + PS + TB + ES; PS + FPL + EL + LA; | | | | |
| | PS + FPL + AH + ES; PS + LA + MM + KQ; | | | | |
| | RT + DS + AH + KQ; PS + RT + DS + TB; | | | | |
| | PS + RT + FPL + MF; FP + MS + KQ + RI; | | | | |
| | SG + OS + AH + FP; PS + EL + LA + AH; | | | | |
| | PS + EL + AH + RI; DS + FPL + AH + MS; | | | | |
| | EC + OS + EL + TB; RT + MF + KQ + RI; | | | | |
| | FPL + LA + KQ + RI; SG + PS + EL + LA; | | | | |
| | FPL + MM + MS + KQ; EC + SG + OS + RI; | | | | |
| | FPL + LA + MS + RI; OS + LA + TB + AH; | | | | |
| | EL + MF + TB + MS; EC + OS + FP + ES; | | | | |
| | SG + PS + ES + MS; PS + DS + MF + FP; | | | | |
| | EC + OS + AH + FP; SG + DS + EL + MS; | | | | |
| | OS + LA + AH + FP; PS + LA + FP + RI; | | | | |
| | OS + LA + TB + ES; EC + EL + MF + KQ; | | | | |
| | PS + FPL + FP + RI; PS + EL + MF + RI; | | | | |
| | RT + TB + MM + MS; OS + EL + TB + ES; | | | | |
| | FPL + MF + ES + KQ; PS + FP + MS + KQ; | | | | |
| | DS + FPL + OS + LA; DS + OS + TB + AH; | | | | |
| | EC + MF + MS + KQ; SG + MM + AH + RI; | | | | |
| | RT + FPL + EL + MF; SG + PS + RT + MM; | | | | |
| | SG + PS + MS + KQ; PS + RT + TB + KQ; | | | | |
| | DS + OS + AH + FP; FPL + FP + KQ + RI; | | | | |
| | TB + MM + AH + RI; EC + OS + TB + RI; | | | | |
| | SG + PS + FP + ES; SG + RT + MF + AH; | | | | |
| | PS + AH + FP + MS; AH + ES + MS + RI; | | | | |
| | EC + EL + MM + AH; SG + MF + FP + KQ; | | | | |
| | EC + LA + MM + MS; EC + LA + MM + KQ; | | | | |
| | FPL + EL + MM + MS; EL + FP + MS + RI; | | | | |
| | SG + PS + FP + KQ; EL + LA + AH + MS; | | | | |
| | SG + EL + LA + RI; FPL + MM + FP + RI; | | | | |
| | RT + MF + ES + MS; PS + RT + AH + FP; | | | | |
| | SG + PS + RT + AH; EC + SG + OS + AH; | | | | |
| | DS + MF + FP + KQ; EC + RT + OS + TB; | | | | |
| | PS + TB + AH + MS; SG + FPL + MM + MS; | | | | |
| | DS + EL + LA + RI; EC + EL + LA + MF; | | | | |
| | EC + EL + AH + MS; PS + RT + EL + MF; | | | | |
| | DS + OS + AH + RI; DS + MF + KQ + RI; | | | | |
| | FPL + MM + KQ + RI; MM + FP + MS + RI; | | | | |
| | SG + DS + TB + KQ; RT + DS + TB + ES; | | | | |
| | DS + OS + TB + ES; EC + FPL + OS + FP; | | | | |
| | RT + MF + AH + FP; FPL + ES + MS + RI; | | | | |
| | EC + DS + OS + MS; SG + DS + TB + MM; | | | | |
| | SG + MF + TB + ES; DS + MF + MM + FP; | | | | |
| | MF + FP + MS + RI; SG + RT + DS + MM; | | | | |
| | SG + LA + MF + KQ; RT + OS + EL + LA; | | | | |
| | DS + EL + TB + MM; SG + MF + AH + MS; | | | | |
| | RT + DS + EL + KQ; FPL + LA + MM + RI; | | | | |
| | FPL + TB + MM + AH; EL + MM + AH + KQ; | | | | |
| | SG + RT + FPL + MS; PS + MF + TB + RI; | | | | |
| | EC + PS + RT + OS; RT + EL + MF + MS | | | | |
| 4 | SG + MF + FP + MS; PS + RT + ES + KQ; | 0.72 | 0.272- | 41.27%- | 41.41%- |
| | RT + DS + AH + FP; DS + LA + MF + TB; | | 0.6162 | 95.24% | 91.92% |
| | EL + ES + MS + KQ; EC + FPL + EL + KQ; | | | | |
| | SG + AH + MS + KQ; DS + MM + FP + KQ; | | | | |
| | EC + DS + OS + LA; PS + DS + EL + TB; | | | | |
| | RT + FPL + EL + MS; DS + TB + MM + KQ; | | | | |
| | EC + RT + EL + AH; EC + FPL + LA + MS; | | | | |
| | SG + DS + LA + MF; SG + EL + MS + KQ; | | | | |
| | PS + LA + MM + AH; RT + LA + AH + RI; | | | | |
| | EC + LA + MM + AH; PS + EL + TB + AH; | | | | |
| | SG + RT + DS + KQ; SG + FP + MS + RI; | | | | |
| | DS + LA + MM + ES; SG + PS + EL + AH; | | | | |
| | PS + FPL + ES + KQ; DS + TB + MS + KQ; | | | | |
| | EC + FPL + MS + KQ; SG + EL + KQ + RI; | | | | |
| | MF + TB + ES + RI; SG + PS + RT + KQ; | | | | |
| | PS + EL + MM + MS; FPL + LA + MF + FP; | | | | |
| | SG + RT + EL + MS; DS + FPL + MM + FP; | | | | |
| | MM + FP + ES + KQ; RT + FPL + LA + AH; | | | | |
| | EC + FPL + LA + MM; PS + FPL + FP + ES; | | | | |
| | EC + MF + ES + KQ; PS + RT + FP + ES; | | | | |
| | PS + EL + MF + TB; RT + FPL + LA + MS; | | | | |
| | SG + EL + TB + RI; PS + TB + AH + RI; | | | | |
| | RT + AH + FP + ES; SG + DS + MF + MS; | | | | |
| | RT + DS + TB + MS; SG + FPL + MF + AH; | | | | |
| | PS + RT + EL + FP; PS + DS + MF + AH; | | | | |
| | EC + PS + MF + MM; DS + EL + FP + MS; | | | | |
| | FPL + LA + TB + RI; LA + MF + TB + RI; | | | | |
| | MM + FP + MS + KQ; PS + FPL + MM + RI; | | | | |
| | RT + FPL + FP + KQ; EC + DS + OS + RI; | | | | |
| | SG + DS + ES + KQ; PS + DS + MM + AH; | | | | |
| | PS + TB + MM + FP; RT + FPL + LA + MF; | | | | |
| | EC + FPL + EL + RI; PS + MF + AH + ES; | | | | |
| | EC + RT + FPL + MM; SG + PS + AH + RI; | | | | |
| | PS + DS + FPL + MM; EC + FPL + LA + RI; | | | | |
| | RT + LA + MM + AH; FPL + LA + MM + KQ; | | | | |
| | SG + EL + MM + RI; DS + LA + MF + FP; | | | | |
| | LA + AH + ES + RI; AH + FP + ES + RI; | | | | |
| | EC + MM + AH + KQ; SG + RT + EL + TB; | | | | |
| | FPL + MF + AH + KQ; RT + EL + MM + RI; | | | | |
| | DS + LA + MM + RI; EC + FPL + EL + LA; | | | | |
| | EC + FPL + MM + RI; EC + RT + MM + KQ; | | | | |
| | EC + MF + FP + RI; SG + FPL + TB + AH; | | | | |
| | PS + EL + AH + FP; FPL + EL + MF + FP; | | | | |
| | MF + TB + MM + MS; PS + DS + EL + ES; | | | | |
| | PS + LA + MM + FP; RT + EL + LA + AH; | | | | |
| | FPL + EL + LA + FP; EC + FPL + EL + MS; | | | | |
| | PS + DS + LA + KQ; PS + FPL + FP + KQ; | | | | |
| | PS + LA + FP + MS; DS + OS + LA + AH; | | | | |
| | DS + MF + AH + MS; FPL + MM + ES + RI; | | | | |
| | LA + MM + FP + KQ; EC + RT + AH + RI; | | | | |
| | EC + DS + OS + AH; PS + AH + FP + KQ; | | | | |
| | RT + MM + FP + MS; EL + LA + FP + ES; | | | | |
| | PS + RT + FPL + KQ; EC + OS + LA + TB; | | | | |
| | EC + EL + TB + RI; SG + DS + MM + ES; | | | | |
| | EC + FPL + MF + RI; EC + MF + FP + KQ; | | | | |
| | SG + PS + DS + MS; PS + FPL + LA + KQ; | | | | |
| | PS + EL + LA + TB; EL + MF + AH + ES; | | | | |
| | EC + OS + TB + FP; EC + MM + FP + RI; | | | | |
| | PS + MM + AH + KQ; RT + DS + EL + AH; | | | | |
| | FPL + EL + MM + KQ; FPL + ES + KQ + RI; | | | | |
| | LA + MS + KQ + RI; PS + DS + KQ + RI; | | | | |
| | DS + TB + KQ + RI; SG + MF + FP + RI; | | | | |
| | PS + RT + MF + RI; PS + TB + MS + RI; | | | | |
| | RT + FPL + TB + MM; FP + ES + MS + KQ; | | | | |
| | EC + RT + EL + MM; EC + MM + ES + MS; | | | | |
| | PS + DS + ES + KQ; PS + TB + FP + KQ; | | | | |
| | RT + DS + OS + RI; LA + MM + AH + MS; | | | | |
| | EC + SG + PS + OS; PS + RT + LA + MF; | | | | |
| | PS + DS + MM + RI; EL + MF + AH + FP; | | | | |
| | MM + FP + ES + RI; EC + MS + KQ + RI; | | | | |
| | PS + FPL + TB + MS; EC + AH + MS + KQ; | | | | |
| | SG + AH + FP + RI; FPL + TB + KQ + RI; | | | | |
| | DS + FPL + MF + MM; LA + AH + MS + KQ; | | | | |
| | EC + RT + FPL + KQ; RT + DS + AH + ES; | | | | |
| | LA + TB + KQ + RI; FPL + TB + AH + RI; | | | | |
| | FPL + LA + TB + MS; SG + MM + ES + MS; | | | | |
| | DS + LA + MM + FP; EC + RT + FPL + MS; | | | | |
| | SG + FPL + EL + MS; PS + DS + OS + LA; | | | | |
| | LA + MF + ES + RI; MF + TB + ES + MS; | | | | |
| | EC + PS + FPL + MS; EC + EL + AH + KQ; | | | | |
| | PS + DS + EL + RI; PS + FPL + LA + RI; | | | | |
| | RT + MM + AH + ES; EC + SG + DS + OS; | | | | |
| | EC + RT + MF + AH; EC + DS + FPL + OS; | | | | |
| | SG + MM + AH + MS; RT + EL + MM + FP; | | | | |
| | FPL + EL + LA + ES; EC + PS + OS + MF; | | | | |
| | EC + EL + LA + MS; SG + RT + EL + MF; | | | | |
| | PS + RT + DS + FP; EC + SG + MF + KQ; | | | | |
| | EC + PS + EL + RI; SG + RT + TB + AH; | | | | |
| | PS + RT + FPL + TB; EC + LA + MF + RI; | | | | |
| | EL + AH + ES + RI; PS + EL + FP + KQ; | | | | |
| | RT + TB + ES + KQ; PS + EL + LA + FP; | | | | |
| | RT + FPL + TB + MS; EC + RT + DS + OS; | | | | |
| | SG + PS + DS + FP; RT + FPL + MM + RI; | | | | |
| | DS + FPL + MM + MS; EC + FPL + LA + AH; | | | | |
| | PS + RT + EL + MS; RT + FPL + EL + MM; | | | | |
| | EC + DS + OS + ES; EC + LA + FP + KQ; | | | | |
| | PS + LA + MF + AH; RT + MM + ES + MS; | | | | |
| | LA + MM + MS + RI; SG + RT + MM + MS; | | | | |
| | SG + DS + EL + ES; RT + MM + MS + RI; | | | | |
| | DS + MF + MS + RI; FPL + LA + AH + KQ; | | | | |
| | SG + FP + KQ + RI; PS + EL + MM + RI; | | | | |
| | SG + RT + FPL + MF; SG + TB + FP + ES; | | | | |
| | FPL + MF + MM + ES; TB + MM + MS + KQ; | | | | |
| | DS + MM + ES + MS; EC + RT + MM + AH; | | | | |
| | SG + RT + EL + MM; RT + MF + AH + ES; | | | | |
| | EC + LA + AH + RI; PS + FPL + EL + RI; | | | | |
| | EC + MF + MS + RI; PS + LA + AH + RI; | | | | |
| | SG + PS + FPL + MM; PS + RT + DS + MS; | | | | |
| | RT + DS + AH + MS; EL + MM + ES + RI; | | | | |
| | RT + MF + FP + KQ; EC + FPL + KQ + RI; | | | | |
| | EC + FPL + MS + RI; EC + MM + AH + FP; | | | | |
| | PS + LA + AH + MS; MM + AH + FP + KQ; | | | | |
| | SG + RT + MF + ES; PS + EL + TB + RI; | | | | |
| | DS + FP + KQ + RI; EC + OS + TB + KQ; | | | | |
| | PS + RT + EL + TB; RT + FPL + AH + RI; | | | | |
| | EL + MM + FP + KQ; TB + MM + AH + KQ; | | | | |
| | AH + ES + MS + KQ; EC + EL + LA + AH; | | | | |
| | SG + ES + MS + RI; PS + ES + MS + KQ; | | | | |
| | EC + SG + OS + FP; EC + FPL + MM + MS; | | | | |
| | RT + DS + TB + AH; DS + AH + FP + KQ; | | | | |
| | EL + LA + FP + RI; SG + PS + RT + MS; | | | | |
| | MM + ES + MS + RI; PS + EL + MM + FP; | | | | |
| | LA + AH + FP + RI; SG + PS + MF + AH; | | | | |
| | SG + MM + MS + RI; DS + EL + MF + AH; | | | | |
| | EL + MF + FP + MS; SG + DS + MM + MS; | | | | |
| | EC + LA + KQ + RI; SG + MM + ES + KQ; | | | | |
| | RT + LA + MF + AH; RT + MM + KQ + RI; | | | | |
| | EC + RT + EL + MF; EC + TB + AH + RI; | | | | |
| | PS + MM + ES + KQ; DS + LA + KQ + RI; | | | | |
| | TB + MM + AH + MS; PS + LA + KQ + RI; | | | | |
| | RT + MF + MS + RI; EL + MF + ES + MS; | | | | |
| | MF + FP + ES + RI; RT + DS + MM + AH; | | | | |
| | DS + FPL + OS + RI; SG + RT + DS + AH; | | | | |
| | PS + MF + TB + ES; SG + DS + MF + TB; | | | | |
| | PS + DS + EL + MS; EC + SG + OS + ES; | | | | |
| | SG + EL + MS + RI; RT + LA + MF + TB; | | | | |
| | EL + TB + AH + MS; EC + MF + MM + FP; | | | | |
| | LA + MF + FP + RI; EC + LA + MM + FP; | | | | |
| | PS + RT + AH + RI; DS + EL + TB + RI; | | | | |
| | FPL + MF + ES + MS; LA + ES + KQ + RI; | | | | |
| | EC + EL + MM + KQ; SG + RT + AH + RI; | | | | |
| | PS + DS + EL + LA; PS + TB + AH + KQ; | | | | |
| | RT + OS + LA + RI; LA + MM + AH + KQ; | | | | |
| | SG + ES + MS + KQ; RT + FPL + MM + FP; | | | | |
| | RT + MM + FP + RI; PS + FPL + MF + RI; | | | | |
| | PS + FPL + MM + KQ; EL + LA + AH + KQ; | | | | |
| | EC + RT + OS + ES; SG + LA + MF + ES; | | | | |
| | SG + LA + MF + RI; DS + MM + FP + RI; | | | | |
| | SG + EL + TB + KQ; RT + AH + ES + MS; | | | | |
| | EC + MF + AH + KQ; SG + LA + MF + MS; | | | | |
| | PS + FPL + EL + FP; DS + AH + KQ + RI; | | | | |
| | FPL + MM + ES + MS; RT + LA + TB + MM; | | | | |
| | LA + MM + FP + RI; RT + TB + AH + FP; | | | | |
| | DS + FPL + MM + AH; EC + LA + MF + MS; | | | | |
| | EL + MM + AH + FP; EL + ES + MS + RI; | | | | |
| | LA + MF + AH + MS; PS + MM + FP + ES; | | | | |
| | RT + EL + AH + FP; FPL + EL + MF + MM; | | | | |
| | EC + FPL + MM + AH | | | | |
| 4 | EC + PS + MM + MS; DS + MM + AH + MS; | 0.73 | 0.2864- | 41.27%- | 35.35%- |
| | EC + PS + FPL + LA; RT + FPL + MF + RI; | | 0.5242 | 98.41% | 91.92% |
| | EC + SG + EL + MS; EC + PS + RT + EL; | | | | |
| | DS + AH + FP + MS; PS + EL + MS + RI; | | | | |
| | EC + RT + EL + LA; EC + SG + MM + MS; | | | | |
| | SG + PS + LA + TB; PS + LA + TB + MS; | | | | |
| | PS + MM + ES + RI; DS + FPL + OS + TB; | | | | |
| | SG + DS + EL + LA; RT + OS + LA + FP; | | | | |
| | RT + TB + AH + ES; FPL + AH + FP + KQ; | | | | |
| | SG + PS + AH + KQ; SG + LA + MS + RI; | | | | |
| | PS + RT + LA + FP; EC + PS + RT + FP; | | | | |
| | EC + LA + MF + FP; SG + RT + MS + KQ; | | | | |
| | PS + RT + DS + FPL; RT + FPL + MM + ES; | | | | |
| | EC + FPL + MF + KQ; SG + DS + FPL + OS; | | | | |
| | PS + AH + ES + KQ; EC + PS + MM + FP; | | | | |
| | EC + RT + FPL + AH; PS + RT + ES + MS; | | | | |
| | RT + EL + AH + ES; RT + MF + FP + RI; | | | | |
| | DS + AH + MS + RI; EL + LA + TB + MS; | | | | |
| | LA + MM + FP + MS; AH + FP + ES + KQ; | | | | |
| | EC + SG + EL + MM; RT + TB + KQ + RI; | | | | |
| | EC + PS + EL + LA; EC + PS + AH + MS; | | | | |
| | EC + AH + ES + RI; SG + MF + FP + ES; | | | | |
| | PS + DS + MF + RI; RT + MF + TB + FP; | | | | |
| | DS + FPL + LA + MS; EL + LA + ES + KQ; | | | | |
| | EC + RT + TB + KQ; DS + LA + AH + KQ; | | | | |
| | DS + MM + AH + FP; PS + RT + LA + TB; | | | | |
| | PS + AH + ES + RI; RT + FP + MS + KQ; | | | | |
| | PS + RT + MM + ES; RT + FPL + LA + RI; | | | | |
| | RT + LA + AH + FP; FPL + LA + MF + TB; | | | | |
| | FPL + MF + TB + MS; EC + PS + AH + RI; | | | | |
| | SG + RT + MM + ES; RT + DS + FPL + AH; | | | | |
| | EL + FP + MS + KQ; EC + AH + FP + KQ; | | | | |
| | TB + AH + FP + RI; FPL + MF + TB + KQ; | | | | |
| | EC + PS + EL + MF; DS + AH + FP + RI; | | | | |
| | FPL + MM + FP + ES; FPL + FP + MS + KQ; | | | | |
| | EC + FPL + MM + KQ; EC + RT + AH + FP; | | | | |
| | SG + DS + FP + RI; DS + MF + AH + FP; | | | | |
| | FPL + EL + MF + ES; FPL + EL + MM + FP; | | | | |
| | LA + AH + FP + KQ; TB + ES + MS + RI; | | | | |
| | FPL + EL + ES + RI; EL + AH + ES + KQ; | | | | |
| | PS + MF + TB + FP; PS + TB + MM + AH; | | | | |
| | RT + MM + FP + KQ; DS + EL + LA + ES; | | | | |
| | FPL + MF + TB + AH; EC + DS + MM + KQ; | | | | |
| | SG + DS + TB + RI; PS + LA + TB + RI; | | | | |
| | PS + DS + EL + AH; SG + PS + FPL + KQ; | | | | |
| | RT + LA + TB + AH; RT + LA + FP + RI; | | | | |
| | EC + LA + MS + RI; PS + FPL + TB + KQ; | | | | |
| | PS + LA + AH + FP; EC + SG + MF + AH; | | | | |
| | EC + FPL + LA + MF; SG + FPL + MM + RI; | | | | |
| | FPL + TB + MM + RI; RT + DS + LA + MF; | | | | |
| | DS + MM + MS + RI; FPL + LA + MM + ES; | | | | |
| | SG + RT + DS + TB; SG + FPL + MM + ES; | | | | |
| | FPL + MM + ES + KQ; EC + RT + LA + MM; | | | | |
| | SG + FP + ES + RI; RT + EL + MF + ES; | | | | |
| | EC + FPL + EL + MF; SG + LA + MM + MS; | | | | |
| | PS + RT + FPL + EL; PS + RT + FPL + FP; | | | | |
| | EC + AH + FP + MS; SG + DS + MS + KQ; | | | | |
| | PS + FPL + ES + RI; DS + EL + ES + RI; | | | | |
| | FPL + MF + FP + RI; LA + MM + AH + FP; | | | | |
| | EC + SG + OS + LA; SG + EL + MM + KQ; | | | | |
| | SG + AH + ES + RI; RT + LA + AH + KQ; | | | | |
| | RT + TB + MM + FP; DS + MM + MS + KQ; | | | | |
| | SG + LA + MM + RI; PS + DS + MF + ES; | | | | |
| | EC + PS + LA + KQ; DS + EL + LA + TB; | | | | |
| | EC + SG + AH + MS; SG + DS + KQ + RI; | | | | |
| | SG + MF + TB + FP; DS + MM + ES + KQ; | | | | |
| | EC + RT + DS + MM; EC + LA + FP + ES; | | | | |
| | EC + RT + MM + RI; SG + FPL + KQ + RI; | | | | |
| | PS + RT + FP + RI; TB + AH + ES + RI; | | | | |
| | EC + PS + DS + OS; EC + DS + LA + FP; | | | | |
| | SG + MM + ES + RI; PS + RT + LA + MM; | | | | |
| | SG + RT + OS + LA; SG + TB + MS + RI; | | | | |
| | DS + MF + AH + RI; EC + SG + MF + MS; | | | | |
| | SG + PS + LA + FP; SG + RT + AH + FP; | | | | |
| | SG + AH + KQ + RI; EC + RT + TB + AH; | | | | |
| | FPL + ES + MS + KQ; RT + MF + FP + MS; | | | | |
| | EL + MM + AH + ES; LA + MF + AH + FP; | | | | |
| | EC + DS + MF + KQ; SG + EL + LA + TB; | | | | |
| | DS + FPL + MF + AH; DS + EL + AH + MS; | | | | |
| | FPL + FP + ES + RI; EC + PS + RT + AH; | | | | |
| | DS + MF + AH + ES; DS + TB + MM + RI; | | | | |
| | EC + SG + RT + MF; RT + FPL + AH + ES; | | | | |
| | EL + TB + AH + KQ; EC + RT + TB + MM; | | | | |
| | RT + DS + FPL + KQ; RT + LA + MM + FP; | | | | |
| | MM + AH + ES + KQ; EC + PS + FPL + FP; | | | | |
| | PS + DS + AH + MS; DS + LA + MS + RI; | | | | |
| | SG + DS + MF + FP; PS + LA + TB + KQ; | | | | |
| | DS + EL + MS + KQ; DS + LA + MF + AH; | | | | |
| | PS + DS + TB + MS; DS + EL + MM + FP; | | | | |
| | SG + RT + MF + RI; RT + TB + MS + RI; | | | | |
| | DS + FPL + EL + AH; DS + EL + MF + TB; | | | | |
| | EC + FPL + LA + KQ; PS + DS + TB + MM; | | | | |
| | EC + FPL + MM + FP; PS + FPL + EL + MM; | | | | |
| | DS + MF + TB + MS; SG + TB + ES + RI; | | | | |
| | SG + EL + FP + MS; PS + DS + ES + MS; | | | | |
| | RT + FPL + LA + MM; FPL + TB + MM + MS; | | | | |
| | EC + FPL + FP + KQ; SG + PS + RT + FP; | | | | |
| | FPL + EL + AH + KQ; FPL + LA + ES + RI; | | | | |
| | LA + TB + AH + RI; EC + EL + MF + FP; | | | | |
| | RT + DS + AH + RI; RT + EL + TB + KQ; | | | | |
| | RT + DS + FP + KQ; SG + PS + EL + MS; | | | | |
| | FPL + EL + ES + KQ; EC + ES + KQ + RI; | | | | |
| | RT + FPL + MF + TB; RT + EL + MF + RI; | | | | |
| | LA + MF + FP + MS; MF + AH + FP + ES; | | | | |
| | TB + AH + ES + KQ; EC + TB + KQ + RI; | | | | |
| | SG + EL + FP + RI; PS + FPL + EL + MF; | | | | |
| | EC + LA + FP + RI; EC + FPL + MF + MS; | | | | |
| | EC + MF + ES + MS; SG + EL + LA + MM; | | | | |
| | EC + RT + AH + KQ; RT + LA + MF + MS; | | | | |
| | EC + RT + LA + MF; EC + FP + KQ + RI; | | | | |
| | PS + RT + AH + ES; EC + PS + RT + MM; | | | | |
| | RT + EL + TB + MS; FPL + LA + FP + KQ; | | | | |
| | EC + FPL + EL + MM; EC + FPL + MF + MM; | | | | |
| | EC + MF + MM + ES; FPL + MF + TB + RI; | | | | |
| | DS + TB + ES + KQ; EC + RT + MF + RI; | | | | |
| | SG + RT + AH + KQ; RT + LA + MM + RI; | | | | |
| | DS + EL + LA + FP; DS + TB + AH + KQ; | | | | |
| | EC + PS + OS + ES; SG + PS + FPL + MF; | | | | |
| | RT + DS + OS + FP; MF + TB + MM + FP; | | | | |
| | EC + EL + TB + AH; EC + TB + AH + MS; | | | | |
| | FPL + LA + MM + FP; FPL + MM + FP + KQ; | | | | |
| | DS + MM + AH + RI; RT + LA + MM + KQ; | | | | |
| | PS + RT + MM + RI; DS + MF + FP + MS; | | | | |
| | EC + RT + MF + MS; SG + TB + ES + MS; | | | | |
| | SG + MM + FP + RI; FPL + LA + TB + FP; | | | | |
| | FPL + LA + ES + MS; DS + AH + ES + KQ; | | | | |
| | SG + FPL + ES + MS; SG + PS + MS + RI; | | | | |
| | SG + FPL + MF + RI; PS + DS + AH + RI; | | | | |
| | EC + LA + AH + MS; DS + EL + FP + KQ; | | | | |
| | FPL + EL + ES + MS; EL + TB + MM + RI; | | | | |
| | PS + DS + EL + MF; RT + DS + FPL + MM; | | | | |
| | RT + FPL + EL + KQ; EL + LA + AH + FP; | | | | |
| | EC + FPL + FP + RI; EC + MF + TB + KQ; | | | | |
| | EC + MM + FP + ES; SG + DS + FP + KQ; | | | | |
| | EL + MM + ES + KQ; EC + FPL + AH + KQ; | | | | |
| | EC + FPL + FP + MS; DS + EL + ES + MS; | | | | |
| | PS + EL + LA + ES; DS + MF + FP + RI; | | | | |
| | EL + AH + ES + MS; MF + TB + FP + KQ; | | | | |
| | EC + PS + FP + RI; EC + EL + LA + FP; | | | | |
| | SG + PS + RT + DS; SG + PS + MM + FP; | | | | |
| | RT + FPL + FP + MS; DS + MS + KQ + RI; | | | | |
| | FPL + MF + FP + KQ; EC + SG + MF + MM; | | | | |
| | EC + PS + OS + KQ; SG + FPL + AH + FP; | | | | |
| | PS + RT + DS + ES; RT + TB + ES + MS; | | | | |
| | DS + LA + MF + ES; DS + MF + TB + ES; | | | | |
| | EC + RT + AH + MS; SG + TB + MM + RI; | | | | |
| | EC + DS + OS + TB; EC + PS + FPL + AH; | | | | |
| | EC + DS + EL + RI; EC + EL + MS + KQ; | | | | |
| | SG + EL + LA + AH; SG + EL + LA + ES; | | | | |
| | SG + MF + TB + AH; FPL + LA + MF + ES; | | | | |
| | EC + RT + MM + MS; EC + MF + AH + FP; | | | | |
| | PS + DS + LA + FP; PS + DS + MM + ES; | | | | |
| | PS + LA + MF + ES; SG + PS + LA + KQ; | | | | |
| | PS + TB + FP + MS; RT + EL + LA + MF; | | | | |
| | EC + PS + RT + MF; EC + LA + MF + AH; | | | | |
| | RT + EL + KQ + RI; FPL + EL + TB + AH; | | | | |
| | SG + PS + MM + RI; RT + EL + LA + MM; | | | | |
| | EC + PS + MF + KQ; PS + LA + MM + RI; | | | | |
| | RT + DS + OS + LA; RT + LA + MF + RI; | | | | |
| | EL + LA + FP + MS; LA + FP + MS + RI; | | | | |
| | AH + FP + ES + MS; EC + EL + MM + FP; | | | | |
| | SG + FPL + MF + MM; PS + EL + AH + KQ; | | | | |
| | EC + PS + FP + KQ; SG + MM + FP + KQ; | | | | |
| | PS + AH + ES + MS; DS + MM + ES + RI; | | | | |
| | SG + FPL + MS + KQ; RT + AH + ES + KQ; | | | | |
| | DS + EL + AH + KQ; EC + EL + AH + FP; | | | | |
| | EC + MM + FP + MS; SG + PS + RT + ES; | | | | |
| | SG + MF + AH + RI; EL + AH + FP + KQ | | | | |
| 4 | EC + DS + AH + RI; RT + FPL + LA + TB; | 0.74 | 0.2921- | 46.03%- | 45.45%- |
| | RT + LA + MS + RI; DS + LA + AH + MS; | | 0.5235 | 93.65% | 89.90% |
| | EC + FPL + MM + ES; SG + DS + MS + RI; | | | | |
| | RT + FPL + EL + TB; RT + FPL + KQ + RI; | | | | |
| | DS + EL + TB + AH; EC + SG + RT + AH; | | | | |
| | SG + FPL + LA + MM; EC + SG + RT + MS; | | | | |
| | SG + PS + TB + AH; SG + RT + DS + RI; | | | | |
| | EC + DS + LA + KQ; RT + LA + KQ + RI; | | | | |
| | SG + RT + MM + RI; PS + DS + LA + TB; | | | | |
| | DS + MF + FP + ES; LA + MM + ES + MS; | | | | |
| | EC + SG + AH + RI; EC + LA + ES + RI; | | | | |
| | EC + RT + MM + ES; SG + RT + DS + EL; | | | | |
| | PS + EL + MF + ES; EC + SG + MF + RI; | | | | |
| | EC + RT + FPL + EL; EC + DS + MM + AH; | | | | |
| | EC + DS + MM + ES; EC + DS + AH + MS; | | | | |
| | SG + RT + EL + RI; PS + DS + TB + RI; | | | | |
| | DS + FPL + EL + RI; EL + AH + FP + ES; | | | | |
| | EC + PS + MM + AH; SG + PS + MM + AH; | | | | |
| | DS + TB + AH + MS; MF + TB + FP + MS; | | | | |
| | EC + PS + EL + TB; SG + PS + FPL + RI; | | | | |
| | LA + FP + ES + RI; EC + SG + FPL + MM; | | | | |
| | EC + DS + MS + KQ; EC + PS + LA + MM; | | | | |
| | EC + RT + MF + ES; EC + DS + FP + RI; | | | | |
| | SG + LA + FP + RI; EC + PS + DS + FP; | | | | |
| | EC + DS + EL + FP; EC + TB + AH + KQ; | | | | |
| | SG + DS + AH + KQ; SG + FPL + TB + MM; | | | | |
| | EC + FPL + ES + KQ; DS + AH + ES + MS; | | | | |
| | EC + RT + FPL + RI; DS + LA + TB + MM; | | | | |
| | EC + PS + FPL + RI; EC + FPL + TB + FP; | | | | |
| | PS + TB + MM + RI; SG + RT + TB + RI; | | | | |
| | EL + MM + FP + ES; RT + LA + FP + KQ; | | | | |
| | FPL + TB + MS + KQ; RT + EL + LA + FP; | | | | |
| | EC + MF + FP + ES; SG + FPL + MM + FP; | | | | |
| | SG + FP + MS + KQ; RT + FPL + MF + FP; | | | | |
| | EC + FPL + TB + AH; EC + DS + MM + RI; | | | | |
| | RT + DS + FPL + MS; RT + LA + TB + KQ; | | | | |
| | FPL + TB + AH + KQ; LA + AH + ES + MS; | | | | |
| | MM + AH + FP + ES; SG + MF + AH + ES; | | | | |
| | SG + RT + MF + FP; DS + MF + ES + RI; | | | | |
| | DS + FP + ES + RI; EC + FPL + TB + RI; | | | | |
| | SG + PS + MM + ES; DS + FP + MS + RI; | | | | |
| | DS + LA + AH + RI; PS + FPL + EL + ES; | | | | |
| | PS + EL + MM + KQ; EC + SG + RT + KQ; | | | | |
| | SG + RT + DS + MS; PS + LA + TB + MM; | | | | |
| | EL + MF + FP + ES; RT + LA + TB + MS; | | | | |
| | RT + TB + FP + RI; EC + PS + MF + FP; | | | | |
| | EC + PS + MF + MS; EC + FPL + ES + MS; | | | | |
| | RT + FP + MS + RI; SG + LA + MF + FP; | | | | |
| | SG + FPL + EL + MM; SG + TB + FP + MS; | | | | |
| | PS + RT + EL + KQ; DS + EL + LA + AH; | | | | |
| | LA + TB + ES + MS; EC + SG + EL + KQ; | | | | |
| | PS + LA + TB + AH; LA + MM + AH + ES; | | | | |
| | EC + PS + FPL + KQ; RT + DS + LA + TB; | | | | |
| | RT + FPL + FP + RI; DS + FPL + MM + RI; | | | | |
| | FPL + LA + AH + FP; PS + FPL + TB + FP; | | | | |
| | EC + PS + RT + DS; EC + PS + MF + AH; | | | | |
| | EC + FPL + MF + ES; EC + EL + TB + MM; | | | | |
| | SG + LA + ES + MS; EC + EL + TB + MS; | | | | |
| | RT + EL + FP + RI; DS + MF + TB + AH; | | | | |
| | EC + SG + FP + KQ; EC + RT + FP + KQ; | | | | |
| | EC + DS + AH + KQ; EC + AH + ES + MS; | | | | |
| | SG + PS + DS + ES; DS + MF + TB + RI; | | | | |
| | EC + SG + EL + MF; SG + PS + FPL + ES; | | | | |
| | SG + DS + EL + RI; PS + MM + AH + ES; | | | | |
| | DS + FPL + FP + MS; EC + MF + TB + MM; | | | | |
| | EC + TB + MM + RI; PS + RT + EL + LA; | | | | |
| | PS + DS + TB + AH; TB + AH + FP + KQ; | | | | |
| | PS + RT + EL + RI; EC + RT + EL + FP; | | | | |
| | EC + RT + MS + KQ; DS + EL + MF + ES; | | | | |
| | EC + RT + LA + AH; EC + EL + ES + MS; | | | | |
| | EC + DS + FP + KQ; SG + LA + TB + RI; | | | | |
| | RT + MF + ES + RI; DS + FP + MS + KQ; | | | | |
| | LA + MM + ES + KQ; EC + SG + KQ + RI; | | | | |
| | EC + PS + FPL + EL; EC + PS + MF + RI; | | | | |
| | EC + RT + DS + MF; EC + EL + LA + ES; | | | | |
| | EC + EL + AH + ES; PS + DS + TB + FP; | | | | |
| | EC + FP + MS + KQ; RT + FPL + TB + FP; | | | | |
| | RT + LA + TB + RI; EC + TB + MS + RI; | | | | |
| | TB + FP + KQ + RI; SG + RT + AH + ES; | | | | |
| | PS + EL + MS + KQ; LA + MF + TB + FP; | | | | |
| | EC + DS + EL + AH; RT + DS + FPL + FP; | | | | |
| | DS + LA + ES + KQ; SG + FPL + EL + RI; | | | | |
| | DS + FPL + MS + KQ; LA + AH + ES + KQ; | | | | |
| | EC + SG + FPL + MF; EC + PS + EL + MM; | | | | |
| | DS + EL + TB + MS; TB + MM + FP + RI; | | | | |
| | EC + LA + TB + FP; DS + EL + MF + RI; | | | | |
| | EC + RT + DS + KQ; SG + FPL + EL + MF; | | | | |
| | TB + MM + MS + RI; EC + PS + FPL + MF; | | | | |
| | SG + TB + MM + ES; EC + RT + DS + EL; | | | | |
| | DS + FPL + LA + MM; DS + EL + AH + FP; | | | | |
| | DS + LA + FP + RI; FPL + MF + FP + ES; | | | | |
| | EC + RT + EL + MS; PS + DS + LA + RI; | | | | |
| | EL + TB + MM + KQ; EC + FPL + FP + ES; | | | | |
| | FPL + EL + MM + ES; SG + PS + FPL + LA; | | | | |
| | SG + AH + FP + MS; EC + RT + FPL + MF; | | | | |
| | EC + LA + MS + KQ; RT + DS + LA + ES; | | | | |
| | RT + FPL + MF + ES; LA + ES + MS + KQ; | | | | |
| | EC + PS + LA + RI; EC + TB + MM + AH; | | | | |
| | RT + DS + KQ + RI; DS + MM + FP + ES; | | | | |
| | EC + ES + MS + KQ; SG + RT + ES + KQ; | | | | |
| | RT + EL + FP + MS; DS + LA + MS + KQ; | | | | |
| | EC + PS + LA + MF; EC + FPL + LA + FP; | | | | |
| | SG + RT + LA + TB; RT + FP + KQ + RI; | | | | |
| | DS + EL + FP + RI; EC + RT + DS + AH; | | | | |
| | RT + FPL + EL + FP; SG + RT + EL + KQ; | | | | |
| | RT + LA + MF + ES; DS + LA + MM + MS; | | | | |
| | FPL + FP + ES + KQ; SG + DS + TB + ES; | | | | |
| | EC + RT + FP + RI; EC + ES + MS + RI; | | | | |
| | SG + FPL + EL + LA; DS + LA + MM + AH; | | | | |
| | EC + EL + MF + ES; EC + FP + ES + RI; | | | | |
| | DS + FPL + FP + KQ; LA + MF + TB + AH; | | | | |
| | EC + SG + MF + TB; EC + SG + MM + KQ; | | | | |
| | EC + EL + TB + KQ; EC + PS + OS + TB; | | | | |
| | EC + DS + EL + LA; DS + FPL + TB + MM; | | | | |
| | SG + DS + TB + MS; EC + PS + KQ + RI; | | | | |
| | EC + DS + FPL + MS; PS + EL + TB + MS; | | | | |
| | EC + RT + TB + RI; EC + FPL + EL + FP; | | | | |
| | SG + EL + LA + KQ; PS + RT + LA + MS; | | | | |
| | RT + DS + TB + RI; DS + FPL + AH + KQ; | | | | |
| | DS + FP + ES + MS; FPL + LA + FP + RI; | | | | |
| | EL + TB + MS + KQ; EC + FPL + TB + MS; | | | | |
| | RT + MS + KQ + RI; EC + MF + ES + RI; | | | | |
| | EC + MM + ES + RI; PS + MM + ES + MS; | | | | |
| | DS + FPL + MF + MS; FPL + EL + TB + RI; | | | | |
| | EC + RT + LA + RI; SG + FPL + TB + RI; | | | | |
| | SG + MM + AH + KQ; PS + RT + KQ + RI; | | | | |
| | RT + EL + MM + ES; SG + FPL + ES + RI; | | | | |
| | PS + DS + FPL + FP; FPL + EL + FP + KQ; | | | | |
| | EL + LA + TB + AH; PS + RT + LA + AH; | | | | |
| | EC + PS + RT + KQ; EC + RT + MF + FP; | | | | |
| | EC + DS + MM + FP; EC + FP + ES + KQ; | | | | |
| | FPL + AH + FP + ES; LA + FP + ES + MS; | | | | |
| | EC + MF + TB + AH; SG + DS + LA + MM; | | | | |
| | RT + EL + LA + RI; EC + MF + TB + RI; | | | | |
| | SG + DS + MF + AH; SG + EL + AH + ES; | | | | |
| | EC + SG + FPL + RI; SG + FP + ES + KQ; | | | | |
| | RT + DS + LA + KQ; EC + PS + RT + MS; | | | | |
| | SG + RT + MM + FP; SG + DS + MF + RI; | | | | |
| | LA + MM + ES + RI; EC + EL + ES + KQ; | | | | |
| | RT + FPL + EL + LA; RT + LA + MM + ES; | | | | |
| | EC + LA + TB + MM; SG + RT + FPL + EL; | | | | |
| | SG + RT + ES + MS; PS + DS + FP + ES; | | | | |
| | EC + RT + EL + RI; PS + EL + TB + KQ; | | | | |
| | RT + FPL + TB + RI; EC + LA + FP + MS; | | | | |
| | SG + DS + EL + KQ; RT + EL + MS + RI; | | | | |
| | EL + FP + ES + RI; TB + MM + FP + ES; | | | | |
| | EC + FPL + MF + FP; EC + LA + MM + ES; | | | | |
| | EC + MF + FP + MS; PS + FPL + TB + MM; | | | | |
| | RT + DS + MF + FP; FPL + EL + LA + TB; | | | | |
| | EC + TB + MM + MS; PS + RT + FPL + LA; | | | | |
| | EC + EL + MM + ES; PS + DS + MS + RI; | | | | |
| | PS + FPL + LA + ES; DS + FP + ES + KQ; | | | | |
| | FPL + LA + AH + ES; EC + EL + FP + MS; | | | | |
| | SG + TB + AH + RI; PS + TB + ES + MS; | | | | |
| | LA + MM + MS + KQ; EC + EL + ES + RI; | | | | |
| | SG + FPL + AH + ES; PS + RT + MS + RI; | | | | |
| | DS + FPL + EL + MS; SG + DS + FP + MS; | | | | |
| | EC + DS + MF + MM; SG + PS + TB + RI; | | | | |
| | SG + FPL + LA + AH; RT + DS + LA + AH; | | | | |
| | FPL + MF + TB + MM; FPL + FP + ES + MS; | | | | |
| | LA + AH + FP + MS; EC + PS + RT + TB; | | | | |
| | RT + DS + MF + RI; MF + TB + AH + ES; | | | | |
| | EC + PS + MM + RI; EC + RT + EL + KQ; | | | | |
| | SG + RT + FPL + KQ; FPL + TB + FP + MS; | | | | |
| | RT + EL + MF + FP; EC + FPL + EL + AH; | | | | |
| | EC + FP + MS + RI; SG + PS + RT + FPL; | | | | |
| | SG + TB + AH + KQ; LA + ES + MS + RI; | | | | |
| | PS + RT + DS + LA; RT + EL + TB + RI; | | | | |
| | DS + MM + AH + ES; EL + LA + TB + ES; | | | | |
| | EC + SG + TB + KQ; PS + RT + TB + RI; | | | | |
| | RT + LA + MM + MS; FPL + EL + TB + MS; | | | | |
| | EL + FP + ES + KQ; SG + PS + FPL + EL; | | | | |
| | SG + TB + FP + RI; EC + SG + EL + RI; | | | | |
| | EC + PS + EL + AH; EC + DS + MM + MS; | | | | |
| | EC + FPL + MF + AH; PS + FPL + MF + FP; | | | | |
| | PS + LA + AH + KQ; RT + FPL + EL + RI; | | | | |
| | EC + RT + KQ + RI; MM + AH + ES + MS; | | | | |
| | EC + MM + ES + KQ; SG + RT + TB + ES; | | | | |
| | SG + FPL + LA + MS; DS + LA + MF + RI; | | | | |
| | EC + DS + LA + MM; FPL + LA + FP + ES; | | | | |
| | SG + DS + TB + FP; PS + LA + ES + MS; | | | | |
| | EC + PS + EL + FP; EC + EL + FP + KQ; | | | | |
| | EC + LA + AH + FP; EC + MF + TB + MS; | | | | |
| | SG + PS + RT + EL; SG + LA + KQ + RI; | | | | |
| | SG + FPL + LA + MF; RT + DS + FP + RI; | | | | |
| | RT + LA + MF + FP; LA + MF + AH + ES; | | | | |
| | DS + MF + ES + MS; EC + RT + EL + TB; | | | | |
| | PS + DS + EL + FP; PS + DS + AH + FP; | | | | |
| | SG + DS + ES + MS; RT + FPL + ES + MS; | | | | |
| | EC + DS + EL + KQ; RT + DS + EL + MS; | | | | |
| | EC + PS + AH + FP; EC + FPL + AH + FP; | | | | |
| | SG + TB + AH + MS; PS + RT + FPL + RI; | | | | |
| | EC + FPL + ES + RI; FPL + LA + ES + KQ; | | | | |
| | EC + RT + MM + FP; EC + EL + MF + TB; | | | | |
| | EC + LA + AH + KQ; SG + DS + EL + AH; | | | | |
| | SG + TB + FP + KQ; DS + EL + AH + RI; | | | | |
| | FPL + AH + ES + KQ | | | | |
| 4 | EC + SG + PS + FPL; EL + TB + ES + KQ; | 0.75 | 0.272- | 50.79%- | 40.40%- |
| | SG + FPL + ES + KQ; DS + FPL + LA + MF; | | 0.5071 | 95.24% | 88.89% |
| | SG + RT + DS + FPL; EC + RT + TB + FP; | | | | |
| | SG + DS + LA + RI; DS + MM + FP + MS; | | | | |
| | TB + FP + ES + KQ; SG + RT + FPL + LA; | | | | |
| | EC + PS + DS + MM; EC + RT + ES + KQ; | | | | |
| | SG + PS + AH + ES; MF + TB + FP + ES; | | | | |
| | FPL + EL + TB + KQ; EC + SG + MM + FP; | | | | |
| | SG + LA + MS + KQ; DS + FPL + TB + AH; | | | | |
| | EC + DS + FP + MS; SG + FPL + LA + ES; | | | | |
| | SG + LA + FP + MS; SG + PS + DS + AH; | | | | |
| | SG + PS + EL + MM; EC + DS + EL + MF; | | | | |
| | EC + PS + DS + EL; EC + PS + FP + MS; | | | | |
| | DS + FPL + MS + RI; EC + SG + MM + RI; | | | | |
| | EC + SG + MF + FP; EC + AH + ES + KQ; | | | | |
| | PS + FPL + EL + TB; TB + MM + ES + RI; | | | | |
| | SG + DS + FPL + MM; EC + SG + TB + MS; | | | | |
| | SG + DS + LA + MS; RT + FP + ES + RI; | | | | |
| | DS + FPL + MM + ES; EC + RT + DS + ES; | | | | |
| | SG + DS + LA + FP; EC + MF + TB + FP; | | | | |
| | RT + LA + ES + MS; EC + SG + RT + MM; | | | | |
| | DS + FPL + LA + FP; EC + SG + EL + TB; | | | | |
| | SG + LA + MM + ES; PS + DS + ES + RI; | | | | |
| | EC + RT + DS + LA; SG + PS + DS + LA; | | | | |
| | SG + RT + FPL + ES; RT + DS + EL + ES; | | | | |
| | SG + RT + FPL + RI; DS + FPL + FP + RI; | | | | |
| | DS + FPL + TB + MS; EC + SG + TB + RI; | | | | |
| | EC + DS + FPL + FP; SG + PS + RT + LA; | | | | |
| | DS + FPL + EL + KQ; DS + AH + ES + RI; | | | | |
| | EC + DS + FPL + EL; SG + RT + MS + RI; | | | | |
| | SG + LA + MM + FP; SG + PS + AH + FP; | | | | |
| | RT + MM + ES + RI; DS + FPL + EL + MM; | | | | |
| | EC + DS + FPL + MM; TB + FP + MS + KQ; | | | | |
| | EC + LA + MF + ES; SG + DS + LA + TB; | | | | |
| | DS + FPL + AH + FP; EC + RT + LA + MS; | | | | |
| | EC + RT + TB + ES; EC + AH + FP + ES; | | | | |
| | FPL + TB + MM + FP; EC + EL + FP + ES; | | | | |
| | RT + MF + FP + ES; EC + FPL + MF + TB; | | | | |
| | EL + MM + ES + MS; EC + LA + MF + TB; | | | | |
| | RT + FPL + FP + ES; DS + FPL + EL + LA; | | | | |
| | PS + DS + FPL + EL; DS + TB + FP + KQ; | | | | |
| | EC + EL + LA + TB; PS + DS + FPL + MF; | | | | |
| | PS + EL + FP + ES; RT + EL + LA + TB; | | | | |
| | SG + RT + KQ + RI; SG + LA + TB + MM; | | | | |
| | EC + DS + LA + RI; EC + LA + ES + MS; | | | | |
| | LA + MM + FP + ES; SG + FPL + FP + MS; | | | | |
| | SG + LA + FP + KQ; PS + LA + FP + ES; | | | | |
| | DS + FPL + LA + RI; EC + RT + DS + RI; | | | | |
| | SG + PS + ES + RI; SG + RT + FP + RI; | | | | |
| | SG + TB + MM + FP; EC + PS + RT + LA; | | | | |
| | EC + LA + TB + RI; LA + FP + MS + KQ; | | | | |
| | DS + TB + MS + RI; EC + SG + PS + EL; | | | | |
| | SG + RT + LA + MM; DS + FPL + ES + MS; | | | | |
| | RT + EL + ES + MS; EC + DS + LA + MF; | | | | |
| | EC + FPL + AH + ES; EC + LA + ES + KQ; | | | | |
| | LA + TB + ES + KQ; EC + PS + DS + KQ; | | | | |
| | LA + TB + AH + MS; EC + PS + LA + AH; | | | | |
| | SG + TB + MM + AH; PS + EL + AH + ES; | | | | |
| | RT + LA + ES + RI; EL + TB + FP + RI; | | | | |
| | PS + DS + FPL + LA; EC + SG + MS + RI; | | | | |
| | DS + LA + FP + MS; EC + RT + FPL + TB; | | | | |
| | RT + LA + TB + FP; RT + FP + ES + KQ; | | | | |
| | EC + DS + EL + MS; PS + RT + ES + RI; | | | | |
| | DS + LA + TB + KQ; TB + FP + ES + RI; | | | | |
| | PS + FPL + MF + TB; EC + FPL + LA + ES; | | | | |
| | SG + DS + FPL + MS; SG + EL + TB + ES; | | | | |
| | EC + SG + PS + RT; DS + LA + AH + FP; | | | | |
| | EC + PS + FPL + MM; PS + TB + MM + MS; | | | | |
| | SG + LA + ES + RI; EL + LA + TB + KQ; | | | | |
| | SG + MM + FP + ES; RT + EL + LA + KQ; | | | | |
| | DS + ES + MS + RI; SG + FPL + EL + ES; | | | | |
| | RT + DS + ES + MS; FPL + LA + TB + MM; | | | | |
| | RT + DS + EL + LA; RT + TB + ES + RI; | | | | |
| | DS + FPL + MF + KQ; LA + MF + FP + ES; | | | | |
| | LA + FP + ES + KQ; EC + RT + FP + MS; | | | | |
| | EC + DS + ES + KQ; PS + TB + ES + RI; | | | | |
| | EL + LA + TB + FP; EC + SG + RT + FPL; | | | | |
| | EC + RT + DS + FP; EC + RT + MS + RI; | | | | |
| | EC + TB + MM + KQ; RT + DS + EL + RI; | | | | |
| | RT + DS + FP + MS; DS + LA + ES + MS; | | | | |
| | FPL + TB + ES + RI; RT + EL + MS + KQ; | | | | |
| | DS + LA + ES + RI; EC + SG + RT + EL; | | | | |
| | EC + FPL + EL + TB; PS + RT + DS + RI; | | | | |
| | EC + SG + TB + AH; EC + RT + FPL + LA; | | | | |
| | SG + DS + LA + KQ; DS + TB + AH + RI; | | | | |
| | SG + RT + EL + ES; RT + ES + MS + RI; | | | | |
| | SG + PS + EL + KQ; SG + RT + DS + FP; | | | | |
| | SG + FPL + LA + KQ; FPL + EL + AH + ES; | | | | |
| | EL + FP + ES + MS; EC + SG + RT + TB; | | | | |
| | EC + PS + MM + KQ; SG + DS + MM + AH; | | | | |
| | PS + DS + FPL + KQ; RT + LA + FP + MS; | | | | |
| | DS + TB + MM + MS; LA + TB + AH + KQ; | | | | |
| | EC + SG + RT + FP; EC + RT + FPL + FP; | | | | |
| | EC + TB + MS + KQ; PS + AH + FP + ES; | | | | |
| | DS + FPL + LA + ES; SG + MF + AH + FP; | | | | |
| | FPL + TB + ES + MS; EC + RT + LA + TB; | | | | |
| | EC + DS + FPL + MF; SG + FPL + TB + KQ; | | | | |
| | RT + FPL + LA + KQ; EC + FPL + EL + ES; | | | | |
| | RT + DS + LA + RI; FPL + TB + MM + KQ; | | | | |
| | EC + DS + FPL + AH; SG + AH + FP + KQ; | | | | |
| | SG + AH + ES + MS; EC + DS + FP + ES; | | | | |
| | EC + DS + FPL + KQ; LA + MF + TB + ES; | | | | |
| | RT + DS + EL + FP; EC + SG + EL + LA; | | | | |
| | EC + DS + MF + MS; EC + DS + KQ + RI; | | | | |
| | RT + DS + TB + FP; EC + SG + AH + KQ; | | | | |
| | EC + PS + LA + FP; SG + EL + AH + FP; | | | | |
| | DS + FPL + LA + AH; DS + FPL + MF + FP; | | | | |
| | SG + FPL + TB + MS; RT + DS + LA + MS; | | | | |
| | EC + RT + AH + ES; SG + RT + FP + MS; | | | | |
| | EC + FPL + TB + KQ; FPL + LA + TB + AH; | | | | |
| | SG + LA + MM + KQ; SG + TB + MM + MS; | | | | |
| | RT + DS + FPL + TB; EC + FPL + LA + TB; | | | | |
| | PS + FPL + MF + ES; TB + AH + FP + MS; | | | | |
| | SG + RT + FP + KQ; PS + FPL + LA + TB; | | | | |
| | RT + EL + TB + FP; RT + FP + ES + MS; | | | | |
| | DS + TB + ES + MS; FPL + LA + TB + KQ; | | | | |
| | SG + RT + DS + ES; SG + FPL + MM + KQ; | | | | |
| | EC + SG + FPL + FP; EC + SG + FPL + MS; | | | | |
| | EC + DS + FPL + LA; SG + PS + FPL + TB; | | | | |
| | DS + TB + MM + FP; EC + MM + AH + ES; | | | | |
| | EC + SG + EL + AH; EC + PS + RT + FPL; | | | | |
| | SG + RT + TB + FP; RT + DS + ES + RI; | | | | |
| | EC + SG + MS + KQ; PS + DS + LA + AH; | | | | |
| | PS + LA + TB + FP; MF + TB + FP + RI; | | | | |
| | EC + DS + AH + FP; PS + FPL + TB + RI; | | | | |
| | PS + LA + ES + KQ; RT + TB + FP + MS; | | | | |
| | DS + EL + MF + FP; LA + TB + MM + AH; | | | | |
| | EC + RT + DS + TB; EC + MF + AH + ES; | | | | |
| | SG + LA + MF + AH; DS + FPL + LA + KQ; | | | | |
| | DS + FPL + KQ + RI; TB + MM + FP + KQ; | | | | |
| | TB + AH + ES + MS; EC + SG + MM + AH; | | | | |
| | EC + DS + MF + AH; SG + EL + ES + KQ; | | | | |
| | SG + DS + FP + ES; SG + MM + AH + FP; | | | | |
| | DS + TB + MM + ES; EC + RT + LA + KQ; | | | | |
| | RT + EL + LA + MS; RT + MM + FP + ES; | | | | |
| | DS + TB + MM + AH; EC + PS + RT + RI; | | | | |
| | EC + PS + AH + KQ; PS + TB + AH + FP; | | | | |
| | RT + LA + MS + KQ; EC + FP + ES + MS; | | | | |
| | DS + AH + FP + ES; EC + PS + EL + MS; | | | | |
| | EC + PS + EL + KQ; SG + PS + LA + RI; | | | | |
| | SG + RT + FPL + TB; PS + TB + FP + ES; | | | | |
| | EC + SG + FPL + EL; EC + RT + TB + MS; | | | | |
| | RT + TB + FP + KQ; DS + LA + FP + KQ; | | | | |
| | SG + RT + LA + MF; RT + DS + FPL + LA; | | | | |
| | EL + LA + AH + ES; SG + FPL + EL + KQ; | | | | |
| | SG + FPL + MF + ES; PS + RT + LA + RI; | | | | |
| | RT + DS + LA + FP; RT + FPL + LA + FP; | | | | |
| | PS + RT + FPL + ES; EL + MF + TB + ES; | | | | |
| | EC + PS + MS + RI; EC + RT + LA + FP; | | | | |
| | PS + FPL + MM + ES; RT + EL + FP + KQ; | | | | |
| | LA + TB + MM + RI; RT + DS + FPL + MF; | | | | |
| | DS + EL + TB + ES | | | | |
| 4 | SG + LA + AH + RI; EC + RT + FPL + ES; | 0.76 | 0.3196- | 52.38%- | 45.45%- |
| | DS + FPL + MF + ES; TB + MM + FP + MS; | | 0.4933 | 95.24% | 88.89% |
| | EC + SG + FPL + TB; EC + PS + DS + RI; | | | | |
| | EC + PS + ES + MS; DS + EL + FP + ES; | | | | |
| | LA + TB + MM + KQ; EC + PS + FPL + ES; | | | | |
| | EC + DS + TB + FP; DS + FPL + AH + ES; | | | | |
| | SG + AH + FP + ES; EC + EL + TB + ES; | | | | |
| | RT + DS + FPL + ES; DS + FPL + MF + RI; | | | | |
| | SG + PS + EL + RI; SG + LA + TB + MS; | | | | |
| | EC + SG + DS + AH; SG + RT + FP + ES; | | | | |
| | RT + FPL + ES + KQ; RT + EL + ES + RI; | | | | |
| | RT + EL + FP + ES; SG + FPL + EL + TB; | | | | |
| | DS + TB + FP + RI; EC + SG + EL + FP; | | | | |
| | PS + EL + ES + RI; DS + TB + AH + FP; | | | | |
| | EC + PS + DS + MS; RT + FPL + EL + ES; | | | | |
| | EC + TB + ES + MS; RT + TB + FP + ES; | | | | |
| | EC + DS + FPL + ES; SG + DS + EL + FP; | | | | |
| | TB + FP + MS + RI; EC + DS + ES + RI; | | | | |
| | SG + PS + DS + FPL; SG + FPL + MF + TB; | | | | |
| | DS + LA + AH + ES; EC + RT + LA + ES; | | | | |
| | SG + PS + EL + FP; EL + TB + ES + MS; | | | | |
| | DS + FPL + FP + ES; FPL + EL + FP + ES; | | | | |
| | EC + DS + MF + ES; EC + SG + RT + DS; | | | | |
| | SG + DS + AH + FP; SG + LA + TB + ES; | | | | |
| | MF + TB + AH + FP; EC + PS + DS + MF; | | | | |
| | EC + SG + PS + TB; SG + EL + ES + RI; | | | | |
| | PS + DS + TB + ES; EC + SG + RT + RI; | | | | |
| | EC + SG + FPL + AH; EC + SG + DS + MM; | | | | |
| | EC + SG + FP + RI; EC + FPL + TB + MM; | | | | |
| | PS + LA + MM + ES; EC + DS + EL + ES; | | | | |
| | EC + DS + EL + TB; PS + DS + FPL + TB; | | | | |
| | RT + LA + ES + KQ; FPL + EL + TB + MM; | | | | |
| | EC + SG + DS + EL; EC + SG + TB + MM; | | | | |
| | SG + EL + TB + FP; RT + EL + LA + ES; | | | | |
| | RT + LA + TB + ES; FPL + TB + FP + KQ; | | | | |
| | RT + ES + KQ + RI; DS + FPL + EL + MF; | | | | |
| | EC + PS + TB + KQ; SG + EL + MF + FP; | | | | |
| | DS + FPL + LA + TB; PS + RT + EL + ES; | | | | |
| | TB + MM + AH + ES; EC + SG + ES + KQ; | | | | |
| | SG + FPL + FP + KQ; DS + FPL + TB + FP; | | | | |
| | FPL + MF + TB + ES; LA + TB + FP + RI; | | | | |
| | EC + SG + AH + FP; EC + PS + TB + FP; | | | | |
| | EC + DS + AH + ES; EC + RT + DS + FPL; | | | | |
| | EC + SG + ES + MS; PS + FPL + TB + ES; | | | | |
| | EC + FPL + TB + ES; EC + TB + FP + RI; | | | | |
| | DS + LA + FP + ES; EC + PS + FPL + TB; | | | | |
| | FPL + TB + FP + ES; EL + TB + AH + ES; | | | | |
| | SG + PS + DS + RI; EC + DS + FPL + TB; | | | | |
| | SG + RT + ES + RI; SG + LA + TB + FP; | | | | |
| | EC + PS + RT + ES; SG + LA + TB + KQ; | | | | |
| | FPL + TB + AH + FP; EC + SG + DS + FP; | | | | |
| | RT + DS + FPL + EL; LA + TB + FP + MS; | | | | |
| | EC + TB + ES + RI; TB + MM + AH + FP; | | | | |
| | SG + RT + LA + AH; SG + LA + AH + MS; | | | | |
| | LA + TB + FP + KQ; EC + PS + DS + FPL; | | | | |
| | EC + TB + MM + FP; PS + LA + ES + RI; | | | | |
| | EL + TB + MM + MS; EC + SG + DS + KQ; | | | | |
| | EC + SG + PS + MF; EC + EL + TB + FP; | | | | |
| | RT + EL + TB + ES; EC + DS + LA + MS; | | | | |
| | EC + PS + TB + RI; EC + DS + MS + RI; | | | | |
| | SG + MM + AH + ES; EC + SG + DS + TB; | | | | |
| | SG + EL + LA + FP; EC + SG + LA + FP; | | | | |
| | SG + RT + DS + LA; EC + LA + AH + ES; | | | | |
| | PS + DS + FPL + ES; EC + PS + MF + TB; | | | | |
| | EC + TB + ES + KQ; EL + TB + AH + FP; | | | | |
| | RT + FPL + ES + RI; TB + FP + ES + MS; | | | | |
| | SG + EL + MM + FP; EC + DS + TB + MM; | | | | |
| | SG + RT + EL + LA; FPL + EL + MF + TB; | | | | |
| | EC + RT + ES + MS; LA + TB + MM + FP; | | | | |
| | EC + SG + PS + KQ; EC + LA + TB + KQ; | | | | |
| | EC + DS + LA + AH; EC + SG + FPL + KQ; | | | | |
| | EC + DS + MF + RI; SG + LA + ES + KQ; | | | | |
| | DS + FPL + MM + KQ; SG + DS + AH + MS; | | | | |
| | SG + EL + FP + KQ; SG + PS + RT + RI; | | | | |
| | SG + FPL + LA + RI; EC + PS + MS + KQ; | | | | |
| | EC + DS + TB + AH; EC + DS + ES + MS; | | | | |
| | EC + TB + FP + KQ; PS + LA + AH + ES; | | | | |
| | RT + DS + MS + RI; EC + DS + MF + FP; | | | | |
| | EC + RT + ES + RI; LA + TB + MS + RI; | | | | |
| | SG + PS + FPL + FP; DS + FPL + ES + KQ; | | | | |
| | FPL + TB + FP + RI; PS + TB + AH + ES; | | | | |
| | RT + DS + FPL + RI; RT + LA + AH + ES; | | | | |
| | EC + SG + DS + MF; LA + AH + FP + ES; | | | | |
| | EC + DS + FPL + RI; RT + EL + ES + KQ; | | | | |
| | SG + RT + EL + FP; RT + FPL + TB + ES; | | | | |
| | DS + EL + AH + ES; EC + SG + DS + FPL; | | | | |
| | DS + MF + TB + FP; EC + PS + FP + ES; | | | | |
| | SG + RT + LA + MS; SG + FPL + AH + KQ; | | | | |
| | PS + RT + LA + KQ; EC + RT + EL + ES; | | | | |
| | PS + EL + TB + FP; EC + PS + TB + AH; | | | | |
| | EC + PS + LA + MS; EC + RT + DS + MS; | | | | |
| | EC + TB + AH + FP; FPL + MF + TB + FP; | | | | |
| | EC + SG + LA + MF; EC + PS + DS + AH; | | | | |
| | EC + RT + FP + ES; EC + LA + TB + AH; | | | | |
| | RT + DS + FP + ES; EC + DS + TB + KQ; | | | | |
| | SG + AH + ES + KQ; RT + FPL + LA + ES; | | | | |
| | SG + PS + LA + ES; PS + DS + AH + ES | | | | |
| 4 | SG + FPL + LA + FP; SG + EL + MM + ES; | 0.77 | 0.3254- | 49.21%- | 47.47%- |
| | DS + FPL + TB + RI; SG + DS + FPL + LA; | | 0.511 | 98.41% | 91.92% |
| | EC + SG + LA + MM; EC + SG + TB + ES; | | | | |
| | EC + DS + TB + RI; DS + LA + TB + MS; | | | | |
| | FPL + EL + TB + ES; EC + LA + TB + MS; | | | | |
| | FPL + TB + MM + ES; DS + LA + TB + ES; | | | | |
| | SG + FPL + FP + RI; PS + TB + MM + ES; | | | | |
| | EC + PS + DS + TB; PS + LA + TB + ES; | | | | |
| | EL + TB + MM + FP; EC + SG + DS + MS; | | | | |
| | SG + DS + FPL + AH; DS + LA + TB + FP; | | | | |
| | EC + PS + ES + KQ; SG + DS + FPL + KQ; | | | | |
| | SG + LA + TB + AH; PS + EL + ES + MS; | | | | |
| | EL + TB + FP + MS; EL + TB + FP + KQ; | | | | |
| | EC + SG + FPL + LA; SG + PS + LA + AH; | | | | |
| | EC + SG + LA + RI; SG + DS + ES + RI; | | | | |
| | EC + SG + FP + MS; SG + RT + LA + ES; | | | | |
| | SG + RT + LA + RI; PS + EL + TB + MM; | | | | |
| | SG + LA + FP + ES; LA + TB + AH + FP; | | | | |
| | EC + PS + AH + ES; SG + LA + AH + FP; | | | | |
| | SG + TB + AH + FP; DS + TB + ES + RI; | | | | |
| | PS + DS + LA + ES; DS + TB + AH + ES; | | | | |
| | DS + EL + TB + FP; EC + SG + PS + MS; | | | | |
| | EC + PS + TB + MS; SG + FPL + MF + FP; | | | | |
| | DS + FPL + EL + FP; EC + SG + LA + AH; | | | | |
| | SG + FPL + TB + ES; SG + DS + AH + RI; | | | | |
| | EC + SG + DS + LA; SG + FPL + LA + TB; | | | | |
| | DS + LA + TB + RI; LA + TB + ES + RI; | | | | |
| | EC + SG + PS + FP; EC + SG + ES + RI; | | | | |
| | FPL + TB + AH + ES; SG + DS + LA + ES; | | | | |
| | SG + DS + TB + AH; EC + TB + FP + ES; | | | | |
| | LA + TB + AH + ES; EC + SG + MF + ES; | | | | |
| | EC + SG + TB + FP; PS + DS + FPL + RI; | | | | |
| | EC + PS + ES + RI; EC + TB + FP + MS; | | | | |
| | EL + MF + TB + FP; SG + RT + LA + FP; | | | | |
| | EC + PS + TB + MM; SG + LA + MM + AH; | | | | |
| | SG + DS + FPL + TB; DS + FPL + ES + RI; | | | | |
| | EC + PS + DS + LA; SG + FPL + FP + ES; | | | | |
| | FPL + LA + TB + ES; EC + DS + LA + ES; | | | | |
| | EC + DS + MF + TB; EC + SG + LA + KQ; | | | | |
| | SG + RT + FPL + FP; EC + SG + DS + RI; | | | | |
| | EC + DS + TB + MS; DS + FPL + TB + KQ; | | | | |
| | DS + LA + TB + AH; LA + TB + MS + KQ; | | | | |
| | EC + SG + PS + AH; EC + MF + TB + ES; | | | | |
| | DS + FPL + EL + ES; EC + TB + AH + ES; | | | | |
| | SG + DS + AH + ES; SG + LA + AH + KQ; | | | | |
| | PS + EL + ES + KQ; SG + RT + LA + KQ; | | | | |
| | PS + RT + LA + ES; RT + LA + FP + ES; | | | | |
| | FPL + TB + ES + KQ | | | | |
| 4 | SG + DS + FPL + FP; LA + TB + FP + ES; | 0.78 | 0.3414- | 57.14%- | 52.53%- |
| | PS + EL + MM + ES; EC + SG + LA + TB; | | 0.4672 | 90.48% | 86.87% |
| | EC + PS + LA + ES; EC + SG + FPL + ES; | | | | |
| | LA + TB + MM + ES; SG + FPL + EL + FP; | | | | |
| | TB + AH + FP + ES; EC + SG + MM + ES; | | | | |
| | SG + EL + FP + ES; DS + FPL + MF + TB; | | | | |
| | SG + DS + FPL + ES; DS + FPL + EL + TB; | | | | |
| | EC + SG + EL + ES; SG + DS + LA + AH; | | | | |
| | TB + MM + ES + MS; DS + TB + FP + MS; | | | | |
| | DS + TB + FP + ES; SG + TB + AH + ES; | | | | |
| | EC + SG + RT + ES; SG + DS + FPL + EL; | | | | |
| | SG + DS + FPL + MF; FPL + EL + TB + FP; | | | | |
| | EC + SG + PS + MM; SG + FPL + TB + FP; | | | | |
| | EC + SG + AH + ES; EC + SG + LA + MS; | | | | |
| | EC + PS + EL + ES; EC + PS + MF + ES; | | | | |
| | EC + PS + DS + ES; EC + DS + TB + ES; | | | | |
| | EC + SG + PS + DS; DS + FPL + TB + ES; | | | | |
| | EC + PS + LA + TB; EC + TB + MM + ES; | | | | |
| | EC + DS + LA + TB; EC + SG + PS + RI; | | | | |
| | EC + SG + RT + LA; EL + TB + ES + RI; | | | | |
| | EC + SG + FP + ES | | | | |
| 4 | SG + PS + EL + ES; EC + PS + TB + ES; | 0.79 | 0.3586- | 66.67%- | 61.62%- |
| | SG + DS + FPL + RI; EL + TB + FP + ES; | | 0.4421 | 88.89% | 81.82% |
| | SG + LA + AH + ES; EC + LA + TB + ES; | | | | |
| | EL + TB + MM + ES; EC + SG + PS + LA; | | | | |
| | EC + SG + DS + ES; EC + PS + MM + ES | | | | |
| 4 | LA + TB + MM + MS; PS + EL + TB + ES; | >=0.8 | 0.377- | 73.02%- | 59.60%- |
| | EC + SG + LA + ES; EC + SG + PS + ES | | 0.4319 | 88.89% | 82.83% |

TABLE 6
The frequency of the 18 markers in the 2207 combinations

| Bacterial marker | Freq |
|---|---|
| Escherichia_{—}coli | 627 |
| Dorea_sp_AF36_15AT | 559 |
| Faecalibacterium prausnitzii | 554 |
| Turicibacter_{—}bilis | 548 |
| Lawsonibacter_{—}asaccharolyticus | 542 |
| Eggerthella_{—}sinensis | 537 |
| Flavonifractor_{—}plautii | 537 |
| Streptococcus_{—}gordonii | 532 |
| Romboutsia_{—}timonensis | 526 |
| Anaerostipes_{—}hadrus | 508 |
| Phascolarctobacterium_SGB4573 | 497 |
| Eggerthella_{—}lenta | 493 |
| Roseburia intestinalis | 489 |
| Klebsiella_{—}quasipneumoniae | 478 |
| Megasphaera_sp_NM10 | 464 |
| Megasphaera_{—}micronuciformis | 430 |
| Megamonas_{—}funiformis | 382 |
| Olsenella_sp_AF16_14LB | 125 |

Referring to Table 7, when choosing 5 markers from the 18 markers, 3994 out of 8568 (46.62%) total possible combinations achieved AUCs>=0.75, and 7178 out of 8568 (83.78%) total possible combinations achieved AUCs>=0.7. For the sake of clarity, the bacteria species combinations shown in Table 7 are represented by abbreviations of the bacteria species as per Table 2. For example, “EC+SG+OS+MM+KQ” refers to the combination of “Escherichia_coli, Streptococcus gordonii, Olsenella_sp_AF16_14LB, Megasphaera_micronuciformis, and Klebsiella_quasipneumoniae”.

As shown in Table 8, among these 7178 combinations, Escherichia coli appeared most frequently in 2296 combinations, followed by Dorea_sp_AF36_15AT which appeared 2184 times, Turicibacter_bilis appeared 2133 times, Faecalibacterium prausnitzii appeared 2121 times, and Lawsonibacter asaccharolyticus appeared 2100 times.

TABLE 7
List of combinations of 5 bacterial markers and the performance.

| | | | Best | | |
|---|---|---|---|---|---|
| Number | | | Thres- | Sensi- | Speci- |
| of | | | hold_ | tivity_ | ficity_ |
| markers | Combinations | AUC | Range | Range | Range |
| 5 | EC + SG + OS + MM + KQ; EC + OS + EL + TB + AH; | 0.7 | 0.2493- | 39.68%- | 35.35%- |
| | PS + RT + OS + KQ + RI; DS + OS + LA + MF + ES; | | 0.5996 | 98.41% | 92.93% |
| | FPL + MF + MS + KQ + RI; EC + OS + LA + MF + KQ; | | | | |
| | EC + OS + MF + AH + ES; SG + FPL + OS + TB + KQ; | | | | |
| | FPL + EL + LA + AH + MS; OS + EL + LA + ES + RI; | | | | |
| | PS + RT + OS + TB + FP; PS + LA + MF + TB + MS; | | | | |
| | PS + MF + TB + ES + KQ; SG + RT + EL + AH + MS; | | | | |
| | PS + RT + FPL + MM + AH; PS + EL + AH + FP + RI; | | | | |
| | RT + FPL + OS + KQ + RI; FPL + LA + MM + AH + RI; | | | | |
| | RT + FPL + OS + ES + KQ; EC + OS + EL + AH + ES; | | | | |
| | DS + OS + FP + ES + MS; SG + DS + EL + MF + MS; | | | | |
| | PS + RT + FPL + MF + MM; DS + FPL + OS + MS + KQ; | | | | |
| | EC + OS + MF + TB + MS; SG + PS + DS + OS + ES; | | | | |
| | PS + RT + LA + MF + MM; PS + MF + ES + KQ + RI; | | | | |
| | PS + RT + OS + AH + FP; EC + MF + MM + KQ + RI; | | | | |
| | PS + RT + EL + MF + AH; PS + EL + LA + MF + KQ; | | | | |
| | PS + MF + TB + KQ + RI; FPL + OS + TB + ES + RI; | | | | |
| | EC + SG + FPL + OS + RI; EC + OS + MM + FP + KQ; | | | | |
| | SG + FPL + OS + MM + FP; SG + LA + MF + TB + MM; | | | | |
| | PS + DS + OS + MF + RI; PS + DS + MF + MS + KQ; | | | | |
| | PS + FPL + MF + MM + AH; | | | | |
| | FPL + EL + MF + AH + MS; FPL + LA + MF + MM + ES; | | | | |
| | PS + OS + EL + LA + TB; DS + EL + MM + ES + MS; | | | | |
| | SG + MF + MS + KQ + RI; PS + OS + LA + TB + KQ; | | | | |
| | DS + EL + MM + MS + RI; SG + FPL + OS + AH + KQ; | | | | |
| | PS + DS + LA + MF + MM; PS + FPL + AH + FP + MS; | | | | |
| | RT + EL + MF + MM + MS; RT + AH + FP + MS + RI; | | | | |
| | LA + MF + TB + MM + ES; MF + MM + FP + MS + RI; | | | | |
| | SG + RT + OS + FP + ES; DS + LA + MF + MM + ES; | | | | |
| | EC + SG + OS + ES + MS; EC + FPL + OS + TB + MS; | | | | |
| | SG + PS + MF + TB + FP; PS + RT + EL + MM + AH; | | | | |
| | OS + LA + AH + FP + RI; EC + OS + MF + FP + RI; | | | | |
| | PS + RT + EL + TB + AH; PS + FPL + MM + AH + FP; | | | | |
| | LA + MF + AH + MS + RI; EC + OS + LA + MM + ES; | | | | |
| | EC + OS + MF + FP + MS; RT + FPL + OS + TB + KQ; | | | | |
| | RT + OS + MM + FP + KQ; DS + EL + LA + MM + RI; | | | | |
| | MF + TB + MM + AH + RI; EC + OS + MF + FP + KQ; | | | | |
| | PS + RT + FPL + OS + FP; RT + LA + MF + MM + RI; | | | | |
| | RT + MF + TB + MM + MS; DS + OS + EL + AH + KQ; | | | | |
| | EC + FPL + OS + MM + ES; EC + OS + EL + ES + KQ; | | | | |
| | SG + RT + OS + LA + MF; MM + AH + FP + MS + RI; | | | | |
| | FPL + MF + MM + KQ + RI; EC + RT + OS + LA + MM; | | | | |
| | EC + OS + TB + AH + RI; SG + FPL + OS + LA + MF; | | | | |
| | FPL + EL + LA + MF + AH; OS + TB + MM + ES + RI; | | | | |
| | EC + OS + EL + LA + ES; EC + OS + LA + MS + RI; | | | | |
| | PS + OS + EL + AH + ES; PS + LA + MF + MS + KQ; | | | | |
| | RT + FPL + OS + TB + RI; MF + TB + MM + KQ + RI; | | | | |
| | EC + OS + EL + FP + RI; PS + EL + MF + MM + AH; | | | | |
| | PS + EL + MF + MS + RI; RT + OS + EL + FP + ES; | | | | |
| | FPL + LA + MF + AH + RI; PS + OS + TB + ES + RI; | | | | |
| | RT + EL + MF + TB + MM; RT + MF + TB + ES + KQ; | | | | |
| | LA + MF + MM + MS + KQ; PS + DS + OS + EL + ES; | | | | |
| | FPL + OS + TB + ES + MS; OS + TB + AH + ES + RI; | | | | |
| | EC + FPL + AH + MS + RI; EC + OS + EL + MF + ES; | | | | |
| | PS + DS + MF + MM + FP; RT + FPL + TB + AH + MS; | | | | |
| | DS + FPL + OS + MM + FP; DS + OS + MM + FP + KQ; | | | | |
| | EC + SG + OS + MS + KQ; DS + OS + MF + TB + ES; | | | | |
| | FPL + LA + MF + MM + RI; SG + OS + MF + TB + AH; | | | | |
| | RT + MF + MM + KQ + RI; RT + TB + AH + FP + MS; | | | | |
| | FPL + OS + EL + TB + FP; SG + RT + FPL + TB + MM; | | | | |
| | DS + OS + EL + AH + FP; MF + MM + FP + KQ + RI; | | | | |
| | SG + FPL + OS + LA + MS; PS + RT + DS + OS + AH; | | | | |
| | PS + RT + MF + TB + KQ; RT + DS + MF + MM + AH; | | | | |
| | EL + LA + MF + MM + AH; RT + FPL + MF + TB + MM; | | | | |
| | FPL + OS + TB + MM + ES; OS + LA + MF + TB + FP; | | | | |
| | MF + MM + AH + ES + RI; EC + FPL + OS + MF + ES; | | | | |
| | EC + OS + LA + AH + RI; RT + OS + LA + FP + MS; | | | | |
| | PS + DS + OS + TB + ES; PS + OS + LA + TB + MM; | | | | |
| | RT + DS + MF + ES + KQ; EC + RT + FPL + OS + MS; | | | | |
| | SG + PS + OS + AH + RI; LA + MF + MM + FP + KQ; | | | | |
| | EC + RT + OS + MM + AH; EC + MF + MM + MS + RI; | | | | |
| | SG + OS + EL + FP + KQ; FPL + OS + LA + TB + KQ; | | | | |
| | FPL + MF + MM + MS + RI; EC + PS + OS + MF + MM; | | | | |
| | SG + PS + FPL + OS + RI; PS + MF + MM + ES + MS; | | | | |
| | DS + FPL + OS + FP + MS; RT + DS + OS + AH + RI; | | | | |
| | EC + PS + OS + AH + MS; EC + FPL + OS + LA + MF; | | | | |
| | EC + OS + ES + MS + RI; EC + MF + MM + MS + KQ; | | | | |
| | SG + OS + AH + FP + KQ; RT + OS + MF + FP + ES; | | | | |
| | FPL + MM + AH + MS + KQ; | | | | |
| | FPL + AH + MS + KQ + RI; EL + MF + AH + MS + KQ; | | | | |
| | SG + EL + MF + MM + KQ; PS + RT + FPL + OS + ES; | | | | |
| | RT + FPL + MM + AH + RI; | | | | |
| | FPL + LA + MF + MM + MS; EC + OS + MF + ES + RI; | | | | |
| | DS + MF + ES + MS + KQ; EC + FPL + OS + EL + LA; | | | | |
| | EC + EL + LA + MF + MM; SG + DS + OS + EL + LA; | | | | |
| | SG + FPL + OS + FP + ES; PS + OS + TB + AH + RI; | | | | |
| | SG + PS + MF + FP + MS; SG + DS + MF + ES + KQ; | | | | |
| | SG + OS + LA + ES + RI; PS + RT + OS + EL + ES; | | | | |
| | RT + FPL + OS + LA + AH; SG + FPL + OS + TB + FP; | | | | |
| | SG + OS + LA + MS + RI; PS + FPL + OS + MF + TB; | | | | |
| | RT + OS + MF + FP + RI; DS + OS + LA + FP + RI; | | | | |
| | DS + OS + TB + FP + MS; FPL + TB + MM + AH + MS; | | | | |
| | EC + OS + EL + AH + KQ; SG + OS + LA + MM + KQ; | | | | |
| | SG + OS + AH + ES + MS; OS + EL + MF + TB + ES; | | | | |
| | LA + MF + MM + FP + RI; SG + DS + OS + LA + MF; | | | | |
| | PS + DS + OS + ES + MS; EC + RT + OS + MF + TB; | | | | |
| | EC + DS + OS + MM + MS; SG + PS + FPL + OS + AH; | | | | |
| | PS + DS + EL + LA + MM; FPL + MM + AH + KQ + RI; | | | | |
| | PS + RT + MF + MM + FP; PS + FPL + EL + MM + AH; | | | | |
| | RT + OS + AH + ES + RI; DS + OS + EL + LA + KQ; | | | | |
| | OS + EL + TB + AH + KQ; RT + OS + MM + ES + RI; | | | | |
| | DS + FPL + OS + EL + RI; EC + OS + LA + AH + MS; | | | | |
| | PS + DS + EL + MM + MS; PS + FPL + OS + TB + MM; | | | | |
| | DS + OS + AH + MS + RI; OS + EL + LA + ES + MS; | | | | |
| | DS + OS + EL + LA + FP; DS + OS + ES + MS + RI; | | | | |
| | OS + EL + LA + AH + FP; SG + FPL + OS + MF + AH; | | | | |
| | PS + DS + OS + EL + MS; EL + LA + MF + AH + RI; | | | | |
| | EC + OS + LA + MF + FP; RT + DS + OS + AH + KQ; | | | | |
| | RT + MF + TB + MM + KQ; EL + LA + TB + MM + RI; | | | | |
| | LA + MF + MM + AH + MS; PS + DS + OS + EL + AH; | | | | |
| | OS + EL + AH + FP + KQ; EC + OS + EL + MF + FP; | | | | |
| | PS + FPL + OS + ES + KQ; RT + MF + MM + AH + FP; | | | | |
| | DS + FPL + OS + EL + AH; OS + EL + TB + MM + FP; | | | | |
| | RT + OS + MF + AH + FP; FPL + EL + MF + AH + RI; | | | | |
| | EC + PS + FPL + OS + MM; SG + OS + EL + TB + ES; | | | | |
| | EC + RT + OS + EL + MF; EC + OS + EL + ES + RI; | | | | |
| | RT + OS + EL + LA + AH; RT + MM + AH + FP + RI; | | | | |
| | OS + EL + MF + TB + FP; SG + RT + OS + TB + AH; | | | | |
| | SG + RT + OS + MS + RI; SG + FPL + OS + TB + MM; | | | | |
| | SG + OS + AH + ES + RI; PS + EL + MM + AH + MS; | | | | |
| | DS + FPL + OS + EL + KQ; DS + OS + LA + MM + MS; | | | | |
| | DS + EL + MM + MS + KQ; EC + PS + OS + MM + MS; | | | | |
| | PS + OS + TB + MM + KQ; RT + OS + EL + MS + RI; | | | | |
| | EC + OS + MF + ES + KQ; SG + DS + EL + MF + KQ; | | | | |
| | PS + FPL + EL + AH + MS; FPL + OS + TB + KQ + RI; | | | | |
| | EC + PS + OS + MM + RI; EC + EL + MM + MS + RI; | | | | |
| | SG + MF + TB + ES + KQ; RT + OS + TB + FP + ES; | | | | |
| | DS + OS + TB + ES + KQ; EC + RT + DS + OS + EL; | | | | |
| | SG + OS + MM + AH + ES; PS + FPL + LA + MF + MM; | | | | |
| | RT + EL + LA + MF + MM; OS + LA + AH + ES + MS; | | | | |
| | EC + PS + OS + LA + MS; EC + OS + LA + MM + KQ; | | | | |
| | SG + FPL + OS + FP + MS; SG + FPL + OS + ES + KQ; | | | | |
| | PS + OS + TB + MM + ES; RT + OS + LA + MF + RI; | | | | |
| | RT + OS + LA + MM + AH; OS + EL + LA + AH + ES; | | | | |
| | EC + RT + OS + TB + MM; DS + FPL + OS + EL + FP; | | | | |
| | DS + OS + MF + TB + RI; PS + FPL + MM + AH + RI; | | | | |
| | OS + LA + FP + MS + RI; PS + FPL + EL + MF + AH; | | | | |
| | PS + OS + TB + MM + AH; OS + EL + TB + MM + ES; | | | | |
| | OS + EL + TB + KQ + RI; OS + LA + TB + MM + FP; | | | | |
| | SG + PS + RT + MF + TB; PS + DS + OS + EL + MF; | | | | |
| | EC + OS + EL + MM + KQ; SG + PS + RT + OS + FP; | | | | |
| | SG + OS + LA + ES + KQ; OS + LA + AH + ES + RI; | | | | |
| | EC + RT + OS + MM + KQ; PS + RT + OS + EL + KQ; | | | | |
| | RT + FPL + OS + ES + MS; FPL + OS + FP + ES + RI; | | | | |
| | EC + FPL + OS + LA + RI; RT + FPL + OS + FP + KQ; | | | | |
| | FPL + LA + MF + MS + RI; EC + OS + LA + MF + AH; | | | | |
| | PS + LA + MF + MM + FP; RT + OS + TB + FP + RI; | | | | |
| | FPL + OS + LA + AH + ES; SG + MF + TB + MM + ES; | | | | |
| | DS + FPL + OS + EL + MS; RT + EL + MF + AH + MS; | | | | |
| | OS + EL + LA + ES + KQ; OS + LA + AH + ES + KQ; | | | | |
| | SG + PS + FPL + OS + FP; SG + OS + AH + KQ + RI; | | | | |
| | PS + OS + TB + MM + RI; RT + DS + OS + FP + ES; | | | | |
| | PS + FPL + OS + TB + RI; OS + TB + AH + ES + KQ; | | | | |
| | EC + FPL + OS + AH + KQ; SG + DS + OS + MS + KQ; | | | | |
| | PS + MF + AH + MS + KQ; RT + OS + FP + ES + MS; | | | | |
| | EC + RT + OS + MM + RI; SG + PS + OS + EL + AH; | | | | |
| | PS + OS + EL + TB + FP; RT + FPL + OS + LA + FP; | | | | |
| | RT + OS + LA + MF + AH; RT + OS + TB + ES + RI; | | | | |
| | OS + LA + FP + ES + KQ; EC + OS + MM + FP + RI; | | | | |
| | PS + MF + TB + MS + KQ; RT + OS + TB + AH + ES; | | | | |
| | SG + DS + MF + TB + KQ; PS + OS + LA + AH + ES; | | | | |
| | OS + TB + AH + ES + MS; PS + MF + ES + MS + KQ; | | | | |
| | EL + AH + MS + KQ + RI; EC + OS + AH + ES + MS; | | | | |
| | SG + PS + FPL + OS + ES; SG + RT + DS + OS + EL; | | | | |
| | RT + OS + EL + TB + ES; SG + RT + OS + TB + KQ; | | | | |
| | FPL + OS + LA + TB + MM; OS + MF + MM + AH + FP; | | | | |
| | RT + FPL + EL + AH + RI; DS + OS + MM + AH + KQ; | | | | |
| | DS + OS + AH + ES + KQ; FPL + OS + FP + MS + KQ; | | | | |
| | EC + OS + MM + AH + ES; SG + DS + OS + FP + RI; | | | | |
| | RT + FPL + AH + MS + RI; RT + OS + MF + ES + RI; | | | | |
| | RT + FPL + AH + FP + MS; PS + DS + OS + AH + MS; | | | | |
| | SG + OS + EL + TB + KQ; PS + MF + FP + ES + MS; | | | | |
| | DS + OS + EL + MF + FP; OS + TB + MM + ES + MS; | | | | |
| | EC + OS + MM + FP + MS; SG + PS + OS + EL + MF; | | | | |
| | RT + MM + AH + MS + KQ; PS + MF + MM + AH + RI; | | | | |
| | RT + OS + LA + ES + KQ; EC + FPL + OS + LA + MS; | | | | |
| | SG + PS + OS + LA + TB; DS + EL + LA + MM + ES; | | | | |
| | EC + SG + OS + EL + MM; EC + OS + TB + MM + MS; | | | | |
| | PS + DS + LA + MF + MS; PS + FPL + OS + FP + ES; | | | | |
| | DS + OS + LA + FP + KQ; PS + RT + DS + OS + ES; | | | | |
| | PS + FPL + OS + EL + TB; DS + OS + MM + FP + RI; | | | | |
| | SG + PS + OS + MM + AH; SG + OS + MF + AH + ES; | | | | |
| | RT + EL + MM + AH + FP; FPL + OS + AH + FP + ES; | | | | |
| | EL + LA + MF + ES + MS; SG + OS + EL + LA + TB; | | | | |
| | RT + TB + AH + MS + RI; EC + PS + FPL + OS + AH; | | | | |
| | PS + RT + OS + FP + ES; PS + DS + FPL + OS + FP; | | | | |
| | RT + DS + LA + MF + MM; EC + OS + EL + LA + AH; | | | | |
| | OS + EL + LA + TB + FP; RT + OS + EL + MF + FP; | | | | |
| | EC + SG + OS + MF + KQ; PS + OS + TB + FP + KQ; | | | | |
| | RT + OS + EL + LA + FP; DS + OS + MM + AH + ES; | | | | |
| | DS + MF + MM + KQ + RI; FPL + OS + MF + TB + KQ; | | | | |
| | SG + RT + OS + MF + RI; OS + MF + TB + FP + ES; | | | | |
| | SG + PS + LA + MF + MM; SG + FPL + OS + KQ + RI; | | | | |
| | SG + PS + OS + EL + TB; OS + LA + FP + KQ + RI; | | | | |
| | EC + RT + OS + MM + MS; OS + TB + FP + ES + KQ; | | | | |
| | LA + MF + MM + AH + RI; EC + FPL + OS + LA + MM; | | | | |
| | SG + FPL + OS + AH + RI; PS + OS + LA + TB + MS; | | | | |
| | RT + DS + MF + MM + MS; PS + DS + OS + MM + ES; | | | | |
| | PS + OS + EL + MF + ES; RT + MF + AH + FP + RI; | | | | |
| | SG + OS + AH + ES + KQ; FPL + OS + MF + TB + MM; | | | | |
| | EC + FPL + OS + EL + AH; SG + DS + MF + MM + MS; | | | | |
| | SG + LA + MF + MM + MS; PS + DS + OS + EL + MM; | | | | |
| | RT + DS + OS + LA + MF; RT + FPL + TB + MM + MS; | | | | |
| | OS + EL + LA + FP + MS; SG + DS + EL + MM + MS; | | | | |
| | PS + LA + MF + MM + ES; DS + FPL + OS + MM + MS; | | | | |
| | FPL + OS + TB + AH + MS; SG + PS + OS + LA + RI; | | | | |
| | OS + LA + AH + KQ + RI; OS + MF + TB + AH + ES; | | | | |
| | EC + OS + EL + LA + FP; SG + PS + OS + AH + KQ; | | | | |
| | PS + OS + EL + FP + RI; PS + OS + LA + AH + RI; | | | | |
| | PS + MF + MS + KQ + RI; EL + MF + ES + KQ + RI; | | | | |
| | PS + FPL + AH + MS + RI; EC + RT + OS + MF + KQ; | | | | |
| | EC + OS + EL + MM + FP; SG + DS + OS + MF + FP; | | | | |
| | PS + DS + FPL + OS + MS; PS + DS + OS + MM + KQ; | | | | |
| | EL + MF + MM + ES + KQ; EC + FPL + OS + MM + KQ; | | | | |
| | EC + OS + LA + MF + RI; SG + RT + OS + FP + MS; | | | | |
| | RT + EL + AH + FP + RI; DS + FPL + OS + LA + MS; | | | | |
| | SG + OS + EL + LA + FP; RT + MF + TB + AH + MS; | | | | |
| | EC + RT + FPL + OS + MM; EC + OS + MF + AH + KQ; | | | | |
| | SG + MF + TB + MM + RI; PS + RT + OS + EL + FP; | | | | |
| | PS + DS + OS + MF + TB; OS + TB + MM + FP + MS; | | | | |
| | SG + PS + FPL + OS + KQ; PS + OS + TB + MS + RI; | | | | |
| | PS + OS + MM + ES + RI; DS + OS + LA + MM + KQ; | | | | |
| | FPL + MM + AH + FP + RI; EC + SG + OS + EL + RI; | | | | |
| | RT + OS + EL + ES + MS; FPL + OS + MF + MM + FP; | | | | |
| | EL + MF + AH + KQ + RI; EL + TB + MM + KQ + RI; | | | | |
| | EC + LA + MF + MM + KQ; DS + OS + MM + KQ + RI; | | | | |
| | PS + OS + EL + LA + RI; RT + OS + EL + KQ + RI; | | | | |
| | DS + EL + MF + ES + KQ | | | | |
| 5 | EC + FPL + OS + TB + MM; PS + DS + OS + AH + KQ; | 0.71 | 0.2442- | 36.51%- | 34.34%- |
| | RT + OS + FP + ES + RI; DS + EL + TB + MM + RI; | | 0.5727 | 98.41% | 94.95% |
| | LA + MF + FP + KQ + RI; EC + RT + OS + EL + KQ; | | | | |
| | EC + RT + OS + MS + RI; EC + OS + TB + MS + KQ; | | | | |
| | EC + MF + MM + AH + MS; OS + EL + LA + TB + MS; | | | | |
| | OS + LA + TB + FP + ES; EC + PS + RT + OS + MF; | | | | |
| | SG + OS + EL + LA + KQ; PS + EL + MM + AH + FP; | | | | |
| | DS + FPL + OS + LA + MM; OS + TB + FP + ES + MS; | | | | |
| | MF + FP + ES + MS + KQ; SG + PS + RT + OS + RI; | | | | |
| | PS + EL + AH + FP + MS; DS + OS + AH + FP + KQ; | | | | |
| | EC + RT + OS + EL + TB; EC + LA + MF + MM + FP; | | | | |
| | SG + OS + TB + AH + KQ; PS + DS + OS + EL + TB; | | | | |
| | RT + FPL + AH + MS + KQ; RT + EL + AH + MS + RI; | | | | |
| | OS + LA + MF + TB + AH; LA + MF + MM + AH + ES; | | | | |
| | EC + SG + OS + MF + AH; SG + RT + EL + MF + AH; | | | | |
| | PS + FPL + AH + MS + KQ; RT + DS + MF + TB + ES; | | | | |
| | SG + RT + MM + AH + MS; RT + MF + AH + KQ + RI; | | | | |
| | EC + DS + EL + MM + RI; EC + OS + FP + MS + KQ; | | | | |
| | PS + RT + OS + ES + RI; PS + RT + MF + TB + AH; | | | | |
| | PS + MF + AH + KQ + RI; DS + FPL + OS + MF + RI; | | | | |
| | EC + PS + OS + MS + RI; EC + OS + TB + MS + RI; | | | | |
| | EC + MM + AH + KQ + RI; RT + DS + EL + MM + KQ; | | | | |
| | RT + FPL + OS + ES + RI; EC + PS + OS + EL + FP; | | | | |
| | SG + PS + RT + TB + KQ; SG + PS + MF + TB + MS; | | | | |
| | PS + FPL + TB + MM + AH; DS + OS + LA + ES + RI; | | | | |
| | DS + OS + TB + ES + RI; FPL + EL + LA + MM + AH; | | | | |
| | EC + RT + OS + EL + LA; EC + RT + OS + EL + RI; | | | | |
| | SG + RT + TB + MM + KQ; SG + DS + EL + TB + MM; | | | | |
| | SG + OS + LA + MF + AH; SG + MF + TB + MS + KQ; | | | | |
| | AH + FP + ES + MS + RI; EC + DS + OS + FP + RI; | | | | |
| | EC + OS + TB + MM + ES; RT + DS + MF + AH + KQ; | | | | |
| | RT + FPL + AH + FP + RI; FPL + OS + TB + ES + KQ; | | | | |
| | EL + LA + MF + FP + KQ; EC + MM + AH + FP + RI; | | | | |
| | SG + DS + OS + LA + MM; EC + RT + FPL + OS + EL; | | | | |
| | SG + EL + LA + MS + RI; DS + MF + MM + ES + RI; | | | | |
| | EC + MF + AH + MS + RI; RT + OS + EL + LA + TB; | | | | |
| | OS + EL + TB + FP + KQ; EL + LA + MF + MS + KQ; | | | | |
| | SG + PS + OS + AH + FP; SG + PS + LA + MF + TB; | | | | |
| | SG + EL + MM + MS + KQ; PS + OS + LA + TB + FP; | | | | |
| | PS + MF + TB + MM + MS; OS + TB + AH + FP + MS; | | | | |
| | SG + PS + LA + MF + MS; SG + DS + OS + EL + AH; | | | | |
| | SG + DS + OS + AH + ES; SG + FPL + OS + LA + ES; | | | | |
| | DS + FPL + OS + EL + MM; DS + OS + LA + TB + MS; | | | | |
| | EC + FPL + OS + EL + TB; SG + OS + LA + TB + FP; | | | | |
| | RT + DS + TB + MM + AH; RT + FPL + MM + AH + KQ; | | | | |
| | DS + EL + MM + KQ + RI; SG + FPL + MM + AH + RI; | | | | |
| | EC + RT + OS + MS + KQ; EC + OS + EL + MF + TB; | | | | |
| | PS + RT + OS + LA + RI; PS + FPL + TB + AH + RI; | | | | |
| | RT + MF + AH + FP + KQ; EC + RT + OS + AH + RI; | | | | |
| | EC + FPL + OS + FP + RI; SG + DS + MF + TB + MM; | | | | |
| | MF + AH + FP + MS + RI; SG + OS + EL + TB + FP; | | | | |
| | LA + MF + TB + MM + KQ; MM + AH + FP + KQ + RI; | | | | |
| | EC + PS + OS + EL + RI; PS + RT + MF + MM + RI; | | | | |
| | EC + RT + DS + OS + MM; EC + EL + MF + MM + AH; | | | | |
| | SG + PS + FPL + OS + LA; SG + PS + MM + FP + RI; | | | | |
| | MF + TB + ES + MS + KQ; EC + DS + OS + MM + KQ; | | | | |
| | SG + RT + FPL + OS + ES; PS + FPL + AH + ES + MS; | | | | |
| | RT + OS + LA + KQ + RI; DS + FPL + OS + LA + FP; | | | | |
| | SG + OS + TB + AH + MS; PS + FPL + MM + FP + MS; | | | | |
| | RT + DS + OS + LA + TB; RT + MF + MM + MS + RI; | | | | |
| | DS + OS + LA + TB + MM; OS + TB + AH + FP + RI; | | | | |
| | SG + RT + OS + AH + ES; SG + EL + TB + KQ + RI; | | | | |
| | PS + DS + EL + TB + MM; EC + RT + OS + MM + FP; | | | | |
| | PS + RT + FPL + MM + MS; OS + EL + LA + TB + RI; | | | | |
| | EL + LA + MF + ES + KQ; EC + PS + RT + OS + MM; | | | | |
| | DS + OS + EL + TB + MS; FPL + EL + LA + KQ + RI; | | | | |
| | EL + LA + AH + FP + RI; EC + SG + OS + TB + MS; | | | | |
| | EC + RT + OS + MM + ES; EC + OS + EL + TB + MM; | | | | |
| | SG + DS + MF + MM + FP; PS + RT + OS + LA + AH; | | | | |
| | PS + RT + MF + MS + KQ; RT + TB + MM + AH + RI; | | | | |
| | PS + MM + AH + FP + RI; RT + FPL + OS + FP + ES; | | | | |
| | DS + OS + TB + MM + RI; EC + PS + FPL + OS + LA; | | | | |
| | EC + PS + OS + MF + AH; PS + RT + AH + MS + KQ; | | | | |
| | DS + OS + TB + MS + KQ; EC + RT + OS + MF + ES; | | | | |
| | EC + OS + FP + KQ + RI; PS + FPL + LA + AH + MS; | | | | |
| | DS + EL + LA + MF + MS; PS + RT + EL + MF + MM; | | | | |
| | PS + RT + MF + FP + KQ; FPL + EL + MF + MM + AH; | | | | |
| | EC + SG + OS + MM + ES; SG + PS + RT + TB + MS; | | | | |
| | SG + RT + MF + MM + AH; SG + DS + EL + MM + ES; | | | | |
| | PS + FPL + MF + MS + KQ; PS + OS + EL + TB + RI; | | | | |
| | DS + FPL + OS + FP + KQ; FPL + LA + MF + AH + MS; | | | | |
| | EL + MF + MM + FP + MS; EC + DS + FPL + OS + MS; | | | | |
| | EC + OS + MF + TB + RI; EC + OS + FP + MS + RI; | | | | |
| | SG + OS + EL + FP + RI; PS + DS + FPL + OS + EL; | | | | |
| | PS + MF + MM + AH + MS; RT + FPL + OS + TB + FP; | | | | |
| | EL + MM + ES + KQ + RI; FPL + MF + ES + MS + KQ; | | | | |
| | OS + EL + TB + ES + MS; MF + MM + FP + ES + RI; | | | | |
| | EC + SG + OS + EL + MS; EC + RT + MF + MM + KQ; | | | | |
| | RT + OS + ES + KQ + RI; OS + EL + LA + TB + ES; | | | | |
| | EC + RT + OS + MF + RI; EC + RT + OS + AH + KQ; | | | | |
| | EC + DS + OS + MF + MS; DS + OS + EL + TB + FP; | | | | |
| | EC + PS + RT + FPL + OS; EC + OS + LA + KQ + RI; | | | | |
| | SG + OS + AH + FP + ES; PS + RT + FPL + OS + LA; | | | | |
| | PS + DS + MF + FP + RI; EL + LA + MF + TB + MS; | | | | |
| | MF + AH + FP + MS + KQ; PS + EL + MF + AH + MS; | | | | |
| | RT + LA + MF + TB + MM; MF + FP + ES + KQ + RI; | | | | |
| | MF + FP + MS + KQ + RI; EC + EL + LA + MF + RI; | | | | |
| | SG + PS + FPL + MM + MS; RT + OS + LA + TB + ES; | | | | |
| | SG + DS + OS + TB + RI; SG + FPL + EL + AH + MS; | | | | |
| | DS + FPL + OS + TB + MS; DS + FPL + OS + ES + KQ; | | | | |
| | DS + EL + ES + MS + KQ; EC + PS + OS + MM + AH; | | | | |
| | FPL + MF + AH + ES + MS; EC + PS + FPL + OS + MF; | | | | |
| | SG + EL + MF + AH + RI; PS + DS + OS + TB + MS; | | | | |
| | PS + EL + MM + FP + RI; DS + OS + EL + AH + RI; | | | | |
| | EC + PS + OS + AH + RI; EC + FPL + OS + EL + ES; | | | | |
| | SG + DS + OS + MF + AH; RT + DS + FPL + OS + AH; | | | | |
| | FPL + MF + AH + MS + KQ; PS + EL + MM + AH + RI; | | | | |
| | RT + EL + LA + AH + MS; DS + FPL + OS + FP + ES; | | | | |
| | DS + OS + MF + TB + MS; OS + LA + FP + MS + KQ; | | | | |
| | OS + TB + MM + AH + FP; SG + DS + EL + TB + KQ; | | | | |
| | RT + MF + TB + MS + KQ; FPL + MF + AH + FP + MS; | | | | |
| | SG + OS + LA + KQ + RI; SG + OS + AH + FP + MS; | | | | |
| | SG + EL + MF + AH + KQ; FPL + EL + LA + MF + MS; | | | | |
| | EC + OS + LA + FP + KQ; SG + FPL + AH + MS + RI; | | | | |
| | RT + EL + MF + TB + AH; RT + TB + MM + AH + FP; | | | | |
| | DS + FPL + OS + EL + MF; FPL + OS + MF + TB + ES; | | | | |
| | FPL + OS + TB + MM + FP; EL + MM + AH + MS + KQ; | | | | |
| | SG + MF + MM + FP + RI; RT + EL + TB + MM + AH; | | | | |
| | FPL + OS + TB + FP + ES; AH + FP + MS + KQ + RI; | | | | |
| | EC + SG + OS + MM + AH; EC + RT + OS + TB + MS; | | | | |
| | TB + MM + AH + MS + RI; EC + RT + OS + LA + MF; | | | | |
| | PS + RT + MM + AH + FP; RT + FPL + TB + MS + RI; | | | | |
| | EC + OS + EL + FP + MS; SG + PS + EL + LA + MF; | | | | |
| | PS + DS + OS + LA + MF; RT + FPL + MF + AH + RI; | | | | |
| | RT + AH + FP + KQ + RI; DS + OS + AH + FP + MS; | | | | |
| | EC + PS + OS + MM + FP; EC + RT + OS + EL + AH; | | | | |
| | EC + OS + AH + ES + KQ; PS + RT + OS + EL + LA; | | | | |
| | OS + LA + AH + FP + KQ; EL + LA + MM + ES + MS; | | | | |
| | LA + MF + MM + FP + MS; EC + SG + OS + EL + ES; | | | | |
| | EC + SG + OS + MF + ES; EC + DS + OS + MF + MM; | | | | |
| | PS + DS + OS + EL + RI; PS + FPL + LA + MM + AH; | | | | |
| | RT + OS + LA + TB + RI; EC + FPL + OS + ES + KQ; | | | | |
| | RT + FPL + OS + LA + RI; EL + MF + TB + AH + RI; | | | | |
| | OS + LA + FP + ES + RI; SG + DS + OS + KQ + RI; | | | | |
| | SG + FPL + OS + TB + ES; RT + OS + LA + MS + RI; | | | | |
| | EC + DS + OS + MM + RI; EC + OS + LA + AH + KQ; | | | | |
| | RT + FPL + MF + AH + MS; FPL + OS + TB + AH + KQ; | | | | |
| | PS + DS + EL + LA + MF; OS + EL + TB + FP + MS; | | | | |
| | MF + AH + ES + MS + RI; RT + FPL + OS + AH + ES; | | | | |
| | FPL + OS + AH + FP + KQ; EC + PS + OS + EL + MM; | | | | |
| | SG + RT + OS + LA + MM; SG + DS + OS + TB + MS; | | | | |
| | DS + OS + LA + ES + KQ; FPL + EL + MM + AH + FP; | | | | |
| | OS + LA + TB + FP + RI; RT + OS + FP + KQ + RI; | | | | |
| | RT + MF + TB + AH + KQ; EC + OS + MM + AH + FP; | | | | |
| | EC + EL + MF + MM + RI; DS + MF + MM + MS + KQ; | | | | |
| | OS + LA + MF + TB + ES; EC + PS + OS + EL + MS; | | | | |
| | PS + RT + TB + MM + AH; RT + AH + MS + KQ + RI; | | | | |
| | EL + LA + MM + KQ + RI; SG + DS + OS + FP + KQ; | | | | |
| | PS + MM + FP + ES + KQ; RT + EL + MF + AH + RI; | | | | |
| | FPL + OS + LA + TB + ES; EC + FPL + OS + ES + RI; | | | | |
| | SG + FPL + OS + EL + LA; RT + FPL + EL + AH + MS; | | | | |
| | RT + OS + LA + AH + RI; FPL + MF + MM + AH + ES; | | | | |
| | EC + FPL + OS + MF + TB; EC + OS + TB + AH + MS; | | | | |
| | PS + FPL + LA + MF + MS; RT + EL + AH + MS + KQ; | | | | |
| | DS + OS + FP + MS + RI; OS + LA + MF + AH + FP; | | | | |
| | MF + TB + MM + MS + KQ; RT + FPL + EL + MF + AH; | | | | |
| | RT + MF + TB + AH + RI; RT + MF + MM + MS + KQ; | | | | |
| | OS + LA + TB + ES + KQ; EC + OS + AH + FP + RI; | | | | |
| | SG + PS + OS + AH + ES; PS + MM + AH + MS + RI; | | | | |
| | RT + FPL + OS + MF + FP; RT + MF + AH + FP + MS; | | | | |
| | DS + MF + TB + ES + KQ; FPL + OS + LA + TB + AH; | | | | |
| | EC + PS + OS + EL + LA; SG + DS + EL + TB + MS; | | | | |
| | OS + EL + TB + FP + RI; OS + LA + AH + FP + ES; | | | | |
| | LA + MF + AH + KQ + RI; DS + OS + EL + MS + RI; | | | | |
| | FPL + MF + MM + ES + RI; EC + EL + MF + MS + RI; | | | | |
| | DS + FPL + OS + MM + RI; FPL + MF + MM + AH + FP; | | | | |
| | SG + OS + MM + AH + FP; RT + DS + OS + EL + LA; | | | | |
| | EL + TB + MM + AH + MS; SG + PS + OS + EL + KQ; | | | | |
| | SG + PS + OS + EL + RI; SG + RT + FPL + OS + AH; | | | | |
| | SG + EL + MF + MS + KQ; SG + MF + AH + MS + KQ; | | | | |
| | DS + OS + EL + LA + AH; EC + RT + FPL + OS + KQ; | | | | |
| | EC + OS + EL + TB + RI; SG + DS + EL + MF + TB; | | | | |
| | SG + FPL + OS + LA + MM; EC + SG + OS + MF + RI; | | | | |
| | SG + RT + DS + MF + MM; PS + AH + FP + MS + RI; | | | | |
| | RT + OS + EL + FP + KQ; RT + MM + AH + FP + MS; | | | | |
| | EC + FPL + OS + EL + FP; SG + PS + MF + FP + RI; | | | | |
| | SG + EL + TB + MS + KQ; OS + LA + TB + MS + RI; | | | | |
| | FPL + EL + LA + MF + KQ; EC + FPL + OS + LA + KQ; | | | | |
| | SG + EL + LA + MF + RI; PS + DS + OS + EL + KQ; | | | | |
| | PS + DS + MF + TB + KQ; RT + EL + MM + AH + KQ; | | | | |
| | OS + TB + FP + ES + RI; EC + MF + MM + AH + RI; | | | | |
| | PS + DS + OS + LA + FP; EC + OS + LA + MS + KQ; | | | | |
| | SG + EL + MM + AH + RI; RT + EL + AH + FP + MS; | | | | |
| | RT + MF + MM + AH + ES; DS + FPL + OS + AH + RI; | | | | |
| | FPL + LA + MM + AH + MS; EC + OS + EL + LA + KQ; | | | | |
| | EC + OS + EL + AH + FP; SG + PS + MM + FP + MS; | | | | |
| | EL + MF + MM + AH + KQ; LA + MM + AH + KQ + RI; | | | | |
| | EC + FPL + OS + FP + MS; SG + EL + LA + MF + TB; | | | | |
| | DS + FPL + OS + MF + MS; FPL + OS + TB + FP + MS; | | | | |
| | FPL + LA + MF + MM + KQ; PS + RT + MF + TB + ES; | | | | |
| | DS + OS + TB + KQ + RI; EC + RT + OS + TB + KQ; | | | | |
| | SG + RT + EL + MM + AH; SG + DS + LA + MF + MM; | | | | |
| | PS + RT + MF + MM + KQ; EC + MM + AH + MS + RI; | | | | |
| | SG + PS + MF + ES + MS; SG + RT + MF + MM + MS; | | | | |
| | PS + FPL + EL + AH + RI; MF + MM + FP + ES + KQ; | | | | |
| | EC + PS + OS + LA + MM; SG + RT + AH + FP + MS; | | | | |
| | SG + OS + LA + TB + ES; SG + OS + TB + AH + RI; | | | | |
| | SG + MF + TB + KQ + RI; DS + EL + LA + MF + KQ; | | | | |
| | SG + OS + EL + FP + MS; OS + EL + FP + ES + RI; | | | | |
| | DS + OS + MF + TB + FP; OS + LA + AH + FP + MS; | | | | |
| | OS + AH + FP + ES + RI; EC + OS + EL + FP + KQ; | | | | |
| | EC + RT + OS + AH + MS; RT + FPL + OS + LA + ES; | | | | |
| | FPL + OS + LA + FP + ES; OS + EL + TB + MM + KQ; | | | | |
| | EC + RT + OS + EL + MS; EC + OS + AH + FP + MS; | | | | |
| | RT + EL + MF + MM + KQ; EC + OS + EL + TB + MS; | | | | |
| | EC + OS + LA + FP + MS; SG + RT + MF + AH + MS; | | | | |
| | PS + RT + TB + AH + MS; PS + FPL + MF + AH + MS; | | | | |
| | RT + FPL + MF + MM + KQ; RT + EL + MF + AH + KQ; | | | | |
| | SG + FPL + EL + AH + RI; PS + RT + OS + LA + MS; | | | | |
| | RT + DS + EL + MF + KQ; RT + OS + FP + MS + RI; | | | | |
| | EL + LA + MM + AH + RI; MF + AH + FP + KQ + RI; | | | | |
| | EC + RT + OS + TB + RI; EC + EL + MM + AH + RI; | | | | |
| | SG + OS + LA + TB + MM; SG + OS + LA + ES + MS; | | | | |
| | DS + OS + AH + KQ + RI; SG + DS + OS + TB + ES; | | | | |
| | SG + EL + TB + MM + MS; EC + EL + MF + AH + RI; | | | | |
| | SG + DS + MF + MS + KQ; SG + OS + EL + AH + FP; | | | | |
| | SG + EL + MF + AH + MS; PS + DS + OS + MM + AH; | | | | |
| | DS + FPL + OS + MF + MM; OS + LA + MM + AH + FP; | | | | |
| | EL + LA + AH + MS + RI; EL + AH + FP + KQ + RI; | | | | |
| | EC + DS + OS + EL + MS; DS + FPL + OS + MM + AH; | | | | |
| | SG + OS + EL + LA + AH; PS + OS + EL + TB + MS; | | | | |
| | EL + MM + AH + KQ + RI; SG + OS + TB + MM + AH; | | | | |
| | SG + OS + TB + AH + ES; FPL + LA + AH + FP + MS; | | | | |
| | SG + PS + MF + MM + RI; EC + PS + FPL + OS + EL; | | | | |
| | EC + RT + OS + MF + MS; FPL + EL + AH + KQ + RI; | | | | |
| | OS + LA + TB + MM + MS; MF + TB + AH + KQ + RI; | | | | |
| | SG + DS + OS + TB + FP; PS + MF + MM + FP + KQ; | | | | |
| | SG + OS + LA + FP + ES; PS + OS + TB + AH + ES; | | | | |
| | EC + OS + EL + MF + KQ; EC + OS + MF + TB + MM; | | | | |
| | EC + OS + ES + KQ + RI; RT + OS + AH + FP + KQ; | | | | |
| | FPL + LA + MF + MS + KQ; PS + RT + FPL + AH + MS; | | | | |
| | DS + FPL + OS + EL + LA; FPL + OS + MF + TB + AH; | | | | |
| | MF + AH + ES + KQ + RI; PS + OS + TB + AH + FP; | | | | |
| | RT + OS + TB + MM + FP; DS + OS + EL + MF + TB; | | | | |
| | OS + TB + FP + MS + RI; EL + MF + AH + FP + RI; | | | | |
| | EC + OS + TB + ES + MS; SG + PS + OS + EL + ES; | | | | |
| | PS + FPL + OS + TB + AH; RT + DS + FPL + OS + TB; | | | | |
| | DS + OS + LA + TB + KQ; EL + MF + TB + MM + AH; | | | | |
| | EC + RT + OS + MF + AH; SG + PS + DS + OS + TB; | | | | |
| | DS + OS + LA + AH + KQ; EC + FPL + OS + LA + AH; | | | | |
| | PS + RT + AH + MS + RI; PS + OS + EL + MF + TB; | | | | |
| | DS + MF + MM + MS + RI; MF + TB + MM + MS + RI; | | | | |
| | EC + OS + TB + MM + AH; SG + PS + OS + LA + KQ; | | | | |
| | SG + RT + DS + OS + AH; PS + RT + DS + TB + MM; | | | | |
| | RT + FPL + OS + AH + FP; RT + OS + ES + MS + RI; | | | | |
| | RT + TB + AH + MS + KQ; FPL + EL + MM + AH + MS; | | | | |
| | EC + OS + TB + MM + RI; EC + OS + AH + ES + RI; | | | | |
| | PS + DS + FPL + OS + MM; PS + FPL + OS + LA + TB; | | | | |
| | PS + FPL + OS + TB + KQ; PS + OS + EL + FP + ES; | | | | |
| | MF + TB + MS + KQ + RI; EC + LA + MF + MM + MS; | | | | |
| | SG + PS + OS + EL + FP; DS + OS + TB + FP + ES; | | | | |
| | FPL + EL + AH + FP + MS; FPL + MM + AH + ES + MS; | | | | |
| | SG + RT + OS + AH + KQ; RT + DS + FPL + OS + MF; | | | | |
| | RT + OS + MF + TB + FP; DS + OS + EL + FP + KQ; | | | | |
| | FPL + OS + TB + MM + KQ; OS + TB + MM + MS + KQ; | | | | |
| | SG + DS + OS + FP + MS; PS + DS + OS + MF + AH; | | | | |
| | EC + SG + OS + MM + RI; PS + DS + OS + KQ + RI; | | | | |
| | EC + RT + LA + MF + MM; EC + DS + OS + EL + MM; | | | | |
| | PS + RT + FPL + OS + RI; RT + EL + MM + AH + RI | | | | |
| 5 | EC + RT + OS + LA + KQ; EC + FPL + EL + AH + RI; | 0.72 | 0.2833- | 39.68%- | 36.36%- |
| | EC + FPL + AH + FP + RI; RT + FPL + AH + KQ + RI; | | 0.6225 | 98.41% | 92.93% |
| | DS + OS + EL + TB + RI; DS + OS + LA + FP + MS; | | | | |
| | FPL + EL + MF + AH + FP; EL + MF + MM + AH + ES; | | | | |
| | EL + MM + FP + KQ + RI; MF + MM + AH + FP + ES; | | | | |
| | MM + FP + ES + KQ + RI; EC + RT + OS + AH + FP; | | | | |
| | EC + OS + TB + AH + KQ; EC + EL + MM + KQ + RI; | | | | |
| | SG + RT + MF + MS + KQ; PS + DS + MF + FP + MS; | | | | |
| | TB + MM + AH + KQ + RI; EC + AH + FP + KQ + RI; | | | | |
| | PS + DS + OS + LA + AH; DS + MM + FP + KQ + RI; | | | | |
| | EL + LA + MM + FP + RI; EC + PS + OS + MF + KQ; | | | | |
| | EC + FPL + MF + MM + RI; SG + DS + FPL + OS + MM; | | | | |
| | PS + FPL + EL + LA + MS; DS + FPL + OS + LA + AH; | | | | |
| | FPL + LA + MF + ES + RI; OS + LA + TB + FP + MS; | | | | |
| | EL + MF + AH + ES + RI; MM + AH + ES + KQ + RI; | | | | |
| | EC + PS + OS + AH + FP; RT + MF + TB + AH + ES; | | | | |
| | DS + EL + TB + ES + KQ; FPL + AH + ES + MS + RI; | | | | |
| | EC + DS + OS + EL + KQ; EC + LA + MF + AH + RI; | | | | |
| | SG + FPL + OS + FP + KQ; EC + PS + RT + OS + EL; | | | | |
| | EC + DS + OS + LA + FP; EC + FPL + AH + KQ + RI; | | | | |
| | EC + OS + TB + MM + KQ; EC + MF + MM + FP + RI; | | | | |
| | PS + OS + EL + ES + MS; PS + RT + MM + FP + MS; | | | | |
| | PS + EL + MF + MM + KQ; EC + LA + AH + MS + RI; | | | | |
| | SG + PS + MF + FP + ES; SG + DS + EL + TB + ES; | | | | |
| | RT + FPL + MF + AH + KQ; OS + EL + TB + AH + FP; | | | | |
| | EL + MF + FP + KQ + RI; EC + FPL + LA + FP + KQ; | | | | |
| | EL + LA + MM + AH + MS; EC + RT + MM + AH + RI; | | | | |
| | SG + OS + LA + AH + ES; SG + EL + TB + AH + MS; | | | | |
| | PS + DS + FPL + MM + MS; PS + FPL + MF + AH + FP; | | | | |
| | RT + OS + LA + MM + FP; EC + PS + FPL + AH + MS; | | | | |
| | EC + DS + OS + MF + AH; EC + FPL + OS + MF + FP; | | | | |
| | PS + RT + DS + EL + MM; RT + DS + OS + MS + RI; | | | | |
| | RT + FPL + MF + TB + MS; EC + DS + OS + MS + KQ; | | | | |
| | EC + FPL + LA + AH + RI; EC + EL + MF + MM + KQ; | | | | |
| | PS + RT + EL + MM + MS; PS + FPL + TB + AH + MS; | | | | |
| | EC + MF + MM + FP + KQ; SG + DS + OS + LA + AH; | | | | |
| | SG + DS + EL + MF + ES; RT + DS + OS + AH + FP; | | | | |
| | DS + EL + MF + MS + KQ; FPL + EL + LA + TB + AH; | | | | |
| | EC + SG + PS + OS + MM; EC + RT + DS + OS + TB; | | | | |
| | PS + RT + FPL + MF + MS; FPL + LA + MM + MS + RI; | | | | |
| | FPL + MF + MM + ES + KQ; | | | | |
| | SG + FPL + LA + MF + MM; PS + FPL + MM + MS + RI; | | | | |
| | PS + MM + AH + KQ + RI; RT + FPL + EL + MF + MS; | | | | |
| | RT + OS + LA + TB + AH; EC + RT + DS + OS + RI; | | | | |
| | SG + RT + FPL + OS + FP; EC + SG + FPL + OS + EL; | | | | |
| | EC + FPL + MF + AH + MS; EC + LA + MF + MM + AH; | | | | |
| | SG + PS + DS + EL + MS; SG + RT + MF + TB + MS; | | | | |
| | PS + LA + MF + MM + AH; RT + EL + TB + AH + RI; | | | | |
| | DS + LA + MF + TB + MM; EC + RT + OS + ES + MS; | | | | |
| | SG + RT + MF + TB + ES; SG + DS + OS + FP + ES; | | | | |
| | FPL + EL + MF + KQ + RI; FPL + AH + FP + MS + KQ; | | | | |
| | EC + PS + RT + OS + FP; PS + RT + MF + ES + MS; | | | | |
| | FPL + EL + LA + AH + KQ; EC + RT + OS + FP + RI; | | | | |
| | SG + DS + MF + MM + AH; SG + FPL + OS + MF + FP; | | | | |
| | EC + SG + OS + EL + AH; EC + FPL + EL + LA + MS; | | | | |
| | SG + DS + EL + MM + AH; PS + MM + AH + MS + KQ; | | | | |
| | DS + OS + LA + TB + FP; FPL + MF + MM + ES + MS; | | | | |
| | FPL + MM + MS + KQ + RI; LA + MM + AH + MS + RI; | | | | |
| | MF + AH + ES + MS + KQ; EC + PS + MM + FP + RI; | | | | |
| | EC + LA + MM + AH + RI; SG + MF + MM + AH + MS; | | | | |
| | PS + DS + FPL + OS + MF; RT + EL + MF + MM + RI; | | | | |
| | RT + EL + AH + KQ + RI; DS + EL + LA + ES + MS; | | | | |
| | EL + MF + TB + AH + MS; EC + SG + OS + LA + FP; | | | | |
| | EC + PS + OS + FP + MS; SG + RT + DS + EL + AH; | | | | |
| | SG + RT + MF + MM + ES; PS + FPL + LA + FP + MS; | | | | |
| | SG + PS + DS + MF + TB; SG + RT + MM + AH + RI; | | | | |
| | PS + DS + OS + EL + LA; DS + OS + MF + TB + AH; | | | | |
| | EC + RT + OS + KQ + RI; EC + OS + LA + AH + FP; | | | | |
| | EC + EL + LA + MM + AH; PS + MF + MM + AH + FP; | | | | |
| | RT + MF + TB + MS + RI; OS + EL + LA + TB + KQ; | | | | |
| | EL + MF + TB + MS + KQ; DS + OS + TB + FP + RI; | | | | |
| | SG + EL + MF + ES + KQ; DS + OS + TB + AH + KQ; | | | | |
| | DS + MM + AH + FP + KQ; EC + FPL + MF + KQ + RI; | | | | |
| | SG + FPL + MF + MM + KQ; PS + EL + MM + FP + MS; | | | | |
| | FPL + TB + AH + FP + MS; LA + MM + FP + KQ + RI; | | | | |
| | RT + EL + TB + AH + KQ; DS + FPL + OS + ES + MS; | | | | |
| | FPL + EL + MF + MS + RI; EL + LA + MM + ES + RI; | | | | |
| | TB + AH + MS + KQ + RI; EC + RT + EL + MF + MM; | | | | |
| | EC + DS + OS + EL + TB; EC + OS + LA + MF + TB; | | | | |
| | SG + PS + RT + MF + MS; SG + PS + DS + MF + MS; | | | | |
| | FPL + EL + MM + FP + RI; OS + LA + TB + AH + MS; | | | | |
| | EC + SG + OS + TB + KQ; SG + PS + LA + MF + KQ; | | | | |
| | SG + DS + TB + MM + KQ; PS + RT + MF + AH + KQ; | | | | |
| | PS + DS + EL + MF + MS; RT + DS + MF + AH + MS; | | | | |
| | RT + EL + MF + TB + MS; FPL + MF + MM + AH + KQ; | | | | |
| | EC + EL + LA + MF + KQ; SG + PS + DS + MM + FP; | | | | |
| | PS + AH + MS + KQ + RI; OS + EL + TB + FP + ES; | | | | |
| | EC + MF + AH + MS + KQ; RT + FPL + OS + FP + RI; | | | | |
| | FPL + MF + FP + KQ + RI; EL + MF + TB + MM + MS; | | | | |
| | SG + EL + LA + MF + ES; PS + FPL + EL + LA + MF; | | | | |
| | RT + DS + MM + ES + KQ; EC + RT + MF + MM + RI; | | | | |
| | PS + RT + DS + FPL + MM; PS + DS + OS + LA + RI; | | | | |
| | PS + EL + LA + MS + RI; PS + EL + MM + KQ + RI; | | | | |
| | RT + LA + AH + MS + RI; EL + MF + TB + KQ + RI; | | | | |
| | EC + OS + AH + FP + ES; EC + EL + AH + KQ + RI; | | | | |
| | EC + MF + MS + KQ + RI; SG + RT + FPL + MF + MS; | | | | |
| | SG + EL + LA + AH + MS; SG + EL + MF + KQ + RI; | | | | |
| | SG + RT + AH + MS + RI; SG + OS + LA + AH + KQ; | | | | |
| | PS + RT + DS + EL + AH; RT + AH + FP + ES + MS; | | | | |
| | DS + OS + MM + FP + MS; MM + FP + ES + MS + KQ; | | | | |
| | AH + ES + MS + KQ + RI; EC + DS + OS + EL + MF; | | | | |
| | SG + PS + RT + MF + AH; RT + OS + LA + AH + FP; | | | | |
| | FPL + LA + MF + KQ + RI; SG + RT + FPL + EL + AH; | | | | |
| | PS + DS + MF + MM + AH; PS + OS + EL + TB + KQ; | | | | |
| | DS + FPL + OS + MS + RI; DS + OS + EL + ES + RI; | | | | |
| | EL + MF + MM + FP + KQ; EC + LA + MF + KQ + RI; | | | | |
| | SG + RT + DS + MM + AH; DS + FPL + OS + EL + ES; | | | | |
| | FPL + MM + FP + MS + RI; EL + AH + FP + MS + KQ; | | | | |
| | SG + PS + DS + OS + EL; DS + OS + TB + MM + MS; | | | | |
| | EC + FPL + OS + LA + ES; EC + OS + TB + ES + KQ; | | | | |
| | PS + OS + EL + ES + RI; PS + LA + AH + MS + RI; | | | | |
| | RT + DS + MF + MS + KQ; RT + FPL + EL + MF + MM; | | | | |
| | RT + MF + MM + FP + KQ; EC + PS + RT + OS + MS; | | | | |
| | EC + RT + OS + EL + FP; PS + OS + LA + TB + AH; | | | | |
| | PS + EL + MS + KQ + RI; PS + MF + FP + ES + KQ; | | | | |
| | DS + EL + MF + ES + MS; FPL + OS + LA + TB + FP; | | | | |
| | OS + LA + TB + MM + KQ; OS + LA + TB + AH + RI; | | | | |
| | EC + OS + LA + AH + ES; SG + FPL + OS + AH + ES; | | | | |
| | PS + RT + OS + LA + ES; FPL + AH + FP + KQ + RI; | | | | |
| | EC + OS + MF + FP + ES; EC + DS + OS + MM + ES; | | | | |
| | EC + EL + LA + KQ + RI; SG + PS + FP + ES + MS; | | | | |
| | PS + FPL + EL + LA + AH; PS + MM + MS + KQ + RI; | | | | |
| | DS + OS + MM + AH + FP; EC + FPL + MM + AH + RI; | | | | |
| | SG + RT + OS + KQ + RI; SG + OS + MF + AH + FP; | | | | |
| | PS + MF + AH + FP + MS; DS + OS + TB + MM + KQ; | | | | |
| | PS + DS + FPL + AH + MS; PS + OS + EL + ES + KQ; | | | | |
| | DS + MF + MM + AH + KQ; DS + MF + AH + FP + KQ; | | | | |
| | FPL + OS + TB + FP + KQ; OS + EL + TB + AH + ES; | | | | |
| | PS + FPL + EL + FP + RI; RT + DS + MM + AH + KQ; | | | | |
| | EC + RT + OS + LA + MS; EC + EL + MM + MS + KQ; | | | | |
| | SG + RT + FPL + AH + MS; SG + RT + OS + EL + RI; | | | | |
| | PS + DS + FPL + EL + AH; PS + EL + MF + FP + RI; | | | | |
| | RT + DS + TB + AH + RI; RT + FPL + TB + MM + KQ; | | | | |
| | DS + FPL + OS + LA + MF; OS + EL + TB + ES + RI; | | | | |
| | OS + LA + TB + ES + RI; EC + OS + MF + TB + KQ; | | | | |
| | SG + RT + MF + MM + RI; RT + DS + FPL + OS + ES; | | | | |
| | FPL + LA + MF + MM + AH; EC + PS + DS + OS + MM; | | | | |
| | SG + OS + LA + FP + KQ; RT + DS + MF + MM + RI; | | | | |
| | FPL + TB + MM + AH + RI; EC + RT + FPL + OS + TB; | | | | |
| | EC + OS + LA + TB + RI; EC + EL + LA + MF + AH; | | | | |
| | SG + MF + FP + ES + KQ; PS + RT + AH + FP + MS; | | | | |
| | FPL + EL + LA + MF + RI; EC + OS + TB + AH + ES; | | | | |
| | EC + OS + TB + KQ + RI; PS + DS + OS + AH + RI; | | | | |
| | PS + FPL + EL + AH + FP; DS + EL + TB + MM + ES; | | | | |
| | FPL + EL + AH + MS + KQ; EC + DS + OS + LA + MM; | | | | |
| | RT + LA + MF + AH + MS; DS + MF + AH + KQ + RI; | | | | |
| | SG + PS + DS + EL + TB; PS + LA + MF + FP + KQ; | | | | |
| | DS + EL + ES + KQ + RI; MF + MM + AH + ES + KQ; | | | | |
| | EC + SG + OS + EL + TB; EC + SG + OS + ES + KQ; | | | | |
| | EC + PS + OS + MF + MS; EC + OS + FP + ES + RI; | | | | |
| | DS + OS + TB + MM + AH; EC + SG + OS + TB + MM; | | | | |
| | EC + FPL + MM + KQ + RI; EC + OS + EL + TB + ES; | | | | |
| | SG + DS + FPL + OS + MS; RT + MM + AH + FP + ES; | | | | |
| | DS + EL + TB + KQ + RI; FPL + TB + MM + AH + FP; | | | | |
| | EC + RT + FPL + OS + AH; SG + OS + LA + AH + MS; | | | | |
| | PS + FPL + FP + MS + RI; RT + MF + TB + ES + MS; | | | | |
| | DS + MF + MM + FP + KQ; EC + SG + RT + FPL + OS; | | | | |
| | SG + PS + TB + FP + RI; SG + FPL + OS + MF + TB; | | | | |
| | SG + MF + MM + AH + KQ; EL + MF + AH + FP + MS; | | | | |
| | SG + FPL + OS + FP + RI; PS + DS + LA + MF + KQ; | | | | |
| | RT + TB + AH + FP + RI; EL + LA + MF + AH + KQ; | | | | |
| | EL + MM + ES + MS + RI; MF + MM + AH + ES + MS; | | | | |
| | EC + DS + FPL + OS + AH; PS + FPL + MF + MM + RI; | | | | |
| | DS + OS + LA + FP + ES; EC + PS + RT + OS + AH; | | | | |
| | SG + RT + DS + TB + AH; SG + MF + AH + MS + RI; | | | | |
| | DS + OS + AH + FP + RI; FPL + MF + MM + MS + KQ; | | | | |
| | EC + PS + OS + EL + MF; EC + PS + EL + FP + RI; | | | | |
| | EC + DS + OS + EL + AH; EC + EL + MF + AH + MS; | | | | |
| | PS + DS + OS + TB + MM; RT + FPL + LA + MF + MM; | | | | |
| | FPL + EL + MF + MM + RI; MF + MM + FP + MS + KQ; | | | | |
| | EC + OS + MF + TB + ES; SG + PS + MF + TB + ES; | | | | |
| | PS + FPL + AH + FP + RI; RT + OS + TB + AH + FP; | | | | |
| | RT + LA + MF + MM + ES; OS + LA + TB + MS + KQ; | | | | |
| | EC + SG + DS + OS + MM; EC + RT + OS + TB + AH; | | | | |
| | PS + OS + LA + TB + ES; PS + MF + AH + FP + RI; | | | | |
| | EC + PS + OS + AH + KQ; PS + FPL + MF + MM + MS; | | | | |
| | EC + OS + LA + ES + RI; EC + EL + MF + KQ + RI; | | | | |
| | EC + MF + AH + KQ + RI; EC + MM + AH + MS + KQ; | | | | |
| | SG + FPL + OS + LA + RI; PS + MF + TB + MS + RI; | | | | |
| | PS + MF + FP + KQ + RI; DS + FPL + OS + MF + AH; | | | | |
| | DS + FPL + OS + AH + FP; EC + MM + MS + KQ + RI; | | | | |
| | SG + PS + RT + TB + MM; DS + MF + MM + AH + ES; | | | | |
| | EC + RT + DS + OS + KQ; PS + RT + MM + AH + KQ; | | | | |
| | PS + FPL + LA + MM + MS; RT + DS + TB + AH + MS; | | | | |
| | DS + FPL + OS + TB + AH; FPL + MF + TB + MM + RI; | | | | |
| | OS + LA + TB + AH + KQ; EC + OS + FP + ES + MS; | | | | |
| | EC + EL + LA + AH + RI; RT + FPL + MM + MS + KQ; | | | | |
| | DS + MF + TB + MM + MS; FPL + EL + LA + MS + RI; | | | | |
| | FPL + MM + AH + ES + RI; EC + SG + OS + KQ + RI; | | | | |
| | EC + MF + MM + ES + MS; SG + EL + MF + TB + KQ; | | | | |
| | SG + MF + TB + MM + MS; PS + FPL + LA + MF + AH; | | | | |
| | EC + RT + OS + LA + RI; SG + PS + FPL + EL + AH; | | | | |
| | SG + RT + EL + AH + RI; SG + DS + OS + LA + MS; | | | | |
| | PS + DS + OS + TB + RI; PS + DS + EL + MM + RI; | | | | |
| | EC + OS + FP + ES + KQ; SG + PS + MF + AH + MS; | | | | |
| | SG + FPL + OS + TB + AH; PS + DS + FPL + OS + TB; | | | | |
| | PS + DS + LA + MM + KQ; DS + EL + LA + MS + RI; | | | | |
| | DS + EL + LA + KQ + RI; DS + MF + FP + KQ + RI; | | | | |
| | FPL + EL + TB + AH + MS; MF + TB + AH + MS + RI; | | | | |
| | EC + DS + OS + MM + AH; EC + OS + LA + FP + ES; | | | | |
| | EC + EL + AH + FP + RI; PS + DS + FPL + MM + AH; | | | | |
| | RT + DS + FPL + OS + MS; DS + OS + EL + FP + MS; | | | | |
| | EC + EL + MM + AH + MS; PS + RT + DS + EL + MF; | | | | |
| | RT + DS + MF + MM + FP; RT + DS + MM + AH + MS; | | | | |
| | RT + FPL + MM + AH + FP; FPL + MF + ES + MS + RI; | | | | |
| | OS + TB + MM + FP + ES; EC + DS + OS + MF + KQ; | | | | |
| | RT + MF + AH + ES + MS; SG + FPL + MF + MM + AH; | | | | |
| | PS + RT + TB + MM + KQ; RT + MF + AH + ES + KQ; | | | | |
| | DS + EL + LA + ES + KQ; LA + MF + MS + KQ + RI; | | | | |
| | EC + OS + EL + LA + TB; EC + OS + LA + TB + MM; | | | | |
| | EC + EL + MF + MM + MS; FPL + OS + LA + AH + FP; | | | | |
| | EC + PS + OS + MM + ES; EC + RT + MF + TB + MM; | | | | |
| | SG + PS + DS + EL + MM; SG + DS + OS + MM + AH; | | | | |
| | RT + EL + TB + MM + MS; LA + MF + ES + MS + KQ; | | | | |
| | PS + MF + MM + FP + MS; RT + LA + MF + MM + MS; | | | | |
| | RT + MM + AH + ES + MS; DS + EL + MM + AH + RI; | | | | |
| | DS + MF + TB + MS + KQ; DS + MF + AH + MS + KQ; | | | | |
| | EC + PS + OS + MS + KQ; EC + FPL + OS + TB + AH; | | | | |
| | SG + RT + MF + TB + KQ; DS + FPL + OS + KQ + RI; | | | | |
| | EC + SG + FPL + OS + MF; PS + RT + DS + MF + MS; | | | | |
| | PS + RT + MF + AH + RI; PS + MF + FP + ES + RI; | | | | |
| | PS + MM + FP + MS + KQ; DS + FPL + MM + AH + MS; | | | | |
| | DS + OS + EL + FP + RI; MF + TB + AH + MS + KQ; | | | | |
| | EC + SG + OS + LA + MM; EL + LA + ES + MS + RI; | | | | |
| | EC + DS + OS + EL + FP; SG + EL + MF + TB + MM; | | | | |
| | EC + PS + OS + LA + FP; SG + MF + MM + AH + RI; | | | | |
| | LA + MF + AH + MS + KQ; SG + DS + MF + FP + KQ; | | | | |
| | DS + LA + MF + MS + KQ; FPL + LA + AH + MS + KQ; | | | | |
| | LA + MF + MM + FP + ES; MF + MM + AH + FP + MS; | | | | |
| | EC + LA + MM + KQ + RI; SG + RT + DS + MF + TB; | | | | |
| | SG + EL + TB + MM + AH; PS + DS + OS + AH + FP; | | | | |
| | DS + EL + MF + TB + KQ; EL + MF + MM + FP + ES; | | | | |
| | TB + MM + ES + KQ + RI; EC + RT + MF + MM + AH; | | | | |
| | SG + PS + RT + EL + AH; SG + PS + OS + TB + AH; | | | | |
| | PS + FPL + OS + TB + ES; PS + OS + EL + MM + ES; | | | | |
| | RT + OS + LA + AH + ES; DS + FPL + OS + EL + TB; | | | | |
| | FPL + LA + AH + KQ + RI; LA + AH + FP + MS + RI; | | | | |
| | EC + FPL + LA + MM + KQ; DS + FPL + AH + MS + RI; | | | | |
| | EC + OS + MF + TB + AH; RT + OS + LA + ES + RI; | | | | |
| | DS + FPL + OS + MF + KQ; FPL + EL + FP + MS + RI; | | | | |
| | EC + DS + OS + MM + FP; EC + OS + AH + FP + KQ; | | | | |
| | RT + FPL + EL + AH + FP; DS + OS + AH + FP + ES; | | | | |
| | DS + OS + FP + ES + RI; FPL + EL + LA + MM + RI; | | | | |
| | EC + PS + FPL + OS + KQ; SG + PS + EL + MM + MS; | | | | |
| | SG + RT + TB + AH + RI; SG + EL + MF + TB + AH; | | | | |
| | EL + TB + AH + MS + RI; EC + OS + TB + ES + RI; | | | | |
| | RT + MM + AH + KQ + RI; DS + FPL + OS + LA + KQ; | | | | |
| | DS + LA + MF + MM + FP; OS + LA + TB + MM + AH; | | | | |
| | MM + FP + MS + KQ + RI; EC + SG + OS + MS + RI; | | | | |
| | SG + PS + RT + MF + KQ; SG + FPL + OS + LA + KQ; | | | | |
| | RT + MM + AH + FP + KQ; DS + OS + EL + AH + ES; | | | | |
| | EL + TB + MM + AH + RI; SG + OS + LA + MM + AH; | | | | |
| | PS + MF + MM + AH + KQ; RT + FPL + MM + MS + RI; | | | | |
| | RT + LA + MF + MM + AH; DS + FPL + OS + AH + KQ; | | | | |
| | LA + MF + TB + MM + FP; EC + OS + MM + FP + ES; | | | | |
| | SG + MF + MM + FP + KQ; PS + DS + EL + MM + ES; | | | | |
| | DS + EL + MF + KQ + RI; EC + PS + OS + LA + MF; | | | | |
| | EC + DS + OS + AH + MS; PS + EL + AH + MS + RI; | | | | |
| | RT + OS + EL + ES + RI; DS + OS + EL + TB + AH; | | | | |
| | LA + MF + MM + AH + KQ; EC + PS + FPL + OS + FP; | | | | |
| | SG + RT + MM + AH + FP; RT + TB + MM + AH + KQ; | | | | |
| | PS + FP + MS + KQ + RI; OS + TB + AH + FP + ES; | | | | |
| | SG + RT + OS + TB + FP; PS + MM + AH + FP + MS; | | | | |
| | RT + MF + MM + FP + RI; DS + MF + MM + FP + RI; | | | | |
| | OS + EL + LA + TB + AH; RT + FPL + MF + MS + KQ; | | | | |
| | DS + OS + TB + MM + ES; DS + OS + AH + ES + RI; | | | | |
| | EC + OS + LA + MF + ES; SG + RT + MF + ES + KQ; | | | | |
| | SG + FPL + EL + MF + AH; DS + OS + TB + MS + RI; | | | | |
| | EC + PS + OS + ES + MS; DS + OS + TB + AH + MS; | | | | |
| | DS + EL + LA + MF + ES; EL + LA + MF + AH + MS; | | | | |
| | EC + SG + OS + EL + MF; SG + RT + OS + FP + RI; | | | | |
| | SG + DS + OS + ES + RI; SG + EL + MF + MM + RI; | | | | |
| | RT + FPL + OS + TB + ES; DS + FPL + OS + MM + ES; | | | | |
| | PS + DS + OS + MS + RI; PS + DS + MF + ES + KQ; | | | | |
| | DS + FPL + OS + MF + ES; DS + EL + LA + MM + MS; | | | | |
| | FPL + OS + EL + TB + ES; EC + DS + OS + ES + MS; | | | | |
| | EC + FPL + OS + TB + KQ; EC + OS + MF + AH + FP; | | | | |
| | SG + DS + OS + EL + KQ; SG + MM + MS + KQ + RI; | | | | |
| | PS + FPL + MM + AH + ES; DS + OS + LA + ES + MS; | | | | |
| | EC + RT + FPL + OS + LA; EC + RT + OS + MF + FP; | | | | |
| | EC + FPL + OS + TB + RI; EC + OS + EL + FP + ES; | | | | |
| | FPL + OS + MF + TB + FP; FPL + MF + AH + KQ + RI; | | | | |
| | SG + FPL + EL + MM + AH; SG + OS + AH + FP + RI; | | | | |
| | SG + MF + FP + ES + MS; DS + FPL + OS + MF + FP; | | | | |
| | EC + FPL + OS + AH + ES; SG + OS + LA + TB + MS; | | | | |
| | PS + DS + OS + AH + ES; PS + MF + FP + MS + KQ; | | | | |
| | EL + MM + AH + FP + MS; LA + MF + ES + KQ + RI; | | | | |
| | MM + ES + MS + KQ + RI; EC + SG + OS + MF + TB; | | | | |
| | EC + PS + OS + MF + FP; EC + PS + OS + FP + RI; | | | | |
| | EC + EL + AH + MS + RI; SG + PS + OS + EL + LA; | | | | |
| | LA + MF + TB + KQ + RI; EC + OS + LA + ES + MS; | | | | |
| | FPL + LA + MF + TB + MM; OS + EL + TB + ES + KQ; | | | | |
| | AH + FP + ES + KQ + RI; EC + PS + OS + EL + AH; | | | | |
| | SG + PS + MF + KQ + RI; PS + DS + FPL + OS + KQ; | | | | |
| | PS + LA + MF + KQ + RI; RT + DS + EL + MM + AH; | | | | |
| | RT + OS + TB + FP + KQ; EL + LA + AH + KQ + RI; | | | | |
| | SG + RT + EL + TB + AH; SG + OS + LA + TB + KQ; | | | | |
| | SG + DS + OS + ES + MS; PS + DS + MF + FP + KQ; | | | | |
| | DS + OS + TB + AH + ES; EC + SG + FPL + OS + ES; | | | | |
| | EC + FPL + MM + AH + MS; RT + AH + FP + MS + KQ; | | | | |
| | DS + OS + TB + ES + MS; EC + PS + FPL + OS + RI; | | | | |
| | EC + DS + FPL + OS + MM; | | | | |
| | FPL + EL + LA + MF + MM; LA + AH + MS + KQ + RI; | | | | |
| | EC + SG + OS + AH + MS; EC + RT + FPL + OS + MF; | | | | |
| | EC + FPL + OS + AH + FP; EC + EL + MM + FP + RI; | | | | |
| | SG + PS + FPL + MM + AH; | | | | |
| | SG + FPL + MM + AH + MS; PS + DS + OS + ES + RI; | | | | |
| | RT + OS + LA + AH + KQ; MF + MM + AH + FP + KQ; | | | | |
| | SG + RT + OS + LA + TB; PS + RT + OS + EL + RI | | | | |
| 5 | EC + SG + EL + MF + RI; EC + PS + OS + MF + TB; | 0.73 | 0.2632- | 42.86%- | 37.37%- |
| | EC + DS + OS + EL + ES; SG + PS + EL + TB + AH; | | 0.5613 | 96.83% | 91.92% |
| | SG + EL + LA + MM + AH; SG + EL + LA + AH + RI; | | | | |
| | SG + EL + AH + KQ + RI; DS + FPL + EL + AH + FP; | | | | |
| | DS + LA + MF + FP + RI; FPL + EL + MF + MM + MS; | | | | |
| | EL + MM + AH + ES + RI; EC + SG + DS + OS + FP; | | | | |
| | EC + SG + OS + TB + AH; EC + SG + MF + MM + AH; | | | | |
| | EC + FPL + LA + MF + AH; EC + FPL + MM + FP + KQ; | | | | |
| | EC + MM + FP + KQ + RI; SG + PS + FPL + MF + MS; | | | | |
| | SG + PS + AH + MS + KQ; PS + RT + DS + FP + KQ; | | | | |
| | RT + DS + FPL + MF + MM; | | | | |
| | RT + FPL + MM + ES + MS; RT + LA + MM + AH + KQ; | | | | |
| | EC + SG + OS + EL + KQ; EC + EL + LA + MM + KQ; | | | | |
| | PS + EL + ES + KQ + RI; RT + TB + AH + ES + RI; | | | | |
| | FPL + EL + MM + AH + KQ; EL + AH + ES + KQ + RI; | | | | |
| | EC + DS + OS + MF + FP; SG + PS + LA + MF + ES; | | | | |
| | PS + DS + MM + MS + KQ; FPL + TB + MM + MS + RI; | | | | |
| | SG + PS + MM + AH + MS; RT + DS + MM + MS + KQ; | | | | |
| | DS + FPL + TB + AH + MS; EC + SG + OS + FP + KQ; | | | | |
| | PS + RT + DS + AH + KQ; RT + EL + TB + MM + RI; | | | | |
| | RT + LA + MF + MS + KQ; DS + FPL + EL + AH + MS; | | | | |
| | DS + AH + ES + KQ + RI; EC + SG + EL + AH + RI; | | | | |
| | DS + MF + TB + KQ + RI; MM + AH + ES + MS + KQ; | | | | |
| | EC + FPL + LA + AH + KQ; SG + RT + TB + MS + KQ; | | | | |
| | SG + DS + MM + ES + KQ; RT + LA + AH + FP + RI; | | | | |
| | LA + MF + AH + ES + KQ; EC + PS + OS + TB + FP; | | | | |
| | EC + DS + OS + TB + RI; EC + EL + LA + AH + MS; | | | | |
| | SG + TB + ES + KQ + RI; PS + RT + MM + FP + RI; | | | | |
| | PS + EL + MF + FP + KQ; EC + EL + MM + ES + RI; | | | | |
| | SG + RT + EL + TB + MS; SG + OS + TB + AH + FP; | | | | |
| | RT + DS + EL + MM + MS; RT + LA + MM + AH + MS; | | | | |
| | RT + MF + MM + ES + RI; FPL + MF + MM + FP + RI; | | | | |
| | EL + MF + FP + MS + KQ; EC + PS + RT + DS + OS; | | | | |
| | EC + DS + EL + MS + RI; SG + LA + MF + ES + MS; | | | | |
| | PS + RT + DS + FPL + AH; PS + RT + DS + TB + MS; | | | | |
| | PS + EL + MF + AH + KQ; RT + LA + TB + MM + RI; | | | | |
| | DS + LA + MF + ES + KQ; EC + PS + RT + FPL + MM; | | | | |
| | EC + RT + DS + OS + AH; EC + RT + AH + FP + RI; | | | | |
| | EC + FPL + EL + MS + RI; SG + RT + MM + AH + KQ; | | | | |
| | PS + RT + LA + AH + MS; RT + DS + OS + ES + RI; | | | | |
| | RT + DS + MM + FP + KQ; EC + RT + EL + AH + RI; | | | | |
| | EC + DS + OS + AH + KQ; EC + MF + TB + MM + RI; | | | | |
| | SG + RT + TB + MM + AH; SG + EL + LA + MM + MS; | | | | |
| | RT + LA + MM + AH + RI; RT + MM + FP + KQ + RI; | | | | |
| | DS + AH + FP + MS + KQ; EC + RT + MF + KQ + RI; | | | | |
| | EC + FPL + EL + LA + MM; SG + RT + LA + MF + MM; | | | | |
| | PS + ES + MS + KQ + RI; FPL + LA + MF + ES + MS; | | | | |
| | EC + RT + MF + TB + AH; PS + DS + AH + MS + KQ; | | | | |
| | FPL + LA + AH + ES + RI; EC + FPL + MF + MM + AH; | | | | |
| | EC + FPL + AH + MS + KQ; EC + TB + AH + MS + RI; | | | | |
| | SG + PS + DS + TB + KQ; SG + PS + DS + MM + MS; | | | | |
| | SG + RT + FPL + MF + MM; SG + EL + LA + TB + AH; | | | | |
| | SG + EL + LA + MM + RI; PS + DS + EL + KQ + RI; | | | | |
| | PS + FPL + MF + FP + KQ; RT + FPL + EL + AH + KQ; | | | | |
| | RT + EL + LA + TB + AH; RT + LA + MF + TB + KQ; | | | | |
| | FPL + EL + MF + ES + KQ; EL + MF + AH + ES + MS; | | | | |
| | SG + PS + MM + ES + MS; SG + PS + AH + FP + MS; | | | | |
| | RT + DS + TB + AH + KQ; FPL + MM + FP + KQ + RI; | | | | |
| | EL + MF + AH + ES + KQ; LA + MF + MM + AH + FP; | | | | |
| | RT + TB + MM + ES + KQ; SG + RT + EL + MF + MS; | | | | |
| | SG + MF + TB + AH + KQ; PS + RT + TB + AH + RI; | | | | |
| | PS + FPL + MF + KQ + RI; PS + EL + LA + MM + AH; | | | | |
| | PS + MF + MM + AH + ES; RT + DS + OS + LA + AH; | | | | |
| | DS + LA + MF + AH + KQ; EL + LA + ES + KQ + RI; | | | | |
| | EC + RT + MF + AH + MS; EC + EL + LA + MM + FP; | | | | |
| | SG + PS + EL + MF + KQ; SG + RT + OS + EL + LA; | | | | |
| | SG + RT + AH + MS + KQ; PS + RT + FPL + TB + AH; | | | | |
| | PS + RT + FPL + MM + FP; RT + FPL + LA + MF + MS; | | | | |
| | RT + AH + FP + ES + RI; DS + LA + MF + MS + RI; | | | | |
| | MF + TB + MM + AH + KQ; EC + PS + EL + AH + RI; | | | | |
| | EC + FPL + LA + FP + MS; EC + EL + MF + FP + RI; | | | | |
| | SG + PS + MF + MS + RI; PS + MM + AH + FP + KQ; | | | | |
| | RT + FPL + EL + MF + TB; RT + FPL + EL + MM + RI; | | | | |
| | DS + FPL + OS + LA + RI; EC + DS + EL + MF + MM; | | | | |
| | EC + LA + MF + FP + KQ; SG + EL + AH + FP + RI; | | | | |
| | PS + RT + MM + AH + ES; RT + DS + MF + TB + MS; | | | | |
| | RT + TB + MM + ES + MS; FP + ES + MS + KQ + RI; | | | | |
| | EC + RT + DS + MF + MM; EC + RT + OS + LA + TB; | | | | |
| | EC + FPL + LA + MM + AH; EC + FPL + MF + MS + RI; | | | | |
| | SG + EL + LA + MF + AH; FPL + EL + TB + MM + AH; | | | | |
| | FPL + AH + ES + MS + KQ; EC + RT + MF + MS + KQ; | | | | |
| | EC + DS + OS + AH + RI; EC + FPL + EL + MF + RI; | | | | |
| | EC + LA + MM + FP + MS; EC + MF + FP + KQ + RI; | | | | |
| | RT + DS + OS + TB + RI; RT + FPL + EL + FP + RI; | | | | |
| | RT + FPL + MF + AH + FP; FPL + EL + MM + AH + ES; | | | | |
| | EC + SG + RT + OS + EL; EC + RT + OS + AH + ES; | | | | |
| | PS + FPL + LA + MF + KQ; RT + DS + AH + MS + RI; | | | | |
| | RT + FPL + TB + MM + RI; DS + EL + LA + MS + KQ; | | | | |
| | EC + SG + OS + AH + KQ; EC + SG + EL + MM + MS; | | | | |
| | SG + RT + FPL + MF + AH; RT + DS + OS + LA + FP; | | | | |
| | FPL + EL + LA + ES + KQ; LA + MF + TB + AH + RI; | | | | |
| | TB + MM + AH + FP + RI; EC + SG + EL + MM + RI; | | | | |
| | EC + PS + MF + MM + RI; PS + RT + MM + FP + ES; | | | | |
| | RT + DS + MF + AH + ES; RT + LA + MF + TB + RI; | | | | |
| | EC + RT + FPL + EL + MF; EC + RT + FPL + MF + KQ; | | | | |
| | EC + FPL + LA + MF + KQ; EC + FPL + LA + MM + FP; | | | | |
| | SG + PS + AH + MS + RI; SG + DS + OS + AH + RI; | | | | |
| | SG + MF + TB + ES + MS; PS + RT + FPL + AH + FP; | | | | |
| | PS + DS + OS + LA + KQ; DS + LA + MM + FP + KQ; | | | | |
| | DS + MM + AH + MS + KQ; | | | | |
| | FPL + TB + MM + AH + KQ; EC + MF + AH + FP + KQ; | | | | |
| | SG + PS + EL + MF + MM; RT + DS + EL + LA + MF; | | | | |
| | RT + DS + EL + MF + AH; FPL + LA + MF + MM + FP; | | | | |
| | EC + SG + RT + OS + MS; EC + RT + EL + MF + KQ; | | | | |
| | SG + MF + TB + MM + AH; RT + DS + TB + MM + MS; | | | | |
| | EC + PS + EL + MM + RI; EC + FPL + EL + AH + MS; | | | | |
| | EC + LA + FP + ES + KQ; SG + RT + DS + TB + MM; | | | | |
| | PS + RT + FPL + AH + RI; RT + MF + FP + MS + KQ; | | | | |
| | EL + MF + TB + MM + KQ; EC + RT + OS + ES + RI; | | | | |
| | SG + LA + MF + TB + RI; PS + RT + MM + ES + MS; | | | | |
| | PS + DS + FPL + FP + MS; EC + DS + OS + MF + RI; | | | | |
| | SG + MF + AH + KQ + RI; RT + LA + MF + FP + KQ; | | | | |
| | SG + DS + MF + FP + ES; PS + DS + MF + TB + MS; | | | | |
| | RT + OS + LA + FP + ES; RT + EL + LA + AH + RI; | | | | |
| | DS + EL + MF + AH + KQ; EC + FPL + EL + MM + MS; | | | | |
| | EC + FPL + LA + MM + MS; PS + EL + LA + ES + MS; | | | | |
| | RT + FPL + EL + MF + KQ; DS + MF + TB + MM + RI; | | | | |
| | DS + MF + AH + ES + KQ; FPL + LA + MF + ES + KQ; | | | | |
| | EC + PS + AH + FP + RI; EC + EL + MS + KQ + RI; | | | | |
| | PS + DS + FPL + TB + AH; PS + MF + TB + MM + AH; | | | | |
| | RT + MF + TB + KQ + RI; SG + DS + OS + AH + KQ; | | | | |
| | SG + FPL + TB + MM + AH; SG + AH + MS + KQ + RI; | | | | |
| | EL + LA + MM + FP + KQ; SG + RT + EL + MM + MS; | | | | |
| | SG + FPL + EL + TB + AH; RT + FPL + TB + AH + RI; | | | | |
| | RT + TB + MM + MS + RI; DS + OS + LA + TB + ES; | | | | |
| | DS + MM + ES + MS + KQ; EC + RT + DS + OS + MS; | | | | |
| | EC + DS + EL + KQ + RI; SG + PS + AH + FP + RI; | | | | |
| | PS + RT + DS + MM + MS; RT + EL + TB + AH + ES; | | | | |
| | RT + TB + MM + AH + ES; FPL + MM + ES + KQ + RI; | | | | |
| | MF + AH + FP + ES + KQ; EC + RT + MF + MM + FP; | | | | |
| | EC + EL + LA + MF + FP; PS + DS + MM + FP + KQ; | | | | |
| | DS + EL + MM + AH + KQ; TB + MM + AH + MS + KQ; | | | | |
| | EC + PS + OS + TB + AH; EC + FPL + OS + LA + TB; | | | | |
| | SG + MF + ES + MS + RI; PS + FPL + EL + AH + KQ; | | | | |
| | PS + LA + MM + MS + RI; PS + TB + MS + KQ + RI; | | | | |
| | RT + MF + AH + ES + RI; DS + EL + MF + TB + MS; | | | | |
| | EC + FPL + EL + MF + KQ; RT + DS + AH + ES + MS; | | | | |
| | EC + PS + RT + MF + MM; SG + DS + OS + AH + MS; | | | | |
| | PS + RT + FPL + MF + KQ; PS + FPL + EL + AH + ES; | | | | |
| | DS + OS + LA + AH + ES; DS + MF + MM + AH + RI; | | | | |
| | SG + PS + DS + MF + FP; PS + FPL + MF + MM + KQ; | | | | |
| | PS + TB + MM + MS + KQ; RT + DS + AH + FP + ES; | | | | |
| | RT + EL + LA + MF + AH; EL + LA + MM + FP + ES; | | | | |
| | EC + SG + OS + FP + RI; EC + DS + OS + EL + LA; | | | | |
| | EC + DS + OS + FP + ES; EC + AH + FP + MS + RI; | | | | |
| | PS + RT + DS + MF + FP; DS + OS + TB + MM + FP; | | | | |
| | DS + OS + TB + AH + RI; EC + SG + OS + LA + MS; | | | | |
| | EC + RT + OS + TB + FP; EL + LA + FP + KQ + RI; | | | | |
| | EL + TB + AH + MS + KQ; SG + TB + MM + KQ + RI; | | | | |
| | RT + DS + AH + KQ + RI; FPL + MF + ES + KQ + RI; | | | | |
| | RT + DS + LA + MM + KQ; EC + DS + OS + MF + ES; | | | | |
| | EC + EL + LA + MM + MS; SG + RT + EL + TB + MM; | | | | |
| | SG + FPL + EL + LA + AH; SG + EL + MM + MS + RI; | | | | |
| | PS + MF + AH + FP + KQ; EC + RT + DS + OS + MF; | | | | |
| | EC + TB + MM + AH + RI; SG + PS + DS + MF + ES; | | | | |
| | RT + DS + MM + AH + FP; RT + OS + EL + TB + FP; | | | | |
| | EC + FPL + LA + FP + RI; SG + OS + LA + AH + RI; | | | | |
| | PS + RT + TB + AH + FP; PS + FPL + EL + MF + KQ; | | | | |
| | RT + DS + TB + ES + KQ; EC + SG + OS + TB + RI; | | | | |
| | EC + FPL + LA + MS + KQ; SG + LA + MF + MM + AH; | | | | |
| | PS + RT + DS + AH + FP; RT + EL + AH + FP + KQ; | | | | |
| | RT + LA + MF + AH + KQ; EC + SG + OS + MF + FP; | | | | |
| | EC + OS + EL + TB + KQ; SG + PS + EL + MF + TB; | | | | |
| | SG + DS + OS + LA + RI; SG + FPL + MF + MM + MS; | | | | |
| | RT + EL + MM + FP + MS; OS + LA + TB + AH + ES; | | | | |
| | AH + FP + ES + MS + KQ; EC + PS + OS + EL + TB; | | | | |
| | EC + FPL + MM + AH + KQ; PS + FPL + MF + MS + RI; | | | | |
| | PS + EL + LA + MF + AH; RT + FPL + MM + FP + KQ; | | | | |
| | FPL + LA + MM + AH + KQ; PS + EL + MF + TB + RI; | | | | |
| | RT + FPL + MF + TB + AH; FPL + EL + MF + MS + KQ; | | | | |
| | EL + MF + AH + FP + KQ; EL + TB + AH + KQ + RI; | | | | |
| | PS + DS + OS + LA + ES; PS + FPL + MF + AH + RI; | | | | |
| | RT + DS + OS + MF + RI; RT + DS + TB + MM + ES; | | | | |
| | RT + FPL + EL + TB + AH; EC + SG + MF + MM + MS; | | | | |
| | EC + PS + RT + OS + RI; TB + ES + MS + KQ + RI; | | | | |
| | EC + RT + MM + AH + MS; SG + PS + RT + TB + AH; | | | | |
| | SG + DS + OS + LA + ES; SG + EL + MF + MS + RI; | | | | |
| | RT + FPL + MM + AH + ES; EC + LA + MF + AH + KQ; | | | | |
| | SG + RT + EL + MF + TB; SG + MF + FP + KQ + RI; | | | | |
| | PS + MF + TB + AH + MS; EC + DS + OS + ES + KQ; | | | | |
| | EC + FPL + MF + MS + KQ; SG + PS + MF + AH + KQ; | | | | |
| | PS + RT + EL + TB + MM; PS + MF + FP + MS + RI; | | | | |
| | DS + OS + MF + AH + FP; EC + EL + AH + MS + KQ; | | | | |
| | PS + EL + LA + TB + RI; RT + DS + AH + FP + KQ; | | | | |
| | EC + RT + OS + TB + ES; EC + FPL + AH + FP + MS; | | | | |
| | SG + DS + EL + LA + MF; SG + EL + LA + KQ + RI; | | | | |
| | PS + DS + FPL + OS + LA; RT + DS + LA + MF + KQ; | | | | |
| | DS + EL + MM + AH + MS; EC + RT + FPL + OS + FP; | | | | |
| | EC + RT + MF + MM + MS; EC + EL + MF + MS + KQ; | | | | |
| | EC + FPL + EL + MS + KQ; EC + FPL + LA + MF + MM; | | | | |
| | EC + FPL + MF + AH + RI; EC + MF + MM + ES + RI; | | | | |
| | EC + MF + ES + KQ + RI; SG + PS + MF + TB + RI; | | | | |
| | SG + RT + OS + LA + MS; SG + EL + MF + TB + MS; | | | | |
| | FPL + LA + TB + MM + AH; EC + MF + MM + AH + FP; | | | | |
| | RT + DS + MF + ES + MS; DS + FPL + MF + MM + AH; | | | | |
| | FPL + MF + TB + AH + RI; PS + FPL + LA + MM + FP; | | | | |
| | PS + FPL + MF + MM + FP; SG + RT + EL + AH + ES; | | | | |
| | PS + RT + EL + MM + FP; PS + DS + FPL + OS + ES; | | | | |
| | PS + LA + MF + FP + RI; RT + FPL + EL + TB + MM; | | | | |
| | EC + SG + PS + OS + MF; RT + DS + OS + KQ + RI; | | | | |
| | RT + MM + ES + MS + KQ; EL + MF + MM + AH + FP; | | | | |
| | EC + DS + OS + TB + MM; EC + EL + MM + AH + KQ; | | | | |
| | DS + OS + LA + AH + RI; EC + SG + OS + TB + ES; | | | | |
| | SG + DS + OS + MS + RI; RT + DS + OS + FP + MS; | | | | |
| | DS + LA + MM + KQ + RI; FPL + LA + MS + KQ + RI; | | | | |
| | PS + DS + MF + AH + MS; PS + MF + TB + AH + KQ; | | | | |
| | PS + EL + MF + MM + FP; RT + FPL + MF + MM + FP; | | | | |
| | RT + EL + MF + MS + KQ; DS + EL + TB + MM + MS; | | | | |
| | DS + MF + ES + KQ + RI; EL + MM + FP + MS + KQ; | | | | |
| | EC + PS + OS + LA + RI; EC + PS + LA + MM + MS; | | | | |
| | SG + PS + EL + LA + MS; PS + RT + MF + AH + ES; | | | | |
| | PS + RT + TB + ES + KQ; RT + DS + FPL + MM + AH; | | | | |
| | RT + FPL + MF + MM + ES; | | | | |
| | DS + MF + MM + AH + MS; EL + LA + MM + AH + KQ; | | | | |
| | LA + MM + AH + FP + RI; EC + OS + TB + MM + FP; | | | | |
| | SG + RT + DS + AH + FP; SG + EL + MF + MM + ES; | | | | |
| | DS + MM + AH + KQ + RI; FPL + FP + MS + KQ + RI; | | | | |
| | SG + RT + DS + AH + RI; PS + RT + DS + OS + FP; | | | | |
| | PS + LA + MF + ES + KQ; RT + OS + LA + MF + FP; | | | | |
| | RT + EL + MF + MM + ES; FPL + MF + TB + MM + AH; | | | | |
| | FPL + TB + AH + KQ + RI; FPL + MF + AH + ES + RI; | | | | |
| | EC + EL + LA + MF + MS; EC + LA + MM + FP + RI; | | | | |
| | SG + RT + MF + AH + RI; PS + DS + MF + AH + KQ; | | | | |
| | DS + AH + MS + KQ + RI; PS + RT + EL + MF + MS; | | | | |
| | PS + MM + FP + KQ + RI; RT + FPL + MF + MM + RI; | | | | |
| | RT + EL + LA + MM + AH; LA + MF + ES + MS + RI; | | | | |
| | LA + MM + AH + MS + KQ; RT + DS + TB + AH + ES; | | | | |
| | FPL + AH + FP + ES + RI; EL + MF + TB + AH + KQ; | | | | |
| | LA + MM + MS + KQ + RI; LA + AH + FP + KQ + RI; | | | | |
| | SG + RT + DS + MF + KQ; RT + FPL + LA + AH + MS; | | | | |
| | EC + SG + RT + OS + MF; EC + RT + MF + AH + RI; | | | | |
| | SG + RT + TB + AH + ES; SG + RT + TB + AH + MS; | | | | |
| | SG + LA + MF + TB + MS; RT + TB + AH + KQ + RI; | | | | |
| | EC + SG + MF + MM + KQ; EC + PS + OS + LA + AH; | | | | |
| | EC + EL + LA + MS + RI; PS + RT + EL + AH + RI; | | | | |
| | RT + DS + MF + TB + AH; EC + PS + FPL + OS + TB; | | | | |
| | EC + PS + OS + FP + KQ; EC + RT + OS + LA + AH; | | | | |
| | EC + FPL + MF + MM + MS; EC + EL + MF + AH + KQ; | | | | |
| | SG + PS + RT + MM + MS; SG + RT + MF + TB + AH; | | | | |
| | SG + LA + MF + KQ + RI; PS + MM + FP + MS + RI; | | | | |
| | PS + AH + FP + MS + KQ; OS + MF + TB + AH + FP; | | | | |
| | EC + SG + PS + OS + MS; SG + PS + DS + OS + AH; | | | | |
| | SG + RT + FPL + OS + KQ; PS + RT + DS + MM + AH; | | | | |
| | PS + RT + MF + TB + RI; SG + PS + DS + OS + LA; | | | | |
| | SG + RT + OS + AH + FP; SG + EL + MM + AH + KQ; | | | | |
| | SG + MM + ES + MS + KQ; PS + RT + AH + FP + RI; | | | | |
| | FPL + AH + FP + ES + MS; EC + PS + OS + AH + ES; | | | | |
| | EC + MF + AH + FP + RI; SG + RT + MF + AH + KQ; | | | | |
| | RT + FPL + EL + MS + RI; DS + FPL + OS + MF + TB; | | | | |
| | FPL + LA + MM + KQ + RI; EC + FPL + LA + MS + RI; | | | | |
| | SG + TB + MM + AH + RI; PS + DS + LA + FP + KQ; | | | | |
| | RT + FPL + MM + FP + MS; DS + OS + LA + AH + MS; | | | | |
| | DS + EL + MM + ES + RI; FPL + MF + MM + FP + MS; | | | | |
| | EL + FP + ES + KQ + RI; EC + SG + RT + MF + MM; | | | | |
| | EC + SG + FPL + OS + AH; EC + FPL + OS + FP + ES; | | | | |
| | EC + OS + LA + TB + FP; EC + LA + MF + AH + MS; | | | | |
| | SG + PS + RT + OS + LA; SG + RT + FPL + OS + RI; | | | | |
| | SG + DS + OS + LA + TB; PS + RT + DS + AH + MS; | | | | |
| | PS + RT + FPL + MF + AH; PS + LA + MM + FP + MS; | | | | |
| | RT + AH + ES + MS + KQ; DS + FPL + MF + AH + MS; | | | | |
| | FPL + EL + MF + TB + AH; FPL + MF + AH + FP + RI; | | | | |
| | EC + OS + LA + TB + AH; EC + OS + TB + FP + RI; | | | | |
| | PS + RT + EL + AH + FP; FPL + EL + AH + ES + MS; | | | | |
| | EC + PS + DS + OS + FP; EC + OS + LA + ES + KQ; | | | | |
| | EC + MF + MM + ES + KQ; SG + RT + OS + MF + FP; | | | | |
| | PS + TB + AH + MS + KQ; RT + MM + AH + ES + KQ; | | | | |
| | EC + PS + OS + LA + KQ; EC + RT + MF + AH + KQ; | | | | |
| | SG + PS + FPL + AH + MS; SG + RT + OS + ES + RI; | | | | |
| | SG + DS + EL + MS + KQ; RT + LA + MF + MM + FP; | | | | |
| | RT + LA + MF + AH + RI; DS + LA + MF + FP + KQ; | | | | |
| | FPL + LA + MM + MS + KQ; PS + RT + EL + MF + TB; | | | | |
| | PS + MF + ES + MS + RI; FPL + LA + MF + AH + KQ; | | | | |
| | EL + MM + AH + FP + KQ; EC + FPL + MS + KQ + RI; | | | | |
| | EC + OS + MF + TB + FP; RT + MF + AH + FP + ES; | | | | |
| | DS + EL + LA + MM + KQ; OS + TB + AH + FP + KQ; | | | | |
| | SG + RT + TB + MM + MS; PS + RT + DS + MF + AH; | | | | |
| | RT + DS + MF + TB + KQ; RT + FPL + EL + LA + AH; | | | | |
| | EC + RT + OS + FP + KQ; EC + EL + FP + MS + RI; | | | | |
| | EC + PS + RT + OS + KQ; PS + DS + FP + KQ + RI; | | | | |
| | PS + OS + EL + TB + MM; EC + SG + DS + FPL + OS; | | | | |
| | SG + RT + OS + LA + KQ; PS + RT + DS + MF + KQ; | | | | |
| | PS + FPL + EL + LA + RI; RT + EL + MF + MM + FP; | | | | |
| | EC + DS + OS + EL + RI; EC + AH + MS + KQ + RI; | | | | |
| | SG + LA + MF + MS + RI; PS + RT + FPL + TB + MM; | | | | |
| | PS + EL + MF + MS + KQ; FPL + OS + TB + AH + FP; | | | | |
| | EL + TB + AH + FP + RI; EC + FPL + EL + MM + KQ; | | | | |
| | SG + PS + TB + FP + KQ; PS + DS + OS + LA + TB; | | | | |
| | PS + FPL + EL + MF + MS; RT + EL + MF + AH + FP; | | | | |
| | RT + TB + AH + ES + MS; EC + SG + FPL + OS + KQ; | | | | |
| | EC + RT + OS + EL + ES; SG + FPL + OS + LA + AH; | | | | |
| | PS + FPL + MF + FP + MS; PS + FPL + MM + AH + KQ; | | | | |
| | RT + EL + LA + MF + KQ; FPL + MM + ES + MS + RI; | | | | |
| | LA + FP + ES + KQ + RI; EC + EL + TB + AH + RI; | | | | |
| | SG + MM + AH + MS + RI; PS + LA + FP + MS + RI; | | | | |
| | DS + OS + LA + KQ + RI; EC + FPL + OS + TB + ES; | | | | |
| | SG + FPL + OS + AH + FP; PS + RT + FPL + MF + TB; | | | | |
| | EL + LA + MF + FP + RI; EC + RT + OS + ES + KQ; | | | | |
| | EC + RT + MF + TB + KQ; EC + FPL + EL + MM + RI; | | | | |
| | PS + RT + AH + ES + MS; PS + FP + ES + MS + KQ; | | | | |
| | EC + FPL + EL + LA + RI; SG + PS + RT + AH + MS; | | | | |
| | PS + RT + FPL + EL + AH; PS + RT + TB + MM + MS; | | | | |
| | PS + DS + MF + MM + RI; EL + LA + AH + MS + KQ; | | | | |
| | EC + SG + PS + FPL + OS; EC + PS + FPL + OS + ES; | | | | |
| | FPL + MM + AH + FP + KQ; EC + PS + OS + MM + KQ; | | | | |
| | SG + PS + FPL + MF + MM; SG + FPL + MF + AH + MS; | | | | |
| | DS + MF + MS + KQ + RI; EC + DS + FPL + OS + RI; | | | | |
| | EC + MF + MM + AH + KQ; | | | | |
| | DS + FPL + OS + MM + KQ; | | | | |
| | FPL + EL + MF + AH + KQ; EC + RT + FPL + OS + RI; | | | | |
| | EC + DS + MF + MM + KQ; SG + FPL + TB + AH + MS; | | | | |
| | DS + OS + LA + MM + AH; FPL + EL + LA + MM + MS; | | | | |
| | FPL + EL + MM + MS + RI; MM + AH + ES + MS + RI; | | | | |
| | EC + SG + OS + MM + FP; EC + LA + MM + MS + RI; | | | | |
| | EC + MF + MM + FP + MS; SG + PS + RT + AH + FP; | | | | |
| | PS + RT + TB + MM + RI; PS + FPL + MF + TB + AH; | | | | |
| | PS + AH + FP + KQ + RI; EL + AH + ES + MS + RI; | | | | |
| | TB + MM + MS + KQ + RI; EC + SG + RT + OS + MM; | | | | |
| | SG + FPL + TB + AH + RI; FPL + OS + TB + AH + ES; | | | | |
| | EC + PS + LA + MF + MM; EC + RT + FPL + MF + MM; | | | | |
| | EC + RT + EL + MF + AH; SG + PS + DS + MM + ES; | | | | |
| | PS + RT + MM + AH + RI; PS + LA + MF + AH + MS; | | | | |
| | OS + LA + TB + ES + MS; SG + PS + RT + MM + AH; | | | | |
| | SG + PS + DS + MM + KQ; PS + FPL + MM + FP + RI; | | | | |
| | DS + FPL + OS + TB + FP; MM + AH + FP + MS + KQ; | | | | |
| | DS + FPL + OS + LA + ES; EC + PS + EL + KQ + RI; | | | | |
| | EC + DS + OS + MS + RI; EC + FPL + MM + MS + RI; | | | | |
| | SG + PS + MF + MM + FP; PS + EL + TB + AH + MS; | | | | |
| | SG + RT + DS + AH + MS; SG + FPL + EL + AH + FP; | | | | |
| | EC + FPL + MF + MM + KQ; PS + RT + DS + TB + AH; | | | | |
| | FPL + LA + AH + ES + MS; EC + FPL + LA + MF + MS; | | | | |
| | PS + FPL + LA + TB + MS; PS + FPL + AH + KQ + RI; | | | | |
| | RT + EL + MM + AH + ES; DS + OS + LA + MF + AH; | | | | |
| | EC + PS + RT + OS + TB; SG + RT + FPL + MM + AH; | | | | |
| | SG + EL + LA + TB + MS; PS + RT + FPL + TB + MS; | | | | |
| | RT + DS + AH + MS + KQ; DS + LA + MF + MM + RI; | | | | |
| | SG + PS + DS + EL + MF; SG + DS + MF + MM + RI; | | | | |
| | OS + LA + TB + FP + KQ; LA + MF + AH + FP + RI; | | | | |
| | EC + FPL + MM + MS + KQ; PS + RT + MF + TB + FP; | | | | |
| | FPL + EL + LA + MS + KQ; EC + SG + OS + LA + MF; | | | | |
| | EC + SG + EL + TB + RI; EC + RT + EL + MM + AH; | | | | |
| | PS + EL + MF + AH + RI; PS + LA + MF + TB + KQ; | | | | |
| | RT + DS + MM + AH + ES; DS + EL + MF + MM + AH; | | | | |
| | EC + SG + OS + ES + RI; SG + FPL + OS + LA + TB; | | | | |
| | DS + MF + TB + MM + KQ; MF + TB + MM + AH + MS; | | | | |
| | EC + PS + OS + MF + RI; SG + OS + LA + TB + AH; | | | | |
| | PS + RT + TB + MM + FP; RT + MF + MM + FP + MS; | | | | |
| | EL + LA + MF + FP + ES; EC + SG + OS + AH + RI; | | | | |
| | EC + OS + EL + TB + FP; SG + PS + FPL + MF + AH; | | | | |
| | SG + PS + MF + MM + AH; SG + RT + FPL + MM + MS; | | | | |
| | RT + OS + LA + FP + RI; EC + EL + LA + FP + RI; | | | | |
| | PS + RT + MF + AH + FP; PS + RT + MF + FP + MS; | | | | |
| | PS + RT + AH + FP + KQ; EL + MF + ES + MS + KQ; | | | | |
| | EC + RT + OS + FP + MS; EC + LA + MM + AH + KQ; | | | | |
| | SG + RT + EL + MF + MM; PS + RT + MM + FP + KQ; | | | | |
| | RT + OS + LA + TB + KQ; FPL + LA + TB + AH + MS; | | | | |
| | LA + MF + FP + ES + MS | | | | |
| 5 | EC + LA + AH + FP + KQ; SG + RT + DS + TB + ES; | 0.74 | 0.2915- | 39.68%- | 40.40%- |
| | SG + MF + MM + AH + FP; PS + RT + FPL + EL + MF; | | 0.5455 | 96.83% | 97.98% |
| | RT + DS + MF + FP + KQ; EL + MM + FP + ES + MS; | | | | |
| | SG + TB + AH + FP + RI; PS + RT + DS + FPL + EL; | | | | |
| | RT + DS + OS + EL + FP; EC + SG + PS + RT + OS; | | | | |
| | EC + PS + AH + FP + KQ; PS + RT + DS + OS + LA; | | | | |
| | PS + RT + LA + AH + FP; PS + FPL + EL + MS + RI; | | | | |
| | DS + FPL + MF + FP + MS; TB + FP + ES + KQ + RI; | | | | |
| | EC + PS + RT + AH + MS; SG + PS + DS + FP + MS; | | | | |
| | SG + PS + FPL + AH + RI; PS + RT + EL + TB + FP; | | | | |
| | RT + FPL + EL + MM + ES; EL + MF + ES + MS + RI; | | | | |
| | EC + PS + FPL + LA + AH; EC + RT + LA + AH + RI; | | | | |
| | SG + LA + MF + ES + RI; DS + ES + MS + KQ + RI; | | | | |
| | EC + PS + FPL + FP + KQ; EC + PS + EL + LA + MM; | | | | |
| | EC + PS + AH + FP + MS; EC + RT + LA + FP + RI; | | | | |
| | EC + FPL + FP + ES + RI; EC + MF + MM + FP + ES; | | | | |
| | SG + PS + TB + ES + MS; SG + RT + FPL + OS + LA; | | | | |
| | SG + DS + MM + KQ + RI; SG + MF + MM + AH + ES; | | | | |
| | RT + FPL + EL + KQ + RI; EL + LA + MF + ES + RI; | | | | |
| | EC + RT + FPL + AH + RI; EC + FPL + EL + LA + FP; | | | | |
| | EC + MM + ES + KQ + RI; SG + EL + AH + ES + MS; | | | | |
| | PS + RT + FP + KQ + RI; PS + FPL + EL + MF + MM; | | | | |
| | FPL + MM + AH + ES + KQ; EC + SG + RT + OS + TB; | | | | |
| | EC + PS + FPL + LA + MF; EC + FPL + ES + MS + RI; | | | | |
| | EC + EL + TB + AH + MS; EC + MM + AH + FP + ES; | | | | |
| | SG + FPL + MF + ES + RI; PS + RT + DS + FPL + MS; | | | | |
| | RT + DS + MF + TB + RI; FPL + EL + MM + ES + KQ; | | | | |
| | EC + RT + EL + MM + KQ; PS + RT + FPL + EL + MS; | | | | |
| | PS + LA + MF + FP + ES; RT + DS + OS + TB + FP; | | | | |
| | RT + LA + MM + MS + RI; RT + MM + MS + KQ + RI; | | | | |
| | FPL + LA + TB + MS + RI; EC + PS + FPL + FP + RI; | | | | |
| | EC + RT + FPL + OS + ES; PS + RT + MF + FP + ES; | | | | |
| | RT + FPL + EL + MM + KQ; FPL + LA + ES + KQ + RI; | | | | |
| | EC + PS + LA + MM + RI; EC + RT + FPL + FP + RI; | | | | |
| | EC + DS + EL + LA + FP; EC + FPL + LA + MF + FP; | | | | |
| | SG + MF + FP + MS + RI; PS + FPL + MM + MS + KQ; | | | | |
| | RT + EL + MM + KQ + RI; DS + MF + TB + AH + KQ; | | | | |
| | EC + PS + RT + DS + MM; EC + FPL + TB + AH + RI; | | | | |
| | SG + DS + OS + AH + FP; PS + RT + DS + EL + FP; | | | | |
| | PS + DS + FP + MS + KQ; PS + MF + AH + ES + RI; | | | | |
| | RT + EL + MF + TB + RI; EC + PS + RT + TB + FP; | | | | |
| | EC + RT + MM + FP + MS; SG + RT + DS + TB + KQ; | | | | |
| | SG + FPL + TB + MS + RI; PS + RT + OS + LA + FP; | | | | |
| | PS + FPL + LA + MM + RI; RT + DS + EL + TB + MM; | | | | |
| | RT + MF + FP + KQ + RI; EC + DS + OS + LA + MS; | | | | |
| | EC + FPL + EL + MM + FP; EC + MM + ES + MS + KQ; | | | | |
| | SG + RT + DS + EL + MM; SG + RT + LA + MF + TB; | | | | |
| | SG + AH + ES + MS + KQ; PS + DS + AH + MS + RI; | | | | |
| | PS + FPL + EL + MS + KQ; PS + FPL + LA + MF + FP; | | | | |
| | PS + FPL + LA + FP + RI; PS + OS + EL + TB + ES; | | | | |
| | RT + FPL + EL + MM + FP; DS + TB + ES + MS + KQ; | | | | |
| | EC + SG + EL + TB + MM; EC + FPL + MM + FP + ES; | | | | |
| | SG + RT + FPL + EL + MS; SG + TB + FP + ES + MS; | | | | |
| | PS + TB + MM + MS + RI; RT + DS + OS + EL + RI; | | | | |
| | RT + FPL + EL + LA + RI; RT + LA + MF + TB + MS; | | | | |
| | RT + LA + MM + FP + KQ; EL + MM + ES + MS + KQ; | | | | |
| | EL + ES + MS + KQ + RI; MM + AH + FP + ES + KQ; | | | | |
| | EC + SG + OS + TB + FP; EC + RT + MM + KQ + RI; | | | | |
| | EC + FPL + MM + AH + FP; SG + PS + RT + DS + MM; | | | | |
| | SG + DS + MM + ES + MS; PS + DS + FPL + AH + ES; | | | | |
| | PS + LA + MM + AH + KQ; PS + TB + MM + KQ + RI; | | | | |
| | RT + DS + TB + MM + KQ; RT + FPL + AH + FP + KQ; | | | | |
| | EC + SG + OS + FP + ES; EC + PS + LA + MF + FP; | | | | |
| | EC + EL + ES + KQ + RI; EC + MF + TB + AH + KQ; | | | | |
| | SG + PS + MM + FP + ES; PS + RT + FPL + MF + FP; | | | | |
| | FPL + LA + MF + AH + ES; FPL + LA + ES + MS + RI; | | | | |
| | EL + LA + MF + FP + MS; EC + PS + FPL + MM + AH; | | | | |
| | EC + OS + LA + TB + ES; EC + LA + MM + AH + FP; | | | | |
| | SG + FPL + EL + LA + RI; SG + FPL + MM + FP + MS; | | | | |
| | SG + FP + ES + KQ + RI; PS + FPL + AH + ES + KQ; | | | | |
| | RT + DS + EL + LA + MM; DS + OS + LA + TB + AH; | | | | |
| | DS + EL + AH + MS + RI; FPL + MF + AH + ES + KQ; | | | | |
| | EC + PS + MF + MM + MS; SG + MF + MM + FP + MS; | | | | |
| | PS + EL + MF + TB + KQ; RT + AH + FP + ES + KQ; | | | | |
| | DS + EL + MM + AH + FP; SG + PS + TB + MS + KQ; | | | | |
| | SG + RT + OS + LA + RI; SG + DS + MF + ES + MS; | | | | |
| | SG + FPL + MF + MS + KQ; DS + MM + AH + FP + RI; | | | | |
| | LA + MM + AH + FP + MS; LA + AH + ES + MS + RI; | | | | |
| | SG + DS + FPL + OS + MF; RT + DS + FPL + OS + RI; | | | | |
| | RT + DS + FPL + FP + KQ; RT + LA + AH + FP + KQ; | | | | |
| | DS + OS + LA + AH + FP; FPL + EL + MM + FP + MS; | | | | |
| | LA + MF + TB + MM + AH; EC + PS + RT + AH + FP; | | | | |
| | SG + PS + FPL + ES + MS; PS + RT + EL + LA + MF; | | | | |
| | PS + FPL + LA + AH + FP; PS + LA + MM + MS + KQ; | | | | |
| | RT + DS + EL + MM + RI; RT + MM + FP + ES + KQ; | | | | |
| | DS + EL + MF + AH + RI; EC + SG + FPL + LA + MM; | | | | |
| | EC + SG + MF + AH + MS; EC + RT + LA + MM + FP; | | | | |
| | EC + RT + MF + TB + MS; EC + DS + EL + MM + FP; | | | | |
| | EC + EL + AH + ES + RI; SG + PS + RT + FPL + MS; | | | | |
| | SG + PS + FPL + EL + MS; SG + PS + MF + TB + AH; | | | | |
| | MF + AH + FP + ES + MS; EC + PS + OS + TB + MM; | | | | |
| | EC + RT + DS + FPL + OS; EC + AH + FP + MS + KQ; | | | | |
| | RT + EL + MF + FP + KQ; FPL + EL + LA + FP + KQ; | | | | |
| | EC + PS + RT + MM + FP; EC + PS + MF + AH + MS; | | | | |
| | EC + TB + MM + AH + MS; PS + RT + FPL + LA + MM; | | | | |
| | DS + FPL + LA + AH + MS; LA + MF + TB + MS + RI; | | | | |
| | EC + RT + FPL + MM + RI; EC + EL + TB + FP + RI; | | | | |
| | SG + PS + TB + MM + KQ; PS + DS + EL + MF + AH; | | | | |
| | PS + LA + MF + AH + RI; RT + EL + MM + FP + KQ; | | | | |
| | DS + FPL + EL + LA + AH; FPL + LA + AH + FP + KQ; | | | | |
| | EC + RT + AH + FP + MS; EC + DS + OS + AH + FP; | | | | |
| | SG + FPL + MM + ES + MS; PS + FPL + EL + MM + FP; | | | | |
| | PS + EL + MM + FP + KQ; RT + FPL + AH + ES + MS; | | | | |
| | DS + OS + TB + FP + KQ; DS + EL + FP + KQ + RI; | | | | |
| | DS + LA + AH + FP + KQ; EC + RT + DS + MM + AH; | | | | |
| | PS + TB + FP + ES + RI; RT + DS + EL + MM + FP; | | | | |
| | DS + LA + MF + KQ + RI; EC + RT + EL + AH + KQ; | | | | |
| | EC + DS + FPL + MS + RI; SG + MM + AH + KQ + RI; | | | | |
| | FPL + MM + FP + MS + KQ; EC + SG + MM + AH + MS; | | | | |
| | EC + MM + ES + MS + RI; PS + FPL + MM + ES + MS; | | | | |
| | PS + EL + MF + KQ + RI; RT + EL + LA + MM + FP; | | | | |
| | DS + OS + TB + AH + FP; EC + SG + FPL + AH + MS; | | | | |
| | EC + RT + EL + MM + FP; EC + DS + MF + FP + KQ; | | | | |
| | SG + DS + MM + MS + KQ; RT + DS + FPL + MM + MS; | | | | |
| | RT + EL + LA + MF + RI; RT + LA + MM + AH + FP; | | | | |
| | DS + EL + AH + FP + KQ; LA + ES + MS + KQ + RI; | | | | |
| | MF + TB + AH + ES + KQ; EC + SG + EL + MF + MM; | | | | |
| | EC + MF + TB + AH + MS; EC + FP + ES + KQ + RI; | | | | |
| | SG + RT + FPL + EL + MM; SG + EL + LA + ES + MS; | | | | |
| | PS + RT + LA + TB + AH; PS + FPL + MF + ES + KQ; | | | | |
| | PS + EL + LA + MM + RI; PS + TB + MM + AH + MS; | | | | |
| | EL + TB + MM + AH + KQ; LA + MF + TB + AH + KQ; | | | | |
| | EC + RT + DS + EL + MM; EC + DS + FPL + FP + KQ; | | | | |
| | EC + FPL + MM + ES + RI; SG + DS + EL + MS + RI; | | | | |
| | SG + FPL + EL + MS + RI; SG + MF + TB + FP + KQ; | | | | |
| | PS + RT + FPL + LA + MS; PS + RT + MM + MS + RI; | | | | |
| | RT + DS + OS + LA + RI; RT + TB + ES + MS + KQ; | | | | |
| | DS + MM + AH + FP + MS; FPL + EL + LA + MF + ES; | | | | |
| | FPL + MF + TB + MS + KQ; EC + FPL + FP + ES + KQ; | | | | |
| | EC + EL + LA + MS + KQ; EC + MF + TB + MM + MS; | | | | |
| | PS + RT + DS + LA + MM; PS + DS + EL + LA + MS; | | | | |
| | PS + DS + EL + TB + RI; RT + LA + TB + MM + AH; | | | | |
| | EC + SG + DS + OS + KQ; EC + PS + RT + EL + MM; | | | | |
| | EC + PS + RT + EL + AH; EC + PS + OS + TB + RI; | | | | |
| | EC + RT + FPL + MS + RI; EC + DS + FPL + MM + FP; | | | | |
| | EC + EL + MF + MM + ES; SG + PS + DS + ES + KQ; | | | | |
| | SG + DS + FPL + OS + AH; SG + FPL + MM + AH + ES; | | | | |
| | PS + RT + LA + MF + MS; PS + RT + TB + MM + ES; | | | | |
| | RT + DS + EL + AH + RI; RT + LA + TB + AH + RI; | | | | |
| | LA + MF + FP + MS + KQ; EC + MF + TB + MS + RI; | | | | |
| | PS + DS + FPL + AH + KQ; DS + EL + AH + KQ + RI; | | | | |
| | LA + MM + ES + KQ + RI; MM + FP + ES + MS + RI; | | | | |
| | EC + SG + DS + EL + RI; EC + RT + FPL + MM + FP; | | | | |
| | PS + RT + LA + MF + FP; PS + FPL + TB + MM + MS; | | | | |
| | PS + TB + FP + KQ + RI; LA + AH + FP + MS + KQ; | | | | |
| | EC + SG + DS + OS + MF; EC + PS + FPL + MM + KQ; | | | | |
| | EC + PS + AH + MS + RI; SG + PS + DS + LA + MF; | | | | |
| | PS + DS + EL + MS + RI; RT + EL + TB + AH + FP; | | | | |
| | DS + FPL + EL + MF + AH; EC + RT + LA + MF + RI; | | | | |
| | EC + FPL + MM + ES + KQ; SG + PS + DS + MS + KQ; | | | | |
| | SG + RT + DS + TB + MS; SG + RT + MF + ES + MS; | | | | |
| | SG + DS + MF + KQ + RI; PS + FPL + LA + MS + RI; | | | | |
| | EC + SG + PS + OS + RI; EC + PS + FPL + AH + FP; | | | | |
| | EC + RT + LA + MM + AH; SG + PS + LA + MF + FP; | | | | |
| | SG + PS + LA + TB + MS; SG + PS + MM + AH + RI; | | | | |
| | SG + DS + MF + TB + MS; SG + MF + TB + AH + MS; | | | | |
| | RT + LA + MF + TB + AH; EC + RT + AH + KQ + RI; | | | | |
| | EC + LA + AH + MS + KQ; SG + EL + LA + ES + RI; | | | | |
| | SG + MM + AH + MS + KQ; PS + FPL + AH + FP + KQ; | | | | |
| | PS + EL + MF + AH + FP; RT + DS + OS + LA + MS; | | | | |
| | RT + DS + LA + TB + MM; RT + FPL + EL + LA + MM; | | | | |
| | RT + EL + AH + ES + RI; DS + EL + TB + ES + MS; | | | | |
| | EC + PS + MF + FP + KQ; EC + RT + LA + MM + RI; | | | | |
| | EC + DS + OS + MF + TB; EC + FPL + MM + ES + MS; | | | | |
| | EC + EL + MF + TB + MM; EC + MF + TB + MM + KQ; | | | | |
| | SG + RT + AH + KQ + RI; PS + RT + DS + MF + ES; | | | | |
| | PS + EL + AH + KQ + RI; RT + DS + AH + FP + MS; | | | | |
| | RT + FPL + LA + MF + KQ; DS + AH + ES + MS + KQ; | | | | |
| | EC + PS + RT + MF + FP; EC + RT + AH + MS + KQ; | | | | |
| | EC + DS + LA + FP + ES; DS + EL + LA + MM + AH; | | | | |
| | DS + TB + AH + KQ + RI; FPL + LA + MF + FP + MS; | | | | |
| | MF + TB + FP + KQ + RI; SG + PS + DS + FP + KQ; | | | | |
| | PS + RT + MF + MS + RI; RT + FPL + EL + MF + FP; | | | | |
| | FPL + LA + MM + FP + KQ; EC + RT + MM + FP + KQ; | | | | |
| | EC + DS + FPL + MF + MM; EC + EL + TB + MM + AH; | | | | |
| | EC + LA + MM + ES + RI; PS + FPL + LA + MF + RI; | | | | |
| | DS + FP + MS + KQ + RI; LA + TB + MM + AH + RI; | | | | |
| | LA + TB + AH + KQ + RI; EC + PS + RT + MF + TB; | | | | |
| | EC + MF + FP + ES + KQ; SG + RT + DS + FPL + OS; | | | | |
| | PS + RT + EL + FP + MS; PS + DS + EL + MM + AH; | | | | |
| | RT + EL + TB + MM + KQ; RT + LA + AH + KQ + RI; | | | | |
| | EC + SG + PS + OS + FP; SG + DS + FPL + OS + ES; | | | | |
| | PS + RT + EL + MF + FP; FPL + LA + MM + FP + MS; | | | | |
| | LA + MM + FP + MS + RI; EC + PS + FPL + LA + FP; | | | | |
| | EC + RT + MF + MM + ES; EC + RT + MF + FP + RI; | | | | |
| | EC + DS + FPL + OS + MF; EC + FPL + EL + FP + RI; | | | | |
| | SG + RT + MM + FP + KQ; PS + EL + LA + MS + KQ; | | | | |
| | RT + FPL + MF + FP + KQ; RT + AH + ES + MS + RI; | | | | |
| | RT + AH + ES + KQ + RI; DS + EL + LA + TB + MM; | | | | |
| | DS + MF + FP + ES + KQ; FPL + EL + ES + MS + KQ; | | | | |
| | EC + RT + EL + TB + MM; SG + DS + EL + AH + RI; | | | | |
| | SG + EL + TB + AH + RI; RT + DS + FPL + OS + FP; | | | | |
| | DS + EL + MF + AH + MS; FPL + LA + MF + TB + KQ; | | | | |
| | EC + DS + OS + AH + ES; EC + FPL + FP + MS + KQ; | | | | |
| | SG + RT + MF + AH + ES; PS + RT + EL + MF + KQ; | | | | |
| | PS + RT + TB + ES + MS; PS + FPL + MF + FP + RI; | | | | |
| | RT + DS + LA + MM + FP; RT + FPL + LA + TB + AH; | | | | |
| | RT + FPL + LA + MM + RI; DS + FPL + TB + MM + AH; | | | | |
| | FPL + EL + LA + MM + FP; EC + SG + OS + FP + MS; | | | | |
| | EC + RT + FPL + MM + MS; | | | | |
| | EC + FPL + MF + MM + FP; EC + OS + TB + FP + MS; | | | | |
| | SG + PS + RT + MM + ES; PS + RT + TB + MS + KQ; | | | | |
| | RT + DS + LA + AH + KQ; RT + DS + TB + AH + FP; | | | | |
| | EC + PS + FP + KQ + RI; EC + RT + EL + AH + FP; | | | | |
| | EC + DS + FPL + MS + KQ; EC + DS + OS + KQ + RI; | | | | |
| | EC + EL + LA + ES + RI; SG + PS + RT + TB + FP; | | | | |
| | SG + RT + DS + MM + KQ; SG + EL + LA + MF + KQ; | | | | |
| | RT + DS + OS + FP + KQ; RT + FPL + TB + AH + KQ; | | | | |
| | DS + EL + LA + AH + KQ; DS + EL + LA + AH + RI; | | | | |
| | DS + MF + AH + FP + RI; PS + RT + DS + FPL + MF; | | | | |
| | PS + RT + FPL + MM + RI; RT + DS + TB + ES + MS; | | | | |
| | RT + OS + LA + FP + KQ; LA + MF + TB + FP + KQ; | | | | |
| | EC + FPL + EL + LA + KQ; RT + MM + FP + MS + RI; | | | | |
| | EC + SG + PS + OS + EL; EC + PS + OS + TB + MS; | | | | |
| | SG + RT + DS + FPL + AH; RT + MM + AH + ES + RI; | | | | |
| | DS + EL + MF + MM + FP; FPL + LA + MF + TB + RI; | | | | |
| | EL + LA + MM + MS + KQ; MM + AH + FP + ES + MS; | | | | |
| | EC + PS + RT + OS + ES; EC + RT + LA + MF + AH; | | | | |
| | SG + PS + EL + MF + AH; PS + DS + LA + MM + FP; | | | | |
| | DS + FPL + MF + MM + FP; DS + MM + AH + ES + KQ; | | | | |
| | FPL + EL + MF + MM + ES; EL + FP + MS + KQ + RI; | | | | |
| | EC + PS + MM + AH + MS; EC + DS + FPL + AH + MS; | | | | |
| | SG + PS + RT + EL + MM; LA + AH + ES + KQ + RI; | | | | |
| | EC + RT + LA + MF + KQ; EC + FPL + FP + MS + RI; | | | | |
| | FPL + TB + AH + MS + KQ; MF + TB + AH + ES + RI; | | | | |
| | EC + RT + AH + MS + RI; RT + FPL + MF + MS + RI; | | | | |
| | DS + LA + MM + FP + RI; LA + TB + AH + MS + RI; | | | | |
| | EC + PS + FPL + MM + FP; EC + DS + OS + LA + MF; | | | | |
| | SG + EL + MS + KQ + RI; PS + LA + MF + FP + MS; | | | | |
| | RT + DS + LA + MM + ES; DS + EL + AH + MS + KQ; | | | | |
| | FPL + MM + FP + ES + RI; EC + PS + MM + MS + RI; | | | | |
| | EC + MM + AH + ES + MS; PS + EL + LA + MF + RI; | | | | |
| | EC + SG + EL + LA + RI; EC + EL + AH + FP + KQ; | | | | |
| | SG + RT + EL + MF + KQ; SG + MM + FP + KQ + RI; | | | | |
| | PS + RT + FP + ES + MS; RT + DS + EL + AH + ES; | | | | |
| | PS + LA + MF + ES + MS; RT + DS + EL + AH + FP; | | | | |
| | DS + FPL + OS + ES + RI; EL + MF + FP + ES + KQ; | | | | |
| | EC + RT + EL + MF + RI; EC + FPL + LA + AH + FP; | | | | |
| | EC + OS + TB + FP + ES; EC + EL + LA + AH + FP; | | | | |
| | EC + TB + MM + MS + RI; SG + MM + AH + FP + RI; | | | | |
| | PS + DS + EL + LA + RI; PS + TB + AH + MS + RI; | | | | |
| | RT + DS + EL + MF + MS; FPL + LA + MM + ES + RI; | | | | |
| | EL + AH + ES + MS + KQ; EC + SG + FPL + LA + MF; | | | | |
| | EC + SG + OS + AH + ES; EC + PS + FPL + AH + RI; | | | | |
| | EC + RT + TB + AH + RI; EC + FPL + EL + LA + AH; | | | | |
| | SG + MF + AH + ES + MS; PS + EL + LA + AH + FP; | | | | |
| | PS + AH + ES + MS + KQ; RT + DS + LA + MM + AH; | | | | |
| | LA + MF + TB + ES + KQ; EC + DS + MM + FP + KQ; | | | | |
| | EC + MM + FP + ES + KQ; SG + PS + RT + DS + MF; | | | | |
| | DS + MF + MM + FP + ES; EC + SG + PS + OS + TB; | | | | |
| | EC + FPL + MF + AH + KQ; SG + PS + DS + TB + MM; | | | | |
| | PS + RT + DS + TB + FP; EC + SG + PS + OS + AH; | | | | |
| | EC + FPL + LA + MF + RI; EC + FPL + TB + MM + RI; | | | | |
| | EC + MM + FP + MS + KQ; SG + PS + LA + MM + MS; | | | | |
| | SG + DS + OS + EL + RI; PS + RT + FPL + EL + MM; | | | | |
| | RT + DS + EL + TB + AH; RT + TB + AH + FP + KQ; | | | | |
| | EC + PS + RT + OS + LA; EC + PS + OS + ES + KQ; | | | | |
| | EC + FPL + MF + FP + KQ; EC + EL + MF + ES + RI; | | | | |
| | SG + DS + MF + AH + MS; RT + DS + FPL + MF + AH; | | | | |
| | RT + LA + MF + KQ + RI; DS + EL + LA + MF + RI; | | | | |
| | EC + RT + FPL + MM + KQ; EC + FPL + OS + TB + FP; | | | | |
| | EC + FPL + LA + MM + RI; EC + EL + LA + AH + KQ; | | | | |
| | PS + LA + AH + FP + RI; RT + FPL + LA + MM + MS; | | | | |
| | DS + AH + FP + KQ + RI; FPL + LA + TB + AH + RI; | | | | |
| | FPL + MF + FP + MS + RI; EC + PS + DS + OS + EL; | | | | |
| | EC + RT + EL + AH + MS; EC + FPL + MM + FP + MS; | | | | |
| | SG + RT + MM + MS + KQ; SG + EL + MF + TB + RI; | | | | |
| | PS + RT + FPL + MM + KQ; RT + DS + MF + KQ + RI; | | | | |
| | EC + SG + MF + MS + KQ; EC + PS + EL + MF + AH; | | | | |
| | SG + RT + MM + ES + MS; SG + DS + TB + ES + KQ; | | | | |
| | SG + FPL + EL + MF + MS; PS + RT + DS + TB + KQ; | | | | |
| | PS + DS + MM + AH + MS; DS + LA + MF + MM + AH; | | | | |
| | EL + MM + AH + ES + MS; TB + AH + FP + KQ + RI; | | | | |
| | EC + RT + MM + MS + KQ; EC + RT + AH + FP + KQ; | | | | |
| | EC + LA + MF + FP + RI; SG + RT + MF + TB + RI; | | | | |
| | PS + FPL + MF + AH + KQ; PS + EL + LA + AH + RI; | | | | |
| | RT + FPL + LA + MF + AH; DS + EL + LA + MF + TB; | | | | |
| | FPL + AH + ES + KQ + RI; EL + LA + AH + FP + KQ; | | | | |
| | EL + AH + FP + ES + RI; EC + SG + RT + OS + AH; | | | | |
| | PS + RT + EL + LA + AH; PS + FPL + EL + MF + RI; | | | | |
| | PS + FPL + FP + KQ + RI; DS + EL + LA + MM + FP; | | | | |
| | EC + PS + DS + OS + AH; EC + PS + EL + MM + MS; | | | | |
| | EC + RT + DS + MF + KQ; SG + RT + OS + EL + FP; | | | | |
| | SG + MF + FP + MS + KQ; PS + DS + AH + FP + KQ; | | | | |
| | PS + MM + FP + ES + MS; RT + DS + MM + FP + RI; | | | | |
| | RT + EL + AH + ES + MS; EC + SG + RT + MF + KQ; | | | | |
| | EC + SG + MF + KQ + RI; EC + DS + OS + LA + RI; | | | | |
| | SG + LA + MF + ES + KQ; SG + PS + FP + ES + KQ; | | | | |
| | PS + DS + FPL + MF + AH; FPL + EL + AH + ES + RI; | | | | |
| | EC + SG + OS + LA + KQ; EC + SG + EL + MF + KQ; | | | | |
| | EC + RT + EL + TB + AH; EC + RT + EL + MM + RI; | | | | |
| | EC + DS + FPL + OS + KQ; EC + DS + OS + ES + RI; | | | | |
| | EC + DS + EL + AH + RI; EC + EL + TB + MS + RI; | | | | |
| | EC + MF + FP + MS + KQ; SG + PS + TB + ES + KQ; | | | | |
| | SG + DS + FPL + MF + MM; SG + FPL + MF + ES + KQ; | | | | |
| | PS + RT + MF + ES + KQ; PS + MF + AH + ES + KQ; | | | | |
| | RT + LA + MF + FP + RI; FPL + EL + LA + AH + ES; | | | | |
| | EC + PS + MF + AH + RI; PS + RT + TB + FP + KQ; | | | | |
| | RT + DS + FPL + MM + FP; RT + DS + MM + ES + MS; | | | | |
| | RT + EL + LA + MM + RI; FPL + MM + AH + FP + ES; | | | | |
| | EC + PS + DS + FPL + OS; EC + PS + OS + FP + ES; | | | | |
| | EC + PS + MF + MM + FP; EC + EL + MM + AH + FP; | | | | |
| | RT + DS + FPL + AH + MS; RT + DS + AH + ES + KQ; | | | | |
| | RT + TB + MM + FP + RI; EC + MF + TB + MS + KQ; | | | | |
| | SG + TB + MM + ES + KQ; PS + MF + TB + AH + RI; | | | | |
| | RT + DS + LA + MM + RI; RT + MF + TB + MM + FP; | | | | |
| | FPL + EL + MM + KQ + RI; EC + SG + FPL + MM + MS; | | | | |
| | EC + RT + MF + TB + RI; PS + RT + AH + FP + ES; | | | | |
| | RT + DS + MF + AH + FP; EL + LA + MF + TB + KQ; | | | | |
| | EC + LA + AH + FP + RI; DS + FPL + OS + AH + ES; | | | | |
| | EC + PS + EL + MF + MM; EC + RT + OS + FP + ES; | | | | |
| | EC + RT + EL + LA + MF; EC + DS + FPL + OS + EL; | | | | |
| | EC + LA + AH + KQ + RI; PS + RT + DS + MM + FP; | | | | |
| | FPL + LA + MF + TB + MS; EC + OS + TB + FP + KQ; | | | | |
| | EC + MF + TB + KQ + RI; SG + FPL + MF + MM + RI; | | | | |
| | RT + DS + EL + AH + MS; RT + FPL + EL + MM + MS; | | | | |
| | RT + EL + MM + MS + KQ; FPL + EL + MF + FP + RI; | | | | |
| | EC + PS + OS + ES + RI; EC + TB + AH + KQ + RI; | | | | |
| | PS + RT + EL + TB + MS; PS + RT + TB + FP + MS; | | | | |
| | RT + FPL + EL + TB + RI; DS + FPL + AH + MS + KQ; | | | | |
| | DS + MM + MS + KQ + RI; FPL + LA + MF + FP + KQ; | | | | |
| | EC + RT + MM + FP + RI; SG + RT + AH + ES + MS; | | | | |
| | SG + AH + FP + MS + RI; PS + DS + MM + AH + KQ; | | | | |
| | PS + FPL + EL + FP + MS; PS + LA + MM + AH + MS; | | | | |
| | LA + MF + FP + MS + RI; EC + RT + MF + FP + KQ; | | | | |
| | EC + EL + FP + KQ + RI; EC + MM + AH + FP + MS; | | | | |
| | SG + RT + OS + LA + ES; SG + RT + EL + AH + FP; | | | | |
| | PS + RT + TB + AH + KQ; PS + DS + OS + TB + AH; | | | | |
| | RT + FPL + LA + MM + AH; | | | | |
| | DS + FPL + OS + TB + MM; EC + FPL + LA + KQ + RI; | | | | |
| | SG + MM + AH + FP + MS; PS + DS + LA + FP + RI; | | | | |
| | RT + TB + MM + KQ + RI; DS + EL + TB + AH + RI; | | | | |
| | EC + SG + FPL + AH + RI; EC + PS + RT + EL + MF; | | | | |
| | EC + EL + MF + FP + MS; EC + LA + MM + AH + MS; | | | | |
| | PS + RT + DS + MM + ES; PS + DS + LA + MM + MS; | | | | |
| | FPL + LA + MM + AH + FP; EL + LA + AH + FP + MS; | | | | |
| | MF + FP + ES + MS + RI; RT + MF + ES + MS + KQ; | | | | |
| | DS + FPL + MM + AH + RI; EC + RT + OS + LA + ES; | | | | |
| | EC + RT + EL + LA + MM; EC + EL + LA + FP + KQ; | | | | |
| | SG + MM + FP + ES + KQ; PS + RT + FPL + MM + ES; | | | | |
| | FPL + EL + LA + ES + RI; FPL + EL + FP + KQ + RI; | | | | |
| | FPL + LA + AH + FP + RI; EC + SG + MF + MM + RI; | | | | |
| | SG + RT + FPL + AH + RI; SG + FPL + EL + MM + MS; | | | | |
| | PS + LA + MF + AH + KQ; DS + OS + LA + TB + RI; | | | | |
| | FPL + LA + MM + AH + ES; MM + AH + FP + ES + RI; | | | | |
| | EC + RT + FPL + AH + MS; PS + MF + AH + ES + MS; | | | | |
| | RT + EL + MF + MS + RI; DS + FPL + EL + MM + AH; | | | | |
| | TB + AH + FP + MS + RI; EC + FPL + EL + LA + MF; | | | | |
| | EC + FPL + EL + KQ + RI; PS + FPL + MM + FP + KQ; | | | | |
| | RT + DS + MM + AH + RI; RT + MM + FP + MS + KQ; | | | | |
| | FPL + MF + FP + ES + KQ; EC + PS + OS + EL + ES; | | | | |
| | EC + RT + EL + MF + TB; EC + OS + LA + TB + KQ; | | | | |
| | SG + RT + FPL + TB + KQ; SG + RT + MM + AH + ES; | | | | |
| | PS + FPL + LA + ES + MS; RT + DS + AH + FP + RI; | | | | |
| | DS + EL + MF + FP + KQ; DS + TB + MM + ES + KQ; | | | | |
| | PS + DS + FPL + MF + MM; PS + FPL + EL + MM + MS; | | | | |
| | PS + EL + MM + MS + RI; RT + DS + FPL + OS + LA; | | | | |
| | DS + TB + MS + KQ + RI; DS + MM + ES + KQ + RI; | | | | |
| | EC + FPL + EL + MM + AH; PS + LA + AH + MS + KQ; | | | | |
| | RT + EL + MF + AH + ES; DS + EL + MM + FP + KQ; | | | | |
| | MF + TB + ES + MS + RI; EC + SG + PS + OS + KQ; | | | | |
| | EC + PS + RT + MF + AH; SG + FPL + AH + FP + MS; | | | | |
| | PS + DS + LA + MF + FP; PS + FPL + MF + AH + ES; | | | | |
| | EC + SG + OS + LA + RI; EC + FPL + EL + MF + MS; | | | | |
| | EC + MF + ES + MS + KQ; SG + RT + EL + AH + KQ; | | | | |
| | PS + LA + FP + KQ + RI; RT + EL + MM + FP + RI; | | | | |
| | FPL + EL + LA + FP + RI; EC + RT + MM + AH + FP; | | | | |
| | EC + DS + LA + MF + MM; PS + RT + DS + MS + KQ; | | | | |
| | PS + FPL + EL + LA + MM; PS + EL + MF + TB + AH; | | | | |
| | EC + SG + DS + OS + MS; EC + RT + MF + AH + FP; | | | | |
| | SG + LA + MF + MS + KQ; PS + DS + FPL + MM + FP; | | | | |
| | LA + MF + AH + FP + KQ; LA + MM + AH + ES + RI; | | | | |
| | SG + PS + TB + FP + ES; SG + FPL + MF + MS + RI; | | | | |
| | PS + MF + TB + MM + FP; FPL + MF + MM + FP + KQ; | | | | |
| | EC + PS + RT + MM + AH; EC + MF + AH + FP + MS; | | | | |
| | SG + DS + MF + TB + ES; PS + RT + DS + MM + KQ; | | | | |
| | PS + RT + FP + MS + KQ; DS + LA + MF + TB + KQ; | | | | |
| | FPL + LA + ES + MS + KQ; EC + PS + DS + OS + MS; | | | | |
| | EC + FPL + LA + AH + MS; SG + TB + AH + KQ + RI; | | | | |
| | PS + EL + AH + MS + KQ; DS + FPL + AH + FP + MS; | | | | |
| | FPL + LA + MM + FP + RI; SG + MF + TB + MS + RI; | | | | |
| | PS + LA + MM + FP + RI; EL + LA + MF + AH + FP; | | | | |
| | EL + LA + MM + AH + FP; EC + PS + OS + KQ + RI; | | | | |
| | EC + FPL + EL + MF + MM; EC + MF + TB + AH + RI; | | | | |
| | EC + EL + AH + FP + MS; SG + DS + LA + MF + KQ; | | | | |
| | SG + LA + MF + TB + KQ; SG + LA + MF + MM + FP; | | | | |
| | PS + DS + EL + MF + KQ; PS + EL + LA + AH + MS; | | | | |
| | DS + EL + MF + MS + RI; LA + MF + FP + ES + KQ; | | | | |
| | EC + PS + OS + MF + ES; EC + PS + MF + MM + KQ; | | | | |
| | SG + PS + MM + MS + KQ; SG + LA + MM + MS + RI; | | | | |
| | PS + DS + MM + ES + KQ; RT + FPL + MM + FP + RI; | | | | |
| | EC + FPL + AH + ES + MS; SG + RT + DS + MM + MS; | | | | |
| | SG + FPL + MM + MS + RI; EC + FPL + MM + FP + RI; | | | | |
| | EC + MM + AH + FP + KQ; SG + RT + DS + AH + ES; | | | | |
| | SG + EL + TB + AH + KQ; PS + RT + MF + KQ + RI; | | | | |
| | PS + DS + MM + FP + RI; RT + FPL + MF + TB + KQ; | | | | |
| | EC + LA + MF + TB + MM; FPL + EL + MS + KQ + RI; | | | | |
| | TB + AH + ES + KQ + RI; SG + MF + AH + ES + KQ; | | | | |
| | PS + DS + TB + MS + KQ; RT + DS + MF + AH + RI; | | | | |
| | RT + DS + MM + KQ + RI; DS + FPL + EL + AH + RI; | | | | |
| | EC + RT + TB + MM + AH; SG + RT + DS + AH + KQ; | | | | |
| | RT + EL + MF + TB + KQ; DS + MF + FP + MS + KQ; | | | | |
| | DS + FP + ES + KQ + RI; MF + AH + FP + ES + RI; | | | | |
| | EC + FPL + FP + KQ + RI; EC + LA + MF + MS + KQ; | | | | |
| | PS + RT + LA + MM + AH; PS + EL + TB + AH + RI; | | | | |
| | PS + FP + ES + MS + RI; RT + DS + EL + AH + KQ; | | | | |
| | DS + LA + FP + KQ + RI; SG + RT + DS + ES + KQ; | | | | |
| | PS + RT + LA + MF + AH; PS + RT + MF + FP + RI; | | | | |
| | PS + RT + TB + MS + RI; RT + EL + MM + MS + RI; | | | | |
| | EC + SG + OS + EL + FP; EC + SG + MF + TB + KQ; | | | | |
| | EC + PS + LA + MF + KQ; PS + FPL + EL + LA + FP; | | | | |
| | EL + LA + TB + AH + RI; EL + LA + AH + ES + RI; | | | | |
| | EC + PS + EL + MF + RI; EC + EL + MF + MM + FP; | | | | |
| | EC + LA + FP + KQ + RI; SG + PS + FPL + LA + MS; | | | | |
| | SG + RT + AH + FP + RI; PS + EL + MF + FP + MS; | | | | |
| | DS + OS + EL + LA + RI; EC + SG + LA + MF + MM; | | | | |
| | EC + EL + MM + FP + KQ; EC + LA + MM + FP + KQ; | | | | |
| | SG + PS + RT + MF + ES; SG + MF + TB + ES + RI; | | | | |
| | PS + RT + LA + MF + TB; PS + FPL + MF + MM + ES; | | | | |
| | PS + LA + MF + TB + RI; DS + LA + AH + MS + RI; | | | | |
| | LA + MF + AH + ES + RI; EC + LA + MF + MS + RI; | | | | |
| | EC + LA + MM + MS + KQ; EC + MM + FP + ES + RI; | | | | |
| | FPL + MF + FP + MS + KQ; EC + SG + FPL + OS + TB; | | | | |
| | EC + SG + OS + EL + LA; EC + OS + TB + AH + FP; | | | | |
| | SG + PS + RT + DS + TB; PS + RT + DS + TB + ES; | | | | |
| | PS + RT + EL + AH + KQ; PS + FPL + ES + MS + KQ; | | | | |
| | RT + LA + MM + FP + RI; DS + EL + TB + MS + KQ; | | | | |
| | EC + RT + MM + AH + KQ; EC + EL + MM + FP + MS; | | | | |
| | EC + MM + FP + MS + RI; SG + DS + OS + EL + FP; | | | | |
| | SG + EL + AH + MS + KQ; EL + MM + FP + MS + RI; | | | | |
| | EC + RT + OS + LA + FP; EC + EL + MF + TB + RI; | | | | |
| | EC + EL + MF + FP + KQ; SG + RT + OS + LA + AH; | | | | |
| | PS + DS + MF + KQ + RI; RT + LA + AH + MS + KQ; | | | | |
| | PS + RT + FPL + FP + MS; PS + RT + MM + MS + KQ; | | | | |
| | RT + MF + MM + FP + ES; DS + MF + AH + FP + MS; | | | | |
| | DS + FP + ES + MS + KQ; EC + OS + LA + TB + MS; | | | | |
| | SG + PS + OS + LA + AH; SG + FPL + OS + LA + FP; | | | | |
| | PS + RT + TB + KQ + RI; RT + FPL + LA + AH + RI; | | | | |
| | LA + MM + AH + FP + KQ; EC + DS + OS + TB + MS; | | | | |
| | RT + MF + TB + AH + FP; LA + MF + AH + FP + MS; | | | | |
| | EC + PS + OS + EL + KQ; EC + FPL + LA + MM + ES; | | | | |
| | EC + EL + MF + TB + AH; EC + EL + TB + MM + RI; | | | | |
| | SG + PS + TB + MM + FP; SG + TB + AH + MS + RI; | | | | |
| | PS + RT + FPL + MS + KQ; PS + FPL + LA + MS + KQ; | | | | |
| | FPL + EL + LA + TB + MS; EC + PS + RT + MF + KQ; | | | | |
| | SG + PS + RT + MS + KQ; SG + PS + EL + AH + MS; | | | | |
| | PS + FPL + LA + AH + RI; DS + AH + FP + MS + RI; | | | | |
| | FPL + EL + LA + MM + KQ | | | | |
| 5 | EC + RT + LA + MM + MS; EC + RT + FP + MS + RI; | 0.75 | 0.3102- | 42.86%- | 40.40%- |
| | PS + TB + MM + AH + RI; DS + FPL + FP + MS + KQ; | | 0.5214 | 96.83% | 94.95% |
| | EC + PS + FPL + EL + MM; EC + PS + EL + LA + AH; | | | | |
| | EC + DS + MM + AH + RI; EC + FPL + EL + MF + ES; | | | | |
| | RT + DS + FPL + AH + KQ; EC + RT + DS + TB + MM; | | | | |
| | EC + DS + MM + AH + KQ; EC + LA + MF + ES + KQ; | | | | |
| | SG + RT + TB + FP + MS; PS + RT + EL + LA + FP; | | | | |
| | LA + MM + AH + ES + KQ; LA + MM + FP + ES + RI; | | | | |
| | LA + AH + FP + ES + KQ; EC + RT + FPL + LA + MF; | | | | |
| | EC + RT + TB + MM + MS; EC + DS + FPL + MF + MS; | | | | |
| | EC + AH + FP + ES + KQ; SG + PS + MF + AH + FP; | | | | |
| | PS + RT + EL + AH + ES; RT + DS + FP + MS + KQ; | | | | |
| | DS + EL + AH + ES + KQ; TB + MM + AH + FP + MS; | | | | |
| | EC + SG + EL + MF + MS; SG + RT + EL + MS + RI; | | | | |
| | SG + FPL + EL + KQ + RI; PS + MF + TB + ES + RI; | | | | |
| | RT + DS + EL + MM + ES; DS + LA + MF + AH + MS; | | | | |
| | EC + SG + LA + MF + MS; EC + EL + LA + ES + KQ; | | | | |
| | EC + EL + MF + FP + ES; SG + PS + DS + MM + AH; | | | | |
| | SG + PS + EL + MM + AH; SG + DS + LA + MF + TB; | | | | |
| | PS + RT + FPL + MF + ES; PS + DS + EL + ES + RI; | | | | |
| | PS + DS + LA + TB + MM; LA + TB + MM + AH + KQ; | | | | |
| | EC + PS + RT + DS + TB; EC + RT + DS + AH + RI; | | | | |
| | SG + MF + TB + FP + RI; PS + DS + LA + MM + ES; | | | | |
| | RT + MF + FP + ES + MS; EC + SG + RT + EL + MF; | | | | |
| | EC + SG + MM + MS + RI; EC + RT + DS + FP + KQ; | | | | |
| | EC + LA + FP + ES + RI; EC + TB + MM + KQ + RI; | | | | |
| | SG + PS + DS + OS + RI; SG + MM + ES + KQ + RI; | | | | |
| | PS + RT + FPL + EL + LA; PS + DS + TB + FP + KQ; | | | | |
| | EC + PS + RT + FPL + FP; EC + PS + FPL + EL + MF; | | | | |
| | EC + RT + FPL + LA + RI; EC + RT + LA + TB + RI; | | | | |
| | EC + DS + AH + FP + RI; EC + FPL + MF + AH + FP; | | | | |
| | EC + FPL + AH + FP + KQ; SG + RT + FPL + TB + MS; | | | | |
| | PS + RT + FPL + ES + MS; RT + LA + TB + MS + RI; | | | | |
| | DS + EL + MF + AH + ES; EC + PS + FPL + MF + AH; | | | | |
| | EC + RT + LA + TB + AH; EC + DS + EL + MF + KQ; | | | | |
| | EC + DS + MM + MS + RI; EC + LA + TB + KQ + RI; | | | | |
| | SG + PS + FPL + TB + AH; SG + DS + EL + LA + MM; | | | | |
| | PS + RT + EL + TB + RI; DS + FPL + EL + MF + MM; | | | | |
| | DS + FPL + LA + MM + MS; DS + FPL + TB + AH + KQ; | | | | |
| | EC + PS + RT + DS + MF; EC + PS + LA + AH + RI; | | | | |
| | EC + DS + MF + KQ + RI; EC + FPL + TB + MM + ES; | | | | |
| | EC + MF + AH + FP + ES; SG + RT + DS + EL + MF; | | | | |
| | SG + RT + DS + EL + TB; SG + RT + DS + EL + MS; | | | | |
| | SG + DS + FPL + MM + MS; SG + DS + FPL + AH + MS; | | | | |
| | PS + RT + AH + ES + KQ; PS + DS + EL + LA + AH; | | | | |
| | PS + TB + MM + FP + ES; RT + DS + FPL + LA + MF; | | | | |
| | RT + DS + TB + FP + ES; RT + FPL + LA + FP + MS; | | | | |
| | DS + EL + LA + MF + FP; DS + MF + TB + MS + RI; | | | | |
| | DS + TB + MM + KQ + RI; FPL + MF + TB + AH + KQ; | | | | |
| | EC + PS + RT + DS + AH; EC + DS + EL + MM + ES; | | | | |
| | EC + FPL + MF + MM + ES; EC + MF + TB + ES + KQ; | | | | |
| | SG + PS + EL + AH + RI; SG + PS + MM + MS + RI; | | | | |
| | SG + RT + EL + MF + RI; SG + FPL + EL + MF + MM; | | | | |
| | SG + LA + ES + KQ + RI; PS + RT + DS + MM + RI; | | | | |
| | PS + TB + AH + FP + RI; DS + FPL + LA + MF + MS; | | | | |
| | DS + EL + MF + AH + FP; EL + LA + MM + ES + KQ; | | | | |
| | EC + SG + DS + MF + MM; EC + RT + DS + AH + FP; | | | | |
| | EC + RT + FPL + EL + FP; EC + EL + AH + ES + KQ; | | | | |
| | SG + RT + DS + LA + TB; SG + RT + DS + MM + FP; | | | | |
| | PS + DS + AH + ES + MS; RT + FPL + EL + FP + MS; | | | | |
| | RT + OS + LA + TB + FP; DS + FPL + MF + FP + KQ; | | | | |
| | DS + FPL + MM + AH + FP; MF + TB + AH + FP + KQ; | | | | |
| | EC + SG + RT + MM + MS; EC + RT + TB + KQ + RI; | | | | |
| | SG + FPL+LA+TB+AH; PS+DS+FPL+EL+MS; | | | | |
| | PS + DS + MM + AH + RI; PS + DS + AH + ES + KQ; | | | | |
| | RT + FPL + TB + ES + MS; RT + TB + MS + KQ + RI; | | | | |
| | DS + FPL + MF + AH + RI; DS + FPL + MF + MS + KQ; | | | | |
| | EC + SG + FPL + OS + LA; EC + SG + LA + MF + KQ; | | | | |
| | EC + SG + MF + MS + RI; EC + PS + EL + MM + FP; | | | | |
| | EC + DS + AH + FP + KQ; EC + DS + AH + MS + KQ; | | | | |
| | EC + EL + LA + MF + ES; SG + PS + AH + KQ + RI; | | | | |
| | SG + RT + DS + MF + MS; PS + RT + LA + MM + RI; | | | | |
| | RT + FPL + FP + MS + RI; RT + EL + LA + MF + FP; | | | | |
| | DS + FPL + EL + KQ + RI; MF + TB + MM + ES + MS; | | | | |
| | EC + DS + EL + AH + KQ; EC + FPL + LA + ES + RI; | | | | |
| | EC + LA + FP + MS + KQ; EC + MF + AH + ES + KQ; | | | | |
| | PS + RT + MM + KQ + RI; RT + MF + TB + FP + RI; | | | | |
| | RT + FP + ES + MS + KQ; EL + MF + AH + FP + ES; | | | | |
| | EC + SG + RT + FPL + MF; EC + RT + LA + AH + FP; | | | | |
| | SG + FPL + TB + FP + MS; PS + RT + DS + TB + RI; | | | | |
| | PS + DS + MM + FP + MS; PS + DS + FP + ES + RI; | | | | |
| | PS + DS + FP + MS + RI; RT + TB + MM + ES + RI; | | | | |
| | EC + RT + DS + OS + ES; EC + FPL + EL + MF + TB; | | | | |
| | EC + EL + LA + FP + ES; EC + EL + MF + ES + MS; | | | | |
| | SG + DS + EL + MM + KQ; PS + RT + FPL + TB + RI; | | | | |
| | PS + DS + MM + ES + MS; DS + MM + AH + MS + RI; | | | | |
| | TB + FP + MS + KQ + RI; EC + EL + MF + AH + ES; | | | | |
| | EC + LA + MF + AH + FP; PS + MM + AH + FP + ES; | | | | |
| | RT + EL + LA + MM + KQ; EC + RT + EL + TB + KQ; | | | | |
| | EC + DS + FPL + FP + MS; PS + RT + LA + AH + KQ; | | | | |
| | PS + DS + TB + AH + MS; RT + DS + FPL + MF + MS; | | | | |
| | RT + FPL + MM + ES + RI; RT + FPL + AH + ES + RI; | | | | |
| | DS + FPL + OS + TB + ES; LA + MM + ES + MS + KQ; | | | | |
| | EC + PS + FPL + MF + KQ; EC + RT + FPL + MF + AH; | | | | |
| | SG + PS + DS + AH + MS; SG + RT + FPL + AH + ES; | | | | |
| | PS + DS + FPL + LA + MS; RT + DS + FPL + MF + KQ; | | | | |
| | RT + DS + TB + MS + KQ; RT + FPL + LA + FP + RI; | | | | |
| | SG + DS + MF + FP + MS; SG + DS + ES + MS + KQ; | | | | |
| | RT + DS + FPL + AH + RI; RT + DS + LA + MF + TB; | | | | |
| | DS + MM + FP + ES + RI; EC + EL + LA + MF + TB; | | | | |
| | SG + PS + RT + DS + ES; PS + RT + DS + LA + TB; | | | | |
| | PS + LA + TB + MM + MS; DS + MF + TB + FP + KQ; | | | | |
| | FPL + EL + MM + FP + ES; PS + RT + FPL + LA + FP; | | | | |
| | PS + LA + TB + FP + RI; RT + TB + AH + FP + ES; | | | | |
| | LA + TB + ES + KQ + RI; EC + SG + DS + OS + ES; | | | | |
| | EC + PS + FPL + LA + RI; EC + RT + EL + FP + RI; | | | | |
| | SG + PS + DS + ES + MS; SG + PS + MM + KQ + RI; | | | | |
| | RT + DS + FPL + MM + RI; EL + MM + AH + ES + KQ; | | | | |
| | TB + AH + ES + MS + RI; EC + SG + MF + ES + KQ; | | | | |
| | EC + RT + DS + OS + LA; EC + RT + DS + EL + KQ; | | | | |
| | EC + DS + LA + FP + RI; EC + FPL + TB + MS + RI; | | | | |
| | EC + LA + AH + FP + MS; SG + PS + RT + DS + FP; | | | | |
| | SG + PS + DS + MF + AH; SG + DS + EL + TB + AH; | | | | |
| | SG + DS + MF + FP + RI; PS + FPL + LA + AH + ES; | | | | |
| | PS + EL + MM + MS + KQ; PS + MM + ES + KQ + RI; | | | | |
| | RT + LA + AH + ES + RI; DS + EL + LA + ES + RI; | | | | |
| | FPL + LA + TB + MM + RI; LA + MF + TB + FP + RI; | | | | |
| | EC + SG + DS + EL + TB; EC + PS + EL + LA + FP; | | | | |
| | EC + DS + FPL + AH + FP; SG + RT + FPL + EL + MF; | | | | |
| | DS + EL + TB + AH + MS; FPL + LA + MM + ES + MS; | | | | |
| | EC + PS + FPL + MS + RI; EC + PS + LA + MM + FP; | | | | |
| | SG + RT + DS + LA + MM; PS + RT + FPL + FP + ES; | | | | |
| | PS + FPL + TB + MM + FP; EC + PS + RT + FPL + KQ; | | | | |
| | EC + DS + FPL + MF + FP; EC + DS + LA + MM + RI; | | | | |
| | EC + EL + FP + ES + KQ; SG + RT + EL + MM + ES; | | | | |
| | SG + RT + TB + MM + ES; SG + MF + TB + FP + MS; | | | | |
| | DS + FPL + OS + TB + RI; DS + FPL + MM + ES + MS; | | | | |
| | MF + TB + FP + MS + KQ; EC + DS + MM + FP + RI; | | | | |
| | EC + EL + MM + ES + KQ; SG + DS + AH + MS + KQ; | | | | |
| | EC + PS + FPL + EL + RI; EC + RT + FPL + MF + RI; | | | | |
| | EC + FPL + EL + MF + AH; EC + FPL + AH + ES + RI; | | | | |
| | SG + RT + LA + MF + KQ; PS + EL + TB + AH + FP; | | | | |
| | PS + TB + MM + AH + KQ; RT + FP + MS + KQ + RI; | | | | |
| | DS + LA + MS + KQ + RI; FPL + MF + TB + MM + MS; | | | | |
| | EC + SG + RT + OS + RI; EC + DS + EL + LA + RI; | | | | |
| | EC + DS + MF + AH + MS; SG + PS + EL + TB + KQ; | | | | |
| | SG + PS + TB + KQ + RI; SG + FPL + EL + LA + MS; | | | | |
| | PS + FPL + AH + FP + ES; RT + DS + FPL + MM + KQ; | | | | |
| | RT + DS + MM + MS + RI; RT + EL + MF + ES + KQ; | | | | |
| | DS + TB + AH + FP + KQ; EC + PS + DS + MM + FP; | | | | |
| | EC + PS + FPL + EL + KQ; EC + PS + OS + LA + TB; | | | | |
| | EC + DS + FPL + OS + ES; EC + DS + MM + AH + MS; | | | | |
| | EC + FPL + ES + KQ + RI; EC + EL + AH + FP + ES; | | | | |
| | SG + DS + FPL + MM + AH; SG + LA + MF + TB + ES; | | | | |
| | SG + TB + FP + KQ + RI; RT + DS + MF + FP + RI; | | | | |
| | FPL + EL + MM + ES + RI; EC + DS + EL + MF + AH; | | | | |
| | EC + FPL + EL + AH + ES; EC + MF + FP + ES + MS; | | | | |
| | SG + MF + MM + FP + ES; RT + DS + LA + AH + MS; | | | | |
| | RT + DS + AH + ES + RI; DS + MF + TB + AH + MS; | | | | |
| | EC + SG + AH + MS + KQ; EC + PS + FPL + EL + AH; | | | | |
| | EC + RT + FPL + MF + MS; EC + DS + EL + LA + MF; | | | | |
| | EC + DS + MF + MM + FP; EC + DS + MM + KQ + RI; | | | | |
| | EC + FPL + MF + TB + MM; SG + RT + MF + MS + RI; | | | | |
| | SG + RT + AH + FP + ES; SG + DS + EL + MM + RI; | | | | |
| | RT + DS + FPL + LA + MS; DS + FPL + EL + LA + RI; | | | | |
| | EC + PS + EL + MF + MS; EC + PS + EL + AH + FP; | | | | |
| | EC + LA + MM + FP + ES; SG + RT + AH + ES + RI; | | | | |
| | FPL + EL + MF + FP + KQ; EC + PS + OS + LA + ES; | | | | |
| | EC + MM + FP + ES + MS; SG + RT + EL + MM + FP; | | | | |
| | RT + EL + MF + ES + MS; RT + EL + TB + MM + FP; | | | | |
| | DS + FPL + EL + LA + MM; LA + MF + FP + ES + RI; | | | | |
| | MF + TB + AH + FP + RI; EC + PS + RT + FPL + MS; | | | | |
| | EC + PS + LA + FP + RI; EC + RT + LA + KQ + RI; | | | | |
| | SG + FPL + AH + ES + RI; PS + DS + ES + MS + KQ; | | | | |
| | RT + DS + OS + LA + ES; RT + DS + EL + MS + KQ; | | | | |
| | FPL + LA + MM + FP + ES; EC + DS + OS + TB + ES; | | | | |
| | PS + RT + DS + FPL + TB; PS + FPL + EL + MM + KQ; | | | | |
| | PS + EL + TB + AH + KQ; EC + RT + EL + LA + KQ; | | | | |
| | EC + FPL + EL + FP + MS; EC + FPL + LA + MF + TB; | | | | |
| | EC + EL + LA + TB + AH; SG + EL + MM + KQ + RI; | | | | |
| | PS + DS + TB + MM + RI; RT + DS + LA + AH + RI; | | | | |
| | DS + FPL + EL + MS + KQ; DS + EL + LA + FP + RI; | | | | |
| | EC + DS + FPL + MF + KQ; EC + EL + AH + ES + MS; | | | | |
| | SG + PS + TB + FP + MS; PS + DS + TB + ES + KQ; | | | | |
| | PS + DS + MS + KQ + RI; RT + DS + MM + ES + RI; | | | | |
| | RT + FPL + MF + TB + RI; EC + SG + MF + AH + RI; | | | | |
| | EC + RT + LA + AH + KQ; EC + FPL + LA + TB + MS; | | | | |
| | SG + MF + TB + AH + RI; PS + RT + FPL + EL + FP; | | | | |
| | PS + DS + MF + AH + RI; RT + LA + FP + KQ + RI; | | | | |
| | EC + RT + FPL + LA + MM; EC + DS + MF + MM + ES; | | | | |
| | SG + PS + MM + AH + FP; SG + RT + DS + EL + KQ; | | | | |
| | SG + RT + FPL + TB + AH; SG + DS + OS + LA + KQ; | | | | |
| | SG + EL + LA + MS + KQ; FPL + MF + AH + FP + ES; | | | | |
| | MF + TB + MM + AH + ES; EC + SG + MF + MM + FP; | | | | |
| | EC + PS + FPL + MF + MM; EC + PS + AH + MS + KQ; | | | | |
| | EC + RT + LA + TB + MM; RT + DS + EL + MF + TB; | | | | |
| | RT + DS + MM + FP + ES; FPL + EL + ES + MS + RI; | | | | |
| | EC + EL + MF + TB + MS; EC + LA + MF + TB + AH; | | | | |
| | SG + DS + AH + FP + KQ; PS + FPL + LA + TB + AH; | | | | |
| | PS + AH + FP + ES + RI; FPL + LA + MF + TB + AH; | | | | |
| | EC + SG + MF + FP + KQ; EC + SG + MM + AH + RI; | | | | |
| | EC + PS + RT + MM + KQ; EC + RT + DS + MF + TB; | | | | |
| | EC + RT + EL + LA + FP; EC + DS + FPL + TB + AH; | | | | |
| | EC + DS + LA + MM + KQ; EC + FPL + EL + ES + KQ; | | | | |
| | SG + MF + TB + MM + FP; FPL + LA + TB + AH + KQ; | | | | |
| | EL + LA + TB + MM + AH; EC + SG + RT + MF + AH; | | | | |
| | EC + RT + FPL + AH + FP; EC + FPL + MF + FP + MS; | | | | |
| | SG + PS + ES + MS + KQ; PS + DS + EL + AH + FP; | | | | |
| | PS + EL + TB + MM + AH; PS + EL + MM + ES + RI; | | | | |
| | PS + DS + FPL + FP + KQ; EC + SG + DS + OS + AH; | | | | |
| | SG + RT + DS + MF + AH; SG + LA + MF + FP + RI; | | | | |
| | PS + DS + AH + FP + RI; FPL + MM + ES + MS + KQ; | | | | |
| | EC + RT + DS + LA + MM; PS + DS + TB + MM + KQ; | | | | |
| | RT + FPL + MF + KQ + RI; EL + LA + MM + FP + MS; | | | | |
| | EC + PS + RT + FPL + MF; EC + PS + FPL + LA + MM; | | | | |
| | EC + LA + TB + AH + RI; SG + PS + MM + FP + KQ; | | | | |
| | SG + RT + DS + MF + ES; SG + LA + MM + ES + KQ; | | | | |
| | SG + MM + ES + MS + RI; PS + RT + DS + AH + ES; | | | | |
| | FPL + LA + MM + ES + KQ; EC + EL + MF + TB + KQ; | | | | |
| | SG + RT + MM + FP + MS; SG + DS + FP + ES + KQ; | | | | |
| | PS + DS + EL + TB + AH; PS + EL + LA + MF + ES; | | | | |
| | PS + LA + AH + FP + KQ; PS + MF + TB + FP + ES; | | | | |
| | RT + EL + MF + FP + RI; FPL + MF + MM + FP + ES; | | | | |
| | EC + PS + DS + OS + RI; EC + RT + LA + MF + MS; | | | | |
| | EC + DS + FPL + OS + TB; EC + FPL + MM + AH + ES; | | | | |
| | SG + PS + AH + ES + MS; SG + RT + DS + MM + ES; | | | | |
| | SG + DS + MM + FP + RI; TB + MM + AH + ES + RI; | | | | |
| | EC + SG + MF + AH + KQ; EC + PS + FPL + AH + KQ; | | | | |
| | EC + PS + EL + AH + MS; EC + DS + FPL + MF + ES; | | | | |
| | SG + DS + TB + MM + FP; PS + RT + EL + FP + RI; | | | | |
| | DS + EL + TB + AH + KQ; DS + LA + AH + FP + RI; | | | | |
| | EC + SG + AH + MS + RI; EC + DS + EL + MM + MS; | | | | |
| | EC + DS + MM + MS + KQ; RT + EL + LA + FP + RI; | | | | |
| | RT + EL + AH + ES + KQ; EL + MF + FP + ES + RI; | | | | |
| | EC + RT + TB + MM + KQ; EC + DS + OS + LA + AH; | | | | |
| | EC + MF + FP + ES + RI; SG + PS + MF + AH + RI; | | | | |
| | SG + PS + FP + MS + KQ; SG + LA + MF + FP + KQ; | | | | |
| | FPL + EL + LA + MF + FP; FPL + EL + MM + FP + KQ; | | | | |
| | LA + FP + MS + KQ + RI; EC + SG + OS + LA + TB; | | | | |
| | EC + SG + OS + LA + ES; SG + EL + MM + AH + ES; | | | | |
| | SG + TB + MM + MS + KQ; PS + DS + TB + FP + RI; | | | | |
| | PS + FPL + ES + MS + RI; RT + DS + ES + MS + KQ; | | | | |
| | DS + MF + AH + ES + MS; EC + DS + FPL + OS + FP; | | | | |
| | SG + PS + LA + MF + RI; SG + RT + AH + ES + KQ; | | | | |
| | SG + FPL + MF + KQ + RI; FPL + LA + MF + FP + RI; | | | | |
| | OS + LA + TB + AH + FP; EC + PS + RT + EL + TB; | | | | |
| | EC + RT + LA + MF + FP; EC + TB + AH + FP + RI; | | | | |
| | RT + DS + LA + AH + FP; EC + PS + LA + FP + KQ; | | | | |
| | EC + PS + MF + AH + KQ; EC + FPL + TB + MM + MS; | | | | |
| | SG + FPL + MF + AH + FP; SG + TB + ES + MS + KQ; | | | | |
| | PS + DS + EL + AH + KQ; PS + FPL + FP + MS + KQ; | | | | |
| | DS + LA + AH + KQ + RI; EC + PS + RT + EL + FP; | | | | |
| | EC + RT + EL + TB + RI; EC + RT + EL + FP + KQ; | | | | |
| | EC + RT + LA + MM + KQ; SG + PS + DS + EL + AH; | | | | |
| | PS + RT + DS + EL + KQ; RT + DS + TB + MM + FP; | | | | |
| | EL + LA + TB + KQ + RI; EC + RT + FPL + MF + FP; | | | | |
| | EC + RT + FPL + KQ + RI; EC + DS + EL + AH + MS; | | | | |
| | EC + DS + MF + MM + RI; SG + RT + TB + ES + KQ; | | | | |
| | PS + DS + AH + KQ + RI; PS + AH + FP + ES + KQ; | | | | |
| | RT + FPL + MM + KQ + RI; RT + FPL + FP + MS + KQ; | | | | |
| | LA + AH + FP + ES + RI; EC + PS + FPL + EL + MS; | | | | |
| | EC + RT + TB + AH + KQ; EC + DS + EL + FP + KQ; | | | | |
| | RT + FPL + LA + MS + RI; DS + AH + FP + ES + KQ; | | | | |
| | LA + MM + FP + ES + KQ; EC + PS + EL + MF + FP; | | | | |
| | SG + PS + RT + ES + MS; SG + DS + TB + FP + KQ; | | | | |
| | PS + RT + FPL + MS + RI; PS + RT + LA + TB + MM; | | | | |
| | PS + RT + TB + FP + ES; RT + DS + FPL + TB + MM; | | | | |
| | RT + LA + MF + ES + KQ; RT + MF + FP + MS + RI; | | | | |
| | EL + MM + AH + FP + ES; EC + PS + RT + DS + FP; | | | | |
| | EC + PS + FPL + KQ + RI; EC + PS + MM + MS + KQ; | | | | |
| | EC + RT + DS + EL + MF; EC + RT + FPL + EL + MM; | | | | |
| | SG + PS + TB + AH + MS; SG + EL + TB + MM + RI; | | | | |
| | RT + FPL + TB + MM + ES; DS + FPL + AH + FP + RI; | | | | |
| | DS + MF + FP + ES + RI; EC + SG + OS + AH + FP; | | | | |
| | EC + PS + RT + TB + MM; SG + PS + RT + AH + KQ; | | | | |
| | SG + FPL + MF + ES + MS; PS + RT + DS + FP + ES; | | | | |
| | RT + EL + MM + ES + MS; EC + PS + DS + OS + ES; | | | | |
| | RT + DS + LA + FP + RI; EC + SG + RT + MF + TB; | | | | |
| | EC + PS + EL + MM + AH; EC + FPL + TB + AH + MS; | | | | |
| | PS + DS + MF + FP + ES; PS + FPL + LA + MM + KQ; | | | | |
| | PS + FPL + MF + TB + KQ; DS + FPL + MF + MM + MS; | | | | |
| | DS + EL + AH + FP + MS; FPL + EL + LA + TB + RI; | | | | |
| | FPL + EL + AH + FP + KQ; EC + FPL + EL + AH + KQ; | | | | |
| | RT + LA + MF + FP + MS; DS + FPL + LA + MM + AH; | | | | |
| | EC + SG + FPL + FP + MS; EC + RT + LA + AH + MS; | | | | |
| | EC + LA + MF + TB + RI; EC + LA + ES + KQ + RI; | | | | |
| | SG + PS + RT + ES + KQ; SG + PS + FPL + MF + KQ; | | | | |
| | SG + MF + AH + FP + KQ; FPL + LA + FP + MS + RI; | | | | |
| | FPL + TB + MS + KQ + RI; EC + PS + RT + TB + KQ; | | | | |
| | EC + PS + DS + OS + KQ; SG + EL + ES + KQ + RI; | | | | |
| | PS + RT + FPL + LA + AH; PS + DS + MF + MS + RI; | | | | |
| | RT + FPL + EL + FP + KQ; RT + MM + FP + ES + MS; | | | | |
| | EC + SG + PS + MF + KQ; EC + RT + FPL + TB + MS; | | | | |
| | EC + LA + FP + MS + RI; EC + MF + FP + MS + RI; | | | | |
| | SG + DS + MM + FP + KQ; PS + FPL + LA + KQ + RI; | | | | |
| | MF + TB + AH + ES + MS; EC + SG + EL + LA + MS; | | | | |
| | EC + PS + RT + FPL + AH; EC + RT + FP + KQ + RI; | | | | |
| | EC + FPL + TB + MM + KQ; SG + PS + DS + LA + MM; | | | | |
| | SG + FPL + LA + MF + MS; PS + MM + FP + ES + RI; | | | | |
| | DS + LA + AH + MS + KQ; DS + MF + TB + ES + MS; | | | | |
| | EC + SG + PS + DS + OS; EC + PS + RT + AH + KQ; | | | | |
| | EC + RT + EL + MF + FP; SG + DS + MM + FP + ES; | | | | |
| | SG + DS + FP + KQ + RI; PS + RT + DS + EL + TB; | | | | |
| | PS + RT + FPL + LA + MF; PS + RT + LA + MM + MS; | | | | |
| | PS + DS + EL + LA + ES; PS + FPL + LA + AH + KQ; | | | | |
| | PS + EL + MF + TB + MS; RT + FPL + EL + AH + ES; | | | | |
| | RT + TB + MM + MS + KQ; FPL + EL + MF + ES + MS; | | | | |
| | EL + AH + FP + ES + KQ; LA + MF + TB + MM + MS; | | | | |
| | EC + PS + OS + TB + ES; EC + RT + LA + MF + TB; | | | | |
| | EC + RT + MF + FP + MS; EC + DS + OS + TB + KQ; | | | | |
| | EC + DS + EL + LA + MM; SG + DS + FPL + OS + EL; | | | | |
| | SG + DS + LA + MF + ES; SG + FPL + EL + MM + RI; | | | | |
| | SG + FPL + EL + AH + KQ; SG + EL + LA + AH + KQ; | | | | |
| | SG + MF + AH + ES + RI; SG + AH + FP + MS + KQ; | | | | |
| | SG + AH + ES + KQ + RI; PS + MF + TB + FP + MS; | | | | |
| | RT + DS + LA + MF + MS; RT + DS + MM + FP + MS; | | | | |
| | DS + EL + AH + FP + RI; DS + TB + MM + AH + KQ; | | | | |
| | EC + SG + EL + TB + KQ; EC + EL + MF + AH + FP; | | | | |
| | SG + PS + RT + MF + FP; RT + FPL + MF + ES + MS; | | | | |
| | EL + LA + MM + AH + ES; EC + DS + MF + MS + KQ; | | | | |
| | RT + FPL + MF + FP + MS; FPL + TB + MM + KQ + RI; | | | | |
| | EC + DS + LA + MF + KQ; EC + AH + ES + KQ + RI; | | | | |
| | SG + PS + EL + LA + MM; PS + RT + DS + LA + FP; | | | | |
| | PS + DS + FP + ES + KQ; PS + FPL + EL + KQ + RI; | | | | |
| | PS + FPL + MS + KQ + RI; PS + MM + ES + MS + KQ; | | | | |
| | RT + LA + AH + FP + MS; FPL + EL + AH + ES + KQ; | | | | |
| | EC + PS + EL + MS + RI; FPL + TB + MM + FP + MS; | | | | |
| | EC + SG + OS + LA + AH; EC + PS + RT + TB + AH; | | | | |
| | EC + PS + FPL + MS + KQ; EC + RT + MF + MS + RI; | | | | |
| | EC + RT + TB + MM + RI; PS + DS + EL + MM + FP; | | | | |
| | PS + LA + FP + MS + KQ; EC + SG + FPL + MF + KQ; | | | | |
| | EC + FPL + EL + MM + ES; EC + FPL + FP + ES + MS; | | | | |
| | SG + RT + DS + OS + FP; PS + TB + AH + FP + MS; | | | | |
| | RT + DS + FPL + EL + MM; DS + EL + LA + FP + ES; | | | | |
| | DS + EL + MF + TB + AH; DS + MF + AH + ES + RI; | | | | |
| | FPL + EL + TB + AH + KQ; EC + DS + MF + AH + KQ; | | | | |
| | EC + EL + TB + MM + MS; SG + FPL + LA + MM + AH; | | | | |
| | PS + DS + AH + FP + MS; RT + DS + OS + MF + FP; | | | | |
| | RT + FPL + EL + LA + MF; RT + MF + TB + ES + RI; | | | | |
| | FPL + LA + AH + ES + KQ; FPL + MM + FP + ES + KQ; | | | | |
| | EC + PS + RT + FP + MS; EC + PS + FPL + MF + MS; | | | | |
| | EC + LA + MF + FP + MS; SG + MF + AH + FP + RI; | | | | |
| | RT + DS + FP + KQ + RI; DS + FPL + MM + FP + MS; | | | | |
| | DS + MF + TB + MM + FP; DS + MF + ES + MS + RI; | | | | |
| | EC + PS + RT + FPL + LA; EC + PS + FPL + EL + LA; | | | | |
| | EC + PS + AH + KQ + RI; EC + RT + DS + AH + KQ; | | | | |
| | SG + RT + TB + MM + RI; SG + EL + LA + TB + ES; | | | | |
| | SG + AH + FP + KQ + RI; PS + RT + DS + LA + AH; | | | | |
| | PS + RT + FPL + MF + RI; PS + RT + FPL + FP + KQ; | | | | |
| | PS + MF + AH + FP + ES; RT + LA + MM + AH + ES; | | | | |
| | DS + LA + MF + FP + MS; FPL + EL + LA + MM + ES; | | | | |
| | FPL + MF + AH + FP + KQ; EC + AH + FP + ES + RI; | | | | |
| | PS + RT + FPL + FP + RI; PS + DS + FP + ES + MS; | | | | |
| | RT + FPL + EL + MF + ES; RT + LA + MF + MS + RI; | | | | |
| | RT + TB + AH + ES + KQ; EL + MF + TB + ES + KQ; | | | | |
| | EC + PS + MF + KQ + RI; EC + DS + OS + FP + KQ; | | | | |
| | SG + PS + DS + TB + FP; PS + RT + FPL + TB + FP; | | | | |
| | PS + RT + FPL + TB + KQ; RT + LA + MM + KQ + RI; | | | | |
| | DS + FPL + OS + FP + RI; DS + LA + MM + AH + KQ; | | | | |
| | EC + RT + DS + MM + FP; EC + RT + FPL + MS + KQ; | | | | |
| | PS + DS + LA + MF + ES; DS + FPL + MM + MS + RI; | | | | |
| | DS + FPL + AH + ES + MS; FPL + MM + FP + ES + MS; | | | | |
| | EL + TB + MM + AH + ES; TB + AH + FP + MS + KQ; | | | | |
| | EC + PS + RT + MF + RI; EC + DS + MF + MM + AH; | | | | |
| | EC + DS + MF + MM + MS; EC + MF + AH + ES + MS; | | | | |
| | EC + MF + AH + ES + RI; SG + PS + RT + EL + MS; | | | | |
| | PS + RT + DS + FPL + KQ; PS + FPL + EL + MM + RI; | | | | |
| | RT + DS + EL + LA + AH; RT + FPL + LA + AH + KQ; | | | | |
| | DS + FPL + MM + AH + KQ; EL + FP + ES + MS + KQ; | | | | |
| | EC + PS + EL + LA + RI; EC + RT + MF + ES + KQ; | | | | |
| | SG + DS + LA + MM + FP; SG + MF + AH + FP + MS; | | | | |
| | SG + MM + AH + ES + RI; PS + DS + MF + ES + MS; | | | | |
| | PS + DS + MM + AH + FP; PS + MF + TB + ES + MS; | | | | |
| | LA + MF + AH + ES + MS; EC + PS + DS + OS + MF; | | | | |
| | EC + DS + FPL + MM + RI; EC + FPL + EL + MF + FP; | | | | |
| | SG + LA + MM + KQ + RI; PS + FPL + LA + MF + ES; | | | | |
| | PS + LA + AH + FP + MS; PS + LA + AH + KQ + RI; | | | | |
| | RT + DS + LA + MF + AH; DS + MM + FP + ES + MS; | | | | |
| | EL + LA + MF + AH + ES; EC + MF + MM + AH + ES; | | | | |
| | SG + EL + LA + TB + MM; SG + LA + MM + ES + MS; | | | | |
| | SG + TB + MS + KQ + RI; PS + RT + DS + FPL + FP; | | | | |
| | PS + RT + LA + FP + RI; PS + DS + EL + LA + TB; | | | | |
| | RT + DS + FPL + AH + FP; RT + LA + TB + AH + MS; | | | | |
| | DS + MF + FP + ES + MS; LA + MF + TB + MS + KQ; | | | | |
| | EC + SG + RT + MF + MS; EC + PS + MM + FP + MS; | | | | |
| | SG + PS + RT + TB + ES; SG + FP + MS + KQ + RI; | | | | |
| | PS + RT + LA + MF + RI; PS + EL + TB + MS + RI; | | | | |
| | RT + EL + LA + MF + TB; RT + EL + LA + MM + MS; | | | | |
| | EC + LA + MS + KQ + RI; SG + OS + LA + AH + FP; | | | | |
| | PS + RT + DS + LA + MF; RT + LA + TB + MM + MS; | | | | |
| | DS + MF + MM + FP + MS; EC + SG + PS + OS + ES; | | | | |
| | EC + SG + MF + TB + MM; EC + PS + LA + MM + KQ; | | | | |
| | EC + MM + AH + ES + RI; EC + FP + MS + KQ + RI; | | | | |
| | SG + DS + EL + AH + MS; PS + DS + LA + MF + AH; | | | | |
| | RT + MF + TB + FP + MS; DS + FPL + MF + MM + KQ; | | | | |
| | EC + SG + RT + OS + KQ; EC + SG + FPL + EL + KQ; | | | | |
| | EC + SG + EL + AH + MS; EC + MF + ES + MS + RI; | | | | |
| | SG + RT + FPL + MF + KQ; SG + RT + EL + MM + KQ; | | | | |
| | DS + FPL + EL + TB + AH; DS + FPL + EL + MM + MS; | | | | |
| | DS + EL + MF + TB + ES; DS + MF + AH + FP + ES; | | | | |
| | SG + PS + RT + FPL + MM; SG + DS + EL + TB + RI; | | | | |
| | SG + FPL + LA + AH + MS; PS + FPL + MM + FP + ES; | | | | |
| | PS + EL + MM + AH + ES; DS + FPL + AH + KQ + RI; | | | | |
| | DS + EL + LA + MF + AH; FPL + EL + LA + AH + FP; | | | | |
| | EC + AH + ES + MS + RI; SG + PS + LA + ES + MS; | | | | |
| | SG + PS + FP + MS + RI; PS + TB + AH + KQ + RI; | | | | |
| | RT + LA + MM + FP + MS; DS + MF + FP + MS + RI; | | | | |
| | EC + PS + MM + AH + RI; FPL + EL + MF + AH + ES; | | | | |
| | EC + PS + EL + MF + KQ; EC + DS + MM + FP + ES; | | | | |
| | EC + EL + MF + ES + KQ; EC + TB + AH + MS + KQ; | | | | |
| | SG + PS + RT + MM + FP; PS + FPL + MF + ES + MS; | | | | |
| | FPL + EL + MF + MM + KQ; EL + MF + FP + ES + MS; | | | | |
| | EC + RT + DS + EL + AH; EC + DS + EL + MM + AH; | | | | |
| | SG + PS + RT + EL + TB; SG + PS + RT + FP + MS; | | | | |
| | SG + RT + MF + AH + FP; SG + DS + TB + KQ + RI; | | | | |
| | SG + FPL + LA + MF + KQ; RT + DS + FPL + EL + AH; | | | | |
| | DS + EL + LA + FP + KQ; DS + LA + FP + MS + RI; | | | | |
| | EC + SG + FPL + MF + MS; EC + RT + TB + AH + MS; | | | | |
| | EC + FPL + MF + FP + RI; SG + RT + TB + AH + KQ; | | | | |
| | SG + RT + AH + FP + KQ; PS + RT + EL + MF + RI; | | | | |
| | PS + RT + EL + MM + RI; PS + DS + EL + AH + MS; | | | | |
| | RT + FPL + LA + AH + FP; RT + FPL + MF + AH + ES; | | | | |
| | RT + FPL + TB + AH + FP; DS + FPL + OS + LA + TB; | | | | |
| | DS + LA + MF + AH + RI; DS + MM + FP + MS + RI; | | | | |
| | EL + MM + FP + ES + KQ; EL + AH + FP + ES + MS; | | | | |
| | EC + PS + FPL + MM + MS; EC + FPL + LA + TB + MM; | | | | |
| | EC + LA + MF + TB + KQ; SG + PS + DS + TB + MS; | | | | |
| | PS + DS + MF + TB + AH; PS + EL + AH + FP + KQ; | | | | |
| | FPL + MF + TB + MM + ES; EC + SG + RT + OS + ES; | | | | |
| | EC + FPL + EL + FP + KQ; SG + RT + MM + ES + KQ; | | | | |
| | PS + EL + LA + MF + TB; DS + MF + MM + AH + FP; | | | | |
| | EC + PS + FPL + MM + RI; EC + DS + OS + LA + KQ; | | | | |
| | SG + PS + DS + EL + LA; SG + TB + AH + MS + KQ; | | | | |
| | SG + ES + MS + KQ + RI; PS + RT + FP + ES + KQ; | | | | |
| | PS + LA + TB + KQ + RI; PS + TB + ES + KQ + RI; | | | | |
| | EC + PS + LA + MF + MS; EC + FPL + LA + FP + ES; | | | | |
| | SG + RT + MF + KQ + RI; RT + FPL + EL + TB + MS; | | | | |
| | RT + MF + TB + FP + KQ; DS + EL + TB + MM + AH; | | | | |
| | FPL + LA + TB + MM + MS; | | | | |
| | EC + SG + FPL + MF + MM; EC + PS + MF + MM + AH; | | | | |
| | EC + DS + FP + ES + KQ; EC + EL + LA + FP + MS; | | | | |
| | EC + TB + MM + MS + KQ; SG + DS + EL + ES + MS; | | | | |
| | SG + DS + MM + AH + KQ; SG + FPL + MF + MM + ES; | | | | |
| | PS + RT + DS + EL + MS; PS + RT + EL + LA + MM; | | | | |
| | PS + DS + LA + MF + RI; PS + MM + ES + MS + RI; | | | | |
| | RT + EL + LA + MF + MS; DS + FPL + MF + MM + ES; | | | | |
| | DS + MF + AH + MS + RI; LA + FP + ES + MS + KQ; | | | | |
| | TB + AH + ES + MS + KQ; EC + DS + OS + FP + MS; | | | | |
| | EC + EL + FP + MS + KQ; SG + FPL + MM + ES + RI; | | | | |
| | PS + RT + DS + FP + MS; PS + RT + TB + FP + RI; | | | | |
| | PS + EL + LA + FP + KQ; PS + EL + MM + AH + KQ; | | | | |
| | PS + LA + MM + FP + KQ; PS + MM + AH + ES + MS; | | | | |
| | RT + EL + LA + AH + KQ; DS + MF + TB + MM + AH; | | | | |
| | FPL + EL + LA + FP + MS; EC + EL + MM + ES + MS; | | | | |
| | SG + PS + RT + DS + AH; SG + DS + EL + MF + AH; | | | | |
| | SG + FPL + MF + AH + RI; PS + RT + AH + KQ + RI; | | | | |
| | PS + FPL + AH + ES + RI; PS + EL + FP + KQ + RI; | | | | |
| | RT + EL + AH + FP + ES; DS + FPL + EL + MS + RI; | | | | |
| | DS + EL + MM + FP + RI; EL + LA + FP + MS + KQ; | | | | |
| | EL + TB + ES + KQ + RI; SG + PS + RT + FP + KQ; | | | | |
| | PS + DS + MM + KQ + RI; RT + FPL + LA + TB + MM; | | | | |
| | DS + FPL + EL + LA + MS; FPL + EL + LA + ES + MS; | | | | |
| | LA + TB + MM + KQ + RI; SG + FPL + AH + ES + MS; | | | | |
| | RT + DS + FPL + TB + AH; RT + EL + MF + FP + MS; | | | | |
| | DS + FPL + OS + TB + KQ; DS + LA + MM + AH + RI; | | | | |
| | EC + SG + MF + TB + MS; SG + DS + TB + MM + ES; | | | | |
| | RT + DS + FPL + LA + MM; DS + LA + MM + ES + KQ; | | | | |
| | FPL + EL + MM + ES + MS; FPL + LA + FP + MS + KQ; | | | | |
| | EC + PS + RT + FP + KQ; EC + PS + MF + FP + RI; | | | | |
| | EC + RT + FPL + TB + MM; EC + FPL + MF + ES + KQ; | | | | |
| | SG + PS + MM + ES + KQ; SG + DS + FPL + OS + FP; | | | | |
| | SG + EL + MF + AH + ES; RT + DS + TB + MM + RI; | | | | |
| | EC + PS + RT + AH + RI; EC + PS + FPL + FP + MS; | | | | |
| | EC + PS + OS + TB + KQ; EC + PS + MM + AH + FP; | | | | |
| | SG + RT + OS + FP + KQ; PS + RT + FP + MS + RI; | | | | |
| | PS + FPL + EL + LA + KQ; RT + FPL + LA + FP + KQ; | | | | |
| | DS + EL + MF + ES + RI; DS + TB + AH + MS + KQ; | | | | |
| | EL + MF + TB + MS + RI; EC + SG + FPL + MM + RI; | | | | |
| | SG + PS + RT + EL + MF; PS + DS + MF + AH + FP; | | | | |
| | PS + AH + ES + KQ + RI; RT + DS + OS + FP + RI; | | | | |
| | RT + LA + TB + AH + KQ; FPL + MF + TB + MS + RI; | | | | |
| | MF + TB + MM + FP + ES; EC + PS + RT + MM + MS; | | | | |
| | EC + PS + RT + FP + RI; EC + RT + DS + MF + AH; | | | | |
| | EC + EL + FP + ES + RI; SG + PS + RT + DS + MS; | | | | |
| | PS + AH + FP + ES + MS; RT + FPL + EL + LA + MS; | | | | |
| | DS + FPL + LA + AH + RI; EL + TB + AH + FP + MS; | | | | |
| | EC + SG + MM + MS + KQ; EC + PS + RT + MF + MS; | | | | |
| | EC + EL + LA + TB + RI; SG + PS + TB + AH + KQ; | | | | |
| | SG + DS + TB + MS + KQ; PS + RT + FPL + AH + KQ; | | | | |
| | PS + RT + LA + MF + KQ; EL + MF + FP + MS + RI; | | | | |
| | EC + PS + EL + LA + MF; EC + RT + EL + MF + MS; | | | | |
| | SG + PS + FPL + AH + FP; SG + RT + EL + MM + RI; | | | | |
| | PS + RT + DS + OS + RI; PS + RT + DS + AH + RI; | | | | |
| | PS + DS + FPL + OS + RI; PS + EL + LA + MF + FP; | | | | |
| | RT + TB + FP + ES + MS; EC + SG + EL + MM + AH; | | | | |
| | SG + PS + EL + MF + MS; PS + LA + MM + AH + RI; | | | | |
| | RT + FPL + EL + MF + RI; RT + FPL + EL + MS + KQ; | | | | |
| | FPL + FP + ES + KQ + RI; EL + TB + MM + MS + KQ; | | | | |
| | EC + SG + EL + LA + MF; EC + RT + MM + MS + RI; | | | | |
| | EC + DS + OS + TB + AH; EC + DS + EL + ES + RI; | | | | |
| | RT + MF + MS + KQ + RI; DS + LA + TB + MM + RI; | | | | |
| | FPL + ES + MS + KQ + RI; EL + LA + TB + MM + ES; | | | | |
| | EC + SG + EL + MF + AH; EC + EL + TB + KQ + RI; | | | | |
| | SG + RT + FPL + MM + KQ; PS + DS + TB + KQ + RI; | | | | |
| | PS + EL + LA + MM + FP; EC + RT + TB + AH + FP; | | | | |
| | PS + RT + DS + FP + RI; PS + FPL + MM + KQ + RI; | | | | |
| | RT + EL + LA + TB + MM; RT + EL + LA + AH + FP; | | | | |
| | DS + TB + ES + KQ + RI; DS + MM + AH + FP + ES; | | | | |
| | DS + MM + FP + ES + KQ; EC + SG + FPL + MF + RI; | | | | |
| | EC + DS + FPL + MM + MS; SG + PS + RT + MM + KQ; | | | | |
| | SG + DS + EL + KQ + RI; SG + DS + LA + MM + KQ; | | | | |
| | PS + DS + FPL + AH + RI; RT + LA + MF + AH + FP; | | | | |
| | FPL + EL + ES + KQ + RI; FPL + LA + MF + AH + FP; | | | | |
| | FPL + LA + FP + KQ + RI; EC + EL + ES + MS + RI; | | | | |
| | PS + RT + LA + AH + RI; EL + LA + MF + TB + AH; | | | | |
| | EC + SG + FPL + MS + KQ; EC + PS + FPL + LA + MS; | | | | |
| | EC + RT + FPL + MM + AH; EC + RT + EL + LA + AH; | | | | |
| | EC + FPL + EL + ES + RI; PS + MF + TB + FP + KQ; | | | | |
| | FPL + MF + TB + AH + MS; EL + LA + FP + MS + RI; | | | | |
| | EC + DS + FPL + MM + KQ; SG + PS + DS + FPL + OS; | | | | |
| | SG + EL + TB + MM + KQ; PS + LA + MM + AH + FP; | | | | |
| | RT + FPL + LA + MM + FP; FPL + EL + MF + MM + FP; | | | | |
| | EL + MM + FP + ES +RI; EC + SG + DS + OS + EL; | | | | |
| | EC + RT + EL + LA + RI; EC + RT + EL + MM + MS; | | | | |
| | EC + DS + FPL + OS + LA; EC + DS + LA + FP + KQ; | | | | |
| | EC + MF + TB + MM + AH; SG + RT + MF + MM + FP; | | | | |
| | RT + FPL + LA + MF + RI; RT + EL + MF + KQ + RI; | | | | |
| | EL + LA + FP + ES + KQ; EC + RT + DS + MM + KQ; | | | | |
| | SG + EL + AH + FP + MS; FPL + EL + MF + FP + MS; | | | | |
| | FPL + MF + TB + KQ + RI; FPL + TB + AH + FP + RI; | | | | |
| | SG + DS + FPL + OS + KQ; SG + DS + OS + TB + AH; | | | | |
| | SG + DS + MF + AH + KQ; SG + EL + MF + ES + MS; | | | | |
| | PS + RT + LA + MM + FP; RT + TB + MM + FP + MS | | | | |
| 5 | SG + RT + LA + MF + FP; RT + DS + MF + TB + FP; | 0.76 | 0.2875- | 42.86%- | 42.42%- |
| | RT + LA + TB + KQ + RI; FPL + EL + LA + TB + MM; | | 0.526 | 93.65% | 92.93% |
| | SG + RT + DS + FPL + FP; PS + FPL + LA + MM + ES; | | | | |
| | RT + LA + MF + AH + ES; EL + LA + MF + TB + FP; | | | | |
| | EC + SG + DS + TB + KQ; EC + SG + TB + FP + ES; | | | | |
| | EC + PS + FP + ES + KQ; SG + RT + FPL + MM + FP; | | | | |
| | SG + LA + AH + FP + RI; DS + LA + ES + MS + RI; | | | | |
| | EC + SG + DS + MM + FP; EC + PS + DS + AH + MS; | | | | |
| | EC + RT + MM + ES + RI; EC + DS + FPL + EL + AH; | | | | |
| | EC + DS + FPL + LA + MF; SG + RT + TB + MS + RI; | | | | |
| | PS + DS + FPL + ES + MS; PS + LA + ES + KQ + RI; | | | | |
| | RT + DS + TB + FP + MS; MF + TB + MM + AH + FP; | | | | |
| | EC + SG + RT + TB + MM; EC + PS + TB + MM + AH; | | | | |
| | SG + PS + FPL + TB + KQ; SG + PS + EL + LA + AH; | | | | |
| | PS + EL + FP + ES + RI; PS + MM + AH + ES + KQ; | | | | |
| | RT + LA + TB + FP + RI; EC + RT + DS + TB + RI; | | | | |
| | EC + DS + LA + MM + MS; SG + RT + DS + FP + MS; | | | | |
| | SG + DS + MM + AH + FP; PS + RT + EL + MF + ES; | | | | |
| | RT + EL + LA + MM + ES; DS + FPL + LA + ES + MS; | | | | |
| | EC + SG + RT + AH + KQ; EC + RT + EL + AH + ES; | | | | |
| | SG + PS + DS + LA + FP; SG + PS + FPL + EL + KQ; | | | | |
| | SG + FPL + EL + LA + MF; SG + AH + FP + ES + KQ; | | | | |
| | RT + FPL + MF + ES + KQ; RT + TB + MM + FP + ES; | | | | |
| | EC + PS + DS + FPL + RI; EC + RT + DS + MS + KQ; | | | | |
| | EC + DS + FPL + AH + KQ; EC + DS + LA + MF + MS; | | | | |
| | EC + DS + LA + TB + MM; EC + FPL + LA + ES + MS; | | | | |
| | EC + LA + AH + ES + KQ; SG + DS + EL + ES + KQ; | | | | |
| | SG + DS + MF + AH + FP; PS + DS + LA + MF + TB; | | | | |
| | DS + LA + MF + TB + MS; DS + LA + FP + MS + KQ; | | | | |
| | FPL + LA + FP + ES + MS; EC + SG + PS + TB + FP; | | | | |
| | EC + SG + DS + FPL + MM; EC + SG + LA + MF + TB; | | | | |
| | SG + RT + DS + MM + RI; PS + TB + AH + ES + MS; | | | | |
| | RT + EL + FP + MS + KQ; LA + TB + MM + AH + MS; | | | | |
| | MF + TB + FP + ES + KQ; EC + PS + RT + MS + RI; | | | | |
| | SG + LA + TB + ES + MS; SG + MM + AH + FP + ES; | | | | |
| | DS + EL + TB + ES + RI; SG + PS + LA + MM + ES; | | | | |
| | EC + SG + DS + FPL + MS; EC + RT + TB + ES + KQ; | | | | |
| | EC + FPL + EL + LA + TB; FPL + MF + TB + MM + FP; | | | | |
| | EC + RT + AH + ES + KQ; SG + DS + EL + LA + RI; | | | | |
| | PS + DS + EL + MF + TB; PS + DS + EL + MM + KQ; | | | | |
| | EC + SG + EL + LA + AH; SG + RT + DS + FPL + EL; | | | | |
| | SG + RT + TB + ES + RI; SG + FPL + MF + TB + MS; | | | | |
| | SG + FPL + ES + MS + KQ; PS + RT + FPL + AH + ES; | | | | |
| | PS + LA + TB + AH + MS; RT + DS + LA + FP + MS; | | | | |
| | EC + SG + PS + TB + KQ; EC + SG + FP + KQ + RI; | | | | |
| | EC + TB + AH + FP + KQ; PS + DS + LA + TB + AH; | | | | |
| | PS + EL + AH + ES + RI; PS + LA + AH + ES + RI; | | | | |
| | RT + DS + FPL + MF + ES; DS + FPL + LA + AH + KQ; | | | | |
| | DS + FPL + LA + KQ + RI; EC + SG + FPL + MF + AH; | | | | |
| | EC + PS + EL + FP + MS; EC + LA + TB + MM + AH; | | | | |
| | EC + MF + TB + FP + RI; SG + PS + FPL + MF + ES; | | | | |
| | RT + EL + MF + TB + ES; EC + DS + MF + FP + RI; | | | | |
| | SG + DS + LA + MM + MS; RT + DS + EL + FP + MS; | | | | |
| | EC + RT + LA + TB + KQ; EC + LA + TB + AH + KQ; | | | | |
| | SG + RT + TB + FP + ES; SG + RT + FP + MS + KQ; | | | | |
| | PS + DS + TB + ES + MS; PS + TB + AH + ES + RI; | | | | |
| | RT + FPL + LA + TB + RI; DS + FPL + EL + MM + KQ; | | | | |
| | EL + FP + ES + MS + RI; EC + SG + TB + KQ + RI; | | | | |
| | EC + PS + DS + OS + LA; EC + PS + DS + MF + MM; | | | | |
| | SG + RT + DS + EL + ES; SG + FPL + LA + MF + RI; | | | | |
| | SG + EL + AH + ES + RI; PS + RT + FPL + TB + ES; | | | | |
| | RT + DS + EL + MF + RI; RT + FPL + LA + ES + RI; | | | | |
| | RT + EL + MM + FP + ES; RT + EL + MM + ES + RI; | | | | |
| | DS + EL + LA + FP + MS; EC + SG + PS + FPL + MF; | | | | |
| | EC + PS + DS + EL + AH; EC + RT + DS + MF + RI; | | | | |
| | EC + RT + AH + ES + MS; SG + PS + RT + LA + MM; | | | | |
| | PS + MF + TB + AH + ES; EC + SG + MM + AH + KQ; | | | | |
| | EC + RT + DS + ES + KQ; EC + RT + TB + MS + RI; | | | | |
| | SG + RT + EL + MS + KQ; SG + MM + FP + ES + MS; | | | | |
| | SG + MM + FP + ES + RI; PS + RT + DS + LA + MS; | | | | |
| | RT + DS + FPL + LA + RI; DS + TB + AH + ES + RI; | | | | |
| | FPL + MF + TB + FP + MS; EC + DS + EL + TB + MS; | | | | |
| | SG + PS + FPL + EL + LA; SG + RT + FPL + EL + LA; | | | | |
| | SG + RT + FP + MS + RI; EC + LA + MF + AH + ES; | | | | |
| | SG + PS + LA + KQ + RI; SG + LA + MF + AH + RI; | | | | |
| | PS + DS + LA + TB + MS; RT + EL + LA + TB + RI; | | | | |
| | DS + TB + AH + ES + KQ; EL + LA + ES + MS + KQ; | | | | |
| | EC + SG + RT + DS + FP; EC + PS + FPL + AH + ES; | | | | |
| | EC + DS + EL + MF + RI; EC + FPL + EL + TB + AH; | | | | |
| | EC + FPL + LA + TB + KQ; SG + PS + RT + FPL + EL; | | | | |
| | SG + RT + FPL + MF + TB; RT + EL + TB + FP + MS; | | | | |
| | LA + MF + TB + ES + RI; EC + SG + DS + EL + MM; | | | | |
| | EC + SG + DS + FP + KQ; EC + RT + DS + EL + MS; | | | | |
| | SG + FPL + MF + AH + ES; PS + DS + FPL + AH + FP; | | | | |
| | PS + DS + FPL + FP + ES; PS + LA + AH + ES + KQ; | | | | |
| | DS + FPL + MF + AH + ES; EC + SG + PS + RT + MF; | | | | |
| | EC + SG + RT + EL + TB; EC + SG + LA + MM + MS; | | | | |
| | EC + DS + EL + TB + AH; EC + PS + RT + FPL + RI; | | | | |
| | EC + FPL + LA + MF + ES; SG + TB + MM + FP + RI; | | | | |
| | PS + EL + AH + ES + MS; RT + EL + LA + KQ + RI; | | | | |
| | RT + LA + MM + ES + KQ; DS + FPL + MF + MM + RI; | | | | |
| | EC + PS + RT + DS + MS; EC + RT + FP + ES + KQ; | | | | |
| | SG + RT + LA + MM + AH; DS + FPL + LA + FP + MS; | | | | |
| | DS + LA + FP + ES + KQ; DS + TB + MM + FP + RI; | | | | |
| | FPL + EL + MF + TB + RI; MF + TB + MM + FP + KQ; | | | | |
| | EC + PS + RT + FPL + EL; EC + RT + FPL + MF + TB; | | | | |
| | EC + RT + TB + FP + MS; EC + DS + LA + AH + KQ; | | | | |
| | SG + PS + RT + AH + ES; SG + DS + MF + TB + RI; | | | | |
| | SG + DS + MF + ES + RI; DS + FPL + LA + MM + KQ; | | | | |
| | DS + EL + LA + TB + KQ; FPL + LA + TB + ES + MS; | | | | |
| | EC + SG + RT + TB + AH; EC + SG + RT + AH + RI; | | | | |
| | EC + SG + MS + KQ + RI; EC + RT + DS + LA + FP; | | | | |
| | SG + RT + EL + MF + ES; PS + DS + FPL + EL + FP; | | | | |
| | DS + FPL + LA + AH + FP; DS + LA + MM + FP + ES; | | | | |
| | EC + PS + FP + ES + RI; SG + DS + AH + MS + RI; | | | | |
| | SG + LA + FP + ES + RI; PS + FPL + FP + ES + RI; | | | | |
| | DS + FPL + LA + FP + ES; DS + LA + FP + ES + RI; | | | | |
| | EC + SG + RT + EL + MS; EC + DS + FPL + LA + MS; | | | | |
| | EC + FPL + LA + TB + AH; SG + RT + LA + MM + MS; | | | | |
| | PS + EL + LA + MM + ES; RT + DS + TB + FP + RI; | | | | |
| | DS + EL + MF + FP + RI; EL + TB + AH + ES + MS; | | | | |
| | LA + AH + ES + MS + KQ; EC + PS + MF + TB + MM; | | | | |
| | EC + MM + AH + ES + KQ; SG + AH + FP + ES + MS; | | | | |
| | PS + DS + FPL + LA + AH; PS + DS + EL + AH + RI; | | | | |
| | FPL + TB + ES + KQ + RI; EC + RT + FPL + LA + KQ; | | | | |
| | SG + EL + LA + MF + FP; PS + DS + MF + TB + RI; | | | | |
| | DS + FPL + EL + LA + KQ; MF + TB + FP + MS + RI; | | | | |
| | EC + SG + MF + MM + ES; EC + PS + DS + MM + MS; | | | | |
| | EC + RT + DS + LA + RI; RT + FPL + TB + FP + RI; | | | | |
| | DS + FPL + EL + LA + ES; EC + SG + TB + AH + MS; | | | | |
| | EC + RT + DS + EL + RI; EC + DS + FPL + TB + MS; | | | | |
| | SG + FPL + LA + MM + RI; PS + RT + FPL + EL + TB; | | | | |
| | PS + LA + AH + FP + ES; RT + DS + TB + FP + KQ; | | | | |
| | DS + FPL + EL + MM + RI; DS + FPL + MS + KQ + RI; | | | | |
| | EC + RT + FPL + EL + KQ; EC + FPL + TB + FP + KQ; | | | | |
| | SG + RT + FPL + LA + MM; SG + TB + MM + MS + RI; | | | | |
| | PS + DS + EL + MS + KQ; PS + LA + MF + TB + FP; | | | | |
| | RT + DS + FPL + FP + RI; RT + FPL + LA + ES + MS; | | | | |
| | RT + TB + FP + MS + RI; EC + FPL + EL + FP + ES; | | | | |
| | EC + LA + TB + MM + RI; SG + RT + MF + FP + MS; | | | | |
| | PS + EL + FP + ES + KQ; DS + MM + AH + ES + RI; | | | | |
| | EL + TB + AH + ES + KQ; EC + SG + MF + ES + MS; | | | | |
| | EC + RT + LA + FP + MS; SG + TB + FP + MS + RI; | | | | |
| | PS + DS + EL + TB + KQ; PS + TB + MM + FP + MS; | | | | |
| | RT + DS + FP + ES + MS; RT + LA + MM + MS + KQ; | | | | |
| | EC + SG + RT + DS + OS; EC + PS + FP + MS + KQ; | | | | |
| | EC + FPL + MF + TB + AH; EC + TB + FP + KQ + RI; | | | | |
| | PS + RT + FPL + LA + RI; RT + FPL + ES + MS + RI; | | | | |
| | RT + MF + TB + FP + ES; RT + TB + FP + KQ + RI; | | | | |
| | EC + RT + MF + FP + ES; EC + DS + FPL + LA + KQ; | | | | |
| | EC + EL + LA + ES + MS; SG + DS + TB + ES + MS; | | | | |
| | SG + DS + MM + MS + RI; PS + RT + DS + EL + ES; | | | | |
| | PS + RT + FPL + KQ + RI; PS + RT + EL + LA + TB; | | | | |
| | PS + RT + EL + MS + KQ; PS + RT + MM + ES + RI; | | | | |
| | RT + EL + FP + MS + RI; EL + LA + FP + ES + MS; | | | | |
| | EC + SG + EL + AH + KQ; EC + DS + LA + MM + AH; | | | | |
| | EC + FPL + TB + AH + KQ; SG + PS + DS + FP + RI; | | | | |
| | PS + LA + ES + MS + KQ; RT + DS + EL + MF + FP; | | | | |
| | RT + DS + MF + MS + RI; LA + MM + FP + ES + MS; | | | | |
| | EC + PS + LA + AH + FP; SG + RT + MM + FP + ES; | | | | |
| | SG + FPL + LA + AH + RI; RT + DS + EL + LA + KQ; | | | | |
| | RT + DS + EL + LA + RI; EC + RT + MM + ES + MS; | | | | |
| | SG + RT + MF + FP + RI; SG + MM + AH + ES + KQ; | | | | |
| | PS + RT + EL + LA + RI; DS + EL + MS + KQ + RI; | | | | |
| | FPL + FP + ES + MS + KQ; EC + SG + RT + OS + FP; | | | | |
| | EC + PS + EL + AH + KQ; EC + PS + LA + MM + AH; | | | | |
| | SG + PS + LA + FP + RI; SG + LA + FP + MS + RI; | | | | |
| | PS + FPL + TB + MS + RI; PS + LA + AH + ES + MS; | | | | |
| | PS + LA + FP + ES + KQ; RT + DS + LA + MF + FP; | | | | |
| | FPL + TB + AH + ES + RI; EC + SG + FPL + FP + KQ; | | | | |
| | EC + PS + DS + MF + FP; EC + RT + DS + MF + ES; | | | | |
| | EC + DS + TB + AH + FP; EC + FPL + EL + TB + MS; | | | | |
| | EC + LA + MF + ES + MS; SG + PS + LA + FP + ES; | | | | |
| | SG + RT + MF + TB + FP; SG + FPL + MM + KQ + RI; | | | | |
| | RT + DS + EL + TB + MS; RT + FPL + TB + MS + KQ; | | | | |
| | DS + LA + MF + AH + ES; LA + MM + AH + FP + ES; | | | | |
| | EC + PS + FPL + EL + FP; EC + PS + LA + KQ + RI; | | | | |
| | SG + FPL + TB + ES + MS; PS + DS + LA + TB + KQ; | | | | |
| | RT + EL + MS + KQ + RI; RT + LA + MF + ES + MS; | | | | |
| | RT + TB + FP + ES + RI; RT + TB + FP + MS + KQ; | | | | |
| | EC + SG + RT + FPL + MM; EC + SG + FPL + MM + FP; | | | | |
| | EC + SG + EL + TB + AH; EC + SG + LA + MF + FP; | | | | |
| | EC + PS + DS + FPL + MS; RT + DS + EL + LA + FP; | | | | |
| | RT + FPL + MF + ES + RI; DS + FPL + LA + MM + RI; | | | | |
| | LA + TB + MM + FP + RI; EC + PS + RT + EL + MS; | | | | |
| | EC + DS + FPL + ES + MS; EC + DS + FP + MS + KQ; | | | | |
| | EC + LA + ES + MS + RI; EC + TB + MM + ES + MS; | | | | |
| | EC + TB + AH + FP + MS; SG + EL + TB + MS + RI; | | | | |
| | PS + EL + LA + TB + MS; PS + EL + MF + ES + KQ; | | | | |
| | RT + FPL + TB + FP + MS; FPL + LA + AH + FP + ES; | | | | |
| | PS + RT + EL + TB + KQ; PS + RT + MF + ES + RI; | | | | |
| | PS + FPL + EL + TB + MS; RT + FPL + LA + MS + KQ; | | | | |
| | RT + FPL + LA + KQ + RI; RT + LA + FP + ES + RI; | | | | |
| | RT + LA + FP + MS + KQ; DS + EL + TB + MS + RI; | | | | |
| | DS + TB + AH + MS + RI; EL + LA + FP + ES + RI; | | | | |
| | EC + RT + DS + MF + FP; EC + RT + DS + KQ + RI; | | | | |
| | EC + RT + EL + MF + ES; SG + PS + RT + AH + RI; | | | | |
| | SG + FPL + LA + TB + MS; SG + FPL + MF + TB + KQ; | | | | |
| | SG + AH + ES + MS + RI; PS + RT + DS + MS + RI; | | | | |
| | PS + FPL + MF + ES + RI; PS + LA + TB + AH + KQ; | | | | |
| | RT + DS + FPL + EL + MS; DS + LA + MM + AH + FP; | | | | |
| | FPL + TB + MM + FP + RI; EL + LA + AH + FP + ES; | | | | |
| | EC + SG + PS + FPL + MS; EC + PS + DS + MM + KQ; | | | | |
| | EC + RT + DS + FP + ES; EC + RT + EL + TB + FP; | | | | |
| | EC + FPL + EL + LA + ES; EC + FPL + TB + MM + FP; | | | | |
| | SG + FPL + MF + TB + RI; SG + FPL + TB + MM + MS; | | | | |
| | SG + FPL + AH + FP + RI; PS + RT + AH + ES + RI; | | | | |
| | EC + RT + FPL + FP + ES; SG + RT + DS + OS + RI; | | | | |
| | SG + RT + DS + ES + MS; SG + DS + FPL + OS + LA; | | | | |
| | SG + DS + FPL + TB + AH; SG + FPL + LA + MM + MS; | | | | |
| | SG + TB + ES + MS + RI; PS + EL + FP + ES + MS; | | | | |
| | DS + FPL + LA + FP + KQ; EC + PS + RT + KQ + RI; | | | | |
| | EC + DS + FPL + MM + ES; SG + PS + MM + AH + KQ; | | | | |
| | SG + RT + FPL + AH + FP; SG + RT + MF + ES + RI; | | | | |
| | SG + EL + LA + MM + ES; SG + FP + ES + MS + RI; | | | | |
| | DS + TB + MM + MS + KQ; EL + TB + AH + ES + RI; | | | | |
| | EC + RT + FPL + LA + FP; EC + DS + MF + TB + KQ; | | | | |
| | SG + DS + TB + FP + MS; SG + EL + MF + TB + ES; | | | | |
| | SG + MM + FP + MS + RI; PS + TB + AH + FP + KQ; | | | | |
| | PS + TB + FP + MS + RI; RT + DS + EL + FP + RI; | | | | |
| | LA + TB + AH + FP + RI; EC + PS + RT + FPL + TB; | | | | |
| | EC + PS + FP + MS + RI; EC + FP + ES + MS + RI; | | | | |
| | SG + RT + EL + LA + FP; SG + DS + EL + AH + FP; | | | | |
| | PS + TB + AH + ES + KQ; RT + FPL + FP + KQ + RI; | | | | |
| | DS + FPL + MF + AH + FP; EC + SG + TB + AH + RI; | | | | |
| | EC + DS + MF + AH + RI; EC + MF + TB + MM + ES; | | | | |
| | SG + PS + TB + MM + ES; PS + TB + MM + ES + KQ; | | | | |
| | PS + TB + ES + MS + KQ; RT + DS + MF + ES + RI; | | | | |
| | RT + DS + TB + KQ + RI; EC + PS + FPL + TB + MS; | | | | |
| | PS + DS + MM + FP + ES; RT + DS + FPL + FP + MS; | | | | |
| | RT + FPL + MF + TB + ES; DS + FPL + EL + MF + MS; | | | | |
| | DS + FPL + EL + AH + KQ; DS + AH + FP + ES + MS; | | | | |
| | FPL + EL + TB + AH + FP; EC + SG + RT + DS + EL; | | | | |
| | EC + PS + FPL + MF + RI; EC + RT + DS + LA + KQ; | | | | |
| | RT + EL + TB + MS + RI; DS + FPL + EL + FP + KQ; | | | | |
| | FPL + AH + FP + ES + KQ; LA + TB + MS + KQ + RI; | | | | |
| | EC + DS + LA + MF + AH; SG + DS + LA + MF + RI; | | | | |
| | SG + FPL + LA + MS + RI; PS + RT + LA + FP + MS; | | | | |
| | PS + FPL + LA + FP + KQ; DS + FPL + MM + MS + KQ; | | | | |
| | EC + PS + RT + TB + MS; EC + PS + DS + MF + KQ; | | | | |
| | EC + RT + MF + ES + MS; SG + RT + DS + MS + KQ; | | | | |
| | SG + LA + MF + AH + KQ; PS + FPL + EL + ES + MS; | | | | |
| | RT + MM + ES + KQ + RI; DS + FPL + TB + MM + MS; | | | | |
| | EC + SG + PS + OS + LA; EC + PS + DS + AH + FP; | | | | |
| | EC + DS + MF + FP + ES; EC + LA + MF + FP + ES; | | | | |
| | SG + DS + AH + KQ + RI; EC + SG + RT + DS + MM; | | | | |
| | EC + RT + DS + EL + TB; EC + DS + FPL + EL + MF; | | | | |
| | EC + EL + FP + ES + MS; EC + TB + MM + AH + KQ; | | | | |
| | SG + DS + LA + FP + RI; SG + LA + TB + MM + MS; | | | | |
| | SG + LA + FP + KQ + RI; FPL + MF + TB + ES + KQ; | | | | |
| | LA + AH + FP + ES + MS; MF + TB + AH + FP + MS; | | | | |
| | EC + DS + FPL + LA + FP; SG + PS + TB + MM + MS; | | | | |
| | SG + RT + FP + ES + MS; PS + EL + AH + FP + ES; | | | | |
| | DS + LA + MF + FP + ES; DS + LA + AH + FP + MS; | | | | |
| | EC + SG + FPL + ES + MS; PS + DS + TB + AH + KQ; | | | | |
| | RT + FPL + LA + MF + FP; EC + RT + TB + FP + ES; | | | | |
| | EC + DS + FPL + AH + RI; EC + FP + ES + MS + KQ; | | | | |
| | SG + PS + DS + FPL + MM; SG + DS + FP + MS + KQ; | | | | |
| | PS + DS + EL + MF + FP; RT + FPL + LA + MF + TB; | | | | |
| | RT + EL + MM + ES + KQ; RT + LA + MF + TB + FP; | | | | |
| | EC + PS + RT + LA + FP; EC + DS + FPL + EL + MM; | | | | |
| | PS + FPL + TB + AH + FP; RT + DS + MF + FP + ES; | | | | |
| | DS + MM + AH + ES + MS; FPL + EL + FP + ES + KQ; | | | | |
| | EL + MF + TB + AH + ES; EC + SG + MM + FP + RI; | | | | |
| | EC + RT + DS + LA + AH; EC + RT + EL + MS + KQ; | | | | |
| | EC + RT + AH + ES + RI; SG + DS + MM + AH + MS; | | | | |
| | SG + FPL + TB + KQ + RI; SG + EL + MF + FP + KQ; | | | | |
| | RT + DS + LA + KQ + RI; RT + EL + LA + FP + MS; | | | | |
| | EC + PS + LA + AH + KQ; SG + RT + EL + TB + RI; | | | | |
| | SG + EL + MM + AH + FP; FPL + EL + MF + TB + KQ; | | | | |
| | FPL + LA + MF + FP + ES; EC + SG + MM + FP + KQ; | | | | |
| | EC + DS + FPL + FP + ES; SG + FPL + EL + LA + KQ; | | | | |
| | SG + LA + MM + FP + RI; PS + LA + MM + ES + MS; | | | | |
| | EC + SG + FPL + LA + TB; EC + RT + DS + MM + MS; | | | | |
| | EC + DS + EL + FP + RI; SG + PS + RT + DS + EL; | | | | |
| | PS + FPL + EL + FP + KQ; RT + LA + AH + FP + ES; | | | | |
| | DS + FPL + LA + MS + RI; DS + FPL + MM + FP + ES; | | | | |
| | FPL + TB + MM + MS + KQ; EC + PS + DS + FP + RI; | | | | |
| | EC + RT + FPL + TB + RI; SG + PS + DS + FPL + MS; | | | | |
| | SG + PS + FPL + MS + KQ; SG + RT + LA + AH + MS; | | | | |
| | SG + RT + MM + MS + RI; SG + RT + MM + KQ + RI; | | | | |
| | PS + DS + MF + TB + FP; TB + MM + AH + ES + KQ; | | | | |
| | EC + SG + RT + MM + AH; EC + SG + FPL + FP + ES; | | | | |
| | EC + RT + MS + KQ + RI; EC + RT + EL + FP + MS; | | | | |
| | EC + DS + LA + AH + RI; SG + RT + FPL + MF + ES; | | | | |
| | SG + DS + LA + MM + ES; SG + DS + MM + ES + RI; | | | | |
| | SG + MF + FP + ES + RI; PS + DS + LA + MS + RI; | | | | |
| | PS + AH + ES + MS + RI; RT + LA + MF + ES + RI; | | | | |
| | DS + FPL + MM + FP + KQ; LA + MF + AH + FP + ES; | | | | |
| | EC + SG + RT + FPL + MS; EC + SG + FPL + EL + MF; | | | | |
| | EC + SG + MM + ES + MS; EC + PS + RT + EL + KQ; | | | | |
| | SG + PS + FPL + EL + MM; SG + RT + FPL + MS + KQ; | | | | |
| | PS + RT + EL + LA + MS; RT + EL + LA + AH + ES; | | | | |
| | RT + LA + MM + ES + RI; RT + LA + FP + ES + MS; | | | | |
| | DS + FPL + MF + AH + KQ; EC + RT + DS + FP + RI; | | | | |
| | SG + PS + DS + FPL + AH; SG + RT + DS + TB + FP; | | | | |
| | SG + RT + TB + ES + MS; RT + LA + TB + ES + RI; | | | | |
| | DS + FPL + LA + MF + AH; EC + SG + RT + AH + MS; | | | | |
| | EC + PS + EL + TB + RI; EC + DS + LA + MM + FP; | | | | |
| | EC + EL + LA + TB + MM; SG + DS + FPL + OS + TB; | | | | |
| | PS + LA + TB + AH + RI; PS + TB + FP + MS + KQ; | | | | |
| | RT + DS + MS + KQ + RI; RT + FPL + MM + FP + ES; | | | | |
| | RT + MF + ES + MS + RI; RT + MF + ES + KQ + RI; | | | | |
| | DS + FPL + AH + FP + KQ; FPL + EL + TB + MS + RI; | | | | |
| | FPL + LA + TB + KQ + RI; FPL + FP + ES + MS + RI; | | | | |
| | LA + MF + TB + ES + MS; EC + DS + MF + TB + MM; | | | | |
| | EC + DS + MF + AH + FP; EC + DS + AH + MS + RI; | | | | |
| | SG + PS + FPL + EL + MF; SG + DS + LA + FP + KQ; | | | | |
| | PS + DS + FPL + MS + KQ; RT + MM + ES + MS + RI; | | | | |
| | EL + LA + TB + AH + MS; EC + SG + DS + FPL + AH; | | | | |
| | EC + PS + MM + AH + KQ; EC + RT + TB + MS + KQ; | | | | |
| | EC + DS + FPL + TB + RI; SG + PS + RT + DS + KQ; | | | | |
| | SG + PS + RT + FP + ES; SG + LA + ES + MS + RI; | | | | |
| | PS + RT + MS + KQ + RI; PS + FPL + EL + LA + ES; | | | | |
| | PS + EL + MF + ES + MS; DS + FPL + LA + MM + FP; | | | | |
| | DS + EL + LA + TB + RI; DS + AH + ES + MS + RI; | | | | |
| | EC + PS + MF + FP + MS; SG + PS + RT + FPL + MF; | | | | |
| | SG + PS + ES + KQ + RI; SG + TB + AH + ES + MS; | | | | |
| | RT + DS + FPL + EL + LA; DS + FPL + EL + LA + MF; | | | | |
| | DS + TB + MM + MS + RI; FPL + MF + TB + ES + RI; | | | | |
| | EC + SG + EL + MS + RI; EC + RT + MM + ES + KQ; | | | | |
| | SG + RT + DS + LA + MF; SG + FPL + MF + AH + KQ; | | | | |
| | PS + DS + MF + AH + ES; RT + DS + LA + TB + KQ; | | | | |
| | EC + PS + DS + EL + FP; EC + RT + TB + FP + RI; | | | | |
| | EC + DS + EL + MF + MS; EC + DS + LA + MM + ES; | | | | |
| | SG + PS + LA + MM + KQ; SG + RT + EL + TB + KQ; | | | | |
| | SG + FPL + MS + KQ + RI; PS + FPL + MF + FP + ES; | | | | |
| | RT + DS + EL + FP + KQ; RT + DS + LA + TB + RI; | | | | |
| | RT + FPL + LA + MM + KQ; DS + AH + FP + ES + RI; | | | | |
| | EC + RT + DS + MF + MS; EC + RT + AH + FP + ES; | | | | |
| | EC + LA + MM + ES + MS; EC + TB + MS + KQ + RI; | | | | |
| | SG + PS + RT + LA + MF; SG + EL + MF + MM + FP; | | | | |
| | PS + EL + LA + KQ + RI; RT + FPL + MF + FP + ES; | | | | |
| | RT + FPL + TB + MM + FP; RT + TB + MM + FP + KQ; | | | | |
| | DS + FPL + MM + FP + RI; DS + EL + LA + AH + FP; | | | | |
| | EC + RT + FPL + MM + ES; EC + RT + MM + FP + ES; | | | | |
| | PS + RT + DS + FPL + LA; FPL + MF + TB + MM + KQ; | | | | |
| | EC + SG + FPL + EL + RI; EC + SG + FPL + MF + FP; | | | | |
| | EC + PS + RT + LA + MF; EC + RT + MF + TB + ES; | | | | |
| | SG + RT + DS + LA + AH; EC + SG + EL + MF + TB; | | | | |
| | EC + SG + MM + KQ + RI; EC + PS + RT + TB + RI; | | | | |
| | EC + PS + DS + FPL + FP; EC + FPL + MF + ES + MS; | | | | |
| | SG + PS + FPL + LA + MF; SG + PS + LA + FP + MS; | | | | |
| | SG + RT + DS + MF + RI; SG + DS + FPL + AH + FP; | | | | |
| | SG + TB + MM + AH + KQ; PS + FPL + LA + ES + KQ; | | | | |
| | DS + FPL + LA + MS + KQ; DS + FP + ES + MS + RI; | | | | |
| | EC + SG + EL + FP + RI; EC + RT + DS + FPL + FP; | | | | |
| | SG + FPL + MF + MM + FP; PS + RT + FPL + LA + TB; | | | | |
| | RT + LA + MM + FP + ES; DS + FPL + FP + MS + RI; | | | | |
| | EC + PS + EL + MF + TB; EC + FPL + LA + ES + KQ; | | | | |
| | RT + FPL + EL + LA + ES; DS + FPL + MF + TB + AH; | | | | |
| | EC + RT + DS + FPL + RI; EC + DS + FPL + ES + RI; | | | | |
| | PS + FPL + LA + TB + FP; RT + EL + LA + FP + KQ; | | | | |
| | RT + LA + TB + MM + FP; EC + SG + LA + MM + AH; | | | | |
| | EC + RT + FP + MS + KQ; EC + FPL + TB + MM + AH; | | | | |
| | SG + RT + EL + LA + MF; SG + DS + MF + MS + RI; | | | | |
| | SG + FPL + EL + MS + KQ; SG + FPL + ES + KQ + RI; | | | | |
| | SG + LA + MF + AH + MS; RT + EL + TB + KQ + RI; | | | | |
| | EC + SG + AH + KQ + RI; EC + DS + FP + KQ + RI; | | | | |
| | SG + PS + RT + MF + RI; SG + DS + LA + MF + MS; | | | | |
| | FPL + EL + FP + ES + RI; MF + TB + MM + FP + RI; | | | | |
| | EC + PS + RT + LA + MM; EC + DS + FPL + LA + MM; | | | | |
| | EC + DS + FPL + FP + RI; EC + FPL + EL + ES + MS; | | | | |
| | EC + FPL + TB + FP + ES; SG + PS + FPL + MS + RI; | | | | |
| | SG + RT + LA + MF + MS; SG + EL + ES + MS + KQ; | | | | |
| | PS + FPL + EL + TB + AH; RT + DS + FPL + MM + ES; | | | | |
| | RT + EL + LA + MF + ES; FPL + EL + LA + TB + KQ; | | | | |
| | EC + SG + RT + MM + FP; EC + PS + MM + FP + ES; | | | | |
| | EC + RT + FPL + FP + MS; EC + RT + EL + MS + RI; | | | | |
| | PS + DS + LA + AH + FP; EC + SG + RT + FPL + EL; | | | | |
| | EC + RT + MF + ES + RI; EC + MF + TB + FP + KQ; | | | | |
| | SG + RT + FPL + MM + ES; SG + DS + EL + LA + TB; | | | | |
| | PS + RT + DS + LA + KQ; PS + RT + DS + ES + MS; | | | | |
| | DS + LA + TB + KQ + RI; DS + TB + MM + AH + RI; | | | | |
| | LA + FP + ES + MS + RI; EC + PS + MF + MS + KQ; | | | | |
| | EC + FPL + LA + TB + RI; SG + PS + FPL + TB + MS; | | | | |
| | RT + EL + FP + KQ + RI; RT + LA + TB + AH + FP; | | | | |
| | RT + MM + FP + ES + RI; FPL + EL + MF + TB + MS; | | | | |
| | EC + SG + FPL + EL + LA; EC + SG + FPL + MM + AH; | | | | |
| | EC + PS + RT + EL + LA; EC + PS + RT + LA + AH; | | | | |
| | EC + DS + MM + ES + KQ; EC + EL + ES + MS + KQ; | | | | |
| | SG + PS + RT + FPL + AH; PS + RT + MM + ES + KQ; | | | | |
| | FPL + EL + MF + ES + RI; LA + MM + AH + ES + MS; | | | | |
| | EC + SG + DS + OS + TB; EC + RT + DS + TB + KQ; | | | | |
| | EC + RT + FPL + EL + AH; EC + RT + FPL + FP + KQ; | | | | |
| | EC + FPL + EL + AH + FP; EC + TB + ES + KQ + RI; | | | | |
| | SG + RT + MM + FP + RI; SG + FPL + MM + AH + FP; | | | | |
| | PS + DS + FPL + LA + FP; PS + DS + LA + AH + KQ; | | | | |
| | RT + DS + FPL + LA + FP; EC + SG + RT + EL + AH; | | | | |
| | EC + SG + DS + OS + RI; EC + SG + FPL + TB + AH; | | | | |
| | EC + RT + DS + MM + RI; EC + FPL + ES + MS + KQ; | | | | |
| | SG + FPL + EL + TB + RI; PS + TB + FP + ES + KQ; | | | | |
| | RT + DS + EL + MF + ES; RT + FPL + LA + TB + MS; | | | | |
| | FPL + EL + LA + MF + TB; EC + SG + RT + FPL + TB; | | | | |
| | EC + FPL + MF + TB + MS; EC + EL + LA + AH + ES; | | | | |
| | SG + PS + EL + TB + MS; SG + PS + MF + AH + ES; | | | | |
| | SG + PS + FP + KQ + RI; PS + RT + EL + MM + KQ; | | | | |
| | DS + FPL + MM + KQ + RI; EC + SG + MF + TB + RI; | | | | |
| | EC + PS + FPL + TB + AH; EC + PS + EL + TB + AH; | | | | |
| | EC + PS + TB + AH + RI; EC + RT + DS + EL + FP; | | | | |
| | EC + DS + MM + FP + MS; EC + FPL + EL + TB + RI; | | | | |
| | SG + PS + MF + ES + RI; SG + DS + LA + MF + FP; | | | | |
| | PS + DS + MM + MS + RI; PS + EL + LA + TB + AH; | | | | |
| | PS + EL + MF + AH + ES; DS + FPL + MF + ES + KQ; | | | | |
| | EL + LA + AH + ES + MS; LA + MF + TB + AH + MS; | | | | |
| | SG + DS + OS + LA + FP; SG + DS + LA + MM + RI; | | | | |
| | SG + LA + TB + MM + RI; PS + RT + EL + MS + RI; | | | | |
| | PS + RT + TB + AH + ES; PS + TB + MM + AH + FP; | | | | |
| | RT + FPL + EL + LA + FP; DS + FPL + LA + MF + MM; | | | | |
| | EC + RT + FPL + LA + AH; EC + RT + MF + AH + ES; | | | | |
| | EC + RT + TB + FP + KQ; EC + DS + EL + LA + AH; | | | | |
| | EC + FPL + AH + FP + ES; SG + FPL + MM + MS + KQ; | | | | |
| | SG + FPL + ES + MS + RI; PS + DS + LA + KQ + RI; | | | | |
| | PS + FPL + FP + ES + MS; RT + EL + LA + MS + RI; | | | | |
| | DS + FPL + MF + MS + RI; DS + EL + MM + FP + ES; | | | | |
| | FPL + MF + FP + ES + RI; EL + LA + TB + AH + KQ; | | | | |
| | EC + SG + DS + MF + KQ; EC + RT + DS + FPL + MM; | | | | |
| | EC + RT + DS + FPL + AH; EC + DS + EL + LA + KQ; | | | | |
| | EC + FPL + MF + TB + RI; PS + FPL + LA + MF + TB; | | | | |
| | EC + SG + FPL + EL + MS; EC + PS + EL + FP + KQ; | | | | |
| | EC + DS + FPL + EL + MS; EC + LA + MF + ES + RI; | | | | |
| | SG + FPL + AH + KQ + RI; PS + EL + LA + AH + KQ; | | | | |
| | PS + LA + MF + TB + AH; PS + DS + LA + MM + AH; | | | | |
| | PS + FPL + MF + TB + MS; DS + FPL + LA + MF + RI; | | | | |
| | EC + PS + FPL + LA + KQ; EC + PS + TB + AH + MS; | | | | |
| | EC + RT + EL + LA + TB; EC + RT + LA + MS + RI; | | | | |
| | EC + EL + TB + AH + KQ; EC + LA + MM + ES + KQ; | | | | |
| | SG + PS + RT + MM + RI; PS + DS + LA + MM + RI; | | | | |
| | PS + EL + LA + FP + RI; PS + TB + MM + FP + KQ; | | | | |
| | RT + DS + LA + MF + ES; DS + FPL + FP + ES + KQ; | | | | |
| | EC + SG + RT + MF + FP; EC + RT + FPL + LA + MS; | | | | |
| | SG + RT + TB + AH + FP; PS + RT + DS + MF + RI; | | | | |
| | EC + SG + MF + TB + AH; EC + PS + RT + DS + EL; | | | | |
| | EC + PS + MM + KQ + RI; EC + RT + FPL + EL + MS; | | | | |
| | EC + RT + EL + KQ + RI; EC + RT + MM + AH + ES; | | | | |
| | EC + AH + FP + ES + MS; SG + PS + MS + KQ + RI; | | | | |
| | SG + TB + MM + AH + MS; PS + RT + LA + TB + RI; | | | | |
| | PS + EL + FP + MS + RI; RT + FPL + LA + MM + ES; | | | | |
| | DS + EL + MM + AH + ES; DS + EL + FP + MS + KQ; | | | | |
| | FPL + EL + FP + MS + KQ; TB + MM + AH + FP + KQ; | | | | |
| | EC + RT + TB + MM + ES; EC + DS + MF + ES + KQ; | | | | |
| | EC + DS + AH + FP + MS; EC + FPL + MF + FP + ES; | | | | |
| | EC + FPL + TB + KQ + RI; SG + EL + AH + FP + KQ; | | | | |
| | PS + LA + TB + FP + KQ; PS + MF + TB + FP + RI; | | | | |
| | FPL + LA + FP + ES + RI; EL + LA + AH + ES + KQ; | | | | |
| | EC + SG + RT + EL + MM; EC + SG + FPL + TB + MM; | | | | |
| | EC + PS + DS + EL + RI; EC + PS + MM + FP + KQ; | | | | |
| | EC + DS + AH + KQ + RI; SG + PS + EL + FP + RI; | | | | |
| | SG + DS + FPL + MF + AH; SG + DS + EL + LA + MS; | | | | |
| | PS + EL + LA + FP + MS; PS + LA + MF + AH + FP; | | | | |
| | RT + LA + TB + MM + KQ; DS + FPL + EL + MF + KQ; | | | | |
| | DS + MM + ES + MS + RI; EC + RT + FPL + EL + LA; | | | | |
| | SG + RT + OS + LA + FP; SG + DS + FPL + EL + AH; | | | | |
| | PS + DS + EL + FP + KQ; PS + LA + MS + KQ + RI; | | | | |
| | PS + MM + AH + ES + RI; RT + DS + FPL + TB + MS; | | | | |
| | DS + LA + MM + MS + RI; FPL + EL + LA + FP + ES; | | | | |
| | FPL + EL + MM + MS + KQ; FPL + MF + FP + ES + MS; | | | | |
| | EC + RT + DS + TB + AH; EC + RT + DS + AH + MS; | | | | |
| | EC + DS + LA + KQ + RI; SG + TB + MM + FP + ES; | | | | |
| | PS + RT + DS + KQ + RI; PS + DS + TB + MM + AH; | | | | |
| | DS + FPL + TB + AH + RI; DS + LA + MF + TB + RI; | | | | |
| | EC + RT + FPL + EL + RI; EC + RT + FPL + AH + KQ; | | | | |
| | EC + RT + LA + FP + KQ; EC + FPL + MF + ES + RI; | | | | |
| | PS + DS + FPL + MF + MS; DS + EL + LA + AH + MS; | | | | |
| | DS + LA + MM + ES + MS; EC + PS + RT + EL + RI; | | | | |
| | EC + PS + DS + FPL + AH; EC + RT + EL + TB + MS; | | | | |
| | SG + PS + RT + FPL + TB; SG + LA + MF + FP + MS; | | | | |
| | SG + MM + AH + FP + KQ; RT + LA + AH + ES + KQ; | | | | |
| | DS + FPL + LA + FP + RI; EC + DS + EL + MF + FP; | | | | |
| | SG + PS + FP + ES + RI; SG + RT + TB + MM + FP; | | | | |
| | SG + FPL + EL + LA + MM; PS + FPL + MF + TB + MM; | | | | |
| | PS + FPL + TB + FP + MS; PS + LA + MM + KQ + RI; | | | | |
| | RT + FPL + LA + MF + ES; DS + MM + FP + MS + KQ; | | | | |
| | EC + PS + RT + DS + KQ; EC + RT + TB + MM + FP; | | | | |
| | EC + DS + FPL + MM + AH; SG + RT + EL + LA + MM; | | | | |
| | LA + TB + AH + MS + KQ; LA + TB + FP + KQ + RI; | | | | |
| | LA + MM + FP + MS + KQ; EC + SG + PS + MF + MM; | | | | |
| | EC + DS + FPL + TB + MM; EC + DS + MM + AH + FP; | | | | |
| | SG + FPL + EL + MF + RI; RT + DS + LA + TB + AH; | | | | |
| | EL + TB + MM + AH + FP; EC + PS + RT + MM + RI; | | | | |
| | EC + EL + LA + MM + ES; EC + EL + MM + AH + ES; | | | | |
| | SG + PS + DS + TB + ES; SG + FPL + EL + AH + ES; | | | | |
| | RT + LA + AH + ES + MS; DS + FPL + LA + MF + KQ; | | | | |
| | DS + LA + MF + ES + MS; DS + LA + MM + ES + RI; | | | | |
| | EL + LA + TB + MM + FP; EC + SG + PS + MF + TB; | | | | |
| | EC + RT + EL + LA + MS; EC + DS + FPL + EL + RI; | | | | |
| | EC + DS + OS + LA + ES; EC + DS + MM + ES + MS; | | | | |
| | EC + EL + MM + FP + ES; SG + DS + TB + FP + ES; | | | | |
| | SG + FPL + EL + MF + KQ; SG + FP + ES + MS + KQ; | | | | |
| | PS + RT + LA + TB + MS; PS + EL + FP + MS + KQ; | | | | |
| | RT + DS + FPL + LA + AH; RT + DS + LA + MF + RI; | | | | |
| | RT + DS + LA + FP + KQ; DS + FPL + EL + LA + FP; | | | | |
| | TB + MM + FP + KQ + RI; SG + PS + LA + MS + KQ; | | | | |
| | TB + MM + ES + MS + KQ; EC + PS + MF + AH + FP; | | | | |
| | SG + LA + TB + KQ + RI; RT + FPL + MF + FP + RI; | | | | |
| | RT + FPL + AH + FP + ES; DS + EL + FP + ES + KQ; | | | | |
| | MF + TB + FP + ES + MS; EC + SG + FPL + OS + FP; | | | | |
| | EC + RT + DS + OS + FP; EC + DS + EL + AH + FP; | | | | |
| | EC + LA + AH + ES + RI; SG + MM + FP + MS + KQ; | | | | |
| | PS + RT + DS + ES + KQ; PS + RT + EL + FP + KQ; | | | | |
| | PS + RT + LA + TB + FP; DS + EL + MM + FP + MS; | | | | |
| | DS + LA + ES + KQ + RI; EC + PS + DS + FP + KQ; | | | | |
| | EC + PS + FPL + FP + ES; EC + PS + LA + MF + AH; | | | | |
| | EC + RT + DS + FPL + KQ; EC + RT + DS + LA + MF; | | | | |
| | SG + PS + RT + EL + FP; SG + DS + EL + LA + AH; | | | | |
| | FPL + LA + FP + ES + KQ; EC + SG + MF + FP + RI; | | | | |
| | EC + PS + MF + MS + RI; EC + RT + DS + EL + LA; | | | | |
| | SG + RT + EL + LA + AH; SG + RT + MF + FP + KQ; | | | | |
| | SG + RT + FP + ES + KQ; SG + EL + LA + TB + RI; | | | | |
| | PS + FPL + FP + ES + KQ; LA + MM + ES + MS + RI; | | | | |
| | EC + PS + MF + TB + KQ; SG + DS + EL + AH + KQ; | | | | |
| | SG + DS + TB + MM + RI; SG + FPL + AH + MS + KQ; | | | | |
| | PS + LA + MF + TB + ES; EC + PS + LA + AH + MS; | | | | |
| | SG + RT + DS + FPL + MM; PS + RT + LA + FP + KQ; | | | | |
| | RT + LA + MF + TB + ES; RT + LA + FP + MS + RI; | | | | |
| | DS + EL + TB + MM + KQ; EC + SG + RT + MM + RI; | | | | |
| | EC + RT + MF + TB + FP; EC + DS + EL + FP + ES; | | | | |
| | SG + PS + LA + TB + FP; SG + DS + MF + TB + FP; | | | | |
| | PS + RT + FPL + EL + RI; RT + DS + MF + FP + MS; | | | | |
| | EC + SG + EL + KQ + RI; EC + DS + EL + MM + KQ; | | | | |
| | EC + AH + ES + MS + KQ; EC + ES + MS + KQ + RI; | | | | |
| | SG + RT + EL + LA + TB; SG + LA + MF + TB + FP; | | | | |
| | PS + FPL + EL + MF + FP; RT + MF + FP + ES + KQ | | | | |
| 5 | EC + SG + RT + MF + ES; EC + SG + DS + LA + AH; | 0.77 | 0.3069- | 50.79%- | 45.45%- |
| | SG + DS + FP + MS + RI; PS + FPL + MF + TB + ES; | | 0.5233 | 96.83% | 91.92% |
| | DS + FPL + LA + ES + RI; DS + FPL + AH + ES + KQ; | | | | |
| | DS + TB + AH + ES + MS; EL + LA + TB + AH + FP; | | | | |
| | EC + SG + TB + MM + FP; EC + PS + DS + TB + AH; | | | | |
| | EC + DS + FPL + LA + TB; SG + RT + TB + KQ + RI; | | | | |
| | SG + EL + FP + MS + KQ; EL + TB + ES + MS + KQ; | | | | |
| | EC + DS + EL + LA + ES; SG + DS + EL + LA + ES; | | | | |
| | SG + DS + LA + FP + ES; PS + TB + MM + AH + ES; | | | | |
| | EC + SG + DS + FPL + FP; SG + PS + LA + TB + ES; | | | | |
| | DS + TB + MM + FP + MS; PS + LA + TB + MM + KQ; | | | | |
| | RT + DS + EL + LA + TB; RT + FPL + LA + TB + ES; | | | | |
| | DS + LA + TB + MM + AH; FPL + EL + MF + FP + ES; | | | | |
| | TB + MM + FP + ES + MS; EC + SG + DS + FPL + EL; | | | | |
| | EC + FPL + EL + TB + KQ; SG + PS + RT + EL + RI; | | | | |
| | RT + FPL + AH + ES + KQ; RT + EL + FP + ES + MS; | | | | |
| | EC + SG + FPL + TB + KQ; SG + PS + RT + LA + MS; | | | | |
| | SG + RT + FPL + MF + RI; SG + RT + LA + AH + FP; | | | | |
| | EC + SG + PS + EL + AH; EC + SG + EL + MM + KQ; | | | | |
| | EC + DS + FPL + MF + RI; EC + DS + FPL + ES + KQ; | | | | |
| | SG + PS + DS + KQ + RI; SG + RT + DS + FPL + TB; | | | | |
| | RT + FPL + FP + ES + KQ; DS + FPL + EL + AH + ES; | | | | |
| | EC + SG + RT + TB + MS; EC + PS + DS + LA + AH; | | | | |
| | EC + PS + DS + FP + MS; EC + PS + EL + TB + KQ; | | | | |
| | EC + RT + DS + TB + ES; EC + DS + MF + TB + MS; | | | | |
| | SG + PS + RT + FPL + KQ; SG + DS + TB + MS + RI; | | | | |
| | SG + DS + FP + ES + RI; SG + FPL + LA + KQ + RI; | | | | |
| | SG + LA + FP + MS + KQ; SG + MF + AH + FP + ES; | | | | |
| | SG + TB + AH + ES + KQ; PS + FPL + TB + ES + KQ; | | | | |
| | RT + FPL + LA + TB + KQ; RT + EL + MF + FP + ES; | | | | |
| | DS + FPL + EL + MF + ES; FPL + LA + TB + MM + KQ; | | | | |
| | EC + SG + EL + FP + MS; EC + PS + DS + EL + TB; | | | | |
| | EC + TB + MM + AH + ES; SG + PS + FPL + MM + RI; | | | | |
| | SG + DS + FPL + TB + MM; PS + DS + TB + FP + ES; | | | | |
| | PS + LA + TB + FP + MS; SG + RT + FPL + EL + FP; | | | | |
| | EC + RT + DS + EL + ES; EC + TB + FP + MS + RI; | | | | |
| | SG + FPL + TB + MS + KQ; PS + RT + EL + TB + ES; | | | | |
| | EC + SG + FPL + EL + MM; EC + SG + EL + MF + FP; | | | | |
| | EC + SG + MF + ES + RI; EC + PS + DS + EL + MF; | | | | |
| | EC + PS + DS + MF + MS; EC + DS + TB + KQ + RI; | | | | |
| | SG + PS + FPL + MM + KQ; SG + LA + AH + KQ + RI; | | | | |
| | EL + LA + TB + FP + KQ; EC + PS + RT + LA + TB; | | | | |
| | EC + RT + EL + ES + KQ; EC + DS + LA + TB + KQ; | | | | |
| | DS + FPL + EL + MM + ES; EL + MF + TB + FP + RI; | | | | |
| | EC + SG + LA + FP + ES; PS + RT + EL + LA + KQ; | | | | |
| | PS + DS + FPL + MM + KQ; RT + FPL + EL + ES + RI; | | | | |
| | EC + SG + PS + RT + MM; EC + SG + LA + MS + KQ; | | | | |
| | EC + DS + AH + ES + RI; RT + DS + EL + LA + ES; | | | | |
| | EC + SG + TB + MS + RI; EC + TB + AH + FP + ES; | | | | |
| | DS + FPL + FP + ES + RI; DS + TB + FP + ES + MS; | | | | |
| | EC + SG + LA + MF + AH; EC + DS + EL + TB + KQ; | | | | |
| | SG + DS + TB + AH + KQ; DS + FPL + MM + ES + KQ; | | | | |
| | EC + SG + EL + ES + MS; SG + PS + FPL + MF + TB; | | | | |
| | DS + EL + MF + TB + RI; DS + MF + TB + FP + ES; | | | | |
| | EC + DS + EL + ES + MS; SG + RT + DS + TB + RI; | | | | |
| | SG + RT + MS + KQ + RI; EC + PS + MF + TB + FP; | | | | |
| | EC + RT + EL + LA + ES; SG + RT + LA + FP + MS; | | | | |
| | SG + DS + FPL + EL + MS; SG + FPL + MM + FP + RI; | | | | |
| | PS + RT + FPL + LA + KQ; PS + RT + LA + MS + KQ; | | | | |
| | PS + EL + MF + TB + FP; RT + FP + ES + MS + RI; | | | | |
| | FPL + TB + MM + ES + RI; EC + SG + DS + EL + FP; | | | | |
| | EC + EL + TB + ES + MS; SG + EL + AH + ES + KQ; | | | | |
| | PS + RT + LA + MM + ES; EC + FPL + LA + TB + FP; | | | | |
| | SG + LA + MM + FP + KQ; PS + LA + MM + AH + ES; | | | | |
| | EC + SG + PS + FP + KQ; EC + SG + EL + MM + FP; | | | | |
| | EC + SG + TB + AH + KQ; SG + EL + MM + ES + MS; | | | | |
| | FPL + MF + TB + AH + ES; EC + RT + LA + ES + KQ; | | | | |
| | PS + DS + TB + MM + FP; DS + LA + FP + ES + MS; | | | | |
| | EC + SG + DS + MF + FP; EC + SG + FPL + KQ + RI; | | | | |
| | EC + PS + DS + KQ + RI; EC + RT + EL + TB + ES; | | | | |
| | SG + RT + LA + TB + AH; SG + EL + LA + FP + RI; | | | | |
| | PS + RT + EL + ES + MS; PS + LA + MM + FP + ES; | | | | |
| | EC + SG + TB + MM + KQ; EC + RT + FPL + MF + ES; | | | | |
| | EC + EL + TB + MM + FP; SG + EL + LA + ES + KQ; | | | | |
| | PS + RT + LA + ES + RI; PS + FPL + LA + TB + MM; | | | | |
| | DS + EL + MF + FP + ES; DS + TB + MM + AH + FP; | | | | |
| | FPL + MF + TB + ES + MS; EC + SG + PS + RT + FPL; | | | | |
| | EC + SG + PS + KQ + RI; EC + SG + FP + ES + KQ; | | | | |
| | EC + RT + DS + FPL + LA; EC + FPL + TB + AH + FP; | | | | |
| | RT + DS + FPL + MF + RI; RT + DS + FP + ES + RI; | | | | |
| | LA + TB + AH + ES + RI; EC + SG + DS + EL + MS; | | | | |
| | EC + SG + DS + MM + MS; EC + LA + FP + ES + MS; | | | | |
| | SG + FPL + MF + FP + KQ; SG + FPL + MM + FP + KQ; | | | | |
| | EC + PS + RT + DS + FPL; EC + DS + FP + MS + RI; | | | | |
| | EC + LA + TB + MS + RI; EC + TB + FP + ES + KQ; | | | | |
| | SG + PS + RT + FPL + FP; SG + PS + DS + FP + ES; | | | | |
| | SG + PS + LA + TB + RI; SG + RT + DS + EL + LA; | | | | |
| | SG + RT + EL + KQ + RI; SG + RT + TB + FP + RI; | | | | |
| | SG + EL + LA + AH + ES; SG + LA + MS + KQ + RI; | | | | |
| | PS + DS + MM + ES + RI; FPL + LA + TB + FP + MS; | | | | |
| | EC + SG + EL + TB + MS; EC + SG + LA + MF + RI; | | | | |
| | EC + SG + LA + MM + ES; EC + PS + DS + FP + ES; | | | | |
| | EC + EL + MF + TB + FP; SG + PS + LA + AH + MS; | | | | |
| | SG + RT + EL + ES + MS; SG + FPL + LA + MM + ES; | | | | |
| | DS + LA + TB + ES + KQ; EC + DS + FPL + TB + KQ; | | | | |
| | SG + RT + LA + MS + RI; SG + DS + MF + AH + ES; | | | | |
| | SG + DS + FP + ES + MS; SG + TB + AH + FP + KQ; | | | | |
| | PS + DS + FPL + MM + ES; DS + FPL + AH + ES + RI; | | | | |
| | FPL + MF + TB + FP + KQ; EC + RT + FPL + LA + TB; | | | | |
| | EC + DS + MF + AH + ES; SG + RT + FPL + TB + RI; | | | | |
| | SG + RT + LA + TB + MS; SG + DS + FPL + OS + RI; | | | | |
| | PS + DS + LA + TB + RI; DS + FPL + EL + ES + MS; | | | | |
| | DS + FPL + TB + MM + RI; DS + FPL + ES + MS + RI; | | | | |
| | EC + SG + RT + DS + AH; EC + SG + RT + EL + KQ; | | | | |
| | EC + PS + EL + TB + MS; EC + RT + LA + AH + ES; | | | | |
| | SG + PS + MM + AH + ES; EC + PS + LA + FP + MS; | | | | |
| | EC + RT + EL + ES + RI; EC + LA + ES + MS + KQ; | | | | |
| | EC + TB + ES + MS + KQ; SG + RT + FPL + LA + MF; | | | | |
| | SG + FPL + TB + MM + RI; SG + EL + LA + FP + ES; | | | | |
| | PS + RT + EL + KQ + RI; RT + EL + ES + KQ + RI; | | | | |
| | EC + SG + PS + FPL + EL; SG + DS + AH + ES + MS; | | | | |
| | SG + FPL + EL + MF + TB; PS + FPL + EL + MF + TB; | | | | |
| | RT + FPL + TB + FP + KQ; FPL + LA + TB + FP + KQ; | | | | |
| | FPL + LA + TB + ES + RI; EL + TB + FP + KQ + RI; | | | | |
| | EC + RT + EL + FP + ES; EC + TB + MM + FP + KQ; | | | | |
| | EC + TB + AH + ES + MS; SG + DS + LA + MF + AH; | | | | |
| | RT + EL + LA + TB + KQ; DS + FPL + LA + AH + ES; | | | | |
| | EL + MF + TB + ES + MS; EC + PS + FPL + ES + MS; | | | | |
| | EC + DS + EL + TB + FP; PS + DS + TB + FP + MS; | | | | |
| | DS + FPL + EL + FP + ES; DS + FPL + EL + FP + MS; | | | | |
| | DS + FPL + TB + FP + MS; EC + SG + DS + TB + MM; | | | | |
| | RT + DS + LA + MS + KQ; DS + TB + ES + MS + RI; | | | | |
| | EC + RT + LA + FP + ES; EC + TB + AH + ES + KQ; | | | | |
| | SG + PS + FPL + EL + RI; SG + PS + LA + MS + RI; | | | | |
| | DS + FPL + ES + MS + KQ; DS + TB + MM + AH + MS; | | | | |
| | EC + SG + DS + MF + MS; RT + DS + FPL + ES + MS; | | | | |
| | EC + SG + PS + MF + MS; EC + SG + RT + TB + RI; | | | | |
| | EC + RT + DS + AH + ES; EC + DS + AH + ES + MS; | | | | |
| | SG + RT + FPL + ES + MS; PS + DS + TB + MS + RI; | | | | |
| | RT + FPL + EL + TB + FP; RT + FPL + FP + ES + MS; | | | | |
| | DS + LA + AH + ES + MS; DS + TB + AH + FP + MS; | | | | |
| | EL + LA + TB + FP + RI; EC + SG + DS + EL + MF; | | | | |
| | EC + PS + EL + TB + FP; EC + PS + LA + MS + RI; | | | | |
| | EC + DS + AH + FP + ES; EC + EL + TB + FP + MS; | | | | |
| | EC + TB + MM + ES + KQ; SG + EL + MF + FP + ES; | | | | |
| | RT + LA + TB + ES + MS; FPL + TB + MM + AH + ES; | | | | |
| | EL + MF + TB + FP + KQ; EC + SG + DS + FP + RI; | | | | |
| | EC + SG + FPL + TB + MS; SG + PS + DS + AH + FP; | | | | |
| | PS + TB + ES + MS + RI; RT + DS + LA + ES + MS; | | | | |
| | EL + TB + MM + FP + MS; MF + TB + FP + ES + RI; | | | | |
| | EC + SG + MF + FP + MS; EC + RT + FP + ES + MS; | | | | |
| | EC + TB + FP + MS + KQ; PS + EL + TB + MM + RI; | | | | |
| | PS + LA + MM + ES + KQ; DS + MF + TB + FP + MS; | | | | |
| | EL + MF + TB + MM + ES; EC + LA + TB + MM + FP; | | | | |
| | EC + MF + TB + FP + ES; RT + DS + EL + MS + RI; | | | | |
| | RT + DS + LA + ES + KQ; RT + LA + ES + MS + RI; | | | | |
| | EC + SG + FP + MS + KQ; EC + RT + DS + LA + TB; | | | | |
| | EC + RT + TB + ES + MS; SG + PS + RT + MS + RI; | | | | |
| | PS + DS + FPL + LA + KQ; PS + FPL + TB + FP + ES; | | | | |
| | PS + EL + LA + ES + RI; RT + FPL + LA + TB + FP; | | | | |
| | TB + AH + FP + ES + RI; EC + SG + FPL + EL + FP; | | | | |
| | SG + RT + LA + TB + FP; SG + LA + MM + AH + KQ; | | | | |
| | PS + RT + DS + FPL + RI; EC + SG + RT + DS + MS; | | | | |
| | SG + DS + ES + KQ + RI; RT + LA + MM + ES + MS; | | | | |
| | EC + FPL + TB + ES + RI; PS + FPL + LA + FP + ES; | | | | |
| | EC + SG + PS + FPL + MM; EC + SG + FPL + MS + RI; | | | | |
| | EC + PS + LA + TB + FP; SG + RT + EL + LA + MS; | | | | |
| | SG + FPL + EL + ES + RI; PS + DS + FPL + MM + RI; | | | | |
| | RT + FPL + TB + AH + ES; EC + SG + EL + MS + KQ; | | | | |
| | EC + SG + AH + ES + MS; EC + RT + ES + MS + KQ; | | | | |
| | SG + PS + RT + LA + FP; SG + DS + LA + TB + MM; | | | | |
| | PS + DS + LA + ES + MS; RT + FPL + LA + FP + ES; | | | | |
| | DS + FPL + EL + FP + RI; DS + TB + MM + FP + KQ; | | | | |
| | EC + SG + PS + FPL + RI; EC + PS + RT + LA + MS; | | | | |
| | EC + PS + RT + LA + KQ; EC + DS + TB + ES + KQ; | | | | |
| | EC + TB + MM + FP + MS; SG + FPL + EL + MM + ES; | | | | |
| | SG + EL + LA + MM + FP; SG + LA + AH + MS + RI; | | | | |
| | PS + DS + FPL + EL + KQ; RT + DS + FPL + KQ + RI; | | | | |
| | RT + DS + LA + ES + RI; RT + DS + LA + MS + RI; | | | | |
| | DS + FPL + MF + TB + MM; EL + TB + MM + FP + RI; | | | | |
| | EC + SG + DS + MF + TB; SG + RT + FPL + EL + RI; | | | | |
| | PS + FPL + TB + ES + MS; PS + FPL + TB + KQ + RI; | | | | |
| | RT + FPL + MM + ES + KQ; DS + LA + MF + AH + FP; | | | | |
| | EC + SG + DS + FPL + LA; EC + SG + FPL + MM + KQ; | | | | |
| | EC + DS + EL + TB + MM; EC + DS + TB + MM + KQ; | | | | |
| | EC + MF + TB + AH + ES; RT + FPL + TB + ES + RI; | | | | |
| | RT + ES + MS + KQ + RI; DS + LA + MF + TB + AH; | | | | |
| | DS + LA + TB + FP + RI; DS + TB + FP + ES + KQ; | | | | |
| | DS + TB + FP + KQ + RI; EC + SG + PS + DS + AH; | | | | |
| | EC + PS + DS + FPL + EL; SG + PS + RT + DS + FPL; | | | | |
| | SG + DS + EL + FP + KQ; PS + FPL + LA + TB + RI; | | | | |
| | RT + DS + FPL + EL + KQ; RT + EL + FP + ES + KQ; | | | | |
| | DS + FPL + ES + KQ + RI; FPL + TB + AH + ES + MS; | | | | |
| | LA + TB + MM + MS + RI; TB + AH + FP + ES + KQ; | | | | |
| | EC + SG + PS + RT + EL; EC + PS + LA + MF + TB; | | | | |
| | EC + PS + TB + FP + RI; EC + RT + TB + AH + ES; | | | | |
| | EC + RT + TB + ES + RI; EC + DS + LA + MF + ES; | | | | |
| | SG + PS + RT + TB + RI; RT + DS + FPL + MS + RI; | | | | |
| | EC + DS + MF + ES + MS; SG + PS + RT + EL + LA; | | | | |
| | PS + DS + LA + FP + MS; PS + LA + FP + ES + MS; | | | | |
| | RT + EL + TB + FP + KQ; DS + EL + AH + ES + RI; | | | | |
| | EC + PS + RT + DS + LA; SG + TB + MM + ES + MS; | | | | |
| | PS + FPL + EL + TB + KQ; PS + EL + TB + KQ + RI; | | | | |
| | PS + EL + AH + ES + KQ; PS + EL + ES + MS + RI; | | | | |
| | DS + FPL + MM + ES + RI; EC + PS + RT + AH + ES; | | | | |
| | EC + LA + MF + TB + FP; SG + PS + EL + MF + ES; | | | | |
| | SG + FPL + EL + TB + MS; RT + DS + ES + KQ + RI; | | | | |
| | FPL + TB + MM + ES + MS; EC + SG + DS + EL + AH; | | | | |
| | EC + PS + EL + FP + ES; SG + PS + RT + DS + LA; | | | | |
| | SG + RT + EL + FP + KQ; RT + DS + EL + TB + RI; | | | | |
| | FPL + TB + ES + MS + KQ; EC + SG + DS + MM + KQ; | | | | |
| | EC + PS + AH + ES + MS; SG + RT + LA + MM + KQ; | | | | |
| | RT + EL + TB + ES + MS; EC + DS + OS + LA + TB; | | | | |
| | SG + PS + EL + TB + RI; SG + RT + LA + AH + RI; | | | | |
| | SG + DS + EL + MF + RI; PS + DS + FPL + TB + MM; | | | | |
| | RT + DS + FPL + TB + RI; RT + FPL + MF + TB + FP; | | | | |
| | FPL + LA + TB + MM + FP; EC + PS + FPL + TB + RI; | | | | |
| | SG + RT + LA + MF + ES; SG + FPL + MF + TB + AH; | | | | |
| | SG + FPL + MM + FP + ES; PS + DS + FPL + MF + FP; | | | | |
| | RT + DS + EL + LA + MS; RT + FP + ES + KQ + RI; | | | | |
| | DS + FPL + AH + FP + ES; EC + SG + RT + AH + FP; | | | | |
| | EC + SG + ES + KQ + RI; EC + DS + TB + AH + MS; | | | | |
| | SG + FPL + EL + MM + FP; RT + DS + FPL + ES + KQ; | | | | |
| | LA + TB + ES + MS + KQ; EC + PS + DS + EL + KQ; | | | | |
| | EC + PS + EL + MM + KQ; EC + DS + FPL + EL + FP; | | | | |
| | EC + DS + MF + MS + RI; EC + TB + AH + ES + RI; | | | | |
| | SG + EL + MF + FP + RI; SG + LA + MM + AH + RI; | | | | |
| | PS + FPL + EL + MF + ES; RT + DS + TB + MS + RI; | | | | |
| | RT + FPL + EL + LA + KQ; EC + SG + MM + FP + MS; | | | | |
| | EC + DS + EL + ES + KQ; PS + RT + ES + MS + RI; | | | | |
| | RT + DS + FPL + TB + FP; EC + SG + LA + FP + KQ; | | | | |
| | EC + SG + LA + KQ + RI; SG + TB + MM + FP + KQ; | | | | |
| | PS + DS + FPL + MS + RI; PS + LA + TB + MS + RI; | | | | |
| | RT + EL + LA + TB + FP; DS + LA + TB + AH + KQ; | | | | |
| | EC + SG + DS + TB + FP; EC + PS + FPL + MM + ES; | | | | |
| | EC + DS + LA + ES + KQ; EC + DS + AH + ES + KQ; | | | | |
| | SG + PS + RT + LA + AH; SG + RT + DS + LA + FP; | | | | |
| | SG + FPL + LA + ES + MS; SG + FPL + MF + TB + MM; | | | | |
| | PS + FPL + MM + ES + KQ; RT + DS + LA + TB + MS; | | | | |
| | EC + SG + DS + EL + LA; EC + SG + EL + LA + TB; | | | | |
| | EC + PS + TB + FP + KQ; EC + RT + DS + LA + MS; | | | | |
| | EC + FPL + TB + FP + MS; PS + LA + TB + MM + RI; | | | | |
| | EL + LA + MF + TB + ES; EC + SG + RT + EL + RI; | | | | |
| | EC + PS + AH + ES + RI; EC + DS + LA + MS + RI; | | | | |
| | EC + EL + TB + AH + FP; PS + RT + LA + MS + RI; | | | | |
| | EC + RT + FPL + TB + FP; SG + RT + LA + TB + MM; | | | | |
| | PS + RT + FPL + LA + ES; RT + DS + TB + ES + RI; | | | | |
| | RT + LA + TB + FP + MS; DS + LA + AH + ES + RI; | | | | |
| | EC + PS + TB + AH + KQ; EC + EL + TB + ES + KQ; | | | | |
| | SG + PS + FPL + TB + MM; SG + RT + LA + MF + RI; | | | | |
| | PS + RT + LA + KQ + RI; PS + DS + EL + FP + RI; | | | | |
| | PS + LA + MF + ES + RI; RT + DS + FPL + TB + KQ; | | | | |
| | DS + LA + MM + MS + KQ; EL + MF + TB + FP + MS; | | | | |
| | EC + SG + PS + MM + MS; EC + PS + FP + ES + MS; | | | | |
| | EC + DS + TB + AH + RI; SG + EL + MM + FP + KQ; | | | | |
| | RT + EL + LA + TB + MS; LA + TB + AH + ES + KQ; | | | | |
| | LA + TB + FP + MS + RI; TB + FP + ES + MS + RI; | | | | |
| | EC + SG + FPL + EL + ES; SG + PS + RT + FP + RI; | | | | |
| | SG + RT + LA + MM + FP; SG + LA + FP + ES + MS; | | | | |
| | PS + DS + LA + ES + KQ; PS + DS + ES + KQ + RI; | | | | |
| | EC + DS + TB + MM + AH; SG + RT + ES + MS + KQ; | | | | |
| | SG + EL + TB + MM + ES; PS + RT + LA + AH + ES; | | | | |
| | EC + DS + EL + LA + MS; SG + PS + RT + EL + KQ; | | | | |
| | PS + RT + FPL + EL + KQ; PS + RT + EL + FP + ES; | | | | |
| | RT + DS + FPL + LA + TB; EC + DS + MM + AH + ES; | | | | |
| | EC + DS + ES + MS + KQ; SG + RT + FPL + EL + TB; | | | | |
| | SG + EL + FP + KQ + RI; SG + TB + AH + ES + RI; | | | | |
| | PS + DS + FPL + MF + KQ; EC + RT + LA + TB + MS; | | | | |
| | EC + SG + RT + TB + KQ; EC + SG + FPL + LA + ES; | | | | |
| | DS + FPL + FP + ES + MS; EC + SG + TB + MM + AH; | | | | |
| | EC + PS + AH + FP + ES; EC + MF + TB + FP + MS; | | | | |
| | EC + MF + TB + ES + MS; SG + PS + TB + AH + RI; | | | | |
| | RT + DS + FPL + MF + TB; RT + EL + LA + MS + KQ; | | | | |
| | RT + EL + TB + FP + RI; MF + TB + MM + FP + MS; | | | | |
| | EC + SG + TB + MS + KQ; EC + PS + FPL + EL + ES; | | | | |
| | SG + LA + TB + FP + RI; DS + LA + MF + TB + FP; | | | | |
| | EC + SG + RT + MM + KQ; EC + PS + MF + TB + RI; | | | | |
| | EC + PS + TB + KQ + RI; EC + DS + FP + ES + MS; | | | | |
| | SG + PS + FPL + FP + MS; SG + PS + EL + AH + FP; | | | | |
| | PS + DS + LA + FP + ES; RT + FPL + TB + KQ + RI; | | | | |
| | RT + FPL + FP + ES + RI; DS + EL + FP + MS + RI; | | | | |
| | EC + SG + FPL + AH + ES; EC + PS + RT + DS + RI; | | | | |
| | EC + RT + FPL + EL + TB; EC + RT + FPL + TB + AH; | | | | |
| | SG + RT + DS + FPL + MF; SG + EL + MM + FP + RI; | | | | |
| | DS + LA + ES + MS + KQ; LA + TB + AH + FP + KQ; | | | | |
| | EC + SG + PS + RT + AH; EC + SG + RT + DS + KQ; | | | | |
| | EC + PS + DS + MF + AH; EC + LA + TB + MM + MS; | | | | |
| | SG + PS + FPL + MM + FP; SG + EL + TB + ES + KQ; | | | | |
| | PS + DS + LA + AH + MS; EC + SG + DS + FPL + MF; | | | | |
| | EC + SG + FPL + MM + ES; EC + PS + DS + FPL + KQ; | | | | |
| | EC + DS + MS + KQ + RI; SG + RT + EL + LA + RI; | | | | |
| | SG + DS + MS + KQ + RI; SG + TB + FP + MS + KQ; | | | | |
| | FPL + LA + TB + MS + KQ; EC + DS + EL + MS + KQ; | | | | |
| | EC + FPL + TB + FP + RI; EC + EL + LA + TB + MS; | | | | |
| | SG + RT + TB + FP + KQ; DS + FPL + LA + TB + AH; | | | | |
| | DS + FPL + MF + ES + MS; EC + PS + DS + FPL + MM; | | | | |
| | EC + PS + LA + TB + RI; EC + RT + LA + MF + ES; | | | | |
| | EC + DS + OS + TB + FP; SG + EL + ES + MS + RI; | | | | |
| | DS + EL + LA + AH + ES; EL + MF + TB + MM + FP; | | | | |
| | EC + RT + DS + MM + ES; EC + LA + TB + MM + KQ; | | | | |
| | SG + RT + LA + MF + AH; SG + DS + EL + MM + FP; | | | | |
| | PS + RT + LA + MF + ES; PS + RT + LA + TB + ES; | | | | |
| | PS + LA + TB + MM + AH; EC + PS + DS + EL + MS; | | | | |
| | EC + PS + TB + MM + MS; EC + RT + DS + TB + MS; | | | | |
| | SG + PS + LA + MF + AH; RT + EL + TB + MM + ES; | | | | |
| | RT + LA + TB + MM + ES; DS + FPL + LA + MF + ES; | | | | |
| | EC + RT + DS + TB + FP; SG + RT + EL + FP + MS; | | | | |
| | PS + DS + EL + MF + ES; RT + DS + FPL + EL + MF; | | | | |
| | RT + DS + LA + FP + ES; EC + SG + RT + DS + MF; | | | | |
| | EC + SG + MF + AH + FP; SG + PS + RT + LA + TB; | | | | |
| | SG + RT + LA + TB + KQ; SG + EL + MM + FP + MS; | | | | |
| | PS + RT + LA + FP + ES; RT + EL + MF + ES + RI; | | | | |
| | DS + LA + TB + MM + ES; FPL + EL + TB + MM + RI; | | | | |
| | EC + SG + PS + EL + MM; EC + TB + MM + FP + RI; | | | | |
| | SG + RT + DS + OS + LA; SG + RT + DS + EL + FP; | | | | |
| | SG + RT + DS + FP + RI; SG + FPL + EL + MM + KQ; | | | | |
| | SG + FPL + LA + MF + ES; SG + LA + AH + FP + KQ; | | | | |
| | PS + DS + LA + AH + RI; PS + FPL + TB + MM + RI; | | | | |
| | PS + LA + FP + ES + RI; PS + MF + TB + AH + FP; | | | | |
| | RT + DS + LA + TB + FP; RT + LA + FP + ES + KQ; | | | | |
| | FPL + EL + FP + ES + MS; EC + SG + DS + MM + RI; | | | | |
| | EC + RT + DS + FPL + MS; SG + DS + MM + FP + MS; | | | | |
| | SG + EL + LA + AH + FP; SG + LA + TB + ES + RI; | | | | |
| | RT + LA + TB + ES + KQ; DS + EL + AH + FP + ES; | | | | |
| | DS + LA + TB + AH + RI; EC + SG + RT + FP + MS; | | | | |
| | EC + SG + DS + LA + MM; EC + DS + FPL + MF + AH; | | | | |
| | EL + TB + AH + FP + KQ; EC + SG + DS + FPL + KQ; | | | | |
| | SG + PS + AH + ES + KQ; SG + RT + DS + FP + KQ; | | | | |
| | SG + RT + EL + FP + RI; RT + DS + EL + TB + KQ; | | | | |
| | RT + FPL + EL + ES + MS; RT + LA + MF + FP + ES; | | | | |
| | FPL + EL + TB + MM + MS; EC + PS + DS + AH + KQ; | | | | |
| | EC + RT + DS + FPL + ES; EC + DS + MF + TB + AH; | | | | |
| | SG + PS + FPL + LA + KQ; SG + RT + LA + MM + RI; | | | | |
| | SG + FPL + TB + MM + FP; EC + RT + LA + MM + ES; | | | | |
| | EC + RT + LA + ES + RI; SG + PS + EL + MS + KQ; | | | | |
| | SG + DS + LA + TB + RI; SG + AH + FP + ES + RI; | | | | |
| | PS + RT + LA + MM + KQ; PS + DS + EL + LA + FP; | | | | |
| | PS + FPL + MM + ES + RI; RT + DS + EL + TB + FP; | | | | |
| | DS + LA + MM + AH + ES; EC + SG + LA + MM + FP; | | | | |
| | EC + PS + DS + AH + RI; EC + PS + TB + MM + FP; | | | | |
| | EC + RT + ES + KQ + RI; EC + DS + FPL + LA + RI; | | | | |
| | SG + RT + LA + FP + RI; SG + TB + FP + ES + KQ; | | | | |
| | PS + FPL + EL + ES + KQ; PS + FPL + LA + ES + RI; | | | | |
| | RT + EL + ES + MS + KQ; TB + FP + ES + MS + KQ; | | | | |
| | EC + SG + RT + MF + RI; EC + PS + EL + LA + MS; | | | | |
| | TB + MM + ES + MS + RI; EC + PS + MF + TB + MS; | | | | |
| | SG + RT + DS + LA + KQ; SG + LA + MM + MS + KQ; | | | | |
| | PS + RT + FP + ES + RI; PS + DS + ES + MS + RI; | | | | |
| | PS + FPL + EL + MM + ES; RT + DS + FPL + EL + RI; | | | | |
| | EC + SG + AH + FP + MS; EC + PS + DS + MM + AH; | | | | |
| | EC + DS + EL + FP + MS; EC + DS + LA + MF + RI; | | | | |
| | EC + DS + MM + ES + RI; EC + LA + TB + AH + MS; | | | | |
| | SG + FPL + MF + FP + RI; SG + TB + MM + FP + MS; | | | | |
| | PS + RT + EL + MM + ES; RT + DS + FPL + MF + FP; | | | | |
| | RT + TB + ES + KQ + RI; DS + FPL + MF + FP + RI; | | | | |
| | DS + FPL + MM + AH + ES; DS + LA + AH + ES + KQ; | | | | |
| | SG + DS + LA + KQ + RI; SG + DS + AH + FP + RI; | | | | |
| | SG + FPL + MF + FP + MS; PS + RT + DS + LA + RI; | | | | |
| | SG + PS + FPL + MM + ES; SG + PS + AH + ES + RI; | | | | |
| | SG + RT + MF + FP + ES; SG + FPL + LA + MF + AH; | | | | |
| | RT + DS + FPL + FP + ES; DS + FPL + EL + TB + MS; | | | | |
| | EC + PS + MS + KQ + RI; EC + RT + DS + FP + MS; | | | | |
| | EC + DS + FPL + LA + AH; EC + DS + LA + AH + MS; | | | | |
| | SG + PS + DS + EL + KQ; SG + PS + FPL + MF + RI; | | | | |
| | SG + PS + EL + LA + TB; SG + RT + DS + FP + ES; | | | | |
| | PS + DS + FPL + TB + MS; PS + FPL + ES + KQ + RI; | | | | |
| | DS + EL + LA + TB + MS; EC + PS + DS + LA + FP; | | | | |
| | EC + EL + TB + MS + KQ; SG + PS + ES + MS + RI; | | | | |
| | SG + EL + TB + AH + ES; PS + DS + MF + ES + RI; | | | | |
| | PS + FPL + EL + LA + TB; RT + DS + FP + ES + KQ; | | | | |
| | RT + EL + LA + FP + ES; RT + EL + TB + MS + KQ; | | | | |
| | DS + FPL + MF + FP + ES; FPL + LA + MF + TB + FP; | | | | |
| | EC + EL + LA + TB + FP; EC + LA + TB + FP + RI; | | | | |
| | SG + FPL + EL + ES + MS; SG + EL + TB + FP + RI; | | | | |
| | PS + FPL + TB + MS + KQ; EC + SG + PS + FPL + FP; | | | | |
| | EC + DS + FP + ES + RI; EC + EL + LA + TB + KQ; | | | | |
| | SG + PS + AH + FP + KQ; SG + DS + AH + ES + KQ; | | | | |
| | PS + RT + ES + MS + KQ; RT + LA + MS + KQ + RI; | | | | |
| | DS + FPL + MF + KQ + RI; EC + PS + TB + AH + FP; | | | | |
| | EC + PS + RT + LA + RI; EC + LA + TB + FP + ES; | | | | |
| | RT + DS + FP + MS + RI; RT + LA + TB + MS + KQ; | | | | |
| | DS + LA + MM + AH + MS; LA + MF + TB + FP + MS; | | | | |
| | EC + SG + FPL + FP + RI; EC + SG + EL + LA + FP; | | | | |
| | EC + MF + TB + ES + RI; SG + PS + LA + MM + RI; | | | | |
| | SG + DS + AH + FP + MS; SG + TB + FP + ES + RI; | | | | |
| | PS + RT + DS + EL + LA; PS + RT + TB + ES + RI; | | | | |
| | PS + DS + MM + AH + ES; PS + FPL + MF + TB + RI; | | | | |
| | RT + DS + EL + KQ + RI; RT + DS + LA + AH + ES; | | | | |
| | EL + TB + ES + MS + RI; EC + SG + RT + OS + LA; | | | | |
| | EC + SG + EL + LA + KQ; EC + SG + AH + FP + KQ; | | | | |
| | EC + PS + LA + MS + KQ; EC + RT + DS + FPL + EL; | | | | |
| | EC + DS + TB + AH + KQ; EC + LA + MF + TB + MS; | | | | |
| | SG + DS + AH + FP + ES; PS + RT + LA + TB + KQ; | | | | |
| | PS + EL + LA + MM + KQ; RT + FPL + LA + AH + ES; | | | | |
| | EC + SG + DS + FP + ES; EC + PS + LA + MF + RI; | | | | |
| | EC + DS + LA + MF + FP; SG + RT + EL + MF + FP; | | | | |
| | SG + DS + TB + MM + MS; SG + FPL + MM + AH + KQ; | | | | |
| | SG + EL + MF + AH + FP; PS + DS + EL + ES + MS; | | | | |
| | RT + DS + LA + TB + ES; DS + FPL + TB + MM + ES; | | | | |
| | EC + PS + FPL + TB + KQ; EC + PS + MF + FP + ES; | | | | |
| | EC + DS + FPL + EL + LA; EC + FPL + LA + AH + ES; | | | | |
| | EC + LA + AH + ES + MS; SG + LA + MF + FP + ES; | | | | |
| | PS + RT + DS + FPL + ES; PS + DS + EL + MF + RI; | | | | |
| | PS + TB + FP + ES + MS; RT + DS + FPL + MS + KQ; | | | | |
| | EC + SG + PS + FPL + KQ; EC + SG + DS + LA + FP; | | | | |
| | EC + MF + TB + MM + FP; SG + PS + FPL + LA + MM; | | | | |
| | SG + RT + FPL + AH + KQ; PS + EL + MF + ES + RI; | | | | |
| | FPL + TB + ES + MS + RI; EC + SG + EL + LA + MM; | | | | |
| | EC + DS + LA + AH + FP; EC + FPL + MF + AH + ES; | | | | |
| | SG + RT + FPL + MM + RI; PS + FPL + TB + AH + KQ; | | | | |
| | PS + TB + MM + FP + RI; RT + DS + FPL + LA + KQ; | | | | |
| | EC + DS + ES + KQ + RI; EC + EL + TB + ES + RI; | | | | |
| | EC + LA + AH + FP + ES; SG + PS + FPL + AH + ES; | | | | |
| | SG + RT + DS + FPL + MS; RT + EL + MF + TB + FP; | | | | |
| | EC + SG + TB + MM + MS; EC + SG + AH + FP + RI; | | | | |
| | EC + PS + RT + MF + ES; EC + PS + LA + FP + ES; | | | | |
| | EC + RT + DS + FPL + MF; EC + EL + TB + MM + KQ; | | | | |
| | SG + PS + DS + AH + KQ; SG + LA + AH + MS + KQ; | | | | |
| | DS + FPL + EL + MM + FP; EC + PS + DS + EL + MM; | | | | |
| | EC + PS + FPL + MF + FP; EC + FPL + MF + TB + KQ; | | | | |
| | SG + DS + MF + TB + AH; RT + DS + FPL + LA + ES; | | | | |
| | RT + DS + LA + MM + MS; RT + FPL + EL + LA + TB; | | | | |
| | RT + LA + TB + FP + KQ; FPL + LA + MF + TB + ES; | | | | |
| | EC + SG + RT + MS + RI; EC + PS + DS + EL + LA; | | | | |
| | EC + DS + EL + MF + TB; EC + FPL + AH + ES + KQ; | | | | |
| | SG + PS + TB + MM + AH; SG + RT + FPL + MS + RI; | | | | |
| | SG + DS + MM + AH + ES; SG + LA + TB + MS + RI; | | | | |
| | PS + DS + FPL + LA + MM; PS + DS + TB + MM + MS; | | | | |
| | PS + DS + TB + AH + RI; PS + FPL + TB + FP + RI; | | | | |
| | DS + MF + TB + AH + RI; FPL + EL + TB + MS + KQ; | | | | |
| | EC + SG + PS + DS + FPL; EC + PS + MF + TB + AH; | | | | |
| | EC + RT + FP + ES + RI; EC + TB + MM + AH + FP; | | | | |
| | SG + FPL + EL + MF + ES; SG + FPL + LA + MS + KQ; | | | | |
| | DS + EL + AH + ES + MS; DS + TB + AH + FP + RI; | | | | |
| | EC + RT + FPL + TB + KQ; EC + RT + LA + TB + FP; | | | | |
| | SG + RT + EL + TB + FP; SG + DS + TB + FP + RI; | | | | |
| | SG + MF + TB + FP + ES; PS + FPL + TB + FP + KQ; | | | | |
| | DS + MF + TB + FP + RI; EC + PS + EL + LA + KQ; | | | | |
| | SG + PS + DS + MM + RI; SG + PS + LA + FP + KQ; | | | | |
| | PS + DS + FPL + EL + LA; PS + DS + EL + TB + MS; | | | | |
| | PS + DS + MF + TB + ES; RT + FPL + MS + KQ + RI; | | | | |
| | DS + FPL + LA + MF + FP; EC + PS + RT + MS + KQ; | | | | |
| | EC + DS + FPL + EL + KQ; SG + PS + DS + LA + MS; | | | | |
| | SG + EL + LA + MM + KQ; PS + DS + FPL + EL + MM; | | | | |
| | PS + DS + EL + TB + FP; PS + DS + AH + FP + ES; | | | | |
| | PS + FPL + LA + TB + KQ; RT + DS + FPL + EL + FP; | | | | |
| | DS + FPL + FP + KQ + RI; EC + RT + EL + MM + ES; | | | | |
| | EC + DS + FPL + KQ + RI; EC + FPL + TB + MS + KQ; | | | | |
| | EC + LA + MM + AH + ES; SG + PS + DS + LA + TB; | | | | |
| | SG + PS + LA + MM + FP; SG + RT + DS + MF + FP; | | | | |
| | DS + EL + MF + FP + MS; FPL + EL + TB + KQ + RI; | | | | |
| | TB + MM + FP + MS + KQ; EC + PS + DS + OS + TB; | | | | |
| | EC + PS + DS + MM + RI; EC + DS + TB + MM + MS; | | | | |
| | SG + MM + AH + ES + MS; PS + DS + TB + AH + FP; | | | | |
| | RT + MF + FP + ES + RI; EC + SG + PS + EL + RI; | | | | |
| | EC + SG + DS + FPL + TB; EC + PS + RT + FP + ES; | | | | |
| | EC + RT + LA + MS + KQ; SG + EL + MF + ES + RI; | | | | |
| | SG + TB + MM + ES + RI; PS + DS + EL + FP + MS; | | | | |
| | PS + LA + ES + MS + RI; TB + MM + FP + MS + RI; | | | | |
| | EC + SG + RT + FP + KQ; SG + FPL + EL + LA + ES; | | | | |
| | PS + RT + DS + EL + RI; PS + LA + MF + AH + ES; | | | | |
| | RT + DS + FPL + AH + ES; RT + FPL + ES + MS + KQ; | | | | |
| | DS + FPL + LA + MM + ES; DS + EL + ES + MS + RI; | | | | |
| | FPL + EL + AH + FP + ES; TB + MM + AH + ES + MS | | | | |
| 5 | EC + LA + TB + ES + KQ; SG + PS + FPL + TB + FP; | 0.78 | 0.3129- | 47.62%- | 46.46%- |
| | SG + DS + EL + FP + RI; FPL + EL + TB + MM + ES; | | 0.5326 | 95.24% | 92.93% |
| | EC + SG + DS + AH + FP; EC + DS + LA + TB + RI; | | | | |
| | SG + PS + TB + MM + RI; SG + FPL + LA + FP + ES; | | | | |
| | DS + FPL + LA + TB + RI; EL + TB + FP + MS + KQ; | | | | |
| | DS + FPL + EL + TB + KQ; FPL + TB + MM + FP + ES; | | | | |
| | EC + SG + DS + AH + RI; EC + SG + LA + TB + FP; | | | | |
| | EC + DS + FPL + TB + FP; SG + FPL + FP + MS + RI; | | | | |
| | FPL + TB + FP + MS + RI; SG + PS + EL + LA + KQ; | | | | |
| | SG + DS + LA + TB + MS; PS + EL + TB + MM + MS; | | | | |
| | FPL + TB + MM + FP + KQ; EC + SG + PS + TB + MM; | | | | |
| | EC + SG + RT + ES + MS; SG + FPL + TB + ES + RI; | | | | |
| | EC + SG + MM + ES + RI; RT + DS + FPL + ES + RI; | | | | |
| | EC + FPL + TB + ES + KQ; SG + RT + EL + ES + RI; | | | | |
| | PS + DS + EL + LA + KQ; RT + DS + EL + ES + MS; | | | | |
| | DS + FPL + EL + MF + TB; EC + SG + PS + AH + RI; | | | | |
| | SG + RT + ES + KQ + RI; SG + DS + FPL + MM + RI; | | | | |
| | DS + EL + TB + AH + ES; EC + SG + PS + AH + KQ; | | | | |
| | EC + SG + RT + TB + ES; PS + RT + FPL + EL + ES; | | | | |
| | RT + FPL + LA + ES + KQ; EC + PS + TB + MS + KQ; | | | | |
| | EC + TB + FP + ES + MS; SG + LA + TB + FP + ES; | | | | |
| | PS + EL + LA + TB + KQ; DS + FPL + LA + TB + KQ; | | | | |
| | DS + EL + TB + FP + KQ; EC + SG + PS + TB + AH; | | | | |
| | EC + PS + LA + MF + ES; EC + PS + ES + MS + RI; | | | | |
| | SG + RT + FPL + LA + RI; SG + DS + FPL + LA + MS; | | | | |
| | PS + DS + EL + ES + KQ; PS + FPL + TB + MM + ES; | | | | |
| | LA + TB + MM + FP + MS; EL + LA + TB + ES + KQ; | | | | |
| | SG + PS + FPL + FP + RI; SG + RT + EL + LA + ES; | | | | |
| | EC + SG + PS + MM + KQ; EC + PS + DS + MS + RI; | | | | |
| | SG + DS + FPL + MF + KQ; SG + DS + FPL + MM + FP; | | | | |
| | PS + RT + EL + ES + RI; EC + SG + FPL + TB + RI; | | | | |
| | EC + PS + LA + TB + KQ; SG + PS + EL + FP + KQ; | | | | |
| | DS + FPL + TB + ES + MS; DS + TB + FP + MS + KQ; | | | | |
| | LA + MF + TB + FP + ES; EC + PS + RT + FPL + ES; | | | | |
| | SG + TB + MM + AH + FP; DS + FPL + EL + LA + TB; | | | | |
| | EC + SG + TB + ES + MS; EC + SG + AH + ES + KQ; | | | | |
| | SG + PS + RT + DS + RI; SG + DS + FPL + LA + MM; | | | | |
| | SG + DS + LA + AH + KQ; FPL + TB + AH + ES + KQ; | | | | |
| | EC + SG + DS + TB + AH; EC + PS + FPL + ES + KQ; | | | | |
| | SG + PS + DS + TB + RI; PS + FPL + EL + TB + MM; | | | | |
| | SG + RT + DS + LA + RI; SG + RT + FPL + EL + KQ; | | | | |
| | FPL + EL + TB + ES + MS; EC + SG + RT + LA + AH; | | | | |
| | EC + SG + MF + AH + ES; EC + PS + TB + FP + MS; | | | | |
| | EC + DS + TB + FP + RI; SG + RT + LA + ES + MS; | | | | |
| | SG + EL + AH + FP + ES; FPL + LA + TB + FP + ES; | | | | |
| | EC + SG + PS + MM + FP; EC + EL + LA + TB + ES; | | | | |
| | SG + PS + DS + EL + FP; LA + TB + MM + FP + KQ; | | | | |
| | EC + PS + LA + MM + ES; EC + TB + MM + ES + RI; | | | | |
| | SG + PS + LA + TB + MM; RT + FPL + EL + ES + KQ; | | | | |
| | EL + LA + TB + ES + RI; EL + TB + MM + FP + KQ; | | | | |
| | EC + SG + DS + FP + MS; EC + DS + EL + LA + TB; | | | | |
| | SG + PS + DS + MS + RI; SG + RT + LA + ES + KQ; | | | | |
| | EC + SG + RT + LA + MF; EC + PS + DS + ES + KQ; | | | | |
| | EC + PS + FPL + MF + TB; EC + SG + PS + DS + KQ; | | | | |
| | EC + PS + LA + ES + RI; SG + RT + LA + FP + ES; | | | | |
| | DS + FPL + TB + KQ + RI; DS + TB + FP + ES + RI; | | | | |
| | EC + SG + DS + MF + RI; EC + PS + EL + MM + ES; | | | | |
| | EC + DS + LA + TB + AH; SG + PS + EL + MS + RI; | | | | |
| | LA + TB + FP + ES + KQ; EC + SG + AH + FP + ES; | | | | |
| | SG + PS + DS + LA + ES; EC + SG + PS + MS + KQ; | | | | |
| | EC + PS + MF + ES + RI; EC + DS + MF + TB + RI; | | | | |
| | SG + RT + DS + FPL + RI; RT + EL + TB + ES + RI; | | | | |
| | EC + SG + EL + MF + ES; EC + PS + EL + ES + KQ; | | | | |
| | SG + DS + TB + ES + RI; EC + SG + ES + MS + RI; | | | | |
| | SG + RT + FPL + FP + RI; LA + TB + MM + ES + MS; | | | | |
| | SG + FPL + AH + FP + KQ; SG + LA + MF + AH + ES; | | | | |
| | SG + LA + TB + AH + KQ; PS + EL + LA + TB + FP; | | | | |
| | DS + LA + TB + MS + RI; SG + DS + FPL + MF + MS; | | | | |
| | PS + FPL + TB + ES + RI; RT + DS + EL + FP + ES; | | | | |
| | LA + TB + MM + AH + ES; EC + SG + DS + EL + KQ; | | | | |
| | EC + PS + DS + MS + KQ; SG + PS + EL + MF + FP; | | | | |
| | DS + LA + TB + FP + MS; EC + SG + RT + EL + LA; | | | | |
| | EC + SG + FPL + LA + RI; EC + SG + EL + TB + FP; | | | | |
| | EC + PS + FPL + LA + ES; EC + DS + FPL + EL + TB; | | | | |
| | EC + DS + TB + MM + RI; SG + RT + LA + ES + RI; | | | | |
| | SG + FPL + LA + FP + MS; PS + RT + EL + LA + ES; | | | | |
| | FPL + EL + MF + TB + FP; FPL + TB + FP + MS + KQ; | | | | |
| | RT + DS + FPL + EL + ES; EC + SG + PS + FPL + AH; | | | | |
| | EC + SG + MM + AH + FP; EC + PS + FPL + EL + TB; | | | | |
| | EC + DS + LA + MF + TB; EC + EL + TB + MM + ES; | | | | |
| | SG + PS + EL + LA + FP; PS + RT + DS + LA + ES; | | | | |
| | EC + SG + PS + DS + MM; EC + SG + MF + TB + ES; | | | | |
| | SG + PS + FPL + LA + TB; SG + FPL + TB + MM + KQ; | | | | |
| | SG + EL + MM + FP + ES; PS + DS + FPL + LA + ES; | | | | |
| | EC + SG + PS + EL + MS; EC + DS + LA + TB + ES; | | | | |
| | SG + RT + FPL + KQ + RI; SG + FPL + LA + AH + FP; | | | | |
| | PS + DS + FPL + TB + FP; PS + EL + TB + FP + KQ; | | | | |
| | EC + SG + PS + RT + MS; SG + DS + LA + TB + ES; | | | | |
| | DS + MF + TB + AH + ES; FPL + LA + TB + MM + ES; | | | | |
| | EC + TB + MM + FP + ES; SG + DS + TB + MM + AH; | | | | |
| | EC + SG + PS + DS + FP; SG + RT + DS + MS + RI; | | | | |
| | SG + RT + FPL + LA + TB; PS + LA + TB + ES + KQ; | | | | |
| | PS + LA + MM + ES + RI; RT + DS + ES + MS + RI; | | | | |
| | EC + SG + PS + DS + MF; EC + SG + PS + FPL + ES; | | | | |
| | EC + SG + FPL + EL + AH; EC + SG + TB + FP + RI; | | | | |
| | SG + DS + FPL + FP + KQ; SG + FPL + MF + FP + ES; | | | | |
| | EC + SG + PS + AH + FP; SG + RT + EL + ES + KQ; | | | | |
| | SG + FPL + LA + MM + KQ; | | | | |
| | SG + LA + MM + AH + MS; PS + EL + TB + MM + KQ; | | | | |
| | DS + LA + TB + AH + MS; DS + MF + TB + ES + RI; | | | | |
| | EC + SG + FPL + LA + AH; EC + SG + FPL + MF + TB; | | | | |
| | EC + FPL + EL + TB + MM; EC + EL + MF + TB + ES; | | | | |
| | SG + FPL + LA + MF + TB; RT + FPL + EL + TB + KQ; | | | | |
| | RT + FPL + TB + FP + ES; DS + FPL + EL + ES + KQ; | | | | |
| | SG + PS + LA + TB + KQ; DS + EL + LA + TB + FP; | | | | |
| | DS + LA + TB + FP + KQ; EC + LA + TB + ES + RI; | | | | |
| | SG + RT + LA + FP + KQ; PS + DS + FPL + LA + RI; | | | | |
| | FPL + EL + LA + TB + FP; FPL + MF + TB + AH + FP; | | | | |
| | SG + DS + LA + ES + MS; DS + EL + LA + TB + ES; | | | | |
| | EC + PS + DS + TB + MM; EC + PS + TB + MM + KQ; | | | | |
| | SG + RT + DS + KQ + RI; SG + EL + LA + FP + MS; | | | | |
| | PS + DS + LA + TB + ES; EC + SG + PS + FP + RI; | | | | |
| | EC + PS + MM + AH + ES; RT + FPL + EL + FP + ES; | | | | |
| | EC + SG + FPL + AH + KQ; EC + SG + EL + ES + KQ; | | | | |
| | EC + FPL + EL + TB + FP; EC + LA + TB + MS + KQ; | | | | |
| | SG + RT + FPL + MF + FP; FPL + EL + TB + ES + KQ; | | | | |
| | EC + PS + DS + LA + RI; EC + PS + EL + AH + ES; | | | | |
| | SG + LA + AH + ES + MS; EC + DS + EL + MF + ES; | | | | |
| | PS + DS + FPL + TB + KQ; FPL + LA + TB + ES + KQ; | | | | |
| | EC + PS + DS + FPL + MF; EC + PS + MF + MM + ES; | | | | |
| | EC + FPL + MF + TB + FP; RT + EL + LA + TB + ES; | | | | |
| | EC + PS + RT + ES + RI; EC + FPL + LA + TB + ES; | | | | |
| | SG + PS + DS + MF + RI; SG + PS + FPL + MF + FP; | | | | |
| | SG + PS + EL + MM + RI; DS + FPL + EL + TB + RI; | | | | |
| | EC + SG + RT + KQ + RI; SG + PS + DS + AH + ES; | | | | |
| | SG + PS + FPL + LA + AH; SG + PS + TB + AH + ES; | | | | |
| | SG + RT + EL + FP + ES; EC + SG + FP + MS + RI; | | | | |
| | EC + PS + EL + ES + RI; SG + RT + LA + MS + KQ; | | | | |
| | SG + LA + TB + FP + MS; PS + RT + DS + ES + RI; | | | | |
| | DS + FPL + EL + MF + RI; EC + SG + PS + MF + FP; | | | | |
| | SG + DS + EL + LA + KQ; SG + DS + EL + MF + FP; | | | | |
| | DS + FPL + TB + FP + ES; EC + SG + LA + FP + MS; | | | | |
| | SG + EL + FP + ES + KQ; EC + SG + DS + ES + RI; | | | | |
| | EC + PS + DS + LA + KQ; SG + PS + FPL + LA + RI; | | | | |
| | SG + RT + DS + FPL + KQ; SG + RT + DS + LA + MS; | | | | |
| | SG + FPL + EL + ES + KQ; PS + EL + TB + MM + FP; | | | | |
| | DS + MF + TB + AH + FP; EC + PS + AH + ES + KQ; | | | | |
| | SG + FPL + EL + TB + MM; PS + EL + MM + ES + MS; | | | | |
| | DS + FPL + EL + TB + MM; DS + FPL + EL + TB + FP; | | | | |
| | LA + TB + MM + ES + KQ; EC + PS + RT + EL + ES; | | | | |
| | EC + PS + LA + TB + AH; SG + DS + LA + TB + KQ; | | | | |
| | EC + SG + PS + RT + DS; SG + FPL + EL + FP + RI; | | | | |
| | DS + FPL + TB + MS + RI; EL + LA + TB + FP + MS; | | | | |
| | SG + RT + FPL + FP + ES; PS + DS + FPL + MF + ES; | | | | |
| | RT + EL + LA + ES + MS; LA + TB + AH + ES + MS; | | | | |
| | EC + SG + DS + AH + MS; EC + PS + EL + MF + ES; | | | | |
| | SG + PS + FPL + LA + ES; SG + PS + LA + TB + AH; | | | | |
| | SG + FPL + LA + TB + RI; SG + FPL + AH + FP + ES; | | | | |
| | PS + DS + TB + AH + ES; EC + SG + PS + MM + AH; | | | | |
| | SG + DS + FPL + EL + MM; SG + EL + TB + ES + MS; | | | | |
| | PS + RT + FPL + ES + KQ; EC + SG + RT + TB + FP; | | | | |
| | EC + DS + FPL + AH + ES; SG + RT + EL + LA + KQ; | | | | |
| | SG + DS + EL + TB + FP; DS + FPL + TB + MM + KQ; | | | | |
| | FPL + EL + MF + TB + MM; EL + LA + TB + AH + ES; | | | | |
| | EC + SG + PS + MF + RI; EC + SG + DS + KQ + RI; | | | | |
| | SG + PS + RT + LA + ES; SG + LA + MF + TB + AH; | | | | |
| | PS + FPL + EL + FP + ES; PS + FPL + TB + MM + KQ; | | | | |
| | PS + EL + MM + FP + ES; EC + SG + DS + MS + KQ; | | | | |
| | SG + FPL + MF + TB + ES; PS + FPL + EL + TB + FP; | | | | |
| | DS + FPL + EL + ES + RI; EC + PS + MF + AH + ES; | | | | |
| | SG + FPL + MM + ES + KQ; EL + TB + FP + MS + RI; | | | | |
| | TB + AH + FP + ES + MS; EC + SG + EL + FP + KQ; | | | | |
| | EC + SG + TB + MM + RI; SG + DS + LA + TB + FP; | | | | |
| | SG + FPL + EL + LA + FP; EC + SG + DS + TB + ES; | | | | |
| | RT + FPL + ES + KQ + RI; EC + SG + PS + MF + AH; | | | | |
| | EC + SG + RT + EL + FP; EC + PS + DS + FPL + LA; | | | | |
| | EC + DS + LA + ES + RI; EC + DS + MF + TB + ES; | | | | |
| | SG + EL + FP + MS + RI; PS + EL + TB + FP + MS; | | | | |
| | DS + LA + MM + FP + MS; EL + LA + TB + MS + KQ; | | | | |
| | EC + SG + MF + FP + ES; EC + PS + TB + MM + RI; | | | | |
| | SG + DS + FPL + MM + ES; SG + FPL + LA + MM + FP; | | | | |
| | SG + EL + MM + ES + KQ; SG + EL + MM + ES + RI; | | | | |
| | SG + PS + LA + MM + AH; SG + RT + DS + LA + ES; | | | | |
| | PS + EL + LA + AH + ES; EC + DS + LA + FP + MS; | | | | |
| | SG + PS + FPL + TB + RI; SG + PS + TB + AH + FP; | | | | |
| | SG + RT + LA + TB + ES; DS + TB + MM + AH + ES; | | | | |
| | LA + TB + ES + MS + RI; EC + SG + PS + RT + KQ; | | | | |
| | EC + SG + EL + ES + RI; SG + RT + FP + KQ + RI; | | | | |
| | PS + DS + FPL + EL + MF; PS + DS + LA + AH + ES; | | | | |
| | PS + FPL + EL + TB + RI; EC + SG + LA + TB + KQ; | | | | |
| | SG + PS + FPL + EL + TB; SG + PS + FPL + FP + KQ; | | | | |
| | DS + FPL + TB + MS + KQ; FPL + LA + TB + AH + ES; | | | | |
| | FPL + MF + TB + FP + RI; EC + DS + FPL + MF + TB; | | | | |
| | SG + FPL + LA + ES + KQ; PS + DS + AH + ES + RI; | | | | |
| | EC + SG + DS + OS + LA; EC + SG + FPL + LA + KQ; | | | | |
| | EC + SG + LA + AH + RI; EC + PS + DS + LA + MF; | | | | |
| | SG + PS + RT + EL + ES; PS + FPL + EL + ES + RI; | | | | |
| | RT + LA + ES + MS + KQ; FPL + LA + TB + FP + RI; | | | | |
| | SG + EL + TB + AH + FP; SG + LA + ES + MS + KQ; | | | | |
| | RT + EL + LA + ES + KQ; EC + PS + MM + ES + RI; | | | | |
| | EC + DS + MF + ES + RI; SG + FPL + FP + ES + KQ; | | | | |
| | PS + LA + TB + AH + FP; EL + MF + TB + AH + FP; | | | | |
| | EC + SG + PS + LA + MF; EC + SG + DS + FPL + ES; | | | | |
| | EC + SG + DS + FPL + RI; EC + SG + LA + MF + ES; | | | | |
| | SG + EL + LA + TB + FP; FPL + EL + LA + TB + ES; | | | | |
| | EC + SG + LA + AH + KQ; EC + SG + TB + ES + KQ; | | | | |
| | EC + SG + MM + ES + KQ; LA + TB + FP + ES + RI; | | | | |
| | EC + SG + LA + MM + RI; EC + PS + DS + TB + KQ; | | | | |
| | EC + RT + LA + TB + ES; SG + PS + FPL + ES + KQ; | | | | |
| | DS + FPL + LA + TB + ES; EC + SG + PS + AH + MS; | | | | |
| | EC + SG + RT + MS + KQ; EC + SG + MF + TB + FP; | | | | |
| | EC + DS + TB + MM + FP; SG + DS + TB + AH + MS; | | | | |
| | RT + EL + TB + ES + KQ; EC + SG + RT + FPL + FP; | | | | |
| | SG + RT + MM + ES + RI; DS + TB + FP + MS + RI; | | | | |
| | EC + SG + LA + MM + KQ; PS + DS + FPL + ES + KQ; | | | | |
| | PS + FPL + TB + AH + ES; EC + RT + FPL + ES + KQ; | | | | |
| | EC + DS + FPL + LA + ES; EC + DS + ES + MS + RI; | | | | |
| | SG + DS + EL + AH + ES; SG + LA + TB + MM + ES; | | | | |
| | PS + DS + LA + MS + KQ; EL + TB + MM + ES + MS; | | | | |
| | EC + SG + DS + LA + ES; SG + TB + AH + FP + MS; | | | | |
| | DS + EL + LA + TB + AH; EC + EL + TB + AH + ES; | | | | |
| | SG + PS + FPL + ES + RI; SG + RT + DS + FPL + ES; | | | | |
| | SG + LA + MM + FP + MS; EC + SG + ES + MS + KQ; | | | | |
| | EC + PS + EL + TB + MM; SG + DS + FPL + FP + ES; | | | | |
| | EC + LA + TB + AH + FP; SG + PS + EL + LA + RI; | | | | |
| | SG + RT + FPL + LA + AH; PS + EL + TB + FP + RI; | | | | |
| | RT + DS + EL + ES + KQ; EC + PS + DS + MF + RI; | | | | |
| | EC + PS + FPL + TB + MM; EC + PS + TB + MS + RI; | | | | |
| | EC + RT + LA + ES + MS; EC + DS + EL + TB + RI; | | | | |
| | EC + DS + TB + MS + KQ; SG + PS + FPL + EL + FP; | | | | |
| | SG + PS + FPL + EL + ES; SG + PS + TB + MS + RI; | | | | |
| | SG + FPL + FP + MS + KQ; SG + LA + AH + FP + MS; | | | | |
| | PS + LA + TB + MS + KQ; EC + SG + DS + ES + KQ; | | | | |
| | SG + PS + AH + FP + ES; SG + RT + DS + ES + RI; | | | | |
| | SG + MF + TB + AH + FP; EC + SG + RT + FPL + AH; | | | | |
| | EC + PS + EL + MS + KQ; EC + RT + DS + LA + ES; | | | | |
| | EC + RT + ES + MS + RI; SG + PS + FPL + AH + KQ; | | | | |
| | DS + LA + MF + ES + RI; EL + LA + TB + MM + MS; | | | | |
| | EC + SG + PS + RT + FP; EC + PS + FPL + ES + RI; | | | | |
| | DS + FPL + MF + TB + KQ; DS + FPL + TB + MM + FP; | | | | |
| | EC + SG + LA + FP + RI; EC + PS + MF + ES + KQ; | | | | |
| | PS + DS + FPL + KQ + RI; DS + FPL + MF + TB + MS; | | | | |
| | EC + RT + DS + ES + MS; EC + SG + DS + MM + AH; | | | | |
| | EC + PS + DS + TB + FP; EC + PS + DS + MM + ES; | | | | |
| | EC + PS + FPL + LA + TB; SG + PS + EL + MF + RI; | | | | |
| | PS + RT + ES + KQ + RI; RT + TB + ES + MS + RI; | | | | |
| | EC + SG + RT + FP + RI; EC + PS + ES + KQ + RI; | | | | |
| | SG + DS + LA + MS + RI; EL + TB + MM + ES + KQ; | | | | |
| | EC + PS + RT + ES + KQ; EC + DS + FPL + EL + ES; | | | | |
| | EC + DS + LA + TB + FP; PS + RT + LA + ES + MS; | | | | |
| | PS + DS + FPL + EL + RI; PS + DS + LA + TB + FP; | | | | |
| | PS + LA + TB + MM + FP; EC + SG + DS + AH + KQ; | | | | |
| | EC + SG + TB + FP + KQ; EC + RT + DS + ES + RI; | | | | |
| | SG + RT + DS + EL + RI; SG + DS + MM + AH + RI; | | | | |
| | SG + LA + MM + ES + RI; EC + SG + EL + AH + FP; | | | | |
| | EC + PS + RT + TB + ES; EC + PS + FPL + MF + ES; | | | | |
| | SG + RT + FPL + FP + MS; SG + EL + LA + FP + KQ; | | | | |
| | RT + EL + FP + ES + RI; EC + SG + PS + EL + MF; | | | | |
| | EC + SG + PS + EL + TB; EC + SG + RT + DS + TB; | | | | |
| | EC + PS + RT + DS + ES; EC + PS + DS + LA + MM; | | | | |
| | EC + PS + MM + ES + MS; SG + DS + LA + FP + MS; | | | | |
| | DS + FPL + LA + ES + KQ; DS + FPL + TB + AH + FP; | | | | |
| | DS + LA + AH + FP + ES; EC + SG + FPL + LA + MS; | | | | |
| | SG + PS + FPL + KQ + RI; SG + LA + TB + MM + FP; | | | | |
| | SG + MF + TB + AH + ES; RT + LA + TB + AH + ES; | | | | |
| | EC + SG + DS + MF + AH; EC + PS + FPL + TB + FP; | | | | |
| | SG + PS + DS + LA + KQ; SG + RT + FPL + LA + MS; | | | | |
| | SG + RT + EL + TB + ES; SG + FPL + FP + KQ + RI; | | | | |
| | SG + LA + AH + ES + KQ; EC + FPL + TB + ES + MS; | | | | |
| | SG + RT + DS + FPL + LA; SG + RT + FPL + TB + FP; | | | | |
| | SG + FPL + LA + ES + RI; PS + DS + FPL + FP + RI; | | | | |
| | PS + DS + TB + MM + ES; RT + LA + ES + KQ + RI; | | | | |
| | FPL + TB + AH + FP + KQ; EC + PS + MF + ES + MS; | | | | |
| | EC + FPL + MF + TB + ES; SG + RT + ES + MS + RI; | | | | |
| | PS + EL + TB + MS + KQ; FPL + LA + TB + AH + FP; | | | | |
| | EC + RT + FPL + AH + ES; EC + RT + FPL + ES + MS; | | | | |
| | EC + RT + FPL + ES + RI; EC + DS + MF + TB + FP; | | | | |
| | RT + DS + FPL + TB + ES; EC + SG + TB + FP + MS; | | | | |
| | EC + RT + DS + MS + RI; EC + RT + EL + ES + MS; | | | | |
| | SG + RT + LA + MM + ES; SG + RT + LA + AH + KQ; | | | | |
| | SG + LA + TB + FP + KQ; PS + DS + FPL + LA + MF; | | | | |
| | PS + EL + MF + FP + ES; DS + FPL + MF + ES + RI; | | | | |
| | TB + MM + FP + ES + KQ; EC + SG + FPL + AH + FP; | | | | |
| | EC + SG + LA + AH + MS; EC + TB + ES + MS + RI; | | | | |
| | SG + DS + FPL + ES + MS; SG + DS + LA + ES + KQ; | | | | |
| | SG + EL + LA + TB + KQ; RT + EL + LA + ES + RI; | | | | |
| | DS + EL + FP + ES + MS; EC + PS + RT + ES + MS; | | | | |
| | SG + LA + FP + ES + KQ; DS + FPL + LA + MF + TB; | | | | |
| | EC + DS + MF + FP + MS; EC + DS + TB + FP + ES; | | | | |
| | EC + MF + TB + AH + FP; RT + TB + FP + ES + KQ; | | | | |
| | DS + LA + MF + TB + ES; EC + DS + EL + AH + ES; | | | | |
| | EC + LA + TB + FP + KQ; SG + RT + LA + TB + RI; | | | | |
| | SG + DS + FPL + EL + TB; DS + EL + FP + ES + RI; | | | | |
| | DS + LA + TB + MM + FP; EL + MF + TB + ES + RI; | | | | |
| | EC + PS + TB + FP + ES; EC + RT + DS + FPL + TB; | | | | |
| | EC + EL + TB + FP + KQ; SG + DS + FPL + MS + KQ; | | | | |
| | SG + FPL + FP + ES + RI; RT + DS + FPL + EL + TB; | | | | |
| | DS + EL + TB + AH + FP; FPL + EL + TB + FP + MS; | | | | |
| | EC + PS + EL + LA + TB; EC + DS + LA + MS + KQ; | | | | |
| | SG + PS + EL + AH + KQ; LA + TB + AH + FP + MS; | | | | |
| | EC + PS + RT + MM + ES; EC + TB + FP + ES + RI; | | | | |
| | SG + PS + DS + TB + AH; PS + RT + FPL + ES + RI; | | | | |
| | EC + SG + PS + RT + TB; EC + RT + FPL + LA + ES; | | | | |
| | EC + DS + TB + FP + KQ; SG + PS + LA + AH + KQ; | | | | |
| | SG + DS + EL + LA + FP; PS + DS + EL + AH + ES; | | | | |
| | FPL + TB + MM + ES + KQ; SG + EL + MF + FP + MS; | | | | |
| | PS + DS + FPL + MF + TB; DS + FPL + LA + TB + MS; | | | | |
| | FPL + TB + FP + KQ + RI; EL + LA + TB + MM + KQ; | | | | |
| | LA + MF + TB + AH + ES | | | | |
| 5 | EC + SG + EL + FP + ES; EC + SG + RT + DS + LA; | 0.79 | 0.321- | 50.79%- | 49.49%- |
| | EC + SG + RT + DS + ES; SG + EL + FP + ES + RI; | | 0.5048 | 96.83% | 94.95% |
| | EC + PS + MF + TB + ES; EC + PS + TB + ES + KQ; | | | | |
| | EL + TB + FP + ES + KQ; LA + TB + MM + ES + RI; | | | | |
| | EC + SG + PS + EL + LA; SG + DS + EL + FP + ES; | | | | |
| | PS + DS + FPL + EL + TB; DS + LA + TB + FP + ES; | | | | |
| | EC + PS + TB + AH + ES; DS + LA + TB + AH + FP; | | | | |
| | EC + SG + PS + MM + RI; SG + PS + DS + EL + ES; | | | | |
| | EC + SG + RT + LA + MS; EC + SG + DS + LA + MF; | | | | |
| | EC + LA + TB + MM + ES; SG + RT + FPL + EL + ES; | | | | |
| | SG + LA + AH + ES + RI; EC + SG + PS + FPL + LA; | | | | |
| | EC + PS + LA + ES + MS; SG + DS + FPL + LA + TB; | | | | |
| | SG + DS + FPL + FP + MS; SG + DS + TB + AH + RI; | | | | |
| | SG + TB + MM + AH + ES; EC + SG + RT + LA + KQ; | | | | |
| | MF + TB + AH + FP + ES; EC + SG + PS + ES + MS; | | | | |
| | SG + RT + FPL + ES + RI; SG + FPL + TB + FP + RI; | | | | |
| | SG + FPL + AH + ES + KQ; DS + TB + AH + FP + ES; | | | | |
| | EC + SG + FPL + TB + ES; PS + DS + FPL + TB + RI; | | | | |
| | SG + DS + LA + AH + MS; EC + SG + PS + LA + FP; | | | | |
| | EC + SG + DS + MS + RI; SG + DS + LA + AH + ES; | | | | |
| | DS + LA + TB + MM + MS; DS + FPL + TB + ES + RI; | | | | |
| | EC + SG + LA + AH + FP; EC + PS + DS + FPL + ES; | | | | |
| | SG + DS + FPL + EL + ES; EC + SG + FPL + TB + FP; | | | | |
| | SG + FPL + LA + TB + KQ; EC + SG + DS + TB + RI; | | | | |
| | SG + FPL + LA + AH + ES; SG + EL + TB + FP + MS; | | | | |
| | EC + SG + DS + MF + ES; DS + LA + TB + ES + RI; | | | | |
| | EC + SG + PS + DS + RI; PS + EL + ES + MS + KQ; | | | | |
| | LA + TB + FP + ES + MS; EC + SG + TB + AH + FP; | | | | |
| | EC + PS + EL + LA + ES; PS + TB + MM + ES + MS; | | | | |
| | EC + SG + PS + DS + MS; SG + DS + FPL + EL + KQ; | | | | |
| | PS + EL + MF + TB + ES; DS + FPL + MF + TB + FP; | | | | |
| | FPL + EL + TB + ES + RI; EC + PS + DS + AH + ES; | | | | |
| | PS + DS + LA + ES + RI; SG + FPL + LA + TB + ES; | | | | |
| | PS + EL + MM + ES + KQ; DS + LA + TB + AH + ES; | | | | |
| | EC + SG + RT + LA + TB; SG + PS + DS + FPL + TB; | | | | |
| | SG + EL + TB + MM + FP; TB + MM + AH + FP + ES; | | | | |
| | EC + FPL + EL + TB + ES; EC + SG + LA + TB + AH; | | | | |
| | EC + SG + RT + LA + RI; SG + RT + FPL + LA + ES; | | | | |
| | PS + LA + TB + AH + ES; SG + PS + FPL + FP + ES; | | | | |
| | SG + EL + TB + FP + KQ; EC + PS + LA + ES + KQ; | | | | |
| | EC + SG + PS + FPL + TB; EC + DS + TB + ES + RI; | | | | |
| | FPL + TB + FP + ES + KQ; SG + PS + EL + ES + MS; | | | | |
| | PS + DS + TB + ES + RI; DS + FPL + TB + ES + KQ; | | | | |
| | SG + PS + RT + LA + RI; SG + DS + FPL + AH + RI; | | | | |
| | FPL + EL + MF + TB + ES; SG + DS + LA + MS + KQ; | | | | |
| | EC + SG + RT + ES + RI; EC + SG + DS + MM + ES; | | | | |
| | SG + RT + LA + AH + ES; PS + TB + AH + FP + ES; | | | | |
| | EL + LA + TB + FP + ES; EC + SG + PS + LA + MM; | | | | |
| | SG + DS + FPL + TB + MS; EC + SG + DS + LA + KQ; | | | | |
| | SG + PS + LA + AH + ES; EC + SG + PS + FP + ES; | | | | |
| | EC + SG + EL + AH + ES; SG + PS + EL + TB + MM; | | | | |
| | DS + EL + TB + FP + RI; EC + PS + DS + ES + RI; | | | | |
| | SG + PS + DS + LA + RI; PS + EL + TB + ES + RI; | | | | |
| | EC + LA + MF + TB + ES; SG + PS + RT + FPL + LA; | | | | |
| | FPL + EL + TB + MM + KQ; EL + LA + TB + ES + MS; | | | | |
| | EC + PS + DS + LA + MS; PS + DS + FPL + ES + RI; | | | | |
| | DS + TB + MM + ES + MS; SG + EL + TB + ES + RI; | | | | |
| | SG + RT + LA + KQ + RI; PS + DS + FPL + LA + TB; | | | | |
| | SG + PS + DS + FPL + KQ; SG + DS + LA + ES + RI; | | | | |
| | DS + LA + TB + MM + KQ; EC + SG + PS + TB + RI; | | | | |
| | EC + PS + DS + TB + MS; SG + PS + RT + FPL + ES; | | | | |
| | SG + DS + FPL + EL + RI; RT + FPL + TB + ES + KQ; | | | | |
| | EC + SG + RT + FPL + RI; EC + PS + RT + LA + ES; | | | | |
| | EC + PS + DS + ES + MS; DS + FPL + TB + FP + RI; | | | | |
| | DS + TB + MM + ES + RI; EC + SG + DS + ES + MS; | | | | |
| | LA + TB + MM + MS + KQ; SG + FPL + TB + FP + KQ; | | | | |
| | PS + LA + TB + ES + MS; RT + DS + EL + TB + ES; | | | | |
| | EC + SG + PS + EL + FP; SG + EL + FP + ES + MS; | | | | |
| | FPL + MF + TB + FP + ES; EC + PS + FPL + TB + ES; | | | | |
| | SG + FPL + LA + FP + KQ; DS + FPL + EL + MF + FP; | | | | |
| | EC + SG + PS + EL + KQ; EC + SG + LA + MS + RI; | | | | |
| | EC + SG + MM + AH + ES; EC + PS + DS + TB + RI; | | | | |
| | SG + DS + LA + MM + AH; SG + PS + EL + MM + KQ; | | | | |
| | SG + PS + LA + ES + RI; SG + RT + FPL + FP + KQ; | | | | |
| | EC + SG + RT + EL + ES; SG + PS + DS + AH + RI; | | | | |
| | EC + SG + FPL + ES + RI; EC + PS + EL + ES + MS; | | | | |
| | SG + PS + TB + ES + RI; SG + DS + ES + MS + RI; | | | | |
| | RT + FPL + EL + TB + ES; EC + EL + TB + FP + ES; | | | | |
| | PS + DS + EL + FP + ES; DS + FPL + TB + AH + ES; | | | | |
| | EC + SG + PS + RT + RI; SG + PS + LA + AH + RI; | | | | |
| | LA + MF + TB + AH + FP; EC + SG + FP + ES + MS; | | | | |
| | SG + PS + EL + KQ + RI; SG + EL + MF + TB + FP; | | | | |
| | DS + LA + TB + ES + MS; EC + SG + RT + MM + ES; | | | | |
| | EC + DS + TB + MM + ES; SG + PS + EL + AH + ES; | | | | |
| | SG + FPL + TB + ES + KQ; DS + EL + TB + FP + MS; | | | | |
| | SG + RT + FPL + TB + ES; EC + SG + PS + TB + MS; | | | | |
| | PS + DS + FPL + EL + ES; EC + SG + RT + FP + ES; | | | | |
| | EC + RT + FPL + EL + ES; SG + DS + FPL + TB + FP; | | | | |
| | PS + RT + LA + ES + KQ; EL + TB + AH + FP + ES; | | | | |
| | EC + PS + LA + TB + MM; EC + RT + FPL + TB + ES; | | | | |
| | SG + FPL + TB + AH + FP; FPL + TB + FP + ES + RI; | | | | |
| | EC + DS + TB + AH + ES; SG + DS + EL + FP + MS; | | | | |
| | DS + FPL + TB + FP + KQ; SG + DS + TB + AH + FP; | | | | |
| | TB + MM + FP + ES + RI; EC + SG + FPL + ES + KQ; | | | | |
| | EC + SG + LA + ES + KQ; EC + DS + TB + FP + MS; | | | | |
| | SG + FPL + EL + TB + KQ; SG + FPL + LA + FP + RI; | | | | |
| | PS + LA + TB + ES + RI; EC + SG + LA + TB + RI; | | | | |
| | SG + PS + DS + FPL + FP; FPL + EL + TB + AH + ES; | | | | |
| | FPL + TB + AH + FP + ES; EC + SG + RT + ES + KQ; | | | | |
| | EC + PS + ES + MS + KQ; EC + DS + EL + TB + ES; | | | | |
| | SG + FPL + EL + FP + MS; SG + RT + FP + ES + RI; | | | | |
| | SG + LA + TB + AH + RI; EC + DS + FPL + TB + ES; | | | | |
| | SG + DS + FPL + EL + LA; PS + DS + FPL + TB + ES; | | | | |
| | SG + DS + FPL + MF + ES; SG + DS + AH + ES + RI; | | | | |
| | SG + LA + MM + AH + FP; SG + PS + FPL + LA + FP; | | | | |
| | SG + PS + MM + ES + RI; PS + EL + LA + FP + ES; | | | | |
| | EC + SG + PS + FP + MS; EC + SG + DS + TB + MS; | | | | |
| | SG + DS + FPL + AH + KQ; SG + FPL + TB + AH + KQ; | | | | |
| | SG + FPL + TB + FP + ES; PS + FPL + LA + TB + ES; | | | | |
| | EC + PS + MM + ES + KQ; SG + FPL + EL + LA + TB; | | | | |
| | EC + SG + RT + AH + ES; SG + RT + FPL + LA + FP; | | | | |
| | RT + EL + TB + FP + ES; EC + FPL + TB + AH + ES; | | | | |
| | SG + DS + FPL + ES + KQ; PS + LA + TB + FP + ES; | | | | |
| | PS + TB + MM + ES + RI; EC + SG + PS + LA + KQ; | | | | |
| | EC + SG + TB + ES + RI; SG + PS + LA + AH + FP; | | | | |
| | SG + DS + FPL + MS + RI; PS + RT + EL + ES + KQ; | | | | |
| | EC + SG + RT + DS + RI; EC + SG + RT + LA + FP; | | | | |
| | EC + DS + LA + ES + MS; SG + PS + FPL + TB + ES; | | | | |
| | SG + PS + RT + LA + KQ; SG + LA + MF + AH + FP; | | | | |
| | RT + LA + TB + FP + ES; FPL + EL + TB + FP + KQ; | | | | |
| | EC + SG + AH + ES + RI; SG + FPL + LA + AH + KQ; | | | | |
| | SG + FPL + TB + MM + ES; PS + FPL + MF + TB + FP; | | | | |
| | DS + FPL + LA + TB + FP; LA + TB + FP + MS + KQ; | | | | |
| | EC + PS + DS + FPL + TB; SG + DS + MF + AH + RI; | | | | |
| | RT + DS + EL + ES + RI; DS + FPL + MF + TB + RI; | | | | |
| | EC + SG + FP + ES + RI; SG + PS + RT + KQ + RI; | | | | |
| | SG + FPL + EL + FP + ES; EC + SG + LA + ES + MS; | | | | |
| | SG + PS + EL + TB + FP; SG + PS + EL + FP + MS; | | | | |
| | SG + PS + LA + ES + KQ; DS + FPL + LA + TB + MM; | | | | |
| | EC + SG + DS + LA + MS; PS + DS + FPL + MF + RI; | | | | |
| | RT + EL + ES + MS + RI; DS + EL + TB + MM + FP; | | | | |
| | EC + SG + PS + MS + RI; EC + SG + RT + FPL + KQ; | | | | |
| | EC + LA + TB + FP + MS; SG + DS + FPL + MM + KQ; | | | | |
| | EC + SG + MM + FP + ES; DS + LA + TB + MS + KQ; | | | | |
| | EC + SG + PS + DS + EL; PS + EL + TB + AH + ES; | | | | |
| | EC + SG + FPL + MF + ES; EC + SG + RT + FPL + ES; | | | | |
| | SG + FPL + LA + MF + FP; LA + TB + MM + AH + FP; | | | | |
| | SG + FPL + FP + ES + MS; EC + PS + DS + MF + TB; | | | | |
| | SG + PS + EL + MM + FP; SG + DS + FPL + EL + MF; | | | | |
| | EC + DS + TB + ES + MS; SG + PS + RT + FPL + RI; | | | | |
| | FPL + EL + TB + FP + RI; EC + SG + PS + DS + TB; | | | | |
| | EC + SG + RT + LA + MM; EC + SG + EL + LA + ES; | | | | |
| | EC + DS + LA + AH + ES; EC + DS + TB + MS + RI | | | | |
| 5 | SG + PS + EL + MM + ES; EC + SG + PS + LA + ES; | >=0.8 | 0.332- | 58.73%- | 47.47%- |
| | EC + SG + PS + MM + ES; SG + PS + EL + ES + RI; | | 0.4815 | 98.41% | 88.89% |
| | SG + PS + DS + FPL + RI; EC + PS + LA + TB + ES; | | | | |
| | PS + EL + TB + MM + ES; SG + PS + EL + FP + ES; | | | | |
| | EC + SG + PS + AH + ES; PS + EL + TB + FP + ES; | | | | |
| | EC + SG + PS + DS + LA; EC + SG + LA + TB + ES; | | | | |
| | PS + EL + TB + ES + MS; EC + SG + LA + AH + ES; | | | | |
| | PS + DS + EL + TB + ES; EC + SG + PS + ES + RI; | | | | |
| | PS + EL + LA + TB + ES; EC + SG + PS + TB + ES; | | | | |
| | LA + TB + MM + FP + ES; SG + LA + TB + AH + ES; | | | | |
| | EC + SG + DS + LA + TB; SG + FPL + EL + TB + FP; | | | | |
| | SG + PS + DS + FPL + ES; EC + SG + PS + EL + ES; | | | | |
| | SG + DS + FPL + TB + RI; DS + EL + TB + FP + ES; | | | | |
| | SG + DS + FPL + EL + FP; EC + SG + PS + DS + ES; | | | | |
| | EC + PS + DS + LA + ES; SG + DS + FPL + LA + AH; | | | | |
| | EC + PS + TB + MM + ES; EL + TB + FP + ES + RI; | | | | |
| | SG + DS + FPL + ES + RI; SG + DS + LA + TB + AH; | | | | |
| | PS + FPL + EL + TB + ES; EC + SG + PS + LA + RI; | | | | |
| | SG + PS + DS + ES + RI; SG + TB + AH + FP + ES; | | | | |
| | EL + MF + TB + FP + ES; SG + DS + FPL + TB + ES; | | | | |
| | SG + PS + EL + TB + ES; SG + PS + DS + FPL + EL; | | | | |
| | EL + TB + MM + ES + RI; SG + DS + FPL + LA + MF; | | | | |
| | SG + DS + EL + ES + RI; EC + SG + RT + LA + ES; | | | | |
| | EC + PS + LA + TB + MS; SG + FPL + EL + TB + ES; | | | | |
| | SG + DS + FPL + MF + RI; DS + FPL + EL + TB + ES; | | | | |
| | EC + SG + PS + RT + LA; EC + SG + PS + LA + TB; | | | | |
| | EC + SG + PS + LA + AH; EC + PS + DS + LA + TB; | | | | |
| | EC + SG + DS + LA + RI; EC + SG + EL + TB + ES; | | | | |
| | EC + SG + DS + EL + ES; EC + SG + TB + MM + ES; | | | | |
| | SG + DS + FPL + FP + RI; SG + DS + FPL + KQ + RI; | | | | |
| | EL + TB + MM + FP + ES; EC + SG + PS + RT + ES; | | | | |
| | EC + SG + TB + AH + ES; SG + PS + DS + FPL + LA; | | | | |
| | EL + TB + FP + ES + MS; EC + PS + DS + TB + ES; | | | | |
| | EC + SG + LA + TB + MS; SG + DS + FPL + AH + ES; | | | | |
| | SG + DS + FPL + MF + TB; EC + SG + PS + LA + MS; | | | | |
| | SG + DS + FPL + MF + FP; SG + LA + TB + AH + FP; | | | | |
| | FPL + EL + TB + FP + ES; EC + SG + FPL + EL + TB; | | | | |
| | PS + EL + TB + ES + KQ; EC + SG + RT + FPL + LA; | | | | |
| | SG + PS + EL + LA + ES; EC + PS + TB + ES + MS; | | | | |
| | SG + LA + AH + FP + ES; EC + SG + PS + MF + ES; | | | | |
| | FPL + EL + TB + MM + FP; SG + FPL + MF + TB + FP; | | | | |
| | DS + EL + MF + TB + FP; EC + PS + TB + ES + RI; | | | | |
| | SG + PS + EL + ES + KQ; EC + PS + EL + TB + ES; | | | | |
| | SG + DS + FPL + LA + RI; EC + SG + RT + DS + FPL; | | | | |
| | SG + PS + RT + ES + RI; SG + DS + LA + AH + RI; | | | | |
| | PS + EL + LA + TB + MM; EC + SG + LA + TB + MM; | | | | |
| | SG + LA + TB + AH + MS; SG + PS + DS + EL + RI; | | | | |
| | SG + FPL + EL + MF + FP; EC + SG + LA + ES + RI; | | | | |
| | SG + FPL + LA + TB + MM; SG + LA + TB + MM + AH; | | | | |
| | DS + TB + MM + FP + ES; EC + SG + PS + ES + KQ; | | | | |
| | SG + LA + TB + ES + KQ; EC + SG + FPL + LA + FP; | | | | |
| | SG + DS + FPL + TB + KQ; SG + DS + LA + AH + FP; | | | | |
| | SG + FPL + TB + AH + ES; SG + LA + MM + AH + ES; | | | | |
| | SG + EL + TB + FP + ES; SG + PS + DS + FPL + MF; | | | | |
| | SG + LA + TB + MS + KQ; EC + PS + LA + AH + ES; | | | | |
| | SG + FPL + EL + FP + KQ; LA + TB + AH + FP + ES; | | | | |
| | EC + SG + DS + AH + ES; EC + SG + EL + MM + ES; | | | | |
| | PS + LA + TB + MM + ES; EC + LA + TB + ES + MS; | | | | |
| | SG + RT + FPL + ES + KQ; EC + PS + DS + MF + ES; | | | | |
| | PS + EL + LA + ES + KQ; EC + DS + LA + TB + MS; | | | | |
| | SG + DS + FPL + LA + ES; FPL + TB + FP + ES + MS; | | | | |
| | SG + DS + FPL + LA + KQ; SG + FPL + LA + TB + FP; | | | | |
| | SG + DS + TB + AH + ES; EC + PS + DS + EL + ES; | | | | |
| | EC + LA + TB + AH + ES; SG + DS + FPL + LA + FP; | | | | |
| | SG + LA + TB + MM + KQ; SG + LA + MM + FP + ES; | | | | |
| | SG + PS + DS + LA + AH; SG + RT + FPL + LA + KQ; | | | | |
| | DS + FPL + MF + TB + ES | | | | |

TABLE 8
The frequency of the 18 markers in the 7178 combinations

| Bacterial marker | Freq |
|---|---|
| Escherichia_{—}coli | 2296 |
| Dorea_sp_AF36_15AT | 2184 |
| Turicibacter_{—}bilis | 2133 |
| Faecalibacterium prausnitzii | 2121 |
| Lawsonibacter_{—}asaccharolyticus | 2100 |
| Streptococcus_{—}gordonii | 2086 |
| Flavonifractor_{—}plautii | 2073 |
| Romboutsia_{—}timonensis | 2057 |
| Eggerthella_{—}sinensis | 2056 |
| Anaerostipes_{—}hadrus | 2053 |
| Phascolarctobacterium_SGB4573 | 2023 |
| Roseburia intestinalis | 1991 |
| Klebsiella_{—}quasipneumoniae | 1989 |
| Eggerthella_{—}lenta | 1978 |
| Megasphaera_sp_NM10 | 1919 |
| Megasphaera_{—}micronuciformis | 1833 |
| Megamonas_{—}funiformis | 1831 |
| Olsenella_sp_AF16_14LB | 1167 |

### Example 8: Workflow

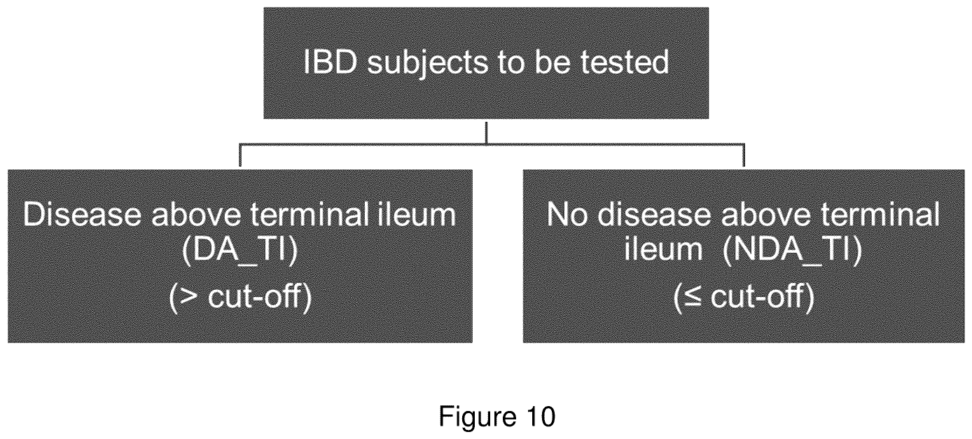

Referring now to FIG. 10, which shows the workflow of the test algorithm for diagnosing whether there is disease above TI or not in an individual suffering from IBD according to an example embodiment as described herein. Using the relative abundance of 18 bacterial biomarkers (Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, Roseburia intestinalis) determined by metagenomic sequencing as the inputs for the location classification model, the probability of disease above the terminal ileum (DA_TI) will be generated for each subject. Predetermined cut-off value is then applied to determine whether the IBD patients have disease above terminal ileum or not. If the probability of DA_TI is greater than the cut-off value (“cut-off”) (for example, range: 0.3-0.6), the subject will be considered to have disease above the terminal ileum (DA_TI), otherwise, it will be considered to have no disease above the TI (NDA_TI). In this embodiment as described in Examples 5-8, the cut-off value is 0.425.

In some embodiments, the test can be applied to individuals with unknown IBD subtypes, diagnosed CD patients, and diagnosed ulcerative colitis (UC) patients who are suspected of actually having CD or those whose disease may have evolved from CD to UC.

The methods of the present disclosure may be implemented in the form of one or more software applications running on one or more computer systems. Further, portions of the methods may be executed on one such computer system, while the other portions are executed on one or more other such computer systems. Examples of the computer system include a mainframe, personal computer, handheld computer, handheld mobile device, server, cloud-based servers, etc. The software application may be stored on a recording media locally accessible by the computer system and accessible via a hard wired or wireless connection to a network, for example, a local area network or on the internet.

The computer system may include, for example, a processor, random access memory (RAM), a printer interface, a display unit, a local area network (LAN) data transmission controller, a LAN interface, a network controller, an internal bus, and one or more input devices, for example, a keyboard, mouse, voice recognition device, etc. The computer system can be connected to a data storage device.

In some embodiments, the different components of the systems may be connected together through one or more networks. The networks can include one or more of a cloud network, the internet, an intranet, an extranet, a cellular network, a local area network (LAN), a home area network (HAN), metropolitan area network (MAN), a wide area network (WAN), a wireless network such as Wi-Fi network, Z-Wave network, Zigbee network, Thread network and Bluetooth network, public and private networks, etc.

The processor can be a central processing unit (CPU), microprocessor, microcontrollers, digital signal processor (DSP), field programmable gate arrays (FPGA), application-specific integrated circuits (ASIC), etc., for controlling the overall operation of memory (such as random access memory (RAM) for temporary data storage, read only memory (ROM) for permanent data storage, and firmware). One or more processors can communicate with each other and memory and perform operations and tasks that implement one or more blocks of the flow diagrams discussed herein.

The memory, for example, stores applications, data, programs, algorithms (including software to implement or assist in implementing example embodiments) and other data. Memory can include dynamic or static random-access memory (DRAM or SRAM) or read-only memory such as Erasable and Programmable Read-Only Memories (EPROMs), Electrically Erasable and Programmable Read-Only Memories (EEPROMs) and flash memories, as well as other memory technologies, singly or jointly combined. The memory, for example, stores applications, data, programs, algorithms (including software to implement or assist in implementing example embodiments) and other data.

The storage typically includes persistence storage such as magnetic disks such as fixed and removable disks; other magnetic media including tape; optical media such as Compact Disks (CDs) or Digital Versatile Disks (DVDs), and semiconductor storage devices such as flash memory cards, solid-state drive, EPROMs, EEPROMS or other storage technologies, singly or in combination. Note that the instructions of the software discussed herein can be provided on computer-readable or machine-readable storage medium, or alternatively, can be provided on multiple computer-readable or machine-readable storage media distributed in a large system having possibly plural nodes. Such computer-readable or machine-readable medium or media is (are) considered to be part of an article (or article of manufacture). An article or article of manufacture can refer to any manufactured single component or multiple components. In some embodiments, a software or instructions of the software are provided in a computer readable medium encoded with computer executable code, which is configured to enable the execution of steps of the methods in the preceding examples when the software or the instructions of the software are executed by a processor.

It should be understood for those skilled in the art that the division between hardware and software is a conceptual division for ease of understanding and is somewhat arbitrary. Moreover, it will be appreciated that peripheral devices in one computer installation may be integrated to the host computer in another. Furthermore, the application software systems may be executed in a distributed computing environment. The software program and its related databases can be stored in a separate file server or database server and is transferred to the local host for execution. Those skilled in the art will appreciate that alternative embodiments can be adopted to implement the present invention.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

### NUMBERED EMBODIMENTS

### Numbered Embodiments 1

Embodiment 1. A method for determining the risk of, diagnosing, preventing, or treating disease above the terminal ileum (TI) in an individual suffering from Inflammatory Bowel Disease (IBD), comprising the steps of: (a) determining the relative abundance of a set of bacterial species in a stool sample from the individual, wherein the set of bacterial species comprises three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis; (b) comparing the relative abundance of each bacterial species in the set of bacterial species from the individual with the relative abundance of each bacterial species in the set of bacterial species in a reference data set to generate a risk score; (c) determining the individual as having increased risk for or is suffering from disease above the TI if the risk score is higher than a cutoff value, or determining the individual as not having increased risk for or is not suffering from disease above the TI if the risk score is equal to or lower than the cutoff value; and (d) if the individual is determined to have an increased risk for or is suffering from disease above the TI, optionally treating the individual with an appropriate systemic therapy for disease above the TI, wherein the appropriate systemic therapy is selected from the group consisting of systemic corticosteroids, thiopurines, parenteral methotrexate, non-JAK inhibitor biologic agents, oral small molecule therapeutic agents for treating disease above the TI, and any combination thereof, or if the individual is determined to not have an increased risk for or is not suffering from disease above the TI, optionally treating the individual with an appropriate localized therapy, wherein the appropriate localized therapy is selected from the group consisting of sulfasalazine, enteric release budesonide, controlled ileal release (CIR) budesonide, oral methotrexate, an oral Janus kinase (JAK) inhibitor, or any combination thereof.

Embodiment 2. The method of embodiment 1, wherein the set of bacterial species comprises a group of three bacterial species selected from Table 3, a group of four bacterial species selected from Table 5, or a group of five bacterial species selected from Table 7.

Embodiment 3. The method of embodiment 1 or claim 2, wherein the reference data set is obtained by determining relative abundances of each bacterial species in the set of bacterial species in stool samples from a group of subjects comprising patients with Crohn's Disease (CD) in a reference cohort, wherein the patients with CD comprise patients with disease above the TI and patients without the disease above TI.

Embodiment 4. The method of any one of embodiments 1-3, wherein the comparing step in step (b) is performed using a machine learning model to generate the risk score.

Embodiment 5. The method of embodiment 4, wherein the machine learning model is constructed by (i) obtaining the reference data set by determining relative abundances of each bacterial species in the set of bacterial species in stool samples from a group of subjects comprising patients with CD in a reference cohort, wherein the patients with CD comprise patients with disease above the TI and patients without disease above the TI; (ii) obtaining a validation data set by determining relative abundances of each bacterial species in the set of bacterial species in stool samples from a second group of subjects comprising patients with CD and patients with Ulcerative Colitis (UC) in a validation cohort, wherein the patients with CD comprise patients with disease above the TI and patients without disease above the TI; (iii) dividing the reference data set into a training data set and a test data set; (iv) training a candidate model with the training data set and evaluating the performance of the candidate model using the validation data set; (v) optionally tuning the candidate model by adjusting hyperparameters in the candidate model; (vi) repeating step (iv) and optionally step (v) to produce a set of candidate models, and selecting an optimal candidate model among the set of candidate models; (vii) evaluating the performance of the optimal candidate model using the test data set; (viii) optionally training a final model with the reference data set using a set of hyperparameters corresponding to the optimal candidate model to produce the final model; and (ix) using the optimal candidate model of step (vii) or if step (viii) is used to train the final model, using the final model of step (viii) as the machine learning model for the comparing step in step (b) to generate the risk score.

Embodiment 6. The method of embodiment 4 or claim 5, wherein the machine learning model is a random forest model, and wherein the risk score in step (b) is generated by the following steps: (b1) generating an ensemble of decision trees by the random forest model using the relative abundance of the set of bacterial species in the reference data set; and (b2) running the relative abundance of the set of bacterial species from the individual along the ensemble of decision trees to generate the risk score.

Embodiment 7. The method of embodiment 5 or claim 6, wherein the optimal candidate model is the candidate model with the highest performance among the set of candidate models.

Embodiment 8. The method of any one of embodiments 5-7, wherein the performance of the candidate model is evaluated by Area Under the Curve (AUC) in Receiving Operating Characteristic (ROC) curve of the candidate model.

Embodiment 9. The method of any one of embodiments 1-8, wherein the cutoff value is in the range of 0.3 to 0.6.

Embodiment 10. The method of any one of embodiments 1-9, wherein the cutoff value is determined by the following steps: (c1) calculating a Youden's index using the risk score data in the reference data set; and (c2) determining the cutoff value based on the Youden's index.

Embodiment 11. The method of any one of embodiments 1-10, wherein the set of bacterial species consists essentially of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 12. The method of any one of embodiments 1-10, wherein the set of bacterial species comprises Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 13. The method of any one of embodiments 1-12, wherein the oral JAK inhibitor is upadacitinib.

Embodiment 14. A kit for determining the risk of or diagnosing disease above terminal ileum (TI) in an individual suffering from Inflammatory Bowel Disease (IBD), comprising a reagent for detecting a set of bacterial species, wherein the set of bacterial species comprises three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 15. The kit of embodiment 14, wherein the set of bacterial species comprises a group of three bacterial species selected from Table 3, a group of four bacterial species selected from Table 5, or a group of five bacterial species selected from Table 7.

Embodiment 16. The kit of embodiment 14 or claim 15, wherein the reagent comprises one or more sets of primers and optionally one or more fluorophore-quencher probes, wherein each set of primers is used for amplifying a target nucleic acid sequence belonging to a bacterial species in the set of bacterial species via PCR to produce an amplification product, and each fluorophore-quencher probe is used for detecting the amplification product of each set of primers.

Embodiment 17. The kit of any one of embodiments 14-16, wherein the set of bacterial species consisting essentially of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 18. The kit of any one of embodiments 14-16, wherein the set of bacterial species comprises Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 19. A composition comprising Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 20. A method of preventing or treating disease above terminal ileum (TI) in an individual suffering from Inflammatory Bowel Disease (IBD), comprising the step of: administering the composition of embodiment 19 to the individual.

### Numbered Embodiments 2

Embodiment 1. A method for identifying a stool sample with an altered microbiome associated with IBD with disease above terminal ileum (TI) comprising the steps of: (a) determining the relative abundance of a set of bacterial species in the stool sample, wherein the set of bacterial species comprises three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis; (b) comparing the relative abundance of each bacterial species in the set of bacterial species from the individual with the relative abundance of each bacterial species in the set of bacterial species in a reference data set to generate a risk score; and (c) determining the stool sample as having an altered microbiome associated with IBD with disease above the TI if the risk score is higher than a cutoff value.

Embodiment 2. The method of embodiment 1, wherein the set of bacterial species comprises a group of three bacterial species selected from Table 3, a group of four bacterial species selected from Table 5, or a group of five bacterial species selected from Table 7.

Embodiment 3. The method of embodiment 1 or claim 2, wherein the reference data set is obtained by determining relative abundances of each bacterial species in the set of bacterial species in stool samples from a group of subjects comprising patients with Crohn's Disease (CD) in a reference cohort, wherein the patients with CD comprise patients with disease above the TI and patients without disease above the TI.

Embodiment 4. The method of any one of embodiments 1-3, wherein the comparing step in step (b) is performed using a machine learning model to generate the risk score.

Embodiment 5. The method of embodiment 4, wherein the machine learning model is constructed by (i) obtaining the reference data set by determining relative abundances of each bacterial species in the set of bacterial species in stool samples from a group of subjects comprising patients with CD in a reference cohort, wherein the patients with CD comprise patients with disease above the TI and patients without disease above the TI; (ii) obtaining a validation data set by determining relative abundances of each bacterial species in the set of bacterial species in stool samples from a second group of subjects comprising patients with CD and patients with Ulcerative Colitis (UC) in a validation cohort, wherein the patients with CD comprise patients with disease above the TI and patients without disease above the TI; (iii) dividing the reference data set into a training data set and a test data set; (iv) training a candidate model with the training data set and evaluating the performance of the candidate model using the validation data set; (v) optionally tuning the candidate model by adjusting hyperparameters in the candidate model; (vi) repeating step (iv) and optionally step (v) to produce a set of candidate models, and selecting an optimal candidate model among the set of candidate models; (vii) evaluating the performance of the optimal candidate model using the test data set; (viii) optionally training a final model with the reference data set using a set of hyperparameters corresponding to the optimal candidate model to produce the final model; and (ix) using the optimal candidate model in step (vii) or if step (viii) is used to train the final model, using the final model in step (viii) as the machine learning model for the comparing step in step (b) to generate the risk score.

Embodiment 6. The method of embodiment 4 or claim 5, wherein the machine learning model is a random forest model, and wherein the risk score in step (b) is generated by the following steps: (b1) generating an ensemble of decision trees by the random forest model using the relative abundance of the set of bacterial species in the reference data set; and (b2) running the relative abundance of the set of bacterial species from the individual along the ensemble of decision trees to generate the risk score.

Embodiment 7. The method of embodiment 5 or claim 6, wherein the optimal candidate model is the candidate model with the highest performance among the set of candidate models.

Embodiment 8. The method of any one of embodiments 5-7, wherein the performance of the candidate model is evaluated by Area Under the Curve (AUC) in Receiving Operating Characteristic (ROC) curve of the candidate model.

Embodiment 9. The method of any one of embodiments 1-8, wherein the cutoff value is in the range of 0.3 to 0.6.

Embodiment 10. The method of any one of embodiments 1-9, wherein the cutoff value is determined by the following steps: (c1) calculating a Youden's index using the risk score data in the reference data set; and (c2) determining the cutoff value based on the Youden's index.

Embodiment 11. The method of any one of embodiments 1-10, wherein the set of bacterial species consists essentially of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 12. The method of any one of embodiments 1-10, wherein the set of bacterial species comprises Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 13. The method of any one of embodiments 1-12, wherein the relative abundance of the set of bacterial species is determined by metagenomic sequencing, quantitative PCR (qPCR) or droplet digital PCR (ddPCR).

Embodiment 14. A kit for determining the risk of or diagnosing disease above terminal ileum (TI) in an individual suffering from Inflammatory Bowel Disease (IBD), comprising a reagent for detecting a set of bacterial species, wherein the set of bacterial species comprises three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 15. The kit of embodiment 14, wherein the set of bacterial species comprises a group of three bacterial species selected from Table 3, a group of four bacterial species selected from Table 5, or a group of five bacterial species selected from Table 7.

Embodiment 16. The kit of embodiment 14 or claim 15, wherein the reagent comprises one or more sets of primers and optionally one or more fluorophore-quencher probes, wherein each set of primers is used for amplifying a target nucleic acid sequence belonging to a bacterial species in the set of bacterial species via PCR to produce an amplification product, and each fluorophore-quencher probe is used for detecting the amplification product of each set of primers.

Embodiment 17. The kit of any one of embodiments 14-16, wherein the set of bacterial species consisting essentially of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 18. The kit of any one of embodiments 14-16, wherein the set of bacterial species comprises Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 19. A composition comprising Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 20. Use of the composition of embodiment 19 for the manufacture of a medicament for the treatment of disease above terminal ileum (TI) in an individual suffering from Inflammatory Bowel Disease (IBD).

Embodiment 21. A computer program product for determining the risk, diagnosing, or suggesting treatment for disease above terminal ileum (TI) in an individual suffering from Inflammatory Bowel Disease (IBD), wherein the computer program product comprises a computer readable medium encoded with computer executable code, wherein the computer executable code is configured to enable the execution of the steps of: (a) determining the relative abundance of a set of bacterial species in a stool sample from the individual, wherein the set of bacterial species comprises three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis; (b) comparing the relative abundance of each bacterial species in the set of bacterial species from the individual with the relative abundance of each bacterial species in the set of bacterial species in a reference data set to generate a risk score; (c) determining the individual as having increased risk for or is suffering from disease above the TI if the risk score is higher than a cutoff value, or determining the individual as not having increased risk for or is not suffering from disease above the TI if the risk score is equal to or lower than the cutoff value; and (d) if the individual is determined to have an increased risk for or is suffering from disease above the TI, suggesting an appropriate systemic therapy for disease above the TI, wherein the appropriate systemic therapy is selected from the group consisting of systemic corticosteroids, thiopurines, parenteral methotrexate, non-JAK inhibitor biologic agents, oral small molecule therapeutic agents for treating disease above the TI, and any combination thereof; or if the individual is determined to not have an increased risk for or is not suffering from disease above the TI, suggesting an appropriate localized therapy, wherein the appropriate localized therapy is selected from the group consisting of sulfasalazine, enteric release budesonide, controlled ileal release (CIR) budesonide, oral methotrexate, an oral Janus kinase (JAK) inhibitor, or any combination thereof.

Embodiment 22. The computer program product of embodiment 21, wherein the set of bacterial species comprises a group of three bacterial species selected from Table 3, a group of four bacterial species selected from Table 5, or a group of five bacterial species selected from Table 7.

Embodiment 23. The computer program product of embodiment 21 or claim 22, wherein the reference data set is obtained by determining relative abundances of each bacterial species in the set of bacterial species in stool samples from a group of subjects comprising patients with Crohn's Disease (CD) in a reference cohort, wherein the patients with CD comprise patients with disease above the TI and patients without disease above the TI.

Embodiment 24. The computer program product of any one of embodiments 21-23, wherein the comparing step in step (b) is performed using a machine learning model to generate the risk score.

Embodiment 25. The computer program product of embodiment 24, wherein the machine learning model is constructed by (i) obtaining the reference data set by determining relative abundances of each bacterial species in the set of bacterial species in stool samples from a group of subjects comprising patients with CD in a reference cohort, wherein the patients with CD comprise patients with disease above the TI and patients without disease above the TI; (ii) obtaining a validation data set by determining relative abundances of each bacterial species in the set of bacterial species in stool samples from a second group of subjects comprising patients with CD and patients with Ulcerative Colitis (UC) in a validation cohort, wherein the patients with CD comprise patients with disease above the TI and patients without disease above the TI; (iii) dividing the reference data set into a training data set and a test data set; (iv) training a candidate model with the training data set and evaluating the performance of the candidate model using the validation data set; (v) optionally tuning the candidate model by adjusting hyperparameters in the candidate model; (vi) repeating step (iv) and optionally step (v) to produce a set of candidate models, and selecting an optimal candidate model among the set of candidate models; (vii) evaluating the performance of the optimal candidate model using the test data set; (viii) optionally training a final model with the reference data set using a set of hyperparameters corresponding to the optimal candidate model to produce the final model; and (ix) using the optimal candidate model of step (vii) or if step (viii) is used to train the final model, using the final model of step (viii) as the machine learning model for the comparing step in step (b) to generate the risk score.

Embodiment 26. The computer program product of embodiment 24 or claim 25, wherein the machine learning model is a random forest model, and wherein the risk score in step (b) is generated by the following steps: (b1) generating an ensemble of decision trees by the random forest model using the relative abundance of the set of bacterial species in the reference data set; and (b2) running the relative abundance of the set of bacterial species from the individual along the ensemble of decision trees to generate the risk score.

Embodiment 27. The computer program product of embodiment 25 or claim 26, wherein the optimal candidate model is the candidate model with the highest performance among the set of candidate models.

Embodiment 28. The computer program product of any one of embodiments 25-27, wherein the performance of the candidate model is evaluated by Area Under the Curve (AUC) in Receiving Operating Characteristic (ROC) curve of the candidate model.

Embodiment 29. The computer program product of any one of embodiments 21-28, wherein the cutoff value is in the range of 0.3-0.6.

Embodiment 30. The computer program product of any one of embodiments 21-29, wherein the cutoff value is determined by the following steps: (c1) calculating a Youden's index using the risk score data in the reference data set; and (c2) determining the cutoff value based on the Youden's index.

Embodiment 31. The computer program product of any one of embodiments 21-30, wherein the set of bacterial species consisting essentially of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

Embodiment 32. The computer program product of any one of embodiments 21-30, wherein the set of bacterial species comprises Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

### Numbered Embodiments 3

Embodiment 1. A method for determining the risk of, diagnosing, preventing, or treating ileum involvement in an individual suffering from Inflammatory Bowel Disease (IBD), comprising the steps of: (a) determining the relative abundance of a set of bacterial species in a stool sample from the individual, wherein the set of bacterial species comprises one or more bacterial species selected from the group consisting of Faecalimonas umbilicate, Akkermansia muciniphila, Enterocloster clostridioformis, Evtepia gabavorous, Ruminococcus gnavus, Enterocloster bolteae, Enterocloster aldenensis, Erysipelatoclostridium ramosum, Escherichia coli, Fusobacterium ulcerans, Enterocloster citroniae, Anaerotruncus colihominis, Clostridium SGB4750, Phascolarctobacterium faecium, Ruminococcus torques, Fusobacterium mortiferum, Clostridium fessum, Bilophila wadsworthia, Fusobacterium nucleatum, Klebsiella pneumoniae, Veillonella parvula, Phascolarctobacterium SGB4573, Allisonella histaminiformans, Lachnospiraceae bacterium NSJ-29, Romboutsia timonensis, Faecalibacterium prausnitzii, Flavonifractor plautii, Faecalibacterium SGB15346, Dorea formicigenerans, Anaerobutyricum hallii, Haemophilus parainfluenzae, and Streptococcus australis; (b) comparing the relative abundance of each bacterial species in the set of bacterial species from the individual with the relative abundance of each bacterial species in the set of bacterial species in a reference data set to generate a risk score; (c) determining the individual as having increased risk for or is suffering from ileum involvement if the risk score is higher than a cutoff value; and (d) if the individual is determined to have an increased risk for or is suffering from ileum involvement, optionally treating the individual.

Embodiment 2. The method of embodiment 1, wherein the set of bacterial species comprises one or more bacterial species selected from the group consisting of Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, SGB4750, Ruminococcus torques, Allisonella histaminiformans, Romboutsia timonensis, Faecalibacterium prausnitzii, and Haemophilus parainfluenzae.

Embodiment 3. The method of embodiment 1 or claim 2, wherein the reference data set is obtained by determining relative abundances of each bacterial species in the set of bacterial species in stool samples from a group of subjects comprising patients with colonic IBD and patients with ileal Crohn's Disease (CD) in a reference cohort, wherein the colonic IBD comprises Ulcerative Colitis and colonic CD.

Embodiment 4. The method of any one of embodiments 1-3, wherein the comparing step in step (b) is performed using a machine learning model to generate the risk score.

Embodiment 5. The method of embodiment 4, wherein the machine learning model is constructed by (i) obtaining the reference data set by determining relative abundances of each bacterial species in the set of bacterial species in stool samples from a group of subjects comprising patients with colonic IBD and patients with ileal Crohn's Disease (CD) in a reference cohort; (ii) dividing the reference data set into a training data set and a test data set; (iii) dividing the training data set into a training subset and a validation subset; (iv) training a candidate model with the training subset and evaluating the performance of the candidate model using the validation subset; (v) optionally tuning the candidate model by adjusting hyperparameters in the candidate model; (vi) repeating step (iv) and optionally step (v) to produce a set of candidate models, and selecting an optimal candidate model among the set of candidate models; (vii) evaluating the performance of the optimal candidate model using the test data set; (viii) optionally training a final model with the reference data set using a set of hyperparameters corresponding to the optimal candidate model to produce the final model; and (ix) using the optimal candidate model of step (vii) or if step (viii) is used to train the final model, using the final model of step (viii) as the machine learning model for the comparing step in step (b) to generate the risk score.

Embodiment 6. The method of embodiment 4 or claim 5, wherein the machine learning model is a random forest model, and wherein the risk score in step (b) is generated by the following steps: (b1) generating an ensemble of decision trees by the random forest model using the relative abundance of the set of bacterial species in the reference data set; and (b2) running the relative abundance of the set of bacterial species from the individual along the ensemble of decision trees to generate the risk score.

Embodiment 7. The method of embodiment 5 or claim 6, wherein the optimal candidate model is the candidate model with the highest performance among the set of candidate models.

Embodiment 8. The method of any one of embodiments 5-7, wherein the performance of the candidate model is evaluated by Area Under the Curve (AUC) in Receiving Operating Characteristic (ROC) curve of the candidate model.

Embodiment 9. The method of any one of embodiments 1-8, wherein the cutoff value is in the range of 0.1 to 0.3.

Embodiment 10. The method of any one of embodiments 1-9, wherein the cutoff value is determined by the following steps: (c1) calculating a Youden's index using the risk score data in the reference data set; and (c2) determining the cutoff value based on the Youden's index.

Embodiment 11. The method of any one of embodiments 1-10, wherein the set of bacterial species consists essentially of Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, Allisonella histaminiformans, Romboutsia timonensis, Faecalibacterium prausnitzii, and Haemophilus parainfluenzae.

Embodiment 12. The method of any one of embodiments 1-10, wherein the set of bacterial species comprises Faecalimonas umbilicate, Akkermansia muciniphila, Enterocloster clostridioformis, Evtepia gabavorous, Ruminococcus gnavus, Enterocloster bolteae, Enterocloster aldenensis, Erysipelatoclostridium ramosum, Escherichia coli, Fusobacterium ulcerans, Enterocloster citroniae, Anaerotruncus colihominis, Clostridium SGB4750, Phascolarctobacterium faecium, Ruminococcus torques, Fusobacterium mortiferum, Clostridium fessum, Bilophila wadsworthia, Fusobacterium nucleatum, Klebsiella pneumoniae, Veillonella parvula, Phascolarctobacterium SGB4573, Allisonella histaminiformans, and Lachnospiraceae bacterium NSJ-29.

Embodiment 13. The method of any one of embodiments 1-10, wherein the set of bacterial species comprises Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, and Allisonella histaminiformans.

Embodiment 14. The method of any one of embodiments 1-10, wherein the set of bacterial species comprises Romboutsia timonensis, Faecalibacterium prausnitzii, Flavonifractor plautii, Faecalibacterium SGB15346, Dorea formicigenerans, Anaerobutyricum hallii, Haemophilus parainfluenzae and Streptococcus australis.

Embodiment 15. The method of any of the embodiments 1-14, wherein the relative abundance of the set of bacterial species is determined by metagenomic sequencing, quantitative PCR (qPCR) or droplet digital PCR (ddPCR).

Embodiment 16. The method of any one of embodiments 1-15, wherein if the individual is determined to have an increased risk for or is suffering from ileum involvement, treating the individual with an appropriate treatment, wherein the appropriate treatment is selected from the group consisting of a therapeutic treatment, fecal microbiota transplantation (FMT), intestinal microbiota transplantation (IMT), lifestyle and diet modifications, and surgical intervention.

Embodiment 17. The method of embodiment 16, wherein the therapeutic treatment is selected from the group consisting of, a steroid, a thiopurine, a TNF inhibitor, an anti-integrin, and an anti-interleukin (IL) 12/23.

Embodiment 18. The method of embodiment 16, wherein the therapeutic treatment is controlled ileal release budesonide.

Embodiment 19. A method for identifying a stool sample with an altered microbiome associated with IBD with ileal involvement comprising the steps of: (a) determining the relative abundance of a set of bacterial species in the stool sample, wherein the set of bacterial species comprises one or more bacterial species selected from the group consisting of Faecalimonas umbilicate, Akkermansia muciniphila, Enterocloster clostridioformis, Evtepia gabavorous, Ruminococcus gnavus, Enterocloster bolteae, Enterocloster aldenensis, Erysipelatoclostridium ramosum, Escherichia coli, Fusobacterium ulcerans, Enterocloster citroniae, Anaerotruncus colihominis, Clostridium SGB4750, Phascolarctobacterium faecium, Ruminococcus torques, Fusobacterium mortiferum, Clostridium fessum, Bilophila wadsworthia, Fusobacterium nucleatum, Klebsiella pneumoniae, Veillonella parvula, Phascolarctobacterium SGB4573, Allisonella histaminiformans, Lachnospiraceae bacterium NSJ-29, Romboutsia timonensis, Faecalibacterium prausnitzii, Flavonifractor plautii, Faecalibacterium SGB15346, Dorea formicigenerans, Anaerobutyricum hallii, Haemophilus parainfluenzae, and Streptococcus australis; (b) comparing the relative abundance of each bacterial species in the set of bacterial species from the individual with the relative abundance of each bacterial species in the set of bacterial species in a reference data set to generate a risk score; and (c) determining the stool sample as having an altered microbiome associated with IBD with ileal involvement if the risk score is higher than a cutoff value.

Embodiment 20. The method of embodiment 19, wherein the set of bacterial species comprises one or more bacterial species selected from the group consisting of Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, Allisonella histaminiformans, Romboutsia timonensis, Faecalibacterium prausnitzii, and Haemophilus parainfluenzae.

Embodiment 21. The method of embodiment 19 or claim 20, wherein the reference data set is obtained by determining relative abundances of each bacterial species in the set of bacterial species in stool samples from a group of subjects comprising patients with colonic IBD and patients with ileal Crohn's Disease (CD) in a reference cohort, wherein the colonic IBD comprises Ulcerative Colitis and colonic CD.

Embodiment 22. The method of any one of embodiments 19-21, wherein the comparing step in step (b) is performed using a machine learning model to generate the risk score.

Embodiment 23. The method of embodiment 22, wherein the machine learning model is constructed by (i) obtaining the reference data set by determining relative abundances of each bacterial species in the set of bacterial species in stool samples from a group of subjects comprising patients with colonic IBD and patients with ileal Crohn's Disease (CD) in a reference cohort; (ii) dividing the reference data set into a training data set and a test data set; (iii) dividing the training data set into a training subset and a validation subset; (iv) training a candidate model with the training subset and evaluating the performance of the candidate model using the validation subset; (v) optionally tuning the candidate model by adjusting hyperparameters in the candidate model; (vi) repeating step (iii) and optionally step (iv) to produce a set of candidate models, and selecting an optimal candidate model among the set of candidate models; (vii) evaluating the performance of the optimal candidate model using the test data set; (viii) optionally training a final model with the reference data set using a set of hyperparameters corresponding to the optimal candidate model to produce the final model; and (ix) using the optimal candidate model in step (vi) or if step (vii) is used to train the final model, using the final model in step (vii) as the machine learning model for the comparing step in step (b) to generate the risk score.

Embodiment 24. The method of embodiment 22 or claim 23, wherein the machine learning model is a random forest model, and wherein the risk score in step (b) is generated by the following steps: (b1) generating an ensemble of decision trees by the random forest model using the relative abundance of the set of bacterial species in the reference data set; and (b2) running the relative abundance of the set of bacterial species from the individual along the ensemble of decision trees to generate the risk score.

Embodiment 25. The method of embodiment 23 or claim 24, wherein the optimal candidate model is the candidate model with the highest performance among the set of candidate models.

Embodiment 26. The method of any one of embodiments 23-25, wherein the performance of the candidate model is evaluated by Area Under the Curve (AUC) in Receiving Operating Characteristic (ROC) curve of the candidate model.

Embodiment 27. The method of any one of embodiments 19-26, wherein the cutoff value is in the range of 0.1 to 0.3.

Embodiment 28. The method of any one of embodiments 19-27, wherein the cutoff value is determined by the following steps: (c1) calculating a Youden's index using the risk score data in the reference data set; and (c2) determining the cutoff value based on the Youden's index.

Embodiment 29. The method of any one of embodiments 19-28, wherein the set of bacterial species consists essentially of Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, Allisonella histaminiformans, Romboutsia timonensis, Faecalibacterium prausnitzii, and Haemophilus parainfluenzae.

Embodiment 30. The method of any one of embodiments 19-28, wherein the set of bacterial species comprises Faecalimonas umbilicate, Akkermansia muciniphila, Enterocloster clostridioformis, Evtepia gabavorous, Ruminococcus gnavus, Enterocloster bolteae, Enterocloster aldenensis, Erysipelatoclostridium ramosum, Escherichia coli, Fusobacterium ulcerans, Enterocloster citroniae, Anaerotruncus colihominis, Clostridium SGB4750, Phascolarctobacterium faecium, Ruminococcus torques, Fusobacterium mortiferum, Clostridium fessum, Bilophila wadsworthia, Fusobacterium nucleatum, Klebsiella pneumoniae, Veillonella parvula, Phascolarctobacterium SGB4573, Allisonella histaminiformans, and Lachnospiraceae bacterium NSJ-29.

Embodiment 31. The method of any one of embodiments 19-28, wherein the set of bacterial species comprises Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, and Allisonella histaminiformans.

Embodiment 32. The method of any one of embodiments 19-28, wherein the set of bacterial species comprises Romboutsia timonensis, Faecalibacterium prausnitzii, Flavonifractor plautii, Faecalibacterium SGB15346, Dorea formicigenerans, Anaerobutyricum hallii, Haemophilus parainfluenzae, and Streptococcus australis.

Embodiment 33. The method of any one of embodiments 19-32, wherein the relative abundance of the set of bacterial species is determined by metagenomic sequencing, quantitative PCR (qPCR) or droplet digital PCR (ddPCR).

Embodiment 34. A kit for determining the risk of or diagnosing ileum involvement in an individual suffering from Inflammatory Bowel Disease (IBD), comprising a reagent for detecting a set of bacterial species, wherein the set of bacterial species comprises one or more bacterial species selected from the group consisting of Faecalimonas umbilicate, Akkermansia muciniphila, Enterocloster clostridioformis, Evtepia gabavorous, Ruminococcus gnavus, Enterocloster bolteae, Enterocloster aldenensis, Erysipelatoclostridium ramosum, Escherichia coli, Fusobacterium ulcerans, Enterocloster citroniae, Anaerotruncus colihominis, Clostridium SGB4750, Phascolarctobacterium faecium, Ruminococcus torques, Fusobacterium mortiferum, Clostridium fessum, Bilophila wadsworthia, Fusobacterium nucleatum, Klebsiella pneumoniae, Veillonella parvula, Phascolarctobacterium SGB4573, Allisonella histaminiformans, Lachnospiraceael bacterium NSJ-29, Romboutsia timonensis, Faecalibacterium prausnitzii, Flavonifractor plautii, Faecalibacterium SGB15346, Dorea formicigenerans, Anaerobutyricum hallii, Haemophilus parainfluenzae, and Streptococcus australis.

Embodiment 35. The kit of embodiment 34, wherein the set of bacterial species comprises one or more bacterial species selected from the group consisting of Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, Allisonella histaminiformans, Romboutsia timonensis, Faecalibacterium prausnitzii, and Haemophilus parainfluenzae.

Embodiment 36. The kit of embodiment 34 or claim 35, wherein the reagent comprises one or more sets of primers and optionally one or more fluorophore-quencher probes, wherein each set of primers is used for amplifying a target nucleic acid sequence belonging to a bacterial species in the first set of bacterial species or the second set of bacterial species via PCR to produce an amplification product, and each fluorophore-quencher probe is used for detecting the amplification product of each set of primers.

Embodiment 37. The kit of any one of embodiments 34-36, wherein the set of bacterial species consisting essentially of Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, Allisonella histaminiformans, Romboutsia timonensis, Faecalibacterium prausnitzii, and Haemophilus parainfluenzae.

Embodiment 38. The kit of any one of embodiments 34-36, wherein the set of bacterial species comprises Faecalimonas umbilicate, Akkermansia muciniphila, Enterocloster clostridioformis, Evtepia gabavorous, Ruminococcus gnavus, Enterocloster bolteae, Enterocloster aldenensis, Erysipelatoclostridium ramosum, Escherichia coli, Fusobacterium ulcerans, Enterocloster citroniae, Anaerotruncus colihominis, Clostridium SGB4750, Phascolarctobacterium faecium, Ruminococcus torques, Fusobacterium mortiferum, Clostridium fessum, Bilophila wadsworthia, Fusobacterium nucleatum, Klebsiella pneumoniae, Veillonella parvula, Phascolarctobacterium SGB4573, Allisonella histaminiformans, and Lachnospiraceae bacterium NSJ-29.

Embodiment 39. The kit of any one of embodiments 34-36, wherein the set of bacterial species comprises Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, and Allisonella histaminiformans.

Embodiment 40. The kit of any one of embodiments 34-36, wherein the set of bacterial species comprises Romboutsia timonensis, Faecalibacterium prausnitzii, Flavonifractor plautii, Faecalibacterium SGB15346, Dorea formicigenerans, Anaerobutyricum hallii, Haemophilus parainfluenzae and Streptococcus australis.

Embodiment 41. A computer program product for determining the risk, diagnosing, or suggesting treatment for ileum involvement in an individual suffering from Inflammatory Bowel Disease (IBD), wherein the computer program product comprises a computer readable medium encoded with computer executable code, wherein the computer executable code is configured to enable the execution of the steps of: (a) determining the relative abundance of a set of bacterial species in a stool sample from the individual, wherein the set of bacterial species comprises one or more bacterial species selected from the group consisting of Faecalimonas umbilicate, Akkermansia muciniphila, Enterocloster clostridioformis, Evtepia gabavorous, Ruminococcus gnavus, Enterocloster bolteae, Enterocloster aldenensis, Erysipelatoclostridium ramosum, Escherichia coli, Fusobacterium ulcerans, Enterocloster citroniae, Anaerotruncus colihominis, Clostridium SGB4750, Phascolarctobacterium faecium, Ruminococcus torques, Fusobacterium mortiferum, Clostridium fessum, Bilophila wadsworthia, Fusobacterium nucleatum, Klebsiella pneumoniae, Veillonella parvula, Phascolarctobacterium SGB4573, Allisonella histaminiformans, Lachnospiraceae bacterium NSJ-29, Romboutsia timonensis, Faecalibacterium prausnitzii, Flavonifractor plautii, Faecalibacterium SGB15346, Dorea formicigenerans, Anaerobutyricum hallii, Haemophilus parainfluenzae, and Streptococcus australis; (b) comparing the relative abundance of each bacterial species in the set of bacterial species from the individual with the relative abundance of each bacterial species in the set of bacterial species in a reference data set to generate a risk score; (c) determining the individual as having increased risk for or is suffering from ileum involvement if the risk score is higher than a cutoff value; and (d) if the individual is determined to have an increased risk for or is suffering from ileum involvement, suggesting an appropriate treatment to the individual.

Embodiment 42. The computer program product of embodiment 41, wherein the set of bacterial species comprises one or more bacterial species selected from the group consisting of Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, Allisonella histaminiformans, Romboutsia timonensis, Faecalibacterium prausnitzii, and Haemophilus parainfluenzae.

Embodiment 43. The computer program product of embodiment 41 or claim 42, wherein the reference data set is obtained by determining relative abundances of each bacterial species in the set of bacterial species in stool samples from a group of subjects comprising patients with colonic IBD and patients with ileal Crohn's Disease (CD) in a reference cohort.

Embodiment 44. The computer program product of any one of embodiments 41-43, wherein the comparing step in step (b) is performed using a machine learning model to generate the risk score.

Embodiment 45. The computer program product of embodiment 44, wherein the machine learning model is constructed by (i) obtaining the reference data set by determining relative abundances of each bacterial species in the set of bacterial species in stool samples from a group of subjects comprising patients with colonic IBD and patients with ileal Crohn's Disease (CD) in a reference cohort; (ii) dividing the reference data set into a training data set and a test data set; (iii) dividing the training data set into a training subset and a validation subset; (iv) training a candidate model with the training subset and evaluating the performance of the candidate model using the validation subset; (v) optionally tuning the candidate model by adjusting hyperparameters in the candidate model; (vi) repeating step (iii) and optionally step (iv) to produce a set of candidate models, and selecting an optimal candidate model among the set of candidate models; (vii) evaluating the performance of the optimal candidate model using the test data set; (viii) optionally training a final model with the reference data set using a set of hyperparameters corresponding to the optimal candidate model to produce the final model; and (ix) using the optimal candidate model of step (vi) or if step (vii) is used to train the final model, using the final model of step (vii) as the machine learning model for the comparing step in step (b) to generate the risk score.

Embodiment 46. The computer program product of embodiment 44 or claim 45, wherein the machine learning model is a random forest model, and wherein the risk score in step (b) is generated by the following steps: (b1) generating an ensemble of decision trees by the random forest model using the relative abundance of the set of bacterial species in the reference data set; and (b2) running the relative abundance of the set of bacterial species from the individual along the ensemble of decision trees to generate the risk score.

Embodiment 47. The computer program product of embodiment 45 or claim 46, wherein the optimal candidate model is the candidate model with the highest performance among the set of candidate models.

Embodiment 48. The computer program product of any one of embodiments 45-47, wherein the performance of the candidate model is evaluated by Area Under the Curve (AUC) in Receiving Operating Characteristic (ROC) curve of the candidate model.

Embodiment 49. The computer program product of any one of embodiments 41-48, wherein the cutoff value is in the range of 0.1-0.3.

Embodiment 50. The computer program product of any one of embodiments 41-49, wherein the cutoff value is determined by the following steps: (c1) calculating a Youden's index using the risk score data in the reference data set; and (c2) determining the cutoff value based on the Youden's index.

Embodiment 51. The computer program product of any one of embodiments 41-50, wherein the set of bacterial species consisting essentially of Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, Allisonella histaminiformans, Romboutsia timonensis, Faecalibacterium prausnitzii, and Haemophilus parainfluenzae.

Embodiment 52. The computer program product of any one of embodiments 41-50, wherein the set of bacterial species comprises Faecalimonas umbilicate, Akkermansia muciniphila, Enterocloster clostridioformis, Evtepia gabavorous, Ruminococcus gnavus, Enterocloster bolteae, Enterocloster aldenensis, Erysipelatoclostridium ramosum, Escherichia coli, Fusobacterium ulcerans, Enterocloster citroniae, Anaerotruncus colihominis, Clostridium SGB4750, Phascolarctobacterium faecium, Ruminococcus torques, Fusobacterium mortiferum, Clostridium fessum, Bilophila wadsworthia, Fusobacterium nucleatum, Klebsiella pneumoniae, Veillonella parvula, Phascolarctobacterium SGB4573, Allisonella histaminiformans, and Lachnospiraceae bacterium NSJ-29.

Embodiment 53. The computer program product of any one of embodiments 41-50, wherein the set of bacterial species comprises Faecalimonas umbilicate, Akkermansia muciniphila, Enterocloster clostridioformis, Evtepia gabavorous, Ruminococcus gnavus, Enterocloster bolteae, Enterocloster aldenensis, Escherichia coli, Anaerotruncus colihominis, Clostridium SGB4750, Ruminococcus torques, and Allisonella histaminiformans.

Embodiment 54. The computer program product of any one of embodiments 41-50, wherein the set of bacterial species comprises Romboutsia timonensis, Faecalibacterium prausnitzii, Flavonifractor plautii, Faecalibacterium SGB15346, Dorea formicigenerans, Anaerobutyricum hallii, Haemophilus parainfluenzae and Streptococcus australis.

What is claimed is:

1. A method for determining the risk of, diagnosing, preventing, or treating disease above the terminal ileum (TI) in an individual suffering from Inflammatory Bowel Disease (IBD), comprising the steps of:
a) determining the relative abundance of a set of bacterial species in a stool sample from the individual, wherein the set of bacterial species comprises three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Law sonibacter asaccharolyticus, Turicibacter bilis, Anaerostipes hadrus, Faecalibacterium prausnitzii, and Roseburia intestinalis;
b) comparing the relative abundance of each bacterial species in the set of bacterial species from the individual with the relative abundance of each bacterial species in the set of bacterial species in a reference data set to generate a risk score;
c) determining the individual as having increased risk for or is suffering from disease above the TI if the risk score is higher than a cutoff value, or determining the individual as not having increased risk for or is not suffering from disease above the TI if the risk score is equal to or lower than the cutoff value; and
d) if the individual is determined to have an increased risk for or is suffering from disease above the TI, optionally treating the individual with an appropriate systemic therapy for disease above the TI, wherein the appropriate systemic therapy is selected from the group consisting of systemic corticosteroids, thiopurines, parenteral methotrexate, non-JAK inhibitor biologic agents, oral small molecule therapeutic agents for treating disease above the TI, and any combination thereof, or
if the individual is determined to not have an increased risk for or is not suffering from disease above the TI, optionally treating the individual with an appropriate localized therapy, wherein the appropriate localized therapy is selected from the group consisting of sulfasalazine, enteric release budesonide, controlled ileal release (CIR) budesonide, oral methotrexate, an oral Janus kinase (JAK) inhibitor, or any combination thereof.

2. The method of claim 1, wherein the set of bacterial species comprises a group of three bacterial species selected from Table 3, a group of four bacterial species selected from Table 5, or a group of five bacterial species selected from Table 7.

3. The method of claim 1, wherein the reference data set is obtained by determining relative abundances of each bacterial species in the set of bacterial species in stool samples from a group of subjects comprising patients with Crohn's Disease (CD) in a reference cohort, wherein the patients with CD comprise patients with disease above the TI and patients without the disease above TI.

4. The method of claim 1, wherein the comparing step in step (b) is performed using a machine learning model to generate the risk score.

5. The method of claim 4, wherein the machine learning model is constructed by
i) obtaining the reference data set by determining relative abundances of each bacterial species in the set of bacterial species in stool samples from a group of subjects comprising patients with CD in a reference cohort, wherein the patients with CD comprise patients with disease above the TI and patients without disease above the TI;
ii) obtaining a validation data set by determining relative abundances of each bacterial species in the set of bacterial species in stool samples from a second group of subjects comprising patients with CD and patients with Ulcerative Colitis (UC) in a validation cohort, wherein the patients with CD comprise patients with disease above the TI and patients without disease above the TI;
iii) dividing the reference data set into a training data set and a test data set;
iv) training a candidate model with the training data set and evaluating the performance of the candidate model using the validation data set;
v) optionally tuning the candidate model by adjusting hyperparameters in the candidate model;
vi) repeating step (iv) and optionally step (v) to produce a set of candidate models, and selecting an optimal candidate model among the set of candidate models;
vii) evaluating the performance of the optimal candidate model using the test data set;
viii) optionally training a final model with the reference data set using a set of hyperparameters corresponding to the optimal candidate model to produce the final model; and
ix) using the optimal candidate model of step (vii) or if step (viii) is used to train the final model, using the final model of step (viii) as the machine learning model for the comparing step in step (b) to generate the risk score.

6. The method of claim 4, wherein the machine learning model is a random forest model, and wherein the risk score in step (b) is generated by the following steps:
(b1) generating an ensemble of decision trees by the random forest model using the relative abundance of the set of bacterial species in the reference data set; and
(b2) running the relative abundance of the set of bacterial species from the individual along the ensemble of decision trees to generate the risk score.

7. The method of claim 5, wherein the optimal candidate model is the candidate model with the highest performance among the set of candidate models.

8. The method of claim 5, wherein the performance of the candidate model is evaluated by Area Under the Curve (AUC) in Receiving Operating Characteristic (ROC) curve of the candidate model.

9. The method of claim 1, wherein the cutoff value is in the range of 0.3 to 0.6.

10. The method of claim 1, wherein the cutoff value is determined by the following steps:
(c1) calculating a Youden's index using the risk score data in the reference data set; and
(c2) determining the cutoff value based on the Youden's index.

11. The method of claim 1, wherein the set of bacterial species consists essentially of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

12. The method of claim 1, wherein the set of bacterial species comprises Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

13. The method of claim 1, wherein the oral JAK inhibitor is upadacitinib.

14. A kit for determining the risk of or diagnosing disease above terminal ileum (TI) in an individual suffering from Inflammatory Bowel Disease (IBD), comprising a reagent for detecting a set of bacterial species, wherein the set of bacterial species comprises three or more bacterial species selected from the group consisting of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Law sonibacter asaccharolyticus, Turicibacter bilis, Anaerostipes hadrus, Faecalibacterium prausnitzii, and Roseburia intestinalis.

15. The kit of claim 14, wherein the set of bacterial species comprises a group of three bacterial species selected from Table 3, a group of four bacterial species selected from Table 5, or a group of five bacterial species selected from Table 7.

16. The kit of claim 14, wherein the reagent comprises one or more sets of primers and optionally one or more fluorophore-quencher probes, wherein each set of primers is used for amplifying a target nucleic acid sequence belonging to a bacterial species in the set of bacterial species via PCR to produce an amplification product, and each fluorophore-quencher probe is used for detecting the amplification product of each set of primers.

17. The kit of claim 14, wherein the set of bacterial species consisting essentially of Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

18. The kit of claim 14, wherein the set of bacterial species comprises Escherichia coli, Phascolarctobacterium SGB4573, Olsenella sp. AF16_14LB, Megamonas funiformis, Megasphaera micronuciformis, Eggerthella sinensis, Megasphaera sp. NM10, Klebsiella quasipneumoniae, Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

19. A composition comprising Streptococcus gordonii, Romboutsia timonensis, Dorea sp. AF36_15AT, Flavonifractor plautii, Eggerthella lenta, Lawsonibacter asaccharolyticus, Turicibacter bilis, Anaerostipes hadrus, Faecalibacterium prausnitzii, and Roseburia intestinalis.

20. A method of preventing or treating disease above terminal ileum (TI) in an individual suffering from Inflammatory Bowel Disease (IBD), comprising the step of:
administering the composition of claim 19 to the individual.
